(19) United States
(12) Patent Application Publication
(10) Pub. No.: US 2026/0226137 A1
(43) Pub. Date: Aug. 6, 2026

(54) SOLUBLE BONE MORPHOGENETIC PROTEIN (BMP) RECEPTOR TYPE-1B PROTEINS AND USES FOR THE TREATMENT OF RETINOPATHIES

(71) Applicant: Lask Pharma, Inc., Waltham, MA (US)

(72) Inventor: John Knopf, Waltham, MA (US)

(21) Appl. No.: 19/529,027

(22) Filed: Feb. 3, 2026

Related U.S. Application Data
(60) Provisional application No. 63/753,279, filed on Feb. 3, 2025.
(60) Provisional application No. 63/757,653, filed on Feb. 12, 2025.

(51) Int. Cl. C07K 14/71; A61K 38/00; A61P 27/02

(52) U.S. Cl. CPC C07K 14/71; A61P 27/02; A61K 38/00; C07K 2319/30

(57) ABSTRACT

This application relates generally to the field of Bone Morphogenetic Protein (BMP) antagonists (soluble ALK6 fusion proteins), compositions thereof, and methods for use in treating retinopathy diseases and/or ocular neo-vascular diseases including age related macular degeneration.

### RELATED APPLICATIONS

This application claims priority to U.S. Provisional Application Ser. No. 63/753,279 filed on 3 Feb. 2025 and U.S. Provisional Application Ser. No. 63/757,653 filed on 12 Feb. 2025, each of which is incorporated by reference into this application in its entirely.

### SEQUENCE LISTING

The instant application contains a sequence listing which has been submitted electronically in XML format via the USPTO Patent Center and hereby incorporated by reference in its entirety. Said XML copy, created on 30 Jan. 2026, is named 1902004-0002-005-101_SL.xml and is 659,456 bytes in size.

### FIELD OF THE DISCLOSURE

This application relates generally to the field of Bone Morphogenetic Protein (BMP) antagonists, compositions thereof, and methods for use in treating diseases related to BMPs.

### BACKGROUND OF THE DISCLOSURE

Blinding eye disease remains a major global health challenge in the 21st century. An estimated 43 million people worldwide were blind in 2020, and another 295 million people had moderate to severe vision impairment1. The economic burden of vision loss in the United States was estimated to be over US$134 billion in 2021. The leading causes of irreversible blindness are neurodegenerative, including diseases caused by death of photoreceptors and associated retinal pigment epithelium (RPE)—such as age-related macular degeneration (AMD) and retinitis pigmentosa (RP), the most common form of inherited retinal disorder—and death of retinal ganglion cells (RGCs), the neurons of the optic nerve, in a condition known as glaucoma. Reversal of vision loss remains an elusive goal.

Diabetic retinopathy (DR), diabetic macular edema (DME), and age related macular degeneration (AMD), such as age-related macular degeneration (AMD) are eye conditions that impact vision and involve neo-vascularization. The most severe form of diabetic retinopathy is proliferative retinopathy which can also lead to vision loss. Both DR and AMD are caused in part by blood vessel damage and/or undesirable proliferation.

Macular degeneration is caused in part by blood vessels that grow under the retina. Retinopathy is caused by damage to the blood vessels of the light-sensitive tissue at the back of the eye (retina). Early diabetic retinopathy, also referred to as non-proliferative diabetic retinopathy (NPDR) is the most common form of diabetic retinopathy. In non-proliferative diabetic retinopathy (NPDR), the walls of the blood vessels in the retina weaken. Tiny bulges protrude from the walls of the smaller vessels, sometimes leaking fluid and blood into the retina. Larger retinal vessels can begin to dilate and become irregular in diameter as well. NPDR can progress from mild to severe as more blood vessels become blocked.

Diabetic retinopathy can progress to proliferative diabetic retinopathy. In this type, damaged blood vessels close off, causing the growth of new, abnormal blood vessels in the retina. These new blood vessels are fragile and can leak into the clear, jellylike substance that fills the center of your eye (vitreous). Eventually, scar tissue from the growth of new blood vessels can cause the retina to detach from the back of your eye. If the new blood vessels interfere with the normal flow of fluid out of the eye, pressure can build in the eye. This buildup can damage the nerve that carries images from your eye to your brain (optic nerve), resulting in glaucoma.

Current standard of care for retinopathies today are intravitreal injections of anti-VEGF drugs to control neovascularization and reduce the area of fluid below the retinal pigment epithelium. These drugs are commonly known as AVASTIN® (Bevacizumab) (mostly used off label), LUCENTIS® (Ranibizumab), MACUGEN® (Pegaptanib) and EYLEA® (aflibercept) and although their effectiveness has been shown to significantly improve visual prognosis with choroidal neovascularization (CNV), the recurrence rate for these neovascular areas remains high. LUCENTIS® is a Fab, AVASTIN® is a monoclonal antibody, MACUGEN® is a pegylated RNA aptamer directed against the VEGF-A 165 isoform and EYLEA® is a VEGF Trap fused to an Fc domain of human IgG1. All bind vascular endothelial growth factor (VEGF) and may be used to treat neo-vascularization; however, only a minority of treated patients experiences a significant improvement in prolonged visual acuity.

The transforming growth factor-beta (TGF-beta) superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, osteogenesis, chondrogenesis, cardiogenesis, hematopoiesis, neurogenesis, and epithelial cell differentiation. The family is divided into two general phylogenetic clades: the more recently evolved members of the superfamily, which includes TGF-betas, Activins, and nodal and the clade of more distantly related proteins of the superfamily, which includes a number of BMPs and GDFs. Hinck (2012) FEBS Letters 586:1860-1870. TGF-beta family members have diverse, often complementary biological effects. By manipulating the activity of a member of the TGF-beta family, it is often possible to cause significant physiological changes in an organism. For example, the Piedmontese and Belgian Blue cattle breeds carry a loss-of-function mutation in the GDF8 (also called myostatin) gene that causes a marked increase in muscle mass. Grobet et al. (1997) Nat Genet., 17(1):71-4. Furthermore, in humans, inactive alleles of GDF8 are associated with increased muscle mass and, reportedly, exceptional strength. Schuelke et al. (2004) N Engl J Med, 350:2682-8. Changes in muscle, bone, fat, red blood cells, and other tissues may be achieved by enhancing or inhibiting signaling (e.g., SMAD 1, 2, 3, 5, and/or 8) that is mediated by ligands of the TGF-beta family. Thus, there is a need for agents that regulate the activity of various ligands of the TGF-beta superfamily, including the bone morphogenetic proteins (BMPs) related to this disclosure.

### SUMMARY OF THE DISCLOSURE

Described herein are polypeptides, compositions, other reagents related to Bone Morphogenetic Protein (BMP) antagonists, and methods for use in treating retinopathy and/or ocular neo-vascular disorders related to BMP ligand and endogenous signaling. In particular, this disclosure relates to soluble bone morphogenetic protein (BMP) receptor type 1B polypeptides comprising an extracellular domain of ALK6 (activin receptor-like kinase 6) and soluble variants thereof (“ALK6 polypeptides”), which bind BMP ligands and sequester the ligands from binding to their receptors (“BMP antagonists”). The ALK-6 polypeptides of this disclosure bind BMP ligands, including, but not limited to BMP 4, 6, 7 and/or 10. In certain embodiments, ALK-6 polypeptides of this disclosure bind BMP10. In certain other embodiments, the ALK-6 polypeptides of this disclosure do not bind BMP10. In certain embodiments, the ALK6 ECD sequence may comprise one or more point mutations, addition or deletion of c-terminal residues, wherein position 60 is glycine (G) as provided in SEQ ID NO:1.

In embodiments, proved herein are methods for treatment of retinopathy in a human subject in need thereof comprising administering an effective amount of a pharmaceutical composition to a subject in need thereof, wherein the composition comprises a soluble recombinant bone morphogenetic protein receptor type-1B fusion protein comprising an ALK6 ECD polypeptide and a Fc sequence, wherein the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 3; SEQ ID NO: 5; SEQ ID NO: 79, SEQ ID NO: 81, SEQ ID NO: 82, SEQ ID NO: 83, SEQ ID NO: 108; SEQ ID NO: 109; SEQ ID NO: 130, SEQ ID NO: 157, SEQ ID NO: 190 and SEQ ID NO: 437.

In certain embodiments provided herein are methods for treatment of retinopathy in a human subject in need thereof, comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102, 104, 110, 113, 132-156, 158-189, 191-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, or 431.

In certain embodiments provided herein are methods for treatment of retinopathy in a human subject in need thereof, comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 3, 5, 79, 81, 82, 83, 108, 109, 112, 130, 157 190, 202, 213, 218, 223, 228, 233, 242, 247, 252, 257, 262, 272, 277 and 283.

In certain embodiments provided herein are method for treatment of retinopathy in a human subject in need thereof, comprising: administering an effective amount of a pharmaceutical composition to the subject, wherein the composition comprises a soluble recombinant bone morphogenetic protein receptor type-1B fusion protein comprising an ALK6 ECD polypeptide, a linker and a heterologous sequence, wherein the ALK6 Fc fusion protein comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 291, 297, 303, 309, 315, 321, 327, 333, 339, 345, 351, 357, 363, 369, 375, 381, 387, 393, 399, 405, 411, 417, 423, 424, 430, and 436.

In certain embodiments, the fusion protein polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 fusion protein sequence selected from SEQ ID NO: 291.

In embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 69; or the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 90, or the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 286.

In embodiments, the retinopathy is selected from any one of diabetic retinopathy, age-related macular degeneration (AMD), choroidal neovascularization (CNV), vision loss, night blindness, photopsia, proliferative retinopathy, non-proliferative retinopathy, and hypertensive retinopathy.

In certain embodiments provided herein are soluble recombinant bone morphogenetic protein receptor type-1B fusion proteins comprising: an ALK6 extra cellular domain (ECD), a linker sequence and an Fc domain sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 46-50, 52-56, 58, 60, 62, 64, 66, 68, 70, 72, 74, 76, 78, 86, 88, 90, 92, 94, 96, 98, 100, 102, 104, 110 and 113, wherein amino acid position 60 is a glycine residue. In embodiments, the polypeptide binds BMP10. In embodiments, the ALK6 ECD sequence selected from SEQ ID NO: 86, 90, 92, 94, 96, 98, 100, and 102. In certain embodiments, the ALK6 ECD comprises one or more point mutations selected from K20Y, K20F, K20V, H22S, H22G, H23S, and H23G. In further embodiments, the ALK6 ECD comprises an amino acid insertion between position 22 and position 23 of an alanine (A) residue.

Provided herein are soluble recombinant bone morphogenetic protein receptor type-1B fusion proteins comprising: an ALK6 extra cellular domain (ECD), a linker sequence and an Fc domain sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 86 or 90, wherein amino acid position 60 is a glycine (G) residue. In embodiments, the ALK6 ECD sequence comprises at least one point mutation as compared to SEQ ID NO: 1. In embodiments, the point mutation is selected from K20Y, K20F, K20V, H22S, H22G, H23S, and H23G. In embodiments, the polypeptide binds BMP10.

Provided herein are pharmaceutical composition comprising an ALK6 fusion protein of this disclosure; and, at least one pharmaceutical acceptable carrier or buffer.

In certain embodiments provided herein are methods for treating retinopathy disorders, comprising administering an effective amount of a pharmaceutical composition comprising an ALK6 fusion protein of this disclosure to a subject in need thereof.

In embodiments provided herein, are methods for treatment of retinopathy in a human subject in need thereof.

In certain embodiments, the disclosure provides a method for treatment of retinopathy in a human subject in need thereof comprising: administering an effective amount of a pharmaceutical composition to the subject, wherein the composition comprises a soluble recombinant bone morphogenetic protein receptor type-1B fusion protein comprising an ALK6 ECD polypeptide and a heterologous sequence, wherein the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 3; SEQ ID NO: 5; SEQ ID NO: 79, SEQ ID NO: 81, SEQ ID NO: 82, SEQ ID NO: 83, SEQ ID NO: 108; SEQ ID NO: 109; SEQ ID NO: 130, SEQ ID NO: 157, SEQ ID NO: 190 and SEQ ID NO: 437.

In certain embodiments, the disclosure provides method for treatment of retinopathy in a human subject in need thereof, comprising: administering an effective amount of a pharmaceutical composition to the subject, wherein the composition comprises a soluble recombinant bone morphogenetic protein receptor type-1B fusion protein comprising an ALK6 ECD polypeptide, a linker and a heterologous sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102, 104, 110, 113, 132-156, 158-189, 191-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, or 431.

In certain embodiments, the disclosure provides method for treatment of retinopathy in a human subject in need thereof, comprising: administering an effective amount of a pharmaceutical composition to the subject, wherein the composition comprises a soluble recombinant bone morphogenetic protein receptor type-1B fusion protein comprising an ALK6 ECD polypeptide, a linker and a heterologous sequence, wherein the ALK6 Fc fusion protein comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 291, 297, 303, 309, 315, 321, 327, 333, 339, 345, 351, 357, 363, 369, 375, 381, 387, 393, 399, 405, 411, 417, 423, 424, 430, and 436.

In some embodiments, the fusion protein polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 fusion protein sequence selected from SEQ ID NO: 291. In some embodiments, the ALK6 ECD sequence comprises at least one point mutation as compared to SEQ ID NO: 1. In some embodiments, the soluble recombinant bone morphogenetic protein receptor type-1B fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 86, SEQ ID NO: 88, SEQ ID NO: 90 or SEQ ID NO: 286. In some embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 69. In some embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 90. In some embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 286. In some embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 233 or SEQ ID NO:242.

In some embodiments, the point mutation is selected from one or more of V16I; K20Y, K20F, H22S, _22′A, H23G, V30I, I33T, I33S, S35I, S49Q and L51M. In some embodiments, the polypeptide binds one or more of BMP6, BMP7 and/or BMP10. In some embodiments, the ALK6 ECD sequence is selected from SEQ ID NO: 190 and the sequence comprises point mutations K20Y and H22S. In some embodiments, the ALK6 ECD sequence is selected from SEQ ID NO: 190 and the sequence comprises point mutations K20Y, H22S and I33T. In some embodiments, the ALK6 ECD sequence is selected from SEQ ID NO: 190 and the sequence comprises point mutations H22S and I33T.

In some embodiments, the heterologous region is an Fc region. In some embodiments, the heterologous region is a hinge region. In some embodiments, the heterologous sequence is an Fc sequence according to SEQ ID NO: 285.

In some embodiments, the retinopathy is selected from diabetic retinopathy, age-related macular degeneration (AMD), choroidal neovascularization (CNV), vision loss, night blindness, photopsia, proliferative retinopathy, non-proliferative retinopathy, retinitis pigmentosa and hypertensive retinopathy.

In certain embodiments, the disclosure provides a method for treatment of age-related macular degeneration (AMD) in a human subject in need thereof comprising: administering an effective amount of a pharmaceutical composition to the subject, wherein the composition comprises a soluble recombinant bone morphogenetic protein receptor type-1B fusion protein comprising an ALK6 ECD polypeptide and a heterologous sequence, wherein the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 3; SEQ ID NO: 5; SEQ ID NO: 79, SEQ ID NO: 81, SEQ ID NO: 82, SEQ ID NO: 83, SEQ ID NO: 108; SEQ ID NO: 109; SEQ ID NO: 130, SEQ ID NO: 157, SEQ ID NO: 190 and SEQ ID NO: 437.

In certain embodiments, the disclosure provides, a method for treatment of age-related macular degeneration (AMD) in a human subject in need thereof, comprising: administering an effective amount of a pharmaceutical composition to the subject, wherein the composition comprises a soluble recombinant bone morphogenetic protein receptor type-1B fusion protein comprising an ALK6 ECD polypeptide, a linker and a heterologous sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102, 104, 110, 113, 132-156, 158-189, 191-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, or 431.

In certain embodiments, the disclosure provides a method for treatment of age-related macular degeneration (AMD) in a human subject in need thereof, comprising: administering an effective amount of a pharmaceutical composition to the subject, wherein the composition comprises a soluble recombinant bone morphogenetic protein receptor type-1B fusion protein comprising an ALK6 ECD polypeptide, a linker and a heterologous sequence, wherein the ALK6 Fc fusion protein comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 291, 297, 303, 309, 315, 321, 327, 333, 339, 345, 351, 357, 363, 369, 375, 381, 387, 393, 399, 405, 411, 417, 423, 424, 430, and 436.

In some embodiments, the fusion protein polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 fusion protein sequence selected from SEQ ID NO: 291. In some embodiments, the ALK6 ECD sequence comprises at least one point mutation as compared to SEQ ID NO: 1. In some embodiments, the soluble recombinant bone morphogenetic protein receptor type-1B fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence of SEQ ID NO: 86, SEQ ID NO: 88, SEQ ID NO: 90 or SEQ ID NO: 286. In some embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 69. In some embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 90. In some embodiments, the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 286.

In embodiments, provided herein are uses of a soluble recombinant bone morphogenetic protein receptor type-1B fusion protein for treatment of retinopathy in a human subject in need thereof.

In certain embodiments, the disclosure provides use of a soluble recombinant bone morphogenetic protein receptor type-1B fusion protein for treatment of retinopathy in a human subject in need thereof, comprising administering an effective amount of the fusion protein, comprising: an ALK6 extra cellular domain (ECD); a linker sequence; and, an Fc domain sequence; wherein the ALK6 ECD sequence comprises or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 286, wherein the position corresponding to position 60 of SEQ ID NO: 69 is a glycine (G) residue, and wherein the ALK6 fusion protein demonstrates retained BMP 10 binding.

In certain embodiments, the disclosure provides use of a soluble recombinant bone morphogenetic protein receptor type-1B fusion protein for treatment of retinopathy in a human subject in need thereof, comprising administering an effective amount of the fusion protein, comprising: an ALK6 extra cellular domain (ECD); a linker sequence; and an Fc domain sequence; wherein the ALK6 Fc fusion protein sequence comprises or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 Fc fusion sequence of SEQ ID NO: 291, wherein the position corresponding to position 60 of SEQ ID NO: 69 is a glycine (G) residue, and wherein the ALK6 fusion protein demonstrates retained BMP10 binding.

In certain embodiments, the disclosure provides use of a soluble recombinant bone morphogenetic protein receptor type-1B fusion protein for treatment of retinopathy in a human subject in need thereof, comprising administering an effective amount of the fusion protein, comprising: an ALK6 extra cellular domain (ECD); a linker sequence; and an Fc domain sequence; wherein the ALK6 ECD sequence comprises or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 437, wherein the position corresponding to position 60 of SEQ ID NO: 69 is a glycine (G) residue, and wherein the ALK6 fusion protein demonstrates complete BMP10 binding.

In some embodiments, the ALK6 fusion protein demonstrates BMP10 binding comparable to SEQ ID NO: 49. In some embodiments, the ALK6 fusion protein demonstrates BMP10 binding comparable to binding of an ALK6 wildtype (wt) Fc fusion protein. In some embodiments, the ALK6 fusion protein demonstrates an nM IC₅₀ value of about 7.2 for BMP10 binding. In some embodiments, the ALK6 fusion protein demonstrates an nM IC₅₀ value of about 4.8 for BMP7 binding. In some embodiments, the retinopathy is age related macular degeneration (AMD).

In embodiments, provided herein are soluble recombinant bone morphogenetic protein receptor type-1B fusion proteins comprising an ALK6 ECD polypeptide and a Fc sequence.

In certain embodiments, the disclosure provides a soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 130: Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Lys₁₅-Xaa₁₆-Leu₁₇-Arg₁₈-Cys₁₉-Xaa₂₀-Cys₂₁-Xaa₂₂-Xaa′₂₂-Xaa″₂₂-Xaa₂₃-Xaa′₂₃-Xaa₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Xaa₁₀₈-Xaa₁₀₉-Xaa₁₁₀-Xaa₁₁₁-Xaa₁₁₂-Xaa₁₁₃; wherein Xaa₁₆ is V or I; Xaa₂₀ is V, F, K, Y, T, or A; Xaa₂₂ is G, E, H or S; Xaa′₂₂ is absent, G, S, or A; Xaa″₂₂ is absent or S; Xaa₂₃ is S, H, G, or L; Xaa₂₃′ is absent, L or H; Xaa₂₄ is H or S; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M; Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is N or L; Xaa₈₈ is absent, E or L; Xaa₉₁ is K or Q; Xaa₉₂ is D or L; Xaa₉₄ is H or Q; Xaa₁₀₇ is absent; Xaa₁₀₈ is absent; Xaa₁₀₉ is absent; Xaa₁₁₀ is absent; Xaa₁₁₁ is absent; Xaa₁₁₂ is absent; and, Xaa₁₁₃ is absent.

In some embodiments Xaa60 is a glycine (G) residue. In some embodiments, the polypeptide binds BMP10. In some embodiments, the fusion protein further comprises a linker sequence. In some embodiments, the fusion protein further comprises a linker sequence selected from SEQ ID NO: 6, SEQ ID NO: 7 or SEQ ID NO: 8. In some embodiments, the Fc sequence comprises an amino acid sequence according to SEQ ID NO: 285.

In certain embodiments, the disclosure provides a soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 157: Xaa₁-Xaa₂-Xaa₃-Xaa₄-Xaa₅-Xaa₆-Xaa₇-Xaa₈-Xaa₉-Xaa₁₀-Xaa₁₁-Xaa₁₂-Xaa₁₃-Xaa₁₄-Xaa₁₅-Xaa₁₆-Leu₁₇-Arg₁₈-Cys₁₉-Xaa₂₀-Cys₂₁-Xaa₂₂-Xaa′₂₂-Xaa″₂₂-Xaa₂₃-Xaa′₂₃-Xaa₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Xaa₁₀₀-Xaa₁₀₁-Xaa₁₀₂-Xaa₁₀₃-Xaa₁₀₄-Xaa₁₀₅-Xaa₁₀₆-Xaa₁₀₇-Xaa₁₀₈-Xaa₁₀₉-Xaa₁₁₀-Xaa₁₁₁-Xaa₁₁₂-Xaa₁₁₃; wherein: Xaa₁ is K or absent; Xaa₂ is K or absent; Xaa₃ is E or absent; Xaa₄ is D or absent; Xaa₅ is G or absent; Xaa₆ is E or absent; Xaa₇ is S or absent; Xaa₈ is T or absent; Xaa₉ is A or absent; Xaa₁₀ is P or absent; Xaa₁₁ is T or absent; Xaa₁₂ is P or absent; Xaa₁₃ is R or absent; Xaa₁₄ is P or absent; Xaa₁₅ is K, P or absent; Xaa₁₆ is V or absence; Xaa₂₀ is V, F, K, A, R, N, D, C, E, Q, G, H, I, L, M, P, S, T, W, or Y; Xaa₂₂ is G, H, V, F, K, A, R, N, D, C, E, Q, I, L, M, P, T, W, Y or S; Xaa′₂₂ is absent, G, S, or A; Xaa″₂₂ is absent or S; Xaa₂₃ is S, H, V, F, K, A, R, N, D, C, E, Q, I, L, M, P, T, W, Y or G; Xaa′₂₃ is absent, L or H; Xaa₂₄ is V, F, K, A, R, N, D, C, E, Q, G, H, I, L, M, P, S, T, W, or Y; Xaa₂₇ is E or D; Xaa₂₉ is S, G or A; Xaa₃₀ is V, A, G or I; Xaa₃₃ is I, S or T; Xaa₃₅ is S, T, G, A or I; Xaa₃₇ is D, A, G or N; Xaa₃₉ is Y, A, G or H; Xaa₄₂ is T or A; Xaa₄₃ is M or I; Xaa₄₉ is S, A, G or Q; Xaa₅₁ is L, A, G or E; Xaa₅₂ is P or T; Xaa₅₃ is V, A, G or T; Xaa₅₄ is V, A, G or L; Xaa₅₅ is T, G or A; Xaa₅₉ is L, M, V, F, K, A, R, N, D, C, E, Q, G, H, I, P, S, T, W, or Y; Xaa₆₀ is G, K, V, F, A, R, N, D, C, E, Q, H, I, L, M, P, S, T, W, or Y; Xaa₆₁ is L, Y, V, F, K, A, R, N, D, C, E, Q, G, H, I, M, P, S, T, or W; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T, R or absent; Xaa₈₅ is absent, T or E; Xaa₈₆ is R, N or absent; Xaa₈₇ is N, L or absent; Xaa₈₈ is absent, E or L; Xaa₉₁ is K, Q or absent; Xaa₉₂ is D, L, Y or absent; Xaa₉₄ is H, Q or absent; Xaa₁₀₀ is absent, V or L; Xaa₁₀₁ is absent, V or K; Xaa₁₀₂ is absent, I or N; Xaa₁₀₃ is absent, G or R; Xaa₁₀₄ is absent, P, D or A; Xaa₁₀₅ is absent or F; Xaa₁₀₆ is absent, F, W, Y, N, Q, K, H, D, G, E or V; Xaa₁₀₇ is absent, L, M, V, F, K, A, R, N, C, E, Q, G, H, I, P, S, T, W, or Y or D; Xaa₁₀₈ is absent, R, T, Q, L, K, W or G; Xaa₁₀₉ is absent, S or P; Xaa₁₁₀ is absent or I; Xaa₁₁₁ is absent, R or H; Xaa₁₁₂ is absent or H; and Xaa₁₁₃ is absent or R.

In some embodiments, Xaa60 is a glycine (G) residue. In some embodiments, the polypeptide binds BMP10. In some embodiments, the fusion protein further comprises a linker sequence. In some embodiments, the fusion protein further comprises a linker sequence selected from SEQ ID NO: 6, SEQ ID NO: 7 or SEQ ID NO: 8. In some embodiments, the Fc sequence comprises an amino acid sequence according to SEQ ID NO: 285. In some embodiments, the ALK6 ECD sequence an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence selected from any one of SEQ ID NO: 132 to 155.

In certain embodiments, the disclosure provides a soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 190: Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Xaa₁₆-Leu₁₇-Arg₁₈-Cys₁₉-Xaa₂₀-Cys₂₁-Xaa₂₂-Xaa′₂₂-Xaa″₂₂-Xaa₂₃-Xaa′₂₃-Xaa₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-Pro₉₈-Pro₉₉-Leu₁₀₀-Lys₁₀₁-Asn₁₀₂-Arg₁₀₃-Xaa₁₀₄-Phe₁₀₅-Val₁₀₆-Xaa₁₀₇-Xaa₁₀₈-Xaa₁₀₉-Xaa₁₁₀-Xaa₁₁₁-Xaa₁₁₂-Xaa₁₁₃; wherein Xaa₁₅ is K or P; Xaa₁₆ is V or I; Xaa₂₀ is V, F, K, A, R, N, D, C, E, Q, G, H, I, L, M, P, S, T, W, or Y; Xaa₂₂ is G, H, V, F, K, A, R, N, D, C, E, Q, I, L, M, P, T, W, Y or S; Xaa′₂₂ is absent, G, S, or A; Xaa″₂₂ is absent or S; Xaa₂₃ is S, H, V, F, K, A, R, N, D, C, E, Q, I, L, M, P, T, W, Y or G; Xaa′₂₃ is absent, L or H; Xaa₂₄ is V, F, K, A, R, N, D, C, E, Q, G, H, I, L, M, P, S, T, W, or Y; Xaa₂₇ is E or D; Xaa₂₉ is S, G or A; Xaa₃₀ is V, A, G or I; Xaa₃₃ is I, S or T; Xaa₃₅ is S, T, G, A or I; Xaa₃₇ is D, A, G or N; Xaa₃₉ is Y, A, G or H; Xaa₄₂ is T or A; Xaa₄₃ is M or I; Xaa₄₉ is S, A, G or Q; Xaa₅₁ is L, A, G or E; Xaa₅₂ is P or T; Xaa₅₃ is V, A, G or T; Xaa₅₄ is V, A, G or L; Xaa₅₅ is T, G or A; Xaa₅₉ is L, M, V, F, K, A, R, N, D, C, E, Q, G, H, I, P, S, T, W, or Y; Xaa₆₀ is G, K, V, F, A, R, N, D, C, E, Q, H, I, L, M, P, S, T, W, or Y; Xaa₆₁ is L, Y, V, F, K, A, R, N, D, C, E, Q, G, H, I, M, P, S, T, or W; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is absent, T or R; Xaa₈₅ is absent, T or E; Xaa₈₆ is absent, R or N; Xaa₈₇ is N or L; Xaa₈₈ is absent, E or L; Xaa₉₁ is absent, K or Q; Xaa₉₂ is absent, D, Y or L; Xaa₉₄ is absent, H or Q; Xaa₁₀₄ is D or A; Xaa₁₀₇ is absent; Xaa₁₀₈ is absent; Xaa₁₀₉ is absent; Xaa₁₁₀ is absent; Xaa₁₁ is absent; Xaa₁₁₂ is absent; and, Xaa₁₁₃ is absent.

In some embodiments, Xaa60 is a glycine (G) residue. In some embodiments, the polypeptide binds BMP10. In some embodiments, the fusion protein further comprises a linker sequence. In some embodiments, the fusion protein further comprises a linker sequence selected from SEQ ID NO: 6, SEQ ID NO: 7 or SEQ ID NO: 8. In some embodiments, the Fc sequence comprises an amino acid sequence according to SEQ ID NO: 285. In some embodiments, the ALK6 ECD sequence an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence selected from any one of SEQ ID NO: 158 to 189.

In certain embodiments, the disclosure provides a soluble recombinant bone morphogenetic protein receptor type-1B fusion protein comprising an ALK6 ECD polypeptide and a heterologous sequence, wherein the ALK6 ECD polypeptide comprises an amino acid sequence selected from a sequence of SEQ ID NO: 437: Lys₁-Lys₂-Glu₃-Asp₄-Gly₅-Glu₆-Ser₇-Thr₈-Ala₉-Pro₁₀-Thr₁₁-Pro₁₂-Arg₁₃-Pro₁₄-Xaa₁₅-Xaa₁₆-Leu₁₇-Arg₁₈-Cys₁₉-Xaa₂₀-Cys₂₁-Xaa₂₂-Xaa₂₃-His₂₄-Cys₂₅-Pro₂₆-Xaa₂₇-Asp₂₈-Xaa₂₉-Xaa₃₀-Asn₃₁-Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-Xaa₈₇-Xaa₈₈-Xaa₈₉-Xaa₉₀-Xaa₉₁-Xaa₉₂-Xaa₉₃-Xaa₉₄-Xaa₉₅-Xaa₉₆-Xaa₉₇-Xaa₉₈-Xaa₉₉-Xaa₁₀₀-Xaa₁₀₁-Xaa₁₀₂-Xaa₁₀₃-Xaa₁₀₄-Xaa₁₀₅-Xaa₁₀₆-Xaa₁₀₇-Xaa₁₀₈-Xaa₁₀₉-Xaa₁₁₀-Xaa₁₁₁-Xaa₁₁₂-Xaa₁₁₃; wherein: Xaa₁₁₅ is K or P; Xaa₁₆ is V or I; Xaa₂₀ is V, F, K or Y; Xaa₂₂ is G, H or S; Xaa′₂₂ is absent, G, S or A; Xaa₂₃ is S, H or G; Xaa₂₇ is E or D; Xaa₂₉ is S or A; Xaa₃₀ is V or I; Xaa₃₃ is I or T; Xaa₃₅ is S or I; Xaa₃₇ is D or N; Xaa₃₉ is Y or H; Xaa₄₂ is T or A; Xaa₄₃ is M or I; Xaa₄₉ is S or Q; Xaa₅₁ is L or E; Xaa₅₂ is P or T; Xaa₅₃ is V or T; Xaa₅₄ is V or L; Xaa₅₅ is T or A; Xaa₅₉ is L or M; Xaa₆₀ is G or K; Xaa₆₁ is L or Y; Xaa₆₉ is R or K; Xaa₇₁ is T or S; Xaa₇₃ is I or K; Xaa₇₄ is P or A; Xaa₇₅ is H or Q; Xaa₇₆ is Q or L; Xaa₇₉ is S or T; Xaa₈₄ is T or R; Xaa₈₅ is absent, T or E; Xaa₈₆ is R or N; Xaa₈₇ is absent, N or L; Xaa₈₈ is absent, E or L; Xaa₈₉ is absent or C; Xaa₉₀ is absent or N; Xaa₉₁ is absent, K or Q; Xaa₉₂ is absent, D or L; Xaa₉₃ is absent or L; Xaa₉₄ is absent, H or Q; Xaa₉₅ is absent or P; Xaa₉₆ is absent or T; Xaa₉₇ is absent or L; Xaa₉₈ is absent or P; Xaa₉₉ is absent or P; Xaa₁₀₀ is absent, V or L; Xaa₁₀₁ is absent, V or K; Xaa₁₀₂ is absent, I or N; Xaa₁₀₃ is absent, G or R; Xaa₁₀₄ is absent, P, D or A; Xaa₁₀₅ is absent or F; Xaa₁₀₆ is absent, F or V; Xaa₁₀₇ is absent or D; Xaa₁₀₈ is absent or G; Xaa₁₀₉ is absent, S or P; Xaa₁₁₀ is absent or I; Xaa₁₁₁ is absent, R or H; Xaa₁₁₂ is absent or H; and, Xaa₁₁₃ is absent or R.

The fusion protein of claim 57, wherein Xaa60 is a glycine (G) residue. In some embodiments, the polypeptide binds one or more of BMP6, BMP7 and/or BMP10. In some embodiments, the fusion protein further comprises a linker sequence. In some embodiments, the fusion protein further comprises a linker sequence selected from SEQ ID NO: 6, SEQ ID NO: 7 or SEQ ID NO: 8. In some embodiments, the Fc sequence comprises an amino acid sequence according to SEQ ID NO: 285. In some embodiments, the ALK6 ECD sequence an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a sequence selected from any one of SEQ ID NO: 90, 286, 292, 298, 304, 310, 316, 322, 328, 334 340, 346, 352, 358, 364, 370, 376, 382, 388, 394, 400, 406, 412, 418, 425 and 431.

In certain embodiments, the disclosure provides a soluble recombinant bone morphogenetic protein receptor type-1B fusion protein comprising an ALK6 extra cellular domain (ECD) polypeptide and a heterologous sequence, wherein the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 132-156, 158-189, 191-194, 202, 213, 218, 223, 228, 233, 242, 247, 252, 257, 262, 272, 277 and 283. In certain embodiments, the disclosure provides a soluble recombinant bone morphogenetic protein receptor type-1B fusion protein comprising: an ALK6 extra cellular domain (ECD) and a heterologous sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 202, 213, 218, 223, 228, 233, 242, 247, 252, 257, 262, 272, 277 and 283. In certain embodiments, the disclosure provides a soluble recombinant bone morphogenetic protein receptor type-1B fusion protein comprising: an ALK6 extra cellular domain (ECD) and a heterologous sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 90, 286, 292, 298, 304, 310, 316, 322, 328, 334 340, 346, 352, 358, 364, 370, 376, 382, 388, 394, 400, 406, 412, 418, 425 and 431. In some embodiments, the disclosure provides a soluble recombinant bone morphogenetic protein receptor type-1B fusion protein comprising: an ALK6 extra cellular domain (ECD) and a heterologous sequence wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence according to SEQ ID NO: 286.

In some embodiments, the polypeptide binds BMP6, BMP7 and/or BMP10. In some embodiments, the fusion protein further comprises a linker sequence. In some embodiments, the fusion protein further comprises a linker sequence selected from SEQ ID NO: 6, SEQ ID NO: 7 or SEQ ID NO: 8. In some embodiments, the Fc sequence comprises an amino acid sequence according to SEQ ID NO: 285.

In certain embodiments, the disclosure provides a soluble recombinant bone morphogenetic protein receptor type-1B fusion protein comprising: an ALK6 extra cellular domain (ECD), a linker sequence and a heterologous sequence wherein the fusion protein sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical SEQ ID NO: 291, 297, 303, 309, 315, 321, 327, 333, 339, 345, 351, 357, 363, 369, 375, 381, 387, 393, 399, 405, 411, 417, 423, 424, 430, and 436.

In certain embodiments, the disclosure provides a soluble recombinant bone morphogenetic protein receptor type-1B fusion protein comprising: an ALK6 extra cellular domain (ECD), a linker sequence and a heterologous sequence wherein the fusion protein sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 291.

In some embodiments, the linker sequence is according to SEQ ID NO: 6. In some embodiments, the heterologous sequence is an Fc sequence comprising an amino acid sequence according to SEQ ID NO: 285. In some embodiments, the ALK6 ECD polypeptide is present as a homodimer.

In certain embodiments, the disclosure provides a soluble recombinant bone morphogenetic protein receptor type-1B fusion protein comprising: (i) an ALK6 extra cellular domain (ECD); (ii) a linker sequence; and (iii) an Fc domain sequence; wherein the ALK6 ECD sequence comprises or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 286, wherein the position corresponding to position 60 of SEQ ID NO: 69 is a glycine (G) residue, and wherein the ALK6 fusion protein demonstrates retained binding to BMP10.

In some embodiments, the ALK6 fusion protein demonstrates BMP10 binding comparable to SEQ ID NO: 49. In some embodiments, the ALK6 fusion protein demonstrates BMP10 binding comparable to binding of an ALK6 wildtype (wt) Fc fusion protein. In some embodiments, the ALK6 fusion protein demonstrates an nM IC₅₀ value of about 7.2 for BMP10 binding. In some embodiments, the ALK6 fusion protein demonstrates an nM IC₅₀ value of about 4.8 for BMP7 binding.

In certain embodiments, the disclosure provides a soluble recombinant bone morphogenetic protein receptor type-1B fusion protein comprising: (i) an ALK6 extra cellular domain (ECD); (ii) a linker sequence; and, (iii) an Fc domain sequence; wherein the fusion protein sequence comprises or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 fusion protein sequence of SEQ ID NO: 291, wherein the position corresponding to position 60 of SEQ ID NO: 69 is a glycine (G) residue, and wherein the ALK6 fusion protein demonstrates retained BMP10 binding.

In some embodiments, the ALK6 fusion protein demonstrates BMP10 binding comparable to SEQ ID NO: 49. In some embodiments, the ALK6 fusion protein demonstrates BMP10 binding comparable to binding of an ALK6 wildtype (wt) Fc fusion protein. In some embodiments, the ALK6 fusion protein demonstrates an nM IC₅₀ value of about 7.2 for BMP10 binding. In some embodiments, the ALK6 fusion protein demonstrates an nM IC₅₀ value of about 4.8 for BMP7 binding. In some embodiments, the position corresponding to position 60 of SEQ ID NO: 69 is a glycine (G) residue, and wherein the ALK6 fusion protein demonstrates retained BMP10 binding. In some embodiments, the ALK6 fusion protein demonstrates BMP10 binding comparable to SEQ ID NO: 49. In some embodiments, the ALK6 fusion protein demonstrates BMP10 binding comparable to binding of an ALK6 wildtype (wt) Fc fusion protein. In some embodiments, the ALK6 fusion protein demonstrates an nM IC₅₀ value of about In some embodiments, the ALK6 fusion protein demonstrates an nM IC₅₀ value of about 4.8 for BMP7 binding. In some embodiments, the linker sequence comprises an amino acid sequence selected from SEQ ID NO: 6, SEQ ID NO: 7 or SEQ ID NO: 8. In some embodiments, the Fc sequence comprises an amino acid sequence selected from SEQ ID NO: 10, SEQ ID NO: 106 or SEQ ID NO: 107.

In embodiments, provided herein are pharmaceutical composition comprising a fusion protein disclosed herein; and at least one pharmaceutical acceptable carrier or buffer. In some embodiments, the pharmaceutical composition comprises an ALK6 ECD polypeptide. In some embodiments, the ALK6 ECD polypeptide is present as a homodimer.

### BRIEF DESCRIPTION OF THE DRAWINGS

FIG. 1A and FIG. 1B show SDS-PAGE gels for a number soluble ALK6 Fc fusion proteins purified using protein A chromatography (SEQ ID NO: 46-50, 70, 72, 74, 76, 84 and 85) and the cleavage fragment indicating a protease cleavage site present in SEQ ID NO: 49, 47 and 76. See Example 1.

FIG. 2A and FIG. 2B show SDS-PAGE gels for a number of soluble ALK6 Fc fusion protein variants using purified using protein A chromatography and demonstrating no cleavage fragments and indicating removal of the protease cleavage site and reduced aggregation for some variants in the non-reduced lanes of the gel. FIG. 2A shows the quantity of soluble ALK6 Fc fusion protein generated in 200 ml of cell culture demonstrating significantly improved expression for some of the variant (e.g. SEQ ID NO: 60 and 62). Those variants have point mutations replacing two histidine residues (aa22 and aa23) with serine and glycine, (SEQ ID NO: 60 also has the lysine at position 20 replaced with a tyrosine) and when compared to the expression of SEQ ID NO: 46, indicates doing so provides dramatic improvement in the expression level of those fusion proteins. See Example 2.

FIG. 2C shows SDS-PAGE gels for a number of soluble ALK6 Fc fusion protein variants using purified using protein A chromatography and demonstrating no cleavage fragments and indicating removal of the protease cleavage site and reduced aggregation in the non-reduced lanes of the gel. The gel also shows the quantity of soluble ALK6 Fc fusion protein generated in 10 ml of cell culture demonstrating significantly improved expression for some of the variants (e.g., SEQ ID NO: 234 and SEQ ID NO: 243. Those variants have point mutations replacing isoleucine (I) at aa33 and serine(S) at aa35 with threonine (T) and isoleucine (I), respectively. Those point mutations on either side of the cystine residue (at position 34) introduce a glycosylation site dramatically improving folding and expression of the fusion protein. See Example 2.

FIG. 3A shows BMP4 and BMP10 binding for soluble ALK6 Fc fusion proteins of this disclosure. The binding data demonstrates two of the variants (SEQ ID NO: 46 and 52) retain BMP4 and BMP10 ligand binding (as compared to the ALK6 wt sequence, which binds BMP2, BMP4, BMP6, BMP7, BMP10 and GDF5, GDF6 and GDF7), but that SEQ ID NO: 55 no longer binds BMP10 indicating the LGL to MKY change at positions 59, 60 and 61, respectively, is responsible for the loss of binding. See Example 3.

FIG. 3B shows ligand binding for soluble ALK6 Fc fusion proteins of this disclosure, wherein NB indicates no binding and a blank box in the table means the ligand binding was not tested. SEQ ID NO: 89 demonstrated a loss of BMP10 binding (as compared to ALK6 wt sequence) and which contains the same G60K point mutation as SEQ ID NO: 55 (which also has a loss of BMP10 binding and a reduction in BMP7 binding) indicating that point mutation is responsible for the change in ligand binding for BMP10. SEQ ID NO: 89 (and SEQ ID NO: 91 and 93) also demonstrates a retention of BMP7 binding (as compared to ALK6 wt sequence), while SEQ ID NO: 55 only demonstrates weak binding to BMP7. See Example 3.

FIG. 4 shows an IC₅₀ test of SEQ ID NO: 87 in a BMP Responsive Luciferase Assay for the BMP2, BMP4, BMP6 and BMP10 ligands. See Example 4.

FIG. 5 shows an IC₅₀ test of SEQ ID NO: 70 in a BMP Responsive Luciferase Assay for the BMP2, BMP4, BMP6 and BMP10 ligands. See Example 4.

FIG. 6 shows representative fundus fluorescein angiography (FFA) images before treatment, wherein no significant differences were observed in either the number of leakage lesions or the total leakage area of the treatment groups (G2: ALK6-Fc fusion comprising SEQ ID NO: 286 and G3: ALK6-Fc fusion comprising SEQ ID NO: 87) as compared to the control group (G1). See Example 9.

FIG. 7 shows representative fundus fluorescein angiography (FFA) images on Day 14 of systemic dosing, wherein the number of leakage lesions and leakage area were significantly decreased in G2 and G3 compared to G1 (PBS). See Example 9.

FIG. 8 shows representative fundus fluorescein angiography (FFA) images on Day 21 of systemic dosing, wherein the number of leakage lesions and leakage area were significantly decreased in G2 and G3 compared to G1 (PBS). See Example 9.

FIG. 9 shows a graph of the number of counted leakage lesions pre-dosing, Day 14 and Day 21 in each of G1, G2 and G3. G1-PBS; G2-ALK6 ECD Fc fusion protein comprising SEQ ID NO: 286; G3-ALK6 ECD Fc fusion protein comprising SEQ ID NO: 87. See Example 9.

FIG. 10 shows a graph of the measured area of the leakage lesions pre-dosing, Day 14 and Day 21 in each of G1, G2 and G3. G1-PBS; G2-ALK6 ECD Fc fusion protein comprising SEQ ID NO: 286; G3-ALK6 ECD Fc fusion protein comprising SEQ ID NO: 87. See Example 9.

FIG. 11 shows a graph of the measured area of the leakage lesions pre-dosing, and on Day 7 and Day 14 of intravitreal dosing of an ALK6 Fc fusion protein comprising SEQ ID NO: 286 as compared to EYLEA. G1-PBS; G2-ALK6 ECD Fc fusion protein comprising SEQ ID NO: 286; G3-EYLEA. See Example 10.

FIG. 12 shows a decrease in leakage area in DL-AAA rabbit model; left graph shows aggregate of all animals treated with an ALK6 ECD Fc fusion protein comprising SEQ ID NO: 286 and the graph on right shows individual animals with same treatment and the bottom image is an FA image of rabbit no. 104 showing decreased leakage area on Day 0, Day 7 and Day 14 of intravitreal dosing of an ALK6 Fc fusion protein comprising SEQ ID NO: 286. See Example 11.

FIG. 13 shows pictures from three SDS-PAGE gels with reduced and not reduced lanes with the ALK6 wt-Fc fusion protein (SEQ ID NO:49), left gel, variant construct SEQ ID NO: 70 (middle gel) and variant construct SEQ ID NO: 287 (right gel), wherein SEQ ID NO: 49 is aggregated and has a protease cleavage site in the c-terminus (indicated on gel); SEQ ID NO: 70 remains aggregated (multiple bands above the * band of the fusion protein) but is no longer cleaved; and SEQ ID NO: 287 is no longer aggregated or cleaved. See Example 1.

FIG. 14 shows the reduced and non-reduced SDS-PAGE gels with a number of ALK6 variant constructs demonstrating those constructs are not aggregated or cleaved. See Example 1b.

FIG. 15 shows the ligand binding (BMP2, BMP7, BMP10, BMP4 and BMP6) for SEQ ID NO: 287 wherein SEQ ID NO: 287 is a weak and partial inhibitor of BMP2 signaling. See Example 4.

### DETAILED DESCRIPTION OF THE DISCLOSURE

### Overview

This disclosure relates to soluble bone morphogenetic protein (BMP) receptor type 1B polypeptides comprising an extracellular domain of ALK6 (activin receptor-like kinase 6) and variants thereof (“ALK6 polypeptides”), soluble polypeptides, dimeric complexes, methods of making and uses thereof. Endogenous ALK6 is a member of the TGF-beta superfamily wherein signaling in vivo is mediated by heteromeric complexes of type I and type II serine/threonine kinase receptors, which phosphorylate and activate downstream SMAD proteins (e.g., SMAD proteins 1, 2, 3, 5, and 8) upon ligand stimulation. See, e.g., Massagué (2000) Nat. Rev. Mol. Cell Biol. 1:169-178. These type I and type II receptors are transmembrane proteins, composed of a ligand-binding extracellular domain (ECD) with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

Ligands for ALK6 are BMPs which are members of the TGF-beta superfamily, and exhibit broad spectra of biological activities in various tissues, including bone, cartilage, blood vessels, endothelia, heart, kidney, neurons, liver and lung. The BMPs and GDFs together form a family of cysteine-knot cytokines sharing the characteristic fold of the TGF-beta superfamily. See, e.g., Rider et al. (2010) Biochem J., 429 (1): 1-12. This family includes, for example, BMP2, BMP4, BMP5, BMP6, BMP7, BMP2a, BMP3, BMP3b (also known as GDF10), BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, and GDF15. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines.

BMP signaling has also been shown to contribute to the degeneration of various cell types in the eye including retinal pigment epithelial cells and inhibition of BMP signaling in various models of eye disease has been shown to improve retinal structure. For example in a murine model of retinitis pigmentosa mice homozygous for the PDE6B mutation, inhibition of BMP signaling decreased glial fibrillary acidic protein (GFAP), a marker of Muller cell stress, and a significant reduction in cell death and significantly increased retinal thickness and electrophysiologic performance (ERG) by the retinas from treated mice.

Many eye diseases lead to loss of retinal structure and function. BMP inhibition has also been shown to promote activation of retinal stem cells providing the potential for restoration of mature retinal cells and improved vision in diseases associated with retinal degeneration.

In addition, BMP signaling has also been shown to promote vascular inflammation which is an important contributor to several diseases including atherosclerosis, Alzheimer's disease multiple sclerosis, and several retinopathies. The endothelium is defined as the thin internal cell layer of blood and lymphatic vessels that are derived from mesodermal lineage. BMP signaling has been shown to lead to endothelial mesenchymal transformation or EMT leading to dysfunctional vasculature and pathological sequela. Healthy endothelium plays an essential role mainly in providing nutrients and oxygen to organs, regulating antioxidant factors, modulating the response to infection, controlling the vascular tone, and the formation of new blood vessels. This last process, named angiogenesis consists of the formation of new blood vessels from the pre-existing ones as a result of angiogenic growth factor induction. In adulthood, the endothelium is quiescent, meaning that the balance between pro- and anti-angiogenic modulators ensures the stability of blood vessel. However, angiogenesis can be reactivated under either physiological (wound healing, placentation, menstruation) or pathological conditions (tumorigenesis, stroke, inflammation, rheumatoid arthritis, etc.). Among the factors contributing to vascular development and homeostasis, vascular endothelial growth factor (VEGF) is a key endothelial cell activator. VEGF triggers proliferation toward VEGF gradient, survival, and permeability of endothelial cell. To ensure the stability of the new formed-vessel, a step of vessel maturation is required through quiescent factors control.

Angiogenesis plays a crucial role in numerous pathological conditions such as inflammation and tumorigenesis. In addition, regenerative medicine for vascular systems has attracted much attention as a potential means of eliminating the vascular-system defects accompanying diabetes and other diseases. Provided herein are molecules that bind selectively to BMP for the treatment of inflammation and vascular dysfunction.

Provided herein in embodiments are BMP trap molecules (“BMP antagonists”) comprising the soluble ALK6 polypeptides of this disclosure, which bind BMP ligands and prevent their binding to endogenous transmembrane receptors thus down regulating the BMP signaling pathway. Inhibiting selective BMPs (those that naturally bind to ALK-6, provided above, or those through point mutation have been engineered to bind by ALK6 variants of this disclosure), are provided herein as treatment for inflammation and vascular dysfunction. ALK6 ligands including BMP 2, 4, 5, 6, and 7 are associated with vascular inflammation, inhibiting any those while not inhibiting/binding to BMP-9 and/or BMP-10 may participate in quiescence of endothelia cells. This down regulation of selected BMP signaling pathways inhibits, for example, the Smad1/5/8 cascade alleviating certain vascular inflammation disorders such as retinopathy. In embodiments, selective inhibition of BMP molecules may shift or restore a balance so that the BMP molecules not bound by the ALK-6 polypeptide of this disclosure (e.g. BMP 9) are now free to bind their target receptor without competition from other BMP ligands (e.g. BMP 2, 4, 5, 6, and 7). In certain embodiments, BMP9 endothelia quiescent activity is increased by inhibiting competing ligands. As used herein, a “trap molecule” sequesters the respective ligand (e.g. BMPs) before it can interact with the endogenous receptor and thus inhibiting the signal transduction pathway. The soluble ALK6 polypeptides, when fused to a heterologous domain (e.g., Fc domain or IgG hinge region) (“fusion polypeptide”) form homodimers. In embodiments, any sequence that will form one or more disulfide bonds can be used to form the homodimers.

Provided in certain embodiments are methods for the treatment of retinopathy, comprising administering the soluble ALK6 polypeptides of this disclosure to a subject in need thereof. In certain embodiments the method for the treatment of retinopathy comprises administering a pharmaceutical composition to a subject in need thereof, wherein the composition comprises a soluble recombinant bone morphogenetic protein receptor type-1B fusion protein comprising an ALK6 ECD polypeptide and a Fc sequence of this disclosure. In embodiments, the Fc sequence is not full length but just long enough to for the fused ALK6 ECD polypeptides to form homodimers.

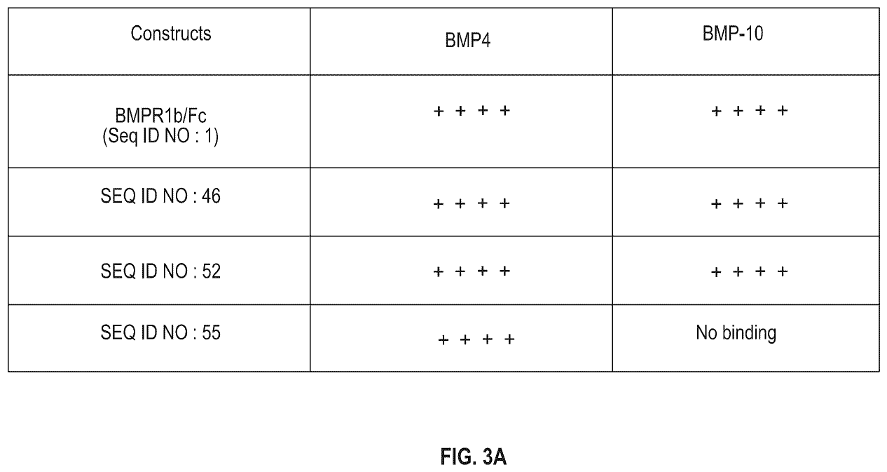

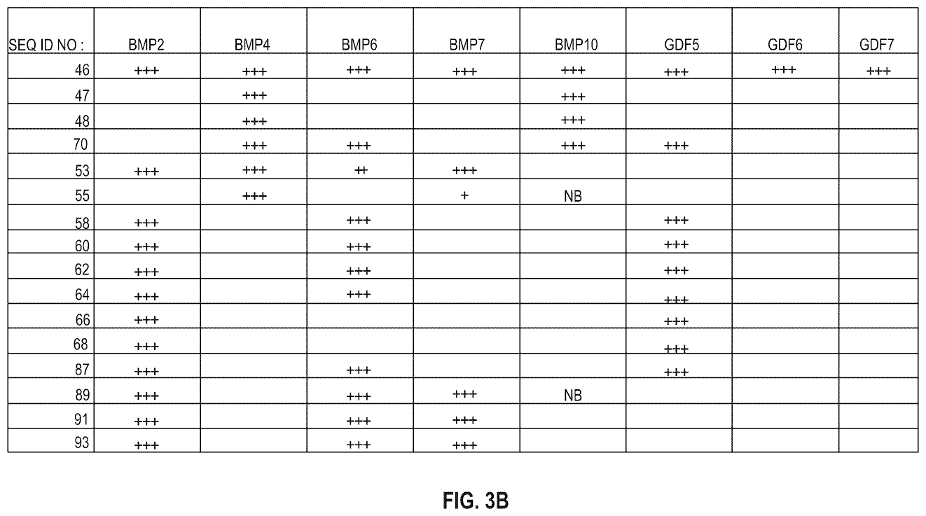

Applicants surprisingly found that maintaining a glycine (G) residue at position 60, according to SEQ ID NO: 1, provides an ALK6 ECD variant that binds BMP10 (even with one or more point mutations or a deletion of C- or N-terminal residues). ALK6 ECD variants, such as SEQ ID NO: 55 and SEQ ID NO: 89, that had a point mutation at that position (e.g., G60K) lost BMP10 binding as compared to the ALK6 wt ECD sequence (SEQ ID NO: 46). See FIG. 3B. In certain embodiments, the ALK6 fusion proteins of this disclosure bind BMP10 (variants with glycine at amino acid position 60). In certain other embodiments, the ALK6 fusion proteins of this disclosure do not bind BMP10 (e.g. contain a point mutation at amino acid position such as G60K). In certain embodiments provided herein are soluble recombinant bone morphogenetic protein receptor type-1B fusion proteins comprising an ALK6 extra cellular domain (ECD) polypeptide according to this disclosure, a linker sequence and a Fc sequence, which partially inhibit BMP2 signaling.

Provided in certain embodiments are methods for treating a retinopathy disease, comprising administering the soluble ALK6 Fc fusion proteins of this disclosure to a subject in need thereof. In certain embodiments the method for treating the retinopathy disease comprises administering a pharmaceutical composition to a subject in need thereof, wherein the composition comprises a soluble recombinant bone morphogenetic protein receptor type-1B fusion protein comprising an ALK6 ECD polypeptide, a linker and a Fc sequence of this disclosure. In certain embodiments the soluble ALK6 ECD-Fc fusion protein is SEQ ID NO: 291, or a sequence having at least 90% identity to SEQ ID NO: 291.

### Definitions

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity. As used herein the ALK6 polypeptides are referred to as antagonists for the endogenous ALK6 transmembrane receptor signaling pathway wherein the ALK6 polypeptides bind and sequester their endogenous BMP ligand.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges are inclusive of the numbers defining the range. Measured and measurable values are understood to be approximate, taking into account significant digits and the error associated with the measurement. Accordingly, unless indicated to the contrary, the numerical parameters set forth in the following description and appended claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “or” is used herein in the inclusive sense, i.e., equivalent to “and/or” unless the context clearly requires otherwise.

The use of “comprise”, “comprises”, “comprising”, “contain”, “contains”, “containing”, “include”, “includes”, and “including” is not intended to be limiting, and means that at least the named compound, element, particle, or method step is present in the composition or article or method, but does not exclude the presence of other compounds, materials, particles, method steps, even if the other such compounds, material, particles, method steps have the same function as what is named.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure is achieved by administering one or more soluble ALK6 polypeptides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

The term “eye disorder” is used herein interchangeable with “retinopathy” and as used herein refers to any eye disorder associated with pathological angiogenesis (also referred to herein interchangeably as “eye condition”). include. Eye disorders can be characterized by alteration or uncontrolled proliferation of new blood vessels and/or invasion of ocular tissue structures such as the retina or cornea. Non-limiting eye disorders include, for example, AMD (e.g., exudative AMD, dry AMD, intermediate AMD, progressive AMD, and map-like atrophy (GA)), macular degeneration, macular edema, DME. Local non-central DME and diffuse central involvement-related DME), retinopathy, diabetic retinopathy (DR) (e.g., proliferative DR (PDR), non-proliferative DR (NPDR), and high altitude DR), and others. Ischemic-related retinopathy, ROP, retinal vein occlusion (RVO) (e.g., central retinal vein occlusion (CRVO) and retinal branch vein occlusion (BRVO) morphology), CNV (e.g., myopic CNV), macular blood vessels Newborn, diseases associated with corneal angiogenesis, retinal angiogenesis, diseases associated with retinal/choroidal angiogenesis, pathological myopia, von Hippel Lindow's disease, eye macular degeneration, FEVR, Coates' disease, Norie's disease, OPPG, subconjunctival bleeding, cutaneous erythema, ocular angiogenesis, angiogenic glaucoma, retinal pigment degeneration (RP), hypertensive retinopathy, macular degeneration, macular degeneration, macular degeneration, eye Internal angiogenesis, retinal degeneration, cyst-like macular edema (CME), vasculitis, optic nerve papilla edema, retinitis, conjunctivitis (e.g., infectious conjunctivitis and non-infectious (e.g., allergic) conjunctivitis), Labor congenital black internal disorder (also known as Labor congenital macular degeneration or LCA), macular degeneration (including infectious and non-infectious macular degeneration), choroiditis (e.g., polyfocal macular degeneration), ocular macular degeneration, eyelid inflammation Includes, dry eyes, traumatic eye damage, Sjogren's disease, and other eye disorders in which the disease or disorder is associated with ocular neovascularization, vascular leakage, and/or retinal edema. Additional exemplary eye disorders include all forms of proliferative vitreoretinopathy, diseases associated with cutaneous neovascularization of the angle, and fibrovascular or fibers. Includes diseases caused by overgrowth of tissue.

The term “retina” refers to a region of the central nervous system with approximately 150 million neurons. It is located at the back of the eye where it rests upon a specialized epithelial tissue called retinal pigment epithelium or RPE. The retina initiates the first stage of visual processing by transducing visual stimuli in specialized neurons called “photoreceptors”. Their synaptic outputs are processed by elaborate neural networks in the retina and then transmitted to the brain. The retina has evolved two specialized classes of photoreceptors to operate under a wide range of light conditions. “Rod” photoreceptors transduce visual images under low light conditions and mediate achromatic vision. “Cone” photoreceptors transduce visual images in dim to bright light conditions and mediate both color vision and high acuity vision.

Every photoreceptor is compartmentalized into two regions called the “outer” and “inner” segment. The inner segment is the neuronal cell body containing the cell nucleus. The inner segment survives for a lifetime in the absence of retinal disease. The outer segment is the region where the light sensitive visual pigment molecules are concentrated in a dense array of stacked membrane structures. Part of the outer segment is routinely shed and regrown in a diurnal process called outer segment renewal. Shed outer segments are ingested and metabolized by RPE cells.

The term “macula” refers to the central region of the retina, which contains the fovea where visual images are processed by long slender cones in high spatial detail (“visual acuity”). “Macular degeneration” is a form of retinal neurodegeneration, which attacks the macula and destroys high acuity vision in the center of the visual field. AMD can be in a “dry form” characterized by residual lysosomal granules called lipofuscin in RPE cells, and by extracellular deposits called “drusen”. Drusen contain cellular waste products excreted by RPE cells. “Lipofuscin” and drusen can be detected clinically by ophthalmologists and quantified using fluorescence techniques. They can be the first clinical signs of macular degeneration.

Lipofuscin contains aggregations of A2E. Lipofuscin accumulates in RPE cells and poisons them by multiple known mechanisms. As RPE cells become poisoned, their biochemical activities decline and photoreceptors begin to degenerate. Extracellular drusen may further compromise RPE cells by interfering with their supply of vascular nutrients. Drusen also trigger inflammatory processes, which leads to choroidal neovascular invasions of the macula in one patient in ten who progresses to wet form AMD. Both the dry form and wet form progress to blindness.

The term “ERG” is an acronym for electroretinogram, which is the measurement of the electric field potential emitted by retinal neurons during their response to an experimentally defined light stimulus. ERG is a non-invasive measurement, which can be performed on either living subjects (human or animal) or a hemisected eye in solution that has been removed surgically from a living animal.

The term “RAL” means retinaldehyde. “Free RAL” is defined as RAL that is not bound to a visual cycle protein. The terms “trans-RAL” and “all-trans-RAL” are used interchangeably and mean all-trans-retinaldehyde.

Also, in describing the embodiments, terminology will be resorted to for the sake of clarity. It is intended that each term contemplates its broadest meaning as understood by those skilled in the art and includes all technical equivalents which operate in a similar manner to accomplish a similar purpose.

### Bone Morphogenetic Protein (BMP) Receptor Type 1B Polypeptides

The present disclosure generally relates to soluble ALK6 polypeptides, variants thereof, compositions, and uses thereof (e.g., treating, preventing or reducing the progression rate and/or severity) of retinopathy disorders (e.g., diabetic retinopathy, age-related macular degeneration (AMD), choroidal neovascularization (CNV), vision loss, night blindness, photopsia, proliferative retinopathy, non-proliferative retinopathy, retinitis pigmentosa and hypertensive retinopathy)). As used herein the term “ALK6 polypeptide” refers to the extracellular domain of human ALK6 provided for example in SEQ ID NO: 1, as well as any variants thereof (including mutants, fragments, fusions, and peptidomimetic forms) that retain a useful activity.

Numbering of amino acids for all ALK6-related polypeptides described herein is based on the numbering of the human ALK6 processed (mature) extracellular domain (ECD) human protein sequence provided below (SEQ ID NO: 1), unless specifically designated otherwise.

(SEQ ID NO: 1)
KKEDGESTAPTPRPKVLRCKCHHHCPEDSVNNICSTDGYCFTMIEEDDS
GLPVVTSGCLGLEGSDFQCRDTPIPHQRRSIECCTERNECNKDLHPTLP
PLKNRDFVDGPIHHR

The C-terminal “tail” of the extracellular domain is indicated by single underline in SEQ ID NO: 1 (above). In certain embodiments the ALK6 ECD sequence with the “tail” deleted (a Δ14 sequence or Δ13 sequence) (“SEQ ID NO: 4 variant”) is as follows:

(SEQ ID NO: 4)
KKEDGESTAPTPRPKVLRCKCHHHCPEDSVNNICSTDGYCFTMIEEDDS
GLPVVTSGCLGLEGSDFQCRDTPIPHQRRSIECCTERNECNKDLHPTLP
P(L)

In certain embodiments the 14 amino acid deleted C terminal tail sequence is replaced with VVIGPFFDGSIR (SEQ ID NO: 2) providing a variant ALK6 ECD polypeptide (“SEQ ID NO: 11 variant”) as follows:

(SEQ ID NO: 11)
KKEDGESTAPTPRPKVLRCKCHHHCPEDSVNNICSTDGYCFTMIEEDDS
GLPVVTSGCLGLEGSDFQCRDTPIPHQRRSIECCTERNECNKDLHPTLP
PVVIGPFFDGSIR

In certain embodiments the C-terminus tail is further deleted for a total of 30 amino acids (SEQ ID NO: 439) and the amino acid deleted C terminal tail sequence is replaced with

(SEQ ID NO: 195)
RTNLCNQYLOPTLPPVVIG

providing a variant ALK6 ECD polypeptide (“SEQ ID NO: 156 variant”) as follows:

KKEDGESTAPTPRPKVLRCKCHHHCPEDSVNNICSTDGYCFTMIEEDDS
GLPVVTSGCLGLEGSDFQCRDTPIPHQRRSIECCRTNLCNQYLQPTLPP
VVIG

In certain embodiments the C-terminus tail is further deleted for a total of 30 amino acids (SEQ ID NO: 439) and the amino acid deleted C terminal tail sequence is replaced with

(SEQ ID NO: 438)
TNLCNQYLOPTLPPVVIGPFFDGSIR

providing a variant ALK6 ECD polypeptide (“SEQ ID NO: 425 variant”) as follows:

KKEDGESTAPTPRPKVLRCKCHHHCPEDSVNNICSTDGYC
FTMIEEDDSGLPVVTSGCLGLEGSDFQCRDTPIPHQRRSI
ECCTNLCNQYLQPTLPPVVIGPFFDGSIR

In certain other embodiments, a seven amino acid portion of the “tail” sequence is deleted (a Δ7 sequence) from SEQ ID NO: 1 (“SEQ ID NO: 69 variant”), providing the following sequence:

(SEQ ID NO: 69)
KKEDGESTAPTPRPKVLRCKCHHHCPEDSVNNICSTDGYC
FTMIEEDDSGLPVVTSGCLGLEGSDFQCRDTPIPHQRRSI
ECCTERNECNKDLHPTLPPLKNRDFV

In certain other embodiments provided herein the last two histidine amino acids of the “tail” sequence from SEQ ID NO: 1 (“SEQ ID NO: 80 variant”) are deleted providing an ALK6 ECD polypeptide having the sequence:

(SEQ ID NO: 80)
KKEDGESTAPTPRPKVLRCKCHHHCPEDSVNNICSTDGYCF
TMIEEDDSGLPVVTSGCLGLEGSDFQCRDTPIPHQRRSIEC
CTERNECNKDLHPTLPPLKNRDFVDGPI R

In certain other embodiments provided herein the mutations K20Y; H22S; _22′A (inserted between aa22 and aa23) H23G are inserted into SEQ ID NO: 69 (“SEQ ID NO: 90 variant”) providing the following sequence:

KKEDGESTAPTPRPKVLRCYCSAGHCPEDSVNNICSTDGYC
FTMIEEDDSGLPVVTSGCLGLEGSDFQCRDTPIPHQRRSIE
CCTERNECNKDLHPTLPPLKNRDFV

In certain other embodiments, a seven amino acid portion of the “tail” sequence is deleted (a Δ7 sequence) from SEQ ID NO: 1 (“SEQ ID NO: 113 variant”), and an arginine amino acid added between aa22 and aa23, providing the following sequence:

(SEQ ID NO: 113)
KKEDGESTAPTPRPKVLRCKCHAHHCPEDSVNNICSTDGYCF
TMIEEDDSGLPVVTSGCLGLEGSDFQCRDTPIPHQRRSIEC
CTERNECNKDLHPTLPPLKNRDFV

In certain embodiments the ALK6 ECD sequence within the N-terminus is deleted (a Δ10 sequence, a Δ13 sequence or a Δ16 sequence) (“SEQ ID NO: 153 variant”, “SEQ ID NO: 154 variant” or “SEQ ID NO: 155 variant”,) is as follows:

(SEQ ID NO: 153)
TPRPKVLRCKCHHHCPEDSVNNICSTDGYCFTMIEEDDSGLPV
VTSGCLGLEGSDFQCRDTPIPHQRRSIECCTERNECNKDLHPT
LPPLKNRDFVDGPIHHR
(SEQ ID NO: 154)
PKVLRCKCHHHCPEDSVNNICSTDGYCFTMIEEDDSGLPVVTS
GCLGLEGSDFQCRDTPIPHQRRSIECCTERNECNKDLHPTLPP
LKNRDFVDGPIHHR
(SEQ ID NO: 155)
LRCKCHHHCPEDSVNNICSTDGYCFTMIEEDDSGLPVVTSGCL
GLEGSDFQCRDTPIPHQRRSIECCTERNECNKDLHPTLPPLKN
RDFVDGPIHHR

In certain embodiments, the ALK6 polypeptide comprises a combination of a modified version of SEQ ID NO: 1 (i.e., the extracellular domain of “wild-type” or naturally occurring BMP antagonist). In embodiments, the wild-type ALK-6 does not include the C-terminal sequence of SEQ ID NO: 1, such as that shown in SEQ ID NO: 4. In embodiments, the ALK6 polypeptide comprises a modified version of SEQ ID NO: 1 wherein a seven amino acid portion of the “tail” sequence is deleted (SEQ ID NO: 69) or wherein two histidine amino acids at positions 111 and 112 of the ALK6 polypeptide ECD sequence are deleted (SEQ ID NO: 80). In certain embodiments, the ALK6 polypeptide comprises SEQ ID NO: 11 wherein the fourteen (14) amino acid “tail” deleted sequence is replaced with SEQ ID NO: 2. In certain embodiments, the ALK6 polypeptide comprises SEQ ID NO: 156 wherein a thirty (30) amino acid “tail” deleted sequence is replaced with SEQ ID NO: 2. In certain embodiments, the ALK6 polypeptide comprises SEQ ID NO: 425 wherein a thirty (30) amino acid “tail” sequence is replaced with SEQ ID NO: 438. In certain embodiments, the ALK6 polypeptide has the N terminus deleted or truncated, by 10 (SEQ ID NO: 153), 13 (SEQ ID NO: 154) or 16 (SEQ ID NO: 155) amino acid positions. As described in more detail below, any of these sequences (e.g., SEQ ID NO: 1, 4, 11, 69, 80, 113, 153, 154, 155, 156 or 425) can be modified to alter and/or optimize ligand binding and/or to remove undesirable properties (e.g., protease cleave site(s) or aggregation).

Accordingly, a general formula for an active portion (e.g., ligand binding) of ALK6 is a polypeptide that comprises, consists essentially of, or consists of SEQ ID NO: 1. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 ECD of SEQ ID NO: 1.

In embodiments, an active portion (e.g., ligand binding) of ALK6 is a polypeptide that comprises, consists essentially of, or consists of SEQ ID NO: 4. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 4.

In embodiments, an active portion (e.g., ligand binding) of ALK6 is a polypeptide that comprises, consists essentially of, or consists of SEQ ID NO: 11. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 11.

In embodiments, an active portion (e.g., ligand binding) of ALK6 is a polypeptide that comprises, consists essentially of, or consists of SEQ ID NO: 69. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 69.

In embodiments, an active portion (e.g., ligand binding) of ALK6 is a polypeptide that comprises, consists essentially of, or consists of SEQ ID NO: 80. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 80.

In embodiments, an active portion (e.g., ligand binding) of ALK6 is a polypeptide that comprises, consists essentially of, or consists of SEQ ID NO: 90. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 90.

In embodiments, an active portion (e.g., ligand binding) of ALK6 is a polypeptide that comprises, consists essentially of, or consists of SEQ ID NO: 113. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 113.

In embodiments, an active portion (e.g., ligand binding) of ALK6 is a polypeptide that comprises, consists essentially of, or consists of SEQ ID NO: 153. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 153.

In embodiments, an active portion (e.g., ligand binding) of ALK6 is a polypeptide that comprises, consists essentially of, or consists of SEQ ID NO: 154. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 154.

In embodiments, an active portion (e.g., ligand binding) of ALK6 is a polypeptide that comprises, consists essentially of, or consists of SEQ ID NO: 155. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 155.

In embodiments, an active portion (e.g., ligand binding) of ALK6 is a polypeptide that comprises, consists essentially of, or consists of SEQ ID NO: 156. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 156.

Provided herein are specific point mutations, additions or point deletions within any of the above disclosed SEQ ID NO: 1, 4, 11, 69, 80, 90, 113, 153, 154, 155 or 156. Those exemplified mutations and/or deletions and/or additions are represented in SEQ ID NO: 3, 5, 79, 81, 82, 83, 108, 109, 130, 157, 190 or 437. Provided herein are ALK6 ECD sequences selected from any sequence according to SEQ ID NO: 3, 5, 79, 81, 82, 83, 108, 109, 130, 157, 190 or 437.

In certain embodiments, the soluble ALK6 polypeptide comprises an ECD sequence of ALK6 according to SEQ ID NO: 3. In certain embodiments, the soluble ALK6 polypeptide comprises an ECD sequence of ALK6 according to SEQ ID NO: 5. In certain embodiments, the soluble ALK6 polypeptide comprises an ECD sequence of ALK6 according to SEQ ID NO: 79. In certain embodiments, the soluble ALK6 polypeptide comprises an ECD sequence of ALK6 according to SEQ ID NO: 81. In certain embodiments, the soluble ALK6 polypeptide comprises an ECD sequence of ALK6 according to SEQ ID NO: 82. In certain embodiments, the soluble ALK6 polypeptide comprises an ECD sequence of ALK6 according to SEQ ID NO: 83. In certain embodiments, the soluble ALK6 polypeptide comprises an ECD sequence of ALK6 according to SEQ ID NO: 108. In certain embodiments, the soluble ALK6 polypeptide comprises an ECD sequence of ALK6 according to SEQ ID NO: 109. In certain embodiments, the soluble ALK6 polypeptide comprises an ECD sequence of ALK6 according to SEQ ID NO: 153. In certain embodiments, the soluble ALK6 polypeptide comprises an ECD sequence of ALK6 according to SEQ ID NO: 154. In certain embodiments, the soluble ALK6 polypeptide comprises an ECD sequence of ALK6 according to SEQ ID NO: 155. In certain embodiments, the soluble ALK6 polypeptide comprises an ECD sequence of ALK6 according to SEQ ID NO: 156. In certain embodiments, the soluble ALK6 polypeptide comprises an ECD sequence of ALK6 according to SEQ ID NO: 157. In certain embodiments, the soluble ALK6 polypeptide comprises an ECD sequence of ALK6 according to SEQ ID NO: 190. In certain embodiments, the soluble ALK6 polypeptide comprises an ECD sequence of ALK6 according to SEQ ID NO: 437.

In certain embodiments, the soluble ALK-6 polypeptide comprises an active portion (e.g., ligand binding) of ALK6 according to SEQ ID NO: 3 as shown below using standard three-letter amino acid abbreviations:

Lys₁-Lys₂-Glu₃-Asp₄-Gly₅-Glu₆-Ser₇-Thr₈-Ala₉-Pro₁₀-
Thr₁₁-Pro₁₂-Arg₁₃-Pro₁₄-Xaa₁₅-Val₁₆-Leu₁₇-Arg₁₈-
Cys₁₉-Xaa₂₀-Cys₂₁-Xaa₂₂-Xaa₂₃-His₂₄-Cys₂₅-Pro₂₆-
Xaa₂₇-Asp₂₈-Xaa₂₉-Xaa₃₀-Asn₃₁-Asn₃₂-Xaa₃₃-Cys₃₄-
Xaa₃₅-Thr₃₆-Xaa₃₇-Gly₃₈-Xaa₃₉-Cys₄₀-Phe₄₁-Xaa₄₂-
Xaa₄₃-Ile₄₄-Glu₄₅-Glu₄₆-Asp₄₇-Asp₄₈-Xaa₄₉-Gly₅₀-
Xaa₅₁-Xaa₅₂-Xaa₅₃-Xaa₅₄-Xaa₅₅-Ser₅₆-Gly₅₇-Cys₅₈-
Xaa₅₉-Xaa₆₀-Xaa₆₁-Glu₆₂-Gly₆₃-Ser₆₄-Asp₆₅-Phe₆₆-
Gln₆₇-Cys₆₈-Xaa₆₉-Asp₇₀-Xaa₇₁-Pro₇₂-Xaa₇₃-Xaa₇₄-
Xaa₇₅-Xaa₇₆-Arg₇₇-Arg₇₈-Xaa₇₉.Ile₈₀-Glu₈₁-Cys₈₂-
Cys₈₃-Xaa₈₄-Xaa₈₅-Xaa₈₆-Xaa₈₇-Xaa₈₈-Cys₈₉-Asn₉₀-
Xaa₉₁-Xaa₉₂-Leu₉₃-Xaa₉₄-Pro₉₅-Thr₉₆-Leu₉₇-Pro₉₈-
Pro₉₉-Xaa₁₀₀-Xaa₁₀₁-Xaa₁₀₂-Xaa₁₀₃-Xaa₁₀₄-Xaa₁₀₅-
Xaa₁₀₆-Xaa₁₀₇-Xaa₁₀₈-Xaa₁₀₉-Xaa₁₁₀-Xaa₁₁₁-Xaa₁₁₂-
Xaa₁₁₃;

wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₀ is absent, V or L;
Xaa₁₀₁ is absent, V or K;
Xaa₁₀₂ is absent, I or N;
Xaa₁₀₃ is absent, G or R;
Xaa₁₀₄ is absent, P, D or A;
Xaa₁₀₅ is absent or F;
Xaa₁₀₆ is absent, F or V;
Xaa₁₀₇ is absent or D;
Xaa₁₀₈ is absent or G;
Xaa₁₀₉ is absent, S or P;
Xaa₁₁₀ is absent or I;
Xaa₁₁ is absent, R or H;
Xaa₁₁₂ is absent or H; and,
Xaa₁₁₃ is absent or R (SEQ ID NO: 3).

In embodiments, the ALK6 ECD polypeptide of the fusion protein comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein comprises one or more substitutions selected from L59M, G60K, and L61Y. In certain embodiments, the ALK6 ECD polypeptide of the fusion protein comprises one or more substitutions selected from K20Y, H22S and H23G. In certain embodiments, Xaa₁₀₀-Xaa₁₁₃ of the ALK6 ECD polypeptide according to SEQ ID NO: 3 are absent. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 3 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 1, 4, 11-45, 57, 59, 61, 63, 65, 67, 69, 71, 73, 75, 77, 80, 86, 88, 92, 94, 96, 98, and 100.

In certain embodiments, the soluble ALK6 polypeptide comprises an ECD sequence of ALK6 according to SEQ ID NO: 5 as shown below using standard three-letter amino acid abbreviations:

Lys₁-Lys₂-Glu₃-Asp₄-Glys-Glu₆-Ser₇-Thr₈-Ala₉-Pro₁₀-
Thr₁₁-Pro₁₂-Arg₁₃-Pro₁₄-Xaa₁₅-Val₁₆-Leu₁₇-Arg₁₈-
Cys₁₉-Xaa₂₀-Cys₂₁-Xaa₂₂-Xaa₂₃-His₂₄-Cys₂₅-Pro₂₆-
Xaa₂₇-Asp₂₈-Xaa₂₉-Xaa₃₀-Asn₃₁-Asn₃₂-Xaa₃₃-Cys₃₄-
Xaa₃₅-Thr₃₆-Xaa₃₇-Gly₃₈-Xaa₃₉-Cys₄₀-Phe₄₁-Xaa₄₂-
Xaa₄₃-Ile₄₄-Glu₄₅-Glu₄₆-Asp₄₇-Asp₄₈-Xaa₄₉-Gly₅₀-
Xaa₅₁-Xaa₅₂-Xaa₅₃-Xaa₅₄-Xaa₅₅-Ser₅₆-Gly₅₇-Cys₅₈-
Xaa₅₉-Xaac₀-Xaa₆₁-Glu₆₂-Gly₆₃-Ser₆₄-Asp₆₅-Phe₆₆-
Gln₆₇-Cys₆₈-Xaa₆₉-Aspr₀-Xaa₇₁-Pro₇₂-Xaa₇₃-Xaa₇₄-
Xaa₇₅-Xaa₇₆-Arg₇₇-Arg₇₈-Xaa₇₉-Ile₈₀-Glu₈₁-Cys₈₂-
Cys₈₃-Xaa₈₄-Xaa₈₅-Xaa₈₆-Xaa₈₇-Xaa₈₈-Cys₈₉-Asn₉₀-
Xaa₉₁-Xaa₉₂-Leu₉₃-Xaa₉₄-Pro₉₅-Thr₉₆-Leu₉₇-Pro₉₈-
Pro₉₉-Leu₁₀₀;

wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L; and,
Xaa₉₄ is H or Q (SEQ ID NO: 5).

In embodiments, the ALK6 ECD polypeptide of the fusion protein according to SEQ ID NO: 5 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, or a combination thereof, wherein _ is a deletion at that position. In certain embodiments, the ALK6 ECD polypeptide of the fusion protein according to SEQ ID NO: 5 comprises one or more substitutions selected from L59M, G60K, and L61Y. In certain embodiments, the ALK6 ECD polypeptide of the fusion protein according to SEQ ID NO: 5 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the soluble ALK6 polypeptide comprises an ECD sequence of ALK6 according to SEQ ID NO: 79 as shown below using standard three-letter amino acid abbreviations:

Lys₁-Lys₂-Glu₃-Asp₄-Glys-Glu₆-Ser₇-Thr₈-Ala₉-Pro₁₀-
Thr₁₁-Pro₁₂-Arg₁₃-Pro₁₄-Xaa₁₅-Val₁₆-Leu₁₇-Arg₁₈-
Cys₁₉-Xaa₂₀-Cys₂₁-Xaa₂₂-Xaa₂₃-His₂₄-Cys₂₅-Pro₂₆-
Xaa₂₇-Asp₂₈-Xaa₂₉-Xaa₃₀-Asn₃₁-Asn₃₂-Xaa₃₃-Cys₃₄-
Xaa₃₅-Thr₃₆-Xaa₃₇-Gly₃₈-Xaa₃₉-Cys₄₀-Phe₄₁-Xaa₄₂-
Xaa₄₃-Ile₄₄-Glu₄₅-Glu₄₆-Asp₄₇-Asp₄₈-Xaa₄₉-Gly₅₀-
Xaa₅₁-Xaa₅₂-Xaa₅₃-Xaa₅₄-Xaa₅₅-Ser₅₆-Gly₅₇-Cys₅₈-
Xaa₅₉-Xaa₆₀-Xaa₆₁-Glu₆₂-Gly₆₃-Ser₆₄-Asp₆₅-Phe₆₆-
Gln₆₇-Cys₆₈-Xaa₆₉-Asp₇₀-Xaa₇₁-Pro₇₂-Xaa₇₃-Xaa₇₄-
Xaa₇₅-Xaa₇₆-Arg₇₇-Arg₇₈-Xaa₇₉-Ile₈₀-Glu₈₁-Cys₈₂-
Cys₈₃-Xaa₈₄-Xaa₈₅-Xaa₈₆-Xaa₈₇-Xaa₈₈-Cys₈₉-Asn₉₀-
Xaa₉₁-Xaa₉₂-Leu₉₃-Xaa₉₄-Pro₉₅-Thr₉₆-Leu₉₇-Pro₉₈-
Pro₉₉-Val₁₀₀-Val₁₀₁-Ile₁₀₂-Gly₁₀₃-Pro₁₀₄-Phe₁₀₅-
Phe₁₀₆-Asp₁₀₇-Gly₁₀₈-Ser₁₀₉-Ile₁₁₀-Arg₁₁₁;

wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L; and,
Xaa₉₄ is H or Q (SEQ ID NO: 79).

In embodiments, the ALK6 ECD polypeptide of the fusion protein according to SEQ ID NO: 79 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 79 comprises one or more substitutions selected from L59M, G60K, and L61Y. In certain embodiments, the ALK6 ECD polypeptide of the fusion protein according to SEQ ID NO: 79 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 79 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 15-45, 57, 59, 61, 63, 65, and 88.

In certain embodiments, the soluble ALK-6 polypeptide comprises an active portion (e.g., ligand binding) of ALK6 according to SEQ ID NO: 81 as shown below using standard three-letter amino acid abbreviations:

Lys₁-Lys₂-Glu₃-Asp₄-Gly₅-Glu₆-Ser₇-Thr₈-Ala₉-Pro₁₀-
Thr₁₁-Pro₁₂-Arg₁₃-Pro₁₄-Xaa₁₅-Val₁₆-Leu₁₇-Arg₁₈-
Cys₁₉-Xaa₂₀-Cys₂₁-Xaa₂₂-Xaa₂₃-His₂₄-Cys₂₅-Pro₂₆-
Xaa₂₇-Asp₂₈-Xaa₂₉-Xaa₃₀-Asn₃₁-Asn₃₂-Xaa₃₃-Cys₃₄-
Xaa₃₅-Thr₃₆-Xaa₃₇-Gly₃₈-Xaa₃₉-Cys₄₀-Phe₄₁-Xaa₄₂-
Xaa₄₃-Ile₄₄-Glu₄₅-Glu₄₆-Asp₄₇-Asp₄₈-Xaa₄₉-Gly₅₀-
Xaa₅₁-Xaa₅₂-Xaa₅₃-Xaa₅₄-Xaa₅₅-Ser₅₆-Gly₅₇-Cys₅₈-
Xaa₅₉-Xaac₀-Xaa₆₁-Glu₆₂-Gly₆₃-Ser₆₄-Asp₆₅-Phe₆₆-
Gln₆₇-Cys₆₈-Xaa₆₉-Asp₇₀-Xaa₇₁-Pro₇₂-Xaa₇₃-Xaa₇₄-
Xaa₇₅-Xaa₇₆-Arg₇₇-Arg₇₈-Xaa₇₉-Ile₈₀-Glu₈₁-Cys₈₂-
Cys₈₃-Xaa₆₄-Xaa₈₅-Xaa₈₆-Xaa₈₇-Xaa₈₈-Cys₈₉-Asn₉₀-
Xaa₉₁-Xaa₉₂-Leu₉₃-Xaa₉₄-Pro₉₅-Thr₉₆-Leu₉₇-Pro₉₈-
Pro₉₉-Leu₁₀₀-Lys₁₀₁-Asn₁₀₂-Arg₁₀₃-Asp₁₀₄-Phe₁₀₅-
Val₁₀₆-Xaa₁₀₇-Xaa₁₀₈-Xaa₁₀₉-Xaa₁₁₀-Xaa₁₁₁-Xaa₁₁₂-
Xaa₁₁₃;

wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is absent;
Xaa₁₀₈ is absent;
Xaa₁₀₉ is absent;
Xaa₁₁₀ is absent;
Xaa₁₁₁ is absent;
Xaa₁₁₂ is absent; and,
Xaa₁₁₃ is absent (SEQ ID NO: 81).

In embodiments, the ALK6 ECD polypeptide of the fusion protein according to SEQ ID NO: 81 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position. In certain embodiments, the ALK6 ECD polypeptide of the fusion protein according to SEQ ID NO: 81 comprises one or more substitutions selected from L59M, G60K, and L61Y. In certain embodiments, the ALK6 ECD polypeptide of the fusion protein according to SEQ ID NO: 81 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 81 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 13, 86, 92, 94, 96, 98, 100, 134, 135, 136, 137, 138, 139, 140, 141, 142, 143, 144, 145, 147, or 149.

In certain embodiments, the ALK6 ECD polypeptide of the fusion protein is selected from a sequence according to SEQ ID NO: 81 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 86 wherein amino acid position 60 is a glycine (G) residue.

In certain embodiments, the ALK6 ECD polypeptide of the fusion protein is selected from a sequence according to SEQ ID NO: 81 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 86 wherein amino acid position 60 is other than a glycine (G) residue, suck lysine (K). The point mutation G60K removes BMP10 binding.

In embodiments, the soluble ALK-6 polypeptide comprises an active portion (e.g., ligand binding) of ALK6 according to SEQ ID NO: 82 as shown below using standard three-letter amino acid abbreviations:

Lys₁-Lys₂-Glu₃-Asp₄-Glys-Glu₆-Ser₇-Thr₈-Ala₉-Pro₁₀-
Thr₁₁-Pro₁₂-Arg₁₃-Pro₁₄-Xaa₁₅-Val₁₆-Leu₁₇-Arg₁₈-
Cys₁₉-Xaa₂₀-Cys₂₁-Xaa₂₂-Xaa₂₃-His₂₄-Cys₂₅-Pro₂₆-
Xaa₂₇-Asp₂₈-Xaa₂₉-Xaa₃₀-Asn₃₁-Asn₃₂-Xaa₃₃-Cys₃₄-
Xaa₃₅-Thr₃₆-Xaa₃₇-Gly₃₈-Xaa₃₉-Cys₄₀-Phe₄₁-Xaa₄₂-
Xaa₄₃-Ile₄₄-Glu₄₅-Glu₄₆-Asp₄₇-Asp₄₈-Xaa₄₉-Gly₅₀-
Xaa₅₁-Xaa₅₂-Xaa₅₃-Xaa₅₄-Xaa₅₅-Ser₅₆-Gly₅₇-Cys₅₈-
Xaa₅₉-Xaa₆₀-Xaa₆₁-Glu₆₂-Gly₆₃-Ser₆₄-Asp₆₅-Phe₆₆-
Gln₆₇-Cys₆₈-Xaa₆₉-Asp₇₀-Xaa₇₁-Pro₇₂-Xaa₇₃-Xaa₇₄-
Xaa₇₅-Xaa₇₆-Arg₇₇-Arg₇₈-Xaa₇₉-Ile₈₀-Glu₈₁-Cys₈₂-
Cys₈₃-Xaa₈₄-Xaa₈₅-Xaa₈₆-Xaa₈₇-Xaa₈₈-Cys₈₉-Asn₉₀-
Xaa₉₁-Xaa₉₂-Leu₉₃-Xaa₉₄-Pro₉₅-Thr₉₆-Leu₉₇-Pro₉₈-
Pro₉₉-Leu₁₀₀-Lys₁₀₁-Asn₁₀₂-Arg₁₀₃-Asp₁₀₄-Phe₁₀₅-
Val₁₀₆-Xaa₁₀₇-Gly₁₀₈-Xaa₁₀₉-Ile₁₁₀-Xaa₁₁₁-Xaa₁₁₂
-Arg₁₁₃;

wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is D or A;
Xaa₁₀₉ is P or S;
Xaa₁₁₁ is absent; and,
Xaa₁₁₂ is absent (SEQ ID NO: 82).

In embodiments, the ALK6 ECD polypeptide of the fusion protein according to SEQ ID NO: 82 comprises a substitution selected from K15P, K20Y, K20V, K20F, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 82 comprises one or more substitutions selected from L59M, G60K, and L61Y. In certain embodiments, the ALK6 ECD polypeptide of the fusion protein according to SEQ ID NO: 82 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 82 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 67, 73, 75, and 77.

In embodiments, the soluble ALK-6 polypeptide comprises an active portion (e.g., ligand binding) of ALK6 according to SEQ ID NO: 83 as shown below using standard three-letter amino acid abbreviations:

Lys₁-Lys₂-Glu₃-Asp₄-Gly₅-Glu₆-Ser₇-Thr₈-Ala₉-Pro₁₀-
Thr₁₁-Pro₁₂-Arg₁₃-Pro₁₄-Xaa₁₅-Val₁₆-Leu₁₇-Arg₁₈-
Cys₁₉-Xaa₂₀-Cys₂₁-Xaa₂₂-Xaa₂₃-His₂₄-Cys₂₅-Pro₂₆-
Xaa₂₇-Asp₂₈-Xaa₂₉-Xaa₃₀-Asn₃₁-Asn₃₂-Xaa₃₃-Cys₃₄-
Xaa₃₅-Thr₃₆-Xaa₃₇-Gly₃₈-Xaa₃₉-Cys₄₀-Phe₄₁-Xaa₄₂-
Xaa₄₃-Ile₄₄-Glu₄₅-Glu₄₆-Asp₄₇-Asp₄₈-Xaa₄₉-Gly₅₀-
Xaa₅₁-Xaa₅₂-Xaa₅₃-Xaa₅₄-Xaa₅₅-Ser₅₆-Gly₅₇-Cys₅₈-
Xaa₅₉-Xaa₆₀-Xaa₆₁-Glu₆₂-Gly₆₃-Ser₆₄-Asp₆₅-Phe₆₆-
Gln₆₇-Cys₆₈-Xaa₆₉-Asp₇₀-Xaa₇₁-Pro₇₂-Xaa₇₃-Xaa₇₄-
Xaa₇₅-Xaa₇₆-Arg₇₇-Arg₇₈-Xaa₇₉-Ile₈₀-Glu₈₁-Cys₈₂-
Cys₈₃-Xaa₈₄-Xaa₈₅-Xaa₈₆-Xaa₈₇-Xaa₈₈-Cys₈₉-Asn₉₀-
Xaa₉₁-Xaa₉₂-Leu₉₃-Xaa₉₄-Pro₉₅-Thr₉₆-Leu₉₇-Pro₉₈-
Pro₉₉-Leu₁₀₀-Lys₁₀₁-Asn₁₀₂-Arg₁₀₃-Xaa₁₀₄-Phe₁₀₅-
Val₁₀₆-Xaa₁₀₇-Gly₁₀₈-Xaa₁₀₉-Ile₁₁₀-His₁₁₁-His₁₁₂
-Arg₁₁₃;

wherein Xaa₁₅ is K or P;
Xaa₂₀ is V, F, K or Y;
Xaa₂₂ is G, H or S;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₄ is D or A;
Xaa₁₀₇ is absent or D; and,
Xaa₁₀₉ is P or S. (SEQ ID NO: 83).

In embodiments, the ALK6 ECD polypeptide of the fusion protein according to SEQ ID NO: 83 comprises a substitution selected from K15P, K20Y, K20F, K20V, H22S, H22G, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 83 comprises one or more substitutions selected from L59M, G60K, and L61Y. In certain embodiments, the ALK6 ECD polypeptide of the fusion protein according to SEQ ID NO: 83 comprises one or more substitutions selected from K20Y, H22S and H23G. In certain other embodiments, the ALK6 ECD polypeptide of the fusion protein comprises one or more substitutions selected from K20Y, H22S, H23G and G60K.

In certain embodiments, the ALK6 ECD polypeptide of the fusion protein according to SEQ ID NO: 83 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 12, 14 and 71.

In embodiments, the soluble ALK-6 polypeptide comprises an active portion (e.g., ligand binding) of ALK6 according to SEQ ID NO: 108 as shown below using standard three-letter amino acid abbreviations:

Lys₁-Lys₂-Glu₃-Asp₄-Gly₅-Glu₆-Ser₇-Thr₈-Ala₉-Pro₁₀-
Thr₁₁-Pro₁₂-Arg₁₃-Pro₁₄-Xaa₁₅-Xaa₁₆-Leu₁₇-Arg₁₈-
Cys₁₉-Xaa₂₀-Cys₂₁-Xaa₂₂-Xaa′₂₂-Xaa₂₃-His₂₄-Cys₂₅
-Pro₂₆-Xaa₂₇-Asp₂₈-Xaa₂₉-Xaa₃₀-Asn₃₁-Asn₃₂-Xaa₃₃
-Cys₃₄-Xaa₃₅-Thr₃₆-Xaa₃₇-Gly₃₈-Xaa₃₉-Cys₄₀-Phe₄₁
-Xaa₄₂-Xaa₄₃-Ile₄₄-Glu₄₅-Glu₄₆-Asp₄₇-Asp₄₈-Xaa₄₉
-Gly₅₀-Xaa₅₁-Xaa₅₂-Xaa₅₃-Xaa₅₄-Xaa₅₅-Ser₅₆-Gly₅₇
-Cys₅₈-Xaa₅₉-Xaac₀-Xaa₆₁-Glu₆₂-Gly₆₃-Ser₆₄-Asp₆₅
-Phe₆₆-Gln₆₇-Cys₆₈-Xaa₆₉-Aspr₀-Xaa₇₁-Pro₇₂-Xaa₇₃
-Xaa₇₄-Xaa₇₅-Xaa₇₆-Arg₇₇-Arg₇₈-Xaa₇₉-Ile₈₀-Glu₈₁
-Cys₈₂-Cys₈₃-Xaa₈₄-Xaa₈₅-Xaa₈₆-Xaa₈₇-Xaa₈₈-Cys₈₉
-Asn₉₀-Xaa₉₁-Xaa₉₂-Leu₉₃-Xaa₉₄-Pro₉₅-Thr₉₆-Leu₉₇
-Pro₉₈-Pro₉₉-Leu₁₀₀-Lys₁₀₁-Asn₁₀₂-Arg₁₀₃-Asp₁₀₄-
Phe₁₀₅-Val₁₀₆-Xaa₁₀₇-Xaa₁₀₈-Xaa₁₀₉-Xaa₁₁₀-Xaa₁₁₁
-Xaa₁₁₂-Xaa₁₁₃;

wherein:
Xaa₁₅ is K or P;
Xaa₁₆ is V or I;
Xaa₂₀ is V, F, K or Y;
Xaa₂₂ is G, H or S;
Xaa′₂₂ is absent or A;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is absent;
Xaa₁₀₈ is absent;
Xaa₁₀₉ is absent;
Xaa₁₁₀ is absent;
Xaa₁₁₁ is absent;
Xaa₁₁₂ is absent; and,
Xaa₁₁₃ is absent (SEQ ID NO: 108)

In embodiments, the ALK6 ECD polypeptide of the fusion protein according to SEQ ID NO: 108 comprises a substitution selected from K15P, V16I, K20Y, K20V, K20F, H22S, H22G, _22′A, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position. In certain embodiments, the ALK6 ECD polypeptide of the fusion protein according to SEQ ID NO: 108 comprises one or more substitutions selected from L59M, G60K, and L61Y. In certain embodiments, the ALK6 ECD polypeptide of the fusion protein according to SEQ ID NO: 108 comprises one or more substitutions selected from K20Y, H22S and H23G. In certain other embodiments, the ALK6 ECD polypeptide of the fusion protein comprises one or more substitutions selected from K20Y, H22S, H23G and G60K. In certain embodiments, the ALK6 ECD polypeptide of the fusion protein according to SEQ ID NO: 108 comprises one or more substitutions selected from K20Y, H22S, _22′A and H23G. In certain embodiments, the ALK6 ECD polypeptide of the fusion protein according to SEQ ID NO: 108 comprises one or more substitutions selected from K20Y, H22G, _22′A and H23S.

In certain embodiments, the ALK6 ECD polypeptide of the fusion protein according to SEQ ID NO: 108 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 13, 86, 90, 92, 94, 96, 98, 100, 102, 104, and 110.

In embodiments, the soluble ALK-6 polypeptide comprises an active portion (e.g., ligand binding) of ALK6 according to SEQ ID NO: 109 as shown below using standard three-letter amino acid abbreviations:

Lys₁-Lys₂-Glu₃-Asp₄-Gly₅-Glu₆-Ser₇-Thr₈-Ala₉-Pro₁₀-
Thr₁₁-Pro₁₂-Arg₁₃-Pro₁₄-Xaa₁₅-Xaa₁₆-Leu₁₇-Arg₁₈-
Cys₁₉-Xaa₂₀-Cys₂₁-Xaa₂₂-Xaa′₂₂-Xaa₂₃-His₂₄-Cys₂₅
-Pro₂₆-Xaa₂₇-Asp₂₈-Xaa₂₉-Xaa₃₀-Asn₃₁-Asn₃₂-Xaa₃₃
-Cys₃₄-Xaa₃₅-Thr₃₆-Xaa₃₇-Gly₃₈-Xaa₃₉-Cys₄₀-Phe₄₁
-Xaa₄₂-Xaa₄₃-Ile₄₄-Glu₄₅-Glu₄₆-Asp₄₇-Asp₄₈-Xaa₄₉
-Gly₅₀-Xaa₅₁-Xaa₅₂-Xaa₅₃-Xaa₅₄-Xaa₅₅-Ser₅₆-Gly₅₇
-Cys₅₈-Xaa₅₉-Xaa₆₀-Xaa₆₁-Glu₆₂-Gly₆₃-Ser₆₄-Asp₆₅
-Phe₆₆-Gln₆₇-Cys₆₈-Xaa₆₉-Asp₇₀-Xaa₇₁-Pro₇₂-Xaa₇₃
-Xaa₇₄-Xaa₇₅-Xaa₇₆-Arg₇₇-Arg₇₈-Xaa₇₉-Ile₈₀-Glu₈₁
-Cys₈₂-Cys₈₃-Xaa₈₄-Xaa₈₅-Xaa₈₆-Xaa₈₇-Xaa₈₈-Cys₈₉
-Asn₉₀-Xaa₉₁-Xaa₉₂-Leu₉₃-Xaa₉₄-Pro₉₅-Thr₉₆-Leu₉₇
-Pro₉₈-Pro₉₉-Val₁₀₀-Val₁₀₁-Ile₁₀₂-Gly₁₀₃-Pro₁₀₄-
Phe₁₀₅-Phe₁₀₆-Asp₁₀₇-Gly₁₀₈-Ser₁₀₉-Ile₁₁₀-Arg₁₁₁
;

wherein Xaa₁₅ is K or P;
Xaa₁₆ is V or I;
Xaa₂₀ is V, F, K or Y;
Xaa₂₂ is G, H or S;
Xaa′₂₂ is absent or A;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L; and,
Xaa₉₄ is H or Q. (SEQ ID NO: 109)

In embodiments, the ALK6 ECD polypeptide of the fusion protein according to SEQ ID NO: 109 comprises a substitution selected from K15P, V16I, K20Y, K20V, K20F, H22S, H22G, _22′A, H23G, H23S, E27D, S29A, V30I, I33T, S35I, D37N, Y39H, T42A, M43I, S49Q, L51E, P52, T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, D107, or a combination thereof, wherein _ is a deletion at that position. In certain embodiments, the ALK6 ECD polypeptide of the fusion protein according to SEQ ID NO: 109 comprises one or more substitutions selected from L59M, G60K, and L61Y. In certain embodiments, the ALK6 ECD polypeptide of the fusion protein according to SEQ ID NO: 109 comprises one or more substitutions selected from K20Y, H22S and H23G. In certain other embodiments, the ALK6 ECD polypeptide of the fusion protein comprises one or more substitutions selected from K20Y, H22S, H23G and G60K. In certain embodiments, the ALK6 ECD polypeptide of the fusion protein according to SEQ ID NO: 109 comprises one or more substitutions selected from K20Y, H22S, _22′A and H23G. In certain embodiments, the ALK6 ECD polypeptide of the fusion protein according to SEQ ID NO: 109 comprises one or more substitutions selected from K20Y, H22G, _22′A and H23S.

In certain embodiments, the ALK6 ECD polypeptide of the fusion protein according to SEQ ID NO: 109 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 15-45, 57, 59, 61, 63, 65, and 88.

In embodiments, the soluble ALK-6 polypeptide comprises an active portion (e.g., ligand binding) of ALK6 according to SEQ ID NO: 130 as shown below using standard three-letter amino acid abbreviations:

Lys₁-Lys₂-Glu₃-Asp₄-Gly₅-Glu₆-Ser₇-Thr₈-Ala₉-Pro₁₀-
Thr₁₁-Pro₁₂-Arg₁₃-Pro₁₄-Lys₁₅-Xaa₁₆-Leu₁₇-Arg₁₈-
Cys₁₉-Xaa₂₀-Cys₂₁-Xaa₂₂-Xaa′₂₂-Xaa″₂₂-Xaa₂₃-Xaa′₂₃-
Xaa₂₄-Cys₂₅-Pro₂₆-Xaa₂₇-Asp₂₈-Xaa₂₉-Xaa₃₀-Asn₃₁-
Asn₃₂-Xaa₃₃-Cys₃₄-Xaa₃₅-Thr₃₆-Xaa₃₇-Gly₃₈-Xaa₃₉-
Cys₄₀-Phe₄₁-Xaa₄₂-Xaa₄₃-Ile₄₄-Glu₄₅-Glu₄₆-Asp₄₇-
Asp₄₈-Xaa₄₉-Gly₅₀-Xaa₅₁-Xaa₅₂-Xaa₅₃-Xaa₅₄-Xaa₅₅-
Ser₅₆-Gly₅₇-Cys₅₈-Xaa₅₉-Xaa₆₀-Xaa₆₁-Glu₆₂-Gly₆₃-
Ser₆₄-Asp₆₅-Phe₆₆-Gln₆₇-Cys₆₈-Xaa₆₉-Asp₇₀-Xaa₇₁-
Pro₇₂-Xaa₇₃-Xaa₇₄-Xaa₇₅-Xaa₇₆-Arg₇₇-Arg₇₈-Xaa₇₉-
Ile₈₀-Glu₈₁-Cys₈₂-Cys₈₃-Xaa₈₄-Xaa₈₅-Xaa₈₆-Xaa₈₇-
Xaa₈₈-Cys₈₉-Asn₉₀-Xaa₉₁-Xaa₉₂-Leu₉₃-Xaa₉₄-Pro₉₅-
Thr₉₆-Leu₉₇-Pro₉₈-Pro₉₉-Leu₁₀₀-Lys₁₀₁-Asn₁₀₂-
Arg₁₀₃-Asp₁₀₄-Phe₁₀₅-Val₁₀₆-Xaa₁₀₇-Xaa₁₀₈-
Xaa₁₀₉-Xaa₁₁₀-Xaa₁₁₁-Xaa₁₁₂-Xaa₁₁₃;

wherein Xaa₁₆ is V or I;
Xaa₂₀ is V, F, K, Y, T, or A;
Xaa₂₂ is G, E, H or S;
Xaa′₂₂ is absent, G, S, or A;
Xaa″₂₂ is absent or S;
Xaa₂₃ is S, H, G, or L;
Xaa₂₃′ is absent, L or H;
Xaa₂₄ is H or S;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is absent;
Xaa₁₀₈ is absent;
Xaa₁₀₉ is absent;
Xaa₁₁₀ is absent;
Xaa₁₁₁ is absent;
Xaa₁₁₂ is absent; and,
Xaa₁₁₃ is absent. (SEQ ID NO: 130)

In embodiments, the ALK6 ECD polypeptide of the fusion protein according to SEQ ID NO: 130 comprises a substitution selected from K15P, V16I, K20Y, K20V, K20F, K20A, K20A, H22S, H22G, H22E, H22S, _22′A, _22′_, _22′G, _22′S, _22″_, _22″S, H23G, H23S, H24S, E27D, S29A, V30I, I33T, I33S, S35I, D37N, Y39H, T42A, M43I, S49Q, L51E, P52T, V53T, T55A, L59M, G60K, L61Y, R69K, T71S, I73K, P74A, H75Q, Q76L, S79T, T84R, E85T, E85_, R86N, N87L, E88_, K91Q, D92L, H94Q, D104A, or a combination thereof, wherein _ is a deletion at that position. In certain embodiments, the ALK6 ECD polypeptide of the fusion protein according to SEQ ID NO: 130 comprises one or more substitutions selected from L59M, G60K, and L61Y. In certain embodiments, the ALK6 ECD polypeptide of the fusion protein according to SEQ ID NO: 130 comprises one or more substitutions selected from K20Y, H22S and H23G. In certain other embodiments, the ALK6 ECD polypeptide of the fusion protein comprises one or more substitutions selected from K20Y, H22S, H23G and G60K. In certain embodiments, the ALK6 ECD polypeptide of the fusion protein according to SEQ ID NO: 130 comprises one or more substitutions selected from K20Y, K20V, K20F, K20A, K20A, H22S, _22′A, _22′_, _22′G, _22′S, _22″_, _22″S and H23G. In certain embodiments, the ALK6 ECD polypeptide of the fusion protein according to SEQ ID NO: 130 comprises one or more substitutions selected from K20Y, H22G, _22′A and H23S.

In certain embodiments, the ALK6 ECD polypeptide of the fusion protein according to SEQ ID NO: 130 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 150, 151 or 152.

In embodiments, the soluble ALK-6 polypeptide comprises an active portion (e.g., ligand binding) of ALK6 according to SEQ ID NO: 157 as shown below using standard three-letter amino acid abbreviations:

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃-Xaa₁₄-Xaa₁₅-Xaa₁₆-Leu₁₇-
Arg₁₈-Cys₁₉-Xaa₂₀-Cys₂₁-Xaa₂₂-Xaa′₂₂-Xaa″₂₂-
Xaa₂₃-Xaa′₂₃-Xaa₂₄-Cys₂₅-Pro₂₆-Xaa₂₇-Asp₂₈-Xaa₂₉-
Xaa₃₀-Asn₃₁-Asn₃₂-Xaa₃₃-Cys₃₄-Xaa₃₅-Thr₃₆-Xaa₃₇-
Gly₃₈-Xaa₃₉-Cys₄₀-Phe₄₁-Xaa₄₂-Xaa₄₃-Ile₄₄-Glu₄₅-
Glu₄₆-Asp₄₇-Asp₄₈-Xaa₄₉-Gly₅₀-Xaa₅₁-Xaa₅₂-Xaa₅₃-
Xaa₅₄-Xaa₅₅-Ser₅₆-Gly₅₇-Cys₅₈-Xaa₅₉-Xaa₆₀-Xaa₆₁-
Glu₆₂-Gly₆₃-Ser₆₄-Asp₆₅-Phe₆₆-Gln₆₇-Cys₆₈-Xaa₆₉-
Asp₇₀-Xaa₇₁-Pro₇₂-Xaa₇₃-Xaa₇₄-Xaa₇₅-Xaa₇₆-Arg₇₇-
Arg₇₈-Xaa₇₉-Ile₈₀-Glu₈₁-Cys₈₂-Cys₈₃-Xaa₈₄-Xaa₈₅-
Xaa₈₆-Xaa₈₇-Xaa₈₈-Cys₈₉-Asn₉₀-Xaa₉₁-Xaa₉₂-Leu₉₃-
Xaa₉₄-Pro₉₅-Thr₉₆-Leu₉₇-Pro₉₈-Pro₉₉-Xaa₁₀₀-Xaa₁₀₁-
Xaa₁₀₂-Xaa₁₀₃-Xaa₁₀₄-Xaa₁₀₅-Xaa₁₀₆-Xaa₁₀₇-Xaa₁₀₈-
Xaa₁₀₉-Xaa₁₁₀-Xaa₁₁₁-Xaa₁₁₂-Xaa₁₁₃

wherein: Xaa₁ is K or absent;
Xaa₂ is K or absent;
Xaa₃ is E or absent;
Xaa₄ is D or absent;
Xaa₅ is G or absent,
Xaa₆ is E or absent;
Xaa₇ is S or absent;
Xaa₈ is T or absent;
Xaa₉ is A or absent;
Xaa₁₀ is P or absent;
Xaa₁₁ is T or absent;
Xaa₁₂ is P or absent;
Xaa₁₃ is R or absent;
Xaa₁₄ is P or absent,
Xaa₁₅ is K, P or absent;
Xaa₁₆ is V or absence;
Xaa₂₀ is V, F, K, A, R, N, D, C, E, Q, G, H, I, L, M, P, S, T, W, or Y;
Xaa₂₂ is G, H, V, F, K, A, R, N, D, C, E, Q, I, L, M, P, T, W, Y or S;
Xaa′₂₂ is absent, G, S, or A;
Xaa″₂₂ is absent or S;
Xaa₂₃ is S, H, V, F, K, A, R, N, D, C, E, Q, I, L, M, P, T, W, Y or G;
Xaa′₂₃ is absent, L or H;
Xaa₂₄ is V, F, K, A, R, N, D, C, E, Q, G, H, I, L, M, P, S, T, W, or Y;
Xaa₂₇ is E or D;
Xaa₂₉ is S, G or A;
Xaa₃₀ is V, A, G or I;
Xaa₃₃ is I, S or T;
Xaa₃₅ is S, T, G, A or I;
Xaa₃₇ is D, A, G or N;
Xaa₃₉ is Y, A, G or H;
Xaa₄₂ is T or A;
Xaa₄₃ is M or I;
Xaa₄₉ is S, A, G or Q;
Xaa₅₁ is L, A, G or E;
Xaa₅₂ is P or T;
Xaa₅₃ is V, A, G or T;
Xaa₅₄ is V, A, G or L;
Xaa₅₅ is T, G or A;
Xaa₅₉ is L, M, V, F, K, A, R, N, D, C, E, Q, G, H, I, P, S, T, W, or Y;
Xaa₆₀ is G, K, V, F, A, R, N, D, C, E, Q, H, I, L, M, P, S, T, W, or Y;
Xaa₆₁ is L, Y, V, F, K, A, R, N, D, C, E, Q, G, H, I, M, P, S, T, or W;
Xaa₆₉ is R or K;
Xaa₇₁ is T or S;
Xaa₇₃ is I or K;
Xaa₇₄ is P or A;
Xaa₇s is H or Q;
Xaa₇₆ is Q or L;
Xaa₇₉ is S or T;
Xaa₈₄ is T, R or absent;
Xaa₈₅ is absent, T or E;
Xaa₈₆ is R, N or absent;
Xaa₈₇ is N, L or absent;
Xaa₈₈ is absent, E or L;
Xaa₉₁ is K, Q or absent;
Xaa₉₂ is D, L, Y or absent;
Xaa₉₄ is H, Q or absent;
Xaa₁₀₀ is absent, V or L;
Xaa₁₀₁ is absent, V or K;
Xaa₁₀₂ is absent, I or N;
Xaa₁₀₃ is absent, G or R;
Xaa₁₀₄ is absent, P, D or A;
Xaa₁₀₅ is absent or F;
Xaa₁₀₆ is absent, F, W, Y, N, Q, K, H, D, G, E or V;
Xaa₁₀₇ is absent, L, M, V, F, K, A, R, N, C, E, Q, G, H, I, P, S, T, W, or Y or D;
Xaa₁₀₈ is absent, R, T, Q, L, K, W or G;
Xaa₁₀₉ is absent, S or P;
Xaa₁₁₀ is absent or I;
Xaa₁₁₁ is absent, R or H;
Xaa₁₁₂ is absent or H; and
Xaa₁₁₃ is absent or R.

In embodiments, the ALK6 ECD polypeptide of the fusion protein according to SEQ ID NO: 157 comprises a substitution selected from K1_, K2_, E3_, D4_, G5_, E6_, S7_, T8_, A9_, P10_, T11, P12_, R13_, P14_, K15P, K15_, V16_, K20Y, K20V, K20F, K20A, K20R, K20N, K20D, K20C, K20E, K20Q, K20G, K20H, K20I, K20L, K20M, K20P, K20S, K20T, K20W, H22S, H22G, H22E, H22V, H22F, H22K, H22A, H22R, H22N, H22D, H22C, H22Q, H22I, H22L, H22M, H22P, H22T, H22W, H22Y, _22′A, _22′_, _22′G, _22′S, _22″_, _22″S, H23G, H23S, H23V, H23F, H23K, H23A, H23R, H23N, H23D, H23C, H23E, H23Q, H23I, H23L, H23M, H23P, H23T, H23W, H23Y, H23G, 23′_, _23′L, 23′H, H24S, H24V, H24F, H24K, H24A, H24R, H24N, H24D, H24C, H24E, H24Q, H24G, H24I, H24L, H24M, H24P, H24T, H24W, H24Y, E27D, S29A, S29G, V30I, V30A, V30G, I33T, I33S, S35I, S35A, S35G, S35T, D37N, D37A, D37G, Y39H, Y39A, Y39G, T42A, M43I, S49Q, S49A, S49G, L51E, L51A, L51G, P52T, V53T, V53A, V53G, V54A, V54G, V54L, T55A, T55G, L59M, L59V, L59F, L59K, L59A, L59R, L59N, L59D, L59C, L59E, L59Q, L59G, L59H, L59I, L59P, L59S, L59T, L59W, L59Y, G60K, G60V, G60F, G60A, G60R, G60N, G60D, G60C, G60E, G60Q, G60H, G60I, G60L, G60M, G60P, G60S, G60T, G60W, G60Y, L61Y, L61V, L61F, L61K, L61A, L61R, L61N, L61D, L61C, L61E, L61Q, L61G, L61H, L61I, L61M, L61P, L61S, L61T, L61W, R69K, T71S, I73K, P74A, H75Q, Q76L, S79T, T84R, T84_ E85T, E85_, R86N, R86_, N87L, N87_, E88_, K91Q, K91_, D92L, D92_, D92Y, H94Q, H94_, D104A, V106F, V106W, V106Y, V106N, V106Q, V106K, V106H, V106D, V106G, V106E, D107L, D107M, D107V, D107F, D107K, D107A, D107R, D107N, D107C, D107E, D107Q, D107G, D107H, D107I, D107P, D107S, D107T, D107W, D107Y, G108R, G108T, G108Q, G108L, G108K, G108W, or a combination thereof, wherein _ is a deletion at that position. In certain embodiments, the ALK6 ECD polypeptide of the fusion protein according to SEQ ID NO: 157 comprises one or more substitutions selected from L59M, G60K, and L61Y. In certain embodiments, the ALK6 ECD polypeptide of the fusion protein according to SEQ ID NO: 157 comprises one or more substitutions selected from K20Y, H22S and H23G. In certain other embodiments, the ALK6 ECD polypeptide of the fusion protein comprises one or more substitutions selected from K20Y, H22S, and H23G wherein position 60 (Xaa₆₀) is glycine. In certain embodiments, the ALK6 ECD polypeptide of the fusion protein according to SEQ ID NO: 157 comprises one or more substitutions selected from K20Y, H22S, _22′A and H23G. In certain embodiments, the ALK6 ECD polypeptide of the fusion protein according to SEQ ID NO: 157 comprises one or more substitutions selected from K20Y, H22G, _22′A and H23S.

In certain embodiments, the ALK6 ECD polypeptide of the fusion protein according to SEQ ID NO: 157 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 132 to 155.

In embodiments, the soluble ALK-6 polypeptide comprises an active portion (e.g., ligand binding) of ALK6 according to SEQ ID NO: 190 as shown below using standard three-letter amino acid abbreviations:

Lys₁-Lys₂-Glu₃-Asp₄-Gly₅-Glu₆-Ser₇-Thr₈-Ala₉-
Pro₁₀-Thr₁₁-Pro₁₂-Arg₁₃-Pro₁₄-Xaa₁₅-Xaa₁₆-Leu₁₇-
Arg₁₈-Cys₁₉-Xaa₂₀-Cys₂₁-Xaa₂₂-Xaa′₂₂-Xaa″₂₂-Xaa₂₃-
Xaa′₂₃-Xaa₂₄-Cys₂₅-Pro₂₆-Xaa₂₇-Asp₂₈-Xaa₂₉-Xaa₃₀-
Asn₃₁-Asn₃₂-Xaa₃₃-Cys₃₄-Xaa₃₅-Thr₃₆-Xaa₃₇-Gly₃₈-
Xaa₃₉-Cys₄₀-Phe₄₁-Xaa₄₂-Xaa₄₃-Ile₄₄-Glu₄₅-Glu₄₆-
Asp₄₇-Asp₄₈-Xaa₄₉-Gly₅₀-Xaa₅₁-Xaa₅₂-Xaa₅₃-Xaa₅₄-
Xaa₅₅-Ser₅₆-Gly₅₇-Cys₅₈-Xaa₅₉-Xaa₆₀-Xaa₆₁-Glu₆₂-
Gly₆₃-Ser₆₄-Asp₆₅-Phe₆₆-Gln₆₇-Cys₆₈-Xaa₆₉-Asp₇₀-
Xaa₇₁-Pro₇₂-Xaa₇₃-Xaa₇₄-Xaa₇₅-Xaa₇₆-Arg₇₇-Arg₇₈-
Xaa₇₉-Ile₈₀-Glu₈₁-Cys₈₂-Cys₈₃-Xaa₈₄-Xaa₈₅-Xaa₈₆-
Xaa₈₇-Xaa₈₈-Cys₈₉-Asn₉₀-Xaa₉₁-Xaa₉₂-Leu₉₃-Xaa₉₄-
Pro₉₅-Thr₉₆-Leu₉₇-Pro₉₈-Pro₉₉-Leu₁₀₀-Lys₁₀₁-
Asn₁₀₂-Arg₁₀₃-Xaa₁₀₄-Phe₁₀₅-Val₁₀₆-Xaa₁₀₇-Xaa₁₀₈-
Xaa₁₀₉-Xaa₁₁₀-Xaa₁₁₁-Xaa₁₁₂-Xaa₁₁₃;

wherein Xaa₁₅ is K or P;
Xaa₁₆ is V or I;
Xaa₂₀ is V, F, K, A, R, N, D, C, E, Q, G, H, I, L, M, P, S, T, W, or Y;
Xaa₂₂ is G, H, V, F, K, A, R, N, D, C, E, Q, I, L, M, P, T, W, Y or S;
Xaa′₂₂ is absent, G, S, or A;
Xaa″₂₂ is absent or S;
Xaa₂₃ is S, H, V, F, K, A, R, N, D, C, E, Q, I, L, M, P, T, W, Y or G;
Xaa′₂₃ is absent, L or H;
Xaa₂₄ is V, F, K, A, R, N, D, C, E, Q, G, H, I, L, M, P, S, T, W, or Y;
Xaa₂₇ is E or D;
Xaa₂₉ is S, G or A;
Xaa₃₀ is V, A, G or I;
Xaa₃₃ is I, S or T;
Xaa₃₅ is S, T, G, A or I;
Xaa₃₇ is D, A, G or N;
Xaa₃₉ is Y, A, G or H;
Xaa₄₂ is T or A;
Xaa₄₃ is M or I;
Xaa₄₉ is S, A, G or Q;
Xaa₅₁ is L, A, G or E;
Xaa₅₂ is P or T;
Xaa₅₃ is V, A, G or T;
Xaa₅₄ is V, A, G or L;
Xaa₅₅ is T, G or A;
Xaa₅₉ is L, M, V, F, K, A, R, N, D, C, E, Q, G, H, I, P, S, T, W, or Y;
Xaa₆₀ is G, K, V, F, A, R, N, D, C, E, Q, H, I, L, M, P, S, T, W, or Y; Xaa₆₁ is L, Y, V, F, K, A, R, N, D, C, E, Q, G, H, I, M, P, S, T, or W;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is absent, T or R;
Xaa₈₅ is absent, T or E;
Xaa₈₆ is absent, R or N;
Xaa₈₇ is N or L;
Xaa₈₈ is absent, E or L;
Xaa₉₁ is absent, K or Q;
Xaa₉₂ is absent, D, Y or L;
Xaa₉₄ is absent, H or Q;
Xaa₁₀₄ is D or A;
Xaa₁₀₇ is absent;
Xaa₁₀₈ is absent;
Xaa₁₀₉ is absent;
Xaa₁₁₀ is absent;
Xaa₁₁ is absent;
Xaa₁₁₂ is absent; and,
Xaa₁₁₃ is absent.

In embodiments, the ALK6 ECD polypeptide of the fusion protein according to SEQ ID NO: 190 comprises a substitution selected from K15P, V16I, K20Y, K20V, K20F, K20A, K20R, K20N, K20D, K20C, K20E, K20Q, K20G, K20H, K20I, K20L, K20M, K20P, K20S, K20T, K20W, H22S, H22G, H22E, H22V, H22F, H22K, H22A, H22R, H22N, H22D, H22C, H22Q, H22I, H22L, H22M, H22P, H22T, H22W, H22Y, _22′A, _22′_, _22′G, _22′S, _22″_, _22″S, H23G, H23S, H23V, H23F, H23K, H23A, H23R, H23N, H23D, H23C, H23E, H23Q, H23I, H23L, H23M, H23P, H23T, H23W, H23Y, _23′_, _23′L, _23′H, H24S, H24V, H24F, H24K, H24A, H24R, H24N, H24D, H24C, H24E, H24Q, H24G, H24I, H24L, H24M, H24P, H24T, H24W, H24Y, E27D, S29A, S29G, V30I, V30A, V30G, I33T, I33S, S35I, S35A, S35G, S35T, D37N, D37A, D37G, Y39H, Y39Q, Y39A, T42A, M43I, S49Q, S49A, S49G, L51E, L51A, L51G, P52T, V53T, V53A, V53G, V54A, V54G, V54L, T55A, T55G, L59M, L59V, L59F, L59K, L59A, L59R, L59N, L59D, L59C, L59E, L59Q, L59G, L59H, L59I, L59P, L59S, L59T, L59W, L59Y, G60K, G60V, G60F, G60A, G60R, G60N, G60D, G60C, G60E, G60Q, G60H, G60I, G60L, G60M, G60P, G60S, G60T, G60W, G60Y, L61Y, L61V, L61F, L61K, L61A, L61R, L61N, L61D, L61C, L61E, L61Q, L61G, L61H, L61I, L61M, L61P, L61S, L61T, L61W, R69K, T71S, I73K, P74A, H75Q, Q76L, S79T, T84R, T84_ E85T, E85_, R86N, R86_, N87L, N87_, E88_, K91Q, K91_, D92L, D92_, D92Y, H94Q, H94_, D104A, or a combination thereof, wherein _ is a deletion at that position. In certain embodiments, the ALK6 ECD polypeptide of the fusion protein according to SEQ ID NO: 190 comprises one or more substitutions selected from L59M, G60K, and L61Y. In certain embodiments, the ALK6 ECD polypeptide of the fusion protein according to SEQ ID NO: 190 comprises one or more substitutions selected from K20Y, H22S and H23G. In certain other embodiments, the ALK6 ECD polypeptide of the fusion protein comprises one or more substitutions selected from K20Y, H22S, and H23G wherein position 60 (Xaa₆₀) is glycine. In certain embodiments, the ALK6 ECD polypeptide of the fusion protein according to SEQ ID NO: 190 comprises one or more substitutions selected from K20Y, H22S, _22′A and H23G. In certain embodiments, the ALK6 ECD polypeptide of the fusion protein according to SEQ ID NO: 190 comprises one or more substitutions selected from K20Y, H22G, _22′A and H23S.

In certain embodiments, the ALK6 ECD polypeptide of the fusion protein according to SEQ ID NO: 190 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 158 to 189.

In embodiments, the soluble ALK-6 polypeptide comprises an active portion (e.g., ligand binding) of ALK6 according to SEQ ID NO: 437 as shown below using standard three-letter amino acid abbreviations:

Lys₁-Lys₂-Glu₃-Asp₄-Gly₅-Glu₆-Ser₇-Thr₈-Ala₉-
Pro₁₀-Thr₁₁-Pro₁₂-Arg₁₃-Pro₁₄-Xaa₁₅-Xaa₁₆-Leu₁₇-
Arg₁₈-Cys₁₉-Xaa₂₀-Cys₂₁-Xaa₂₂-Xaa′₂₂-Xaa23-His₂₄-
Cys₂₅-Pro₂₆-Xaa₂₇-Asp₂₈-Xaa₂₉-Xaa₃₀-Asn₃₁-Asn₃₂-
Xaa₃₃-Cys₃₄-Xaa₃₅-Thr₃₆-Xaa₃₇-Gly₃₈-Xaa₃₉-Cys₄₀-
Phe₄₁-Xaa₄₂-Xaa₄₃-Ile₄₄-Glu₄₅-Glu₄₆-Asp₄₇-Asp₄₈-
Xaa₄₉-Gly₅₀-Xaa₅₁-Xaa₅₂-Xaa₅₃-Xaa₅₄-Xaa₅₅-Ser₅₆-
Gly₅₇-Cys₅₈-Xaa₅₉-Xaa₆₀-Xaa₆₁-Glu₆₂-Gly₆₃-Ser₆₄-
Asp₆₅-Phe₆₆-Gln₆₇-Cys₆₈-Xaa₆₉-Asp₇₀-Xaa₇₁-Pro₇₂-
Xaa₇₃-Xaa₇₄-Xaa₇₅-Xaa₇₆-Arg₇₇-Arg₇₈-Xaa₇₉-Ile₈₀-
Glu₈₁-Cys₈₂-Cys₈₃-Xaa₈₄-Xaa₈₅-Xaa₈₆-Xaa₈₇-Xaa₈₈-
Xaa₈₉-Xaa₉₀-Xaa₉₁-Xaa₉₂-Xaa₉₃-Xaa₉₄-Xaa₉₅-Xaa₉₆-
Xaa₉₇-Xaa₉₈-Xaa₉₉-Xaa₁₀₀-Xaa₁₀₁-Xaa₁₀₂-Xaa₁₀₃-
Xaa₁₀₄-Xaa₁₀₅-Xaa₁₀₆-Xaa₁₀₇-Xaa₁₀₈-Xaa₁₀₉-Xaa₁₁₀-
Xaa₁₁₁-Xaa₁₁₂-Xaa₁₁₃;

wherein: Xaa₁₅ is K or P;
Xaa₁₆ is V or I;
Xaa₂₀ is V, F, K or Y;
Xaa₂₂ is G, H or S;
Xaa′₂₂ is absent, G, S or A;
Xaa₂₃ is S, H or G;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is absent, N or L;
Xaa₈₈ is absent, E or L;
Xaa₈₉ is absent or C;
Xaa₉₀ is absent or N;
Xaa₉₁ is absent, K or Q;
Xaa₉₂ is absent, D or L;
Xaa₉₃ is absent or L;
Xaa₉₄ is absent, H or Q;
Xaa₉₅ is absent or P;
Xaa₉₆ is absent or T;
Xaa₉₇ is absent or L;
Xaa₉₈ is absent or P;
Xaa₉₉ is absent or P;
Xaa₁₀₀ is absent, V or L;
Xaa₁₀₁ is absent, V or K;
Xaa₁₀₂ is absent, I or N;
Xaa₁₀₃ is absent, G or R;
Xaa₁₀₄ is absent, P, D or A;
Xaa₁₀₅ is absent or F;
Xaa₁₀₆ is absent, F or V;
Xaa₁₀₇ is absent or D;
Xaa₁₀₈ is absent or G;
Xaa₁₀₉ is absent, S or P;
Xaa₁₁₀ is absent or I;
Xaa₁₁₁ is absent, R or H;
Xaa₁₁₂ is absent or H; and,
Xaa₁₁₃ is absent or R.

In embodiments, the ALK6 ECD polypeptide of the fusion protein according to SEQ ID NO: 437 comprises a substitution selected from V16I, K20Y, H22S, _22′A, H23G, E27D, V30I, I33T, S35I, D37N, Y39H, T42A, M43I, L51M, T55A, L59M, G60K, L61Y, R68K, T71S, I73K, P74A, H75Q, Q76L, S78T or a combination thereof, wherein _ is a deletion at that position.

In certain embodiments, the ALK6 ECD polypeptide of the fusion protein according to SEQ ID NO: 437 comprises one or more substitutions selected from K20Y, H22S, _22′A, and H23G. In certain embodiments, the ALK6 ECD polypeptide of the fusion protein according to SEQ ID NO: 437 comprises one or more substitutions selected from V16I, K20Y, H22S, _22′A, H23G and L51M. In certain other embodiments, the ALK6 ECD polypeptide of the fusion protein comprises one or more substitutions selected from V16I, K20Y, H22S, _22′A, H23G and L51M wherein position 60 (Xaa₆₀) is glycine.

In certain embodiments, the ALK6 ECD polypeptide of the fusion protein according to SEQ ID NO: 437 comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 90, 286, 292, 298, 304, 310, 316, 322, 328, 334 340, 346, 352, 358, 364, 370, 376, 382, 388, 394, 400, 406, 412, 418, 425 and 431.

In certain embodiments, the soluble ALK-6 polypeptide comprises an active portion (e.g., ligand binding) of SEQ ID NO: 3 (“SEQ ID NO: 1 variants”). In certain embodiments, the soluble ALK-6 polypeptide comprises an active portion (e.g., ligand binding) comprises an ECD sequence of ALK6 selected from exemplified SEQ ID NO: 1, 4, 11-45, 57, 59, 61, 63, 65, 67, 69, 71, 73, 75, 77, 80, 86, 88, 92, 94, 96, 98, and 100.

In certain embodiments, the soluble ALK-6 polypeptide comprises an active portion (e.g., ligand binding) of SEQ ID NO: 108 (“SEQ ID NO: 113 variants”). In certain embodiments, the soluble ALK-6 polypeptide comprises an active portion (e.g., ligand binding) comprises an ECD sequence of ALK6 selected from exemplified SEQ ID NO: 90, 102, 104, and 110.

In certain embodiments, the soluble ALK-6 polypeptide comprises an active portion (e.g., ligand binding) of SEQ ID NO: 83 (“SEQ ID NO:1 variants”). In certain embodiments, the soluble ALK-6 polypeptide comprises an active portion (e.g., ligand binding) comprises an ECD sequence of ALK6 selected from exemplified SEQ ID NO: 12; SEQ ID NO: 14; and SEQ ID NO: 71.

In certain embodiments, the soluble ALK6 polypeptide comprises an ECD sequence of ALK6 according to SEQ ID NO: 12. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 12.

In certain embodiments, the soluble ALK6 polypeptide comprises an ECD sequence of ALK6 according to SEQ ID NO: 14. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 14.

In certain embodiments, the soluble ALK6 polypeptide comprises an ECD sequence of ALK6 according to SEQ ID NO: 71. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 71.

In certain embodiments, the soluble ALK-6 polypeptide comprises an active portion (e.g., ligand binding) of SEQ ID NO: 79 (“SEQ ID NO:11 variants”). In certain embodiments, the soluble ALK-6 polypeptide comprises an active portion (e.g., ligand binding) comprises an ECD sequence of ALK6 selected from exemplified SEQ ID NOs: 15-45, 57, 59, 61, 63, 65 and 88.

In certain embodiments, the soluble ALK6 polypeptide comprises an ECD sequence of ALK6 according to SEQ ID NO: 15. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 15.

In certain embodiments, the soluble ALK6 polypeptide comprises an ECD sequence of ALK6 according to SEQ ID NO: 16. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 16.

In certain embodiments, the soluble ALK6 polypeptide comprises an ECD sequence of ALK6 according to SEQ ID NO: 17. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 17.

In certain embodiments, the soluble ALK6 polypeptide comprises an ECD sequence of ALK6 according to SEQ ID NO: 18. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 18.

In certain embodiments, the soluble ALK6 polypeptide comprises an ECD sequence of ALK6 according to SEQ ID NO: 19. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 19.

In certain embodiments, the soluble ALK6 polypeptide comprises an ECD sequence of ALK6 according to SEQ ID NO: 20. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 20.

In certain embodiments, the soluble ALK6 polypeptide comprises an ECD sequence of ALK6 according to SEQ ID NO: 21. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 21.

In certain embodiments, the soluble ALK6 polypeptide comprises an ECD sequence of ALK6 according to SEQ ID NO: 22. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 22.

In certain embodiments, the soluble ALK6 polypeptide comprises an ECD sequence of ALK6 according to SEQ ID NO: 23. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 23.

In certain embodiments, the soluble ALK6 polypeptide comprises an ECD sequence of ALK6 according to SEQ ID NO: 24. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 24.

In certain embodiments, the soluble ALK6 polypeptide comprises an ECD sequence of ALK6 according to SEQ ID NO: 25. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 25.

In certain embodiments, the soluble ALK6 polypeptide comprises an ECD sequence of ALK6 according to SEQ ID NO: 26. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 26.

In certain embodiments, the soluble ALK6 polypeptide comprises an ECD sequence of ALK6 according to SEQ ID NO: 27. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 27.

In certain embodiments, the soluble ALK6 polypeptide comprises an ECD sequence of ALK6 according to SEQ ID NO: 28. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 28.

In certain embodiments, the soluble ALK6 polypeptide comprises an ECD sequence of ALK6 according to SEQ ID NO: 29. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 29.

In certain embodiments, the soluble ALK6 polypeptide comprises an ECD sequence of ALK6 according to SEQ ID NO: 30. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 30.

In certain embodiments, the soluble ALK6 polypeptide comprises an ECD sequence of ALK6 according to SEQ ID NO: 31. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 31.

In certain embodiments, the soluble ALK6 polypeptide comprises an ECD sequence of ALK6 according to SEQ ID NO: 32. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 32.

In certain embodiments, the soluble ALK6 polypeptide comprises an ECD sequence of ALK6 according to SEQ ID NO: 33. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 33.

In certain embodiments, the soluble ALK6 polypeptide comprises an ECD sequence of ALK6 according to SEQ ID NO: 34. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 34.

In certain embodiments, the soluble ALK6 polypeptide comprises an ECD sequence of ALK6 according to SEQ ID NO: 35. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 35.

In certain embodiments, the soluble ALK6 polypeptide comprises an ECD sequence of ALK6 according to SEQ ID NO: 36. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 36.

In certain embodiments, the soluble ALK6 polypeptide comprises an ECD sequence of ALK6 according to SEQ ID NO: 37. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 37.

In certain embodiments, the soluble ALK6 polypeptide comprises an ECD sequence of ALK6 according to SEQ ID NO: 38. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 38.

In certain embodiments, the soluble ALK6 polypeptide comprises an ECD sequence of ALK6 according to SEQ ID NO: 39. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 39.

In certain embodiments, the soluble ALK6 polypeptide comprises an ECD sequence of ALK6 according to SEQ ID NO: 40. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 40.

In certain embodiments, the soluble ALK6 polypeptide comprises an ECD sequence of ALK6 according to SEQ ID NO: 41. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 41.

In certain embodiments, the soluble ALK6 polypeptide comprises an ECD sequence of ALK6 according to SEQ ID NO: 42. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 42.

In certain embodiments, the soluble ALK6 polypeptide comprises an ECD sequence of ALK6 according to SEQ ID NO: 43. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 43.

In certain embodiments, the soluble ALK6 polypeptide comprises an ECD sequence of ALK6 according to SEQ ID NO: 44. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 44.

In certain embodiments, the soluble ALK6 polypeptide comprises an ECD sequence of ALK6 according to SEQ ID NO: 45. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 45.

In certain embodiments, the soluble ALK6 polypeptide comprises an ECD sequence of ALK6 according to SEQ ID NO: 57. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 57.

In certain embodiments, the soluble ALK6 polypeptide comprises an ECD sequence of ALK6 according to SEQ ID NO: 59. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 59.

In certain embodiments, the soluble ALK6 polypeptide comprises an ECD sequence of ALK6 according to SEQ ID NO: 61. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 61.

In certain embodiments, the soluble ALK6 polypeptide comprises an ECD sequence of ALK6 according to SEQ ID NO: 63. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 63.

In certain embodiments, the soluble ALK6 polypeptide comprises an ECD sequence of ALK6 according to SEQ ID NO: 65. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 65.

In certain embodiments, the soluble ALK6 polypeptide comprises an ECD sequence of ALK6 according to SEQ ID NO: 88. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 88.

In certain embodiments, the soluble ALK-6 polypeptide comprises an active portion (e.g., ligand binding) of SEQ ID NO: 82 (“SEQ ID NO:80 variants”). In certain embodiments, the soluble ALK-6 polypeptide comprises an active portion (e.g., ligand binding) comprises an ECD sequence of ALK6 selected from exemplified SEQ ID NO: 67, 73, 75, and 77.

In certain embodiments, the soluble ALK6 polypeptide comprises an ECD sequence of ALK6 according to SEQ ID NO: 67. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 67.

In certain embodiments, the soluble ALK6 polypeptide comprises an ECD sequence of ALK6 according to SEQ ID NO: 73. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 73.

In certain embodiments, the soluble ALK6 polypeptide comprises an ECD sequence of ALK6 according to SEQ ID NO: 75. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 75.

In certain embodiments, the soluble ALK6 polypeptide comprises an ECD sequence of ALK6 according to SEQ ID NO: 77. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 77.

In certain embodiments, the soluble ALK-6 polypeptide comprises an active portion (e.g., ligand binding) of SEQ ID NO: 81 (“SEQ ID NO:69 variants”). In certain embodiments, the soluble ALK-6 polypeptide comprises an active portion (e.g., ligand binding) comprises an ECD sequence of ALK6 selected from exemplified according to SEQ ID NO: 13, 86, 90, 92, 94, 96, 98, 100, 102, 104, 110, 113, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 202, 213, 218, 223, 228, 233, 242, 247, 252, 257, 262, 267, 272, 277 and 283.

In certain embodiments, the soluble ALK6 polypeptide comprises an ECD sequence of ALK6 according to SEQ ID NO: 13. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 13.

In certain embodiments, the soluble ALK6 polypeptide comprises an ECD sequence of ALK6 according to SEQ ID NO: 86. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 86.

In certain embodiments, the soluble ALK6 polypeptide comprises an ECD sequence of ALK6 according to SEQ ID NO: 90. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 90.

In certain embodiments, the soluble ALK6 polypeptide comprises an ECD sequence of ALK6 according to SEQ ID NO: 92. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 92.

In certain embodiments, the soluble ALK6 polypeptide comprises an ECD sequence of ALK6 according to SEQ ID NO: 94. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 94.

In certain embodiments, the soluble ALK6 polypeptide comprises an ECD sequence of ALK6 according to SEQ ID NO: 96. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 96.

In certain embodiments, the soluble ALK6 polypeptide comprises an ECD sequence of ALK6 according to SEQ ID NO: 98. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 98.

In certain embodiments, the soluble ALK6 polypeptide comprises an ECD sequence of ALK6 according to SEQ ID NO: 100. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 100.

In certain embodiments, the soluble ALK6 polypeptide comprises an ECD sequence of ALK6 according to SEQ ID NO: 102. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 102.

In certain embodiments, the soluble ALK6 polypeptide comprises an ECD sequence of ALK6 according to SEQ ID NO: 104. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 104.

In certain embodiments, the soluble ALK6 polypeptide comprises an ECD sequence of ALK6 according to SEQ ID NO: 110. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 110.

In certain embodiments, the soluble ALK6 polypeptide comprises an ECD sequence of ALK6 according to SEQ ID NO: 113. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 113.

In certain embodiments, the soluble ALK-6 polypeptide comprises an active portion (e.g., ligand binding) of SEQ ID NO: 157 (“SEQ ID NO:1 variants”). In certain embodiments, the soluble ALK-6 polypeptide comprises an active portion (e.g., ligand binding) comprises an ECD sequence of ALK6 selected from exemplified according to SEQ ID NO: 132 to 155.

In certain embodiments, the soluble ALK6 polypeptide comprises an ECD sequence of ALK6 according to SEQ ID NO: 132. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 132.

In certain embodiments, the soluble ALK6 polypeptide comprises an ECD sequence of ALK6 according to SEQ ID NO: 133. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 133.

In certain embodiments, the soluble ALK6 polypeptide comprises an ECD sequence of ALK6 according to SEQ ID NO: 134. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 134.

In certain embodiments, the soluble ALK6 polypeptide comprises an ECD sequence of ALK6 according to SEQ ID NO: 135. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 135.

In certain embodiments, the soluble ALK6 polypeptide comprises an ECD sequence of ALK6 according to SEQ ID NO: 136. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 136.

In certain embodiments, the soluble ALK6 polypeptide comprises an ECD sequence of ALK6 according to SEQ ID NO: 137. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 137.

In certain embodiments, the soluble ALK6 polypeptide comprises an ECD sequence of ALK6 according to SEQ ID NO: 138. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 138.

In certain embodiments, the soluble ALK6 polypeptide comprises an ECD sequence of ALK6 according to SEQ ID NO: 139. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 139.

In certain embodiments, the soluble ALK6 polypeptide comprises an ECD sequence of ALK6 according to SEQ ID NO: 140. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 140.

In certain embodiments, the soluble ALK6 polypeptide comprises an ECD sequence of ALK6 according to SEQ ID NO: 141. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 141.

In certain embodiments, the soluble ALK6 polypeptide comprises an ECD sequence of ALK6 according to SEQ ID NO: 142. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 142.

In certain embodiments, the soluble ALK6 polypeptide comprises an ECD sequence of ALK6 according to SEQ ID NO: 143. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 143.

In certain embodiments, the soluble ALK6 polypeptide comprises an ECD sequence of ALK6 according to SEQ ID NO: 144. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 144.

In certain embodiments, the soluble ALK6 polypeptide comprises an ECD sequence of ALK6 according to SEQ ID NO: 145. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 145.

In certain embodiments, the soluble ALK6 polypeptide comprises an ECD sequence of ALK6 according to SEQ ID NO: 146. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 146.

In certain embodiments, the soluble ALK6 polypeptide comprises an ECD sequence of ALK6 according to SEQ ID NO: 147. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 147.

In certain embodiments, the soluble ALK6 polypeptide comprises an ECD sequence of ALK6 according to SEQ ID NO: 148. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 148.

In certain embodiments, the soluble ALK6 polypeptide comprises an ECD sequence of ALK6 according to SEQ ID NO: 149. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 149.

In certain embodiments, the soluble ALK6 polypeptide comprises an ECD sequence of ALK6 according to SEQ ID NO: 150. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 150.

In certain embodiments, the soluble ALK6 polypeptide comprises an ECD sequence of ALK6 according to SEQ ID NO: 151. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 151.

In certain embodiments, the soluble ALK6 polypeptide comprises an ECD sequence of ALK6 according to SEQ ID NO: 152. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 152.

In certain embodiments, the soluble ALK6 polypeptide comprises an ECD sequence of ALK6 according to SEQ ID NO: 153. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 153.

In certain embodiments, the soluble ALK6 polypeptide comprises an ECD sequence of ALK6 according to SEQ ID NO: 154. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 154.

In certain embodiments, the soluble ALK6 polypeptide comprises an ECD sequence of ALK6 according to SEQ ID NO: 155. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 155.

In certain embodiments, the soluble ALK6 polypeptide comprises an ECD sequence of ALK6 according to SEQ ID NO: 156. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 156.

In certain embodiments, the soluble ALK-6 polypeptide comprises an active portion (e.g., ligand binding) of SEQ ID NO: 190 (“SEQ ID NO:69 variants”). In certain embodiments, the soluble ALK-6 polypeptide comprises an active portion (e.g., ligand binding) comprises an ECD sequence of ALK6 selected from exemplified according to SEQ ID NO: 158-189.

In certain embodiments, the soluble ALK6 polypeptide comprises an ECD sequence of ALK6 according to SEQ ID NO: 158. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 158.

In certain embodiments, the soluble ALK6 polypeptide comprises an ECD sequence of ALK6 according to SEQ ID NO: 159. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 159.

In certain embodiments, the soluble ALK6 polypeptide comprises an ECD sequence of ALK6 according to SEQ ID NO: 160. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 160.

In certain embodiments, the soluble ALK6 polypeptide comprises an ECD sequence of ALK6 according to SEQ ID NO: 161. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 161.

In certain embodiments, the soluble ALK6 polypeptide comprises an ECD sequence of ALK6 according to SEQ ID NO: 162. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 162.

In certain embodiments, the soluble ALK6 polypeptide comprises an ECD sequence of ALK6 according to SEQ ID NO: 163. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 163.

In certain embodiments, the soluble ALK6 polypeptide comprises an ECD sequence of ALK6 according to SEQ ID NO: 164. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 164.

In certain embodiments, the soluble ALK6 polypeptide comprises an ECD sequence of ALK6 according to SEQ ID NO: 165. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 165.

In certain embodiments, the soluble ALK6 polypeptide comprises an ECD sequence of ALK6 according to SEQ ID NO: 166. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 166.

In certain embodiments, the soluble ALK6 polypeptide comprises an ECD sequence of ALK6 according to SEQ ID NO: 167. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 167.

In certain embodiments, the soluble ALK6 polypeptide comprises an ECD sequence of ALK6 according to SEQ ID NO: 168. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 168.

In certain embodiments, the soluble ALK6 polypeptide comprises an ECD sequence of ALK6 according to SEQ ID NO: 169. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 169.

In certain embodiments, the soluble ALK6 polypeptide comprises an ECD sequence of ALK6 according to SEQ ID NO: 170. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 170.

In certain embodiments, the soluble ALK6 polypeptide comprises an ECD sequence of ALK6 according to SEQ ID NO: 171. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 171.

In certain embodiments, the soluble ALK6 polypeptide comprises an ECD sequence of ALK6 according to SEQ ID NO: 172. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 172.

In certain embodiments, the soluble ALK6 polypeptide comprises an ECD sequence of ALK6 according to SEQ ID NO: 173. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 173.

In certain embodiments, the soluble ALK6 polypeptide comprises an ECD sequence of ALK6 according to SEQ ID NO: 174. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 174.

In certain embodiments, the soluble ALK6 polypeptide comprises an ECD sequence of ALK6 according to SEQ ID NO: 175. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 175.

In certain embodiments, the soluble ALK6 polypeptide comprises an ECD sequence of ALK6 according to SEQ ID NO: 176. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 176.

In certain embodiments, the soluble ALK6 polypeptide comprises an ECD sequence of ALK6 according to SEQ ID NO: 177. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 177.

In certain embodiments, the soluble ALK6 polypeptide comprises an ECD sequence of ALK6 according to SEQ ID NO: 178. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 178.

In certain embodiments, the soluble ALK6 polypeptide comprises an ECD sequence of ALK6 according to SEQ ID NO: 179. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 179.

In certain embodiments, the soluble ALK6 polypeptide comprises an ECD sequence of ALK6 according to SEQ ID NO: 180. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 180.

In certain embodiments, the soluble ALK6 polypeptide comprises an ECD sequence of ALK6 according to SEQ ID NO: 181. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 181.

In certain embodiments, the soluble ALK6 polypeptide comprises an ECD sequence of ALK6 according to SEQ ID NO: 182. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 182.

In certain embodiments, the soluble ALK6 polypeptide comprises an ECD sequence of ALK6 according to SEQ ID NO: 183. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 183.

In certain embodiments, the soluble ALK6 polypeptide comprises an ECD sequence of ALK6 according to SEQ ID NO: 184. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 184.

In certain embodiments, the soluble ALK6 polypeptide comprises an ECD sequence of ALK6 according to SEQ ID NO: 185. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 185.

In certain embodiments, the soluble ALK6 polypeptide comprises an ECD sequence of ALK6 according to SEQ ID NO: 186. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 186.

In certain embodiments, the soluble ALK6 polypeptide comprises an ECD sequence of ALK6 according to SEQ ID NO: 187. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 187.

In certain embodiments, the soluble ALK6 polypeptide comprises an ECD sequence of ALK6 according to SEQ ID NO: 188. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 188.

In certain embodiments, the soluble ALK6 polypeptide comprises an ECD sequence of ALK6 according to SEQ ID NO: 189. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 189.

In certain embodiments, the soluble ALK6 polypeptide comprises an ECD sequence of ALK6 according to SEQ ID NO: 191. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 191.

In certain embodiments, the soluble ALK6 polypeptide comprises an ECD sequence of ALK6 according to SEQ ID NO: 192. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 192.

In certain embodiments, the soluble ALK6 polypeptide comprises an ECD sequence of ALK6 according to SEQ ID NO: 193. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 193.

In certain embodiments, the soluble ALK6 polypeptide comprises an ECD sequence of ALK6 according to SEQ ID NO: 202. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 202.

In certain embodiments, the soluble ALK6 polypeptide comprises an ECD sequence of ALK6 according to SEQ ID NO: 213. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 213.

In certain embodiments, the soluble ALK6 polypeptide comprises an ECD sequence of ALK6 according to SEQ ID NO: 218. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 218.

In certain embodiments, the soluble ALK6 polypeptide comprises an ECD sequence of ALK6 according to SEQ ID NO: 223. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 223.

In certain embodiments, the soluble ALK6 polypeptide comprises an ECD sequence of ALK6 according to SEQ ID NO: 228. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 228.

In certain embodiments, the soluble ALK6 polypeptide comprises an ECD sequence of ALK6 according to SEQ ID NO: 233. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 233.

In certain embodiments, the soluble ALK6 polypeptide comprises an ECD sequence of ALK6 according to SEQ ID NO: 242. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 242.

In certain embodiments, the soluble ALK6 polypeptide comprises an ECD sequence of ALK6 according to SEQ ID NO: 247. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 247.

In certain embodiments, the soluble ALK6 polypeptide comprises an ECD sequence of ALK6 according to SEQ ID NO: 252. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 252.

In certain embodiments, the soluble ALK6 polypeptide comprises an ECD sequence of ALK6 according to SEQ ID NO: 257. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 257.

In certain embodiments, the soluble ALK6 polypeptide comprises an ECD sequence of ALK6 according to SEQ ID NO: 223. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 262.

In certain embodiments, the soluble ALK6 polypeptide comprises an ECD sequence of ALK6 according to SEQ ID NO: 267. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 267.

In certain embodiments, the soluble ALK6 polypeptide comprises an ECD sequence of ALK6 according to SEQ ID NO: 272. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 272.

In certain embodiments, the soluble ALK6 polypeptide comprises an ECD sequence of ALK6 according to SEQ ID NO: 277. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 277.

In certain embodiments, the soluble ALK6 polypeptide comprises an ECD sequence of ALK6 according to SEQ ID NO: 283. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 283.

In certain embodiments, the soluble ALK-6 polypeptide comprises an active portion (e.g., ligand binding) of SEQ ID NO: 437 (“SEQ ID NO:90 variants”). In certain embodiments, the soluble ALK-6 polypeptide comprises an active portion (e.g., ligand binding) comprising an ECD sequence of ALK6 selected from exemplified sequences according to SEQ ID NO: 286, 292, 298, 304, 310, 316, 322, 328, 334, 340, 346, 352, 358, 364, 370, 376, 382, 388, 394, 400, 406, 412, 418, 425 and 431.

In certain embodiments, the soluble ALK6 polypeptide comprises an ECD sequence of ALK6 according to SEQ ID NO: 286. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 286.

In certain embodiments, the soluble ALK6 polypeptide comprises an ECD sequence of ALK6 according to SEQ ID NO: 292. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 292.

In certain embodiments, the soluble ALK6 polypeptide comprises an ECD sequence of ALK6 according to SEQ ID NO: 298. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 298.

In certain embodiments, the soluble ALK6 polypeptide comprises an ECD sequence of ALK6 according to SEQ ID NO: 304. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 304.

In certain embodiments, the soluble ALK6 polypeptide comprises an ECD sequence of ALK6 according to SEQ ID NO: 310. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 310.

In certain embodiments, the soluble ALK6 polypeptide comprises an ECD sequence of ALK6 according to SEQ ID NO: 316. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 316.

In certain embodiments, the soluble ALK6 polypeptide comprises an ECD sequence of ALK6 according to SEQ ID NO: 322. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 322.

In certain embodiments, the soluble ALK6 polypeptide comprises an ECD sequence of ALK6 according to SEQ ID NO: 328. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 328.

In certain embodiments, the soluble ALK6 polypeptide comprises an ECD sequence of ALK6 according to SEQ ID NO: 334. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 334.

In certain embodiments, the soluble ALK6 polypeptide comprises an ECD sequence of ALK6 according to SEQ ID NO: 340. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 340.

In certain embodiments, the soluble ALK6 polypeptide comprises an ECD sequence of ALK6 according to SEQ ID NO: 346. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 346.

In certain embodiments, the soluble ALK6 polypeptide comprises an ECD sequence of ALK6 according to SEQ ID NO: 352. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 352.

In certain embodiments, the soluble ALK6 polypeptide comprises an ECD sequence of ALK6 according to SEQ ID NO: 358. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 358.

In certain embodiments, the soluble ALK6 polypeptide comprises an ECD sequence of ALK6 according to SEQ ID NO: 364. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 364.

In certain embodiments, the soluble ALK6 polypeptide comprises an ECD sequence of ALK6 according to SEQ ID NO: 370. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 370.

In certain embodiments, the soluble ALK6 polypeptide comprises an ECD sequence of ALK6 according to SEQ ID NO: 376. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 376.

In certain embodiments, the soluble ALK6 polypeptide comprises an ECD sequence of ALK6 according to SEQ ID NO: 382. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 382.

In certain embodiments, the soluble ALK6 polypeptide comprises an ECD sequence of ALK6 according to SEQ ID NO: 388. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 388.

In certain embodiments, the soluble ALK6 polypeptide comprises an ECD sequence of ALK6 according to SEQ ID NO: 394. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 394.

In certain embodiments, the soluble ALK6 polypeptide comprises an ECD sequence of ALK6 according to SEQ ID NO: 400. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 400.

In certain embodiments, the soluble ALK6 polypeptide comprises an ECD sequence of ALK6 according to SEQ ID NO: 406. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 406.

In certain embodiments, the soluble ALK6 polypeptide comprises an ECD sequence of ALK6 according to SEQ ID NO: 412. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 412.

In certain embodiments, the soluble ALK6 polypeptide comprises an ECD sequence of ALK6 according to SEQ ID NO: 418. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 418.

In certain embodiments, the soluble ALK6 polypeptide comprises an ECD sequence of ALK6 according to SEQ ID NO: 425. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 425.

In certain embodiments, the soluble ALK6 polypeptide comprises an ECD sequence of ALK6 according to SEQ ID NO: 431. Therefore ALK6 polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 431.

In certain embodiments, the ALK6 ECD polypeptide portion of the soluble ALK6 fusion protein (“BMP antagonist fusion protein”) is selected from the group consisting of: SEQ ID NOs: 1, 3-5, 11-45, 57, 59, 61, 63, 65, 67, 69, 71, 73, 75, 77, 79-83, 86, 88, 90, 92, 94, 96, 98, 100, 102, 104, 108, 109, 110, 113, 132-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, 431 and 437. For example, the ALK6 ECD polypeptide portion of the BMP antagonist fusion protein may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 1, 3-5, 11-45, 57, 59, 61, 63, 65, 67, 69, 71, 73, 75, 77, 79-83, 86, 88, 90, 92, 94, 96, 98, 100, 102, 104, 108, 109, 110, 113, 132-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, 431 and 437.

In certain embodiments, ALK6 ECD polypeptide portion of the soluble ALK6 fusion protein (“BMP antagonist fusion protein”) is selected from the group consisting of: SEQ ID NO: 33, 86, 88 90, 233, 242, 283 and 286. For example, the ALK6 ECD polypeptide portion of the BMP antagonist fusion protein may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 33, 86, 88 90, 233, 242, 283 and 286.

In embodiments, the ALK6 ECD polypeptide can be adjoined to a heterologous domain sequence which aid formation and stability of homodimer soluble ALK6 polypeptides. In certain embodiments, the heterologous domain can be derived from the constant region of an antibody (i.e., the Fc region or the hinge region between the CH1 and CH2 domains). In certain embodiments, the Fc region of the fusion protein is exemplified as:

(SEQ ID NO: 10)
THTCPPCPAPELLGGPSVFLFPPKPKDTLMISRTPEVTCVVVDVSH
EDPEVKFNWYVDGVEVHNAKTKPREEQYNSTYRVVSVLTVLHQDWL
NGKEYKCKVSNKALPAPIEKTISKAKGQPREPQVYTLPPSREEMTK
NQVSLTCLVKGFYPSDIAVEWESNGQPENNYKTTPPVLDSDGSFFL
YSKLTVDKSRWQQGNVFSCSVMHEALHNHYTQKSLSLSPGK.

In certain embodiments, the Fc region of the fusion protein is exemplified as:

(SEQ ID NO: 106)
THTCPPCPAPEAAGGPSVFLFPPKPKDTLMISRTPEVTCVVVDVSH
EDPEVKFNWYVDGVEVHNAKTKPREEQYNSTYRVVSVLTVLHQDWL
NGKEYKCKVSNKALPAPIEKTISKAKGQPREPQVYTLPPSREEMTK
NQVSLTCLVKGFYPSDIAVEWESNGQPENNYKTTPPVLDSDGSFFL
YSKLTVDKSRWQQGNVFSCSVMHEALHNHYTQKSLSLSPG_

In certain embodiments, the Fc region of the fusion protein is exemplified as:

(SEQ ID NO: 107)
THTCPPCPAPEAAGGPSVFLFPPKPKDTLMISRTPEVTCVVVDVSH
EDPEVKFNWYVDGVEVHNAKTKPREEQYNSTYRVVSVLTVLHQDWL
NGKEYKCKVSNKALPAPIEKTISKAKGQPREPQVYTLPPSREEMTK
NQVSLTCLVKGFYPSDIAVEWESNGQPENNYKTTPPVLDSDGSFFL
YSKLTVDKSRWQQGNVFSCSVMHEALHNHYTQKSLSLSPGK

In certain embodiments, the Fc region of the fusion protein is exemplified as:

(SEQ ID NO: 285)
THTCPPCPAPEAAGGPSVFLFPPKPKDTLMASRTPEVTCVVVDVSH
EDPEVKFNWYVDGVEVHNAKTKPREEQYNSTYRVVSVLTVLAQDWL
NGKEYKCKVSNKALPAPIEKTISKAKGQPREPQVYTLPPSREEMTK
NQVSLTCLVKGFYPSDIAVEWESNGQPENNYKTTPPVLDSDGSFFL
YSKLTVDKSRWQQGNVFSCSVMHEALHNHYTQKSLSLSPG

In certain embodiments, the Fc sequence SEQ ID NO: 285 is combined with any of the ALK6 ECD sequences of this disclosure to provide an ALK6 Fc fusion protein for intravitreal (“IVT”) administration, wherein SEQ ID NO: 285 does not bind complement decreasing the fusion protein's half-life in circulation. The ALK6 Fc fusion proteins of this disclosure can be administered systemically or intravitreally for the treatment of a retinopathy disease.

In certain embodiments, the heterologous domain of the fusion protein comprises the cysteine core of the amino acid sequence from the hinge region of IgG1, IgG2, IgG3 or IgG4. For example, EPKSCDKTHTCPPCPAPELLGGP (SEQ ID NO: 196); ERKCCVECPPCPAPPVAGP (SEQ ID NO: 197); and ESKYGPPCPSCPAPEFLGGP (SEQ ID NO: 198); THTCPPCPAPEAAGGP (SEQ ID NO: 199); THTCPPCKCPEAAGGP (SEQ ID NO: 200); and PTIKPCPPCKCPAP (SEQ ID NO: 201).

Other heterologous domain sequences may also be suitable to aid in dimerization of the extracellular domains of the ALK6 ECD and the linker regions as would be understood by those of ordinary skill in the art. It is also understood that for certain animal studies murine or the like, homologous Fc sequences are used as needed to test the ALK6 ECD polypeptides of this disclosure.

In embodiments, the ALK6 ECD polypeptide can be adjoined to a heterologous domain sequence as a fusion protein using a linker peptide. In certain embodiments, the linker peptide can be selected from the group consisting of:

(SEQ ID NO: 6)
TGGGGSGGGGSGGGGSGGGGS;
(SEQ ID NO: 7)
TGGG;
and,
(SEQ ID NO: 8)
EAAAKEAAAKEAAAKEAAAK.

Other linker peptides may also be suitable as would be understood by those of ordinary skill in the art. See for example SEQ ID NO: 84 and 85

In embodiments, the fusion protein can include a leader peptide. In certain embodiments, the leader peptide can be MGWSCIILFLVATATGVHS (SEQ ID NO: 9). Other leader peptides may also be suitable as would be understood by those of ordinary skill in the art. One of skill in the art also understands the leader sequence is cleaved as part of processing resulting in a mature processed polypeptide sequence (e.g. SEQ ID NO: 1).

Provided herein are a number of exemplified soluble ALK6 fusion polypeptides (proteins) comprising an ALK6 ECD sequence portion, a linker sequence portion and an Fc sequence portion according to any of the portion sequences (e.g., ALK6 ECD, linker and FC) described above and herein. In certain embodiments, the soluble ALK6 fusion polypeptide comprises an amino acid sequence selected from SEQ ID NO: 46-50, 52-56, 58, 60, 62, 64, 66, 68, 70, 72, 74, 76, 78, 84, 85, 87, 89, 91, 93, 95, 97, 99, 101, 103, 105, 111, 112, 114, 115, 116, 117, 118, 119, 120, 121, 122, 123, 124, 125, 126, 127, 128, 129, 203, 214, 219, 224, 229, 234, 238, 243, 248, 253, 258, 263, 268, 273, 278, 282, and 284. In certain other embodiments, the soluble ALK6 fusion polypeptide comprises an amino acid sequence selected from SEQ ID NO: 291, 297, 303, 309, 315, 321, 327, 333, 339, 345, 351, 357, 363, 369, 375, 381, 387, 393, 399, 405, 411, 417, 423, 424, 430, and 436.

In certain embodiments, the soluble ALK6 fusion polypeptide comprises an amino acid sequence according to SEQ ID NO: 46. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 46.

In certain embodiments, the soluble ALK6 fusion polypeptide comprises an amino acid sequence according to SEQ ID NO: 47. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 47.

In certain embodiments, the soluble ALK6 fusion polypeptide comprises an amino acid sequence according to SEQ ID NO: 48. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 48.

In certain embodiments, the soluble ALK6 fusion polypeptide comprises an amino acid sequence according to SEQ ID NO: 49. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 49.

In certain embodiments, the soluble ALK6 fusion polypeptide comprises an amino acid sequence according to SEQ ID NO: 50. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 50.

In certain embodiments, the soluble ALK6 fusion polypeptide comprises an amino acid sequence according to SEQ ID NO: 52. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 52.

In certain embodiments, the soluble ALK6 fusion polypeptide comprises an amino acid sequence according to SEQ ID NO: 53. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 53.

In certain embodiments, the soluble ALK6 fusion polypeptide comprises an amino acid sequence according to SEQ ID NO: 54. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 54.

In certain embodiments, the soluble ALK6 fusion polypeptide comprises an amino acid sequence according to SEQ ID NO: 55. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 55.

In certain embodiments, the soluble ALK6 fusion polypeptide comprises an amino acid sequence according to SEQ ID NO: 56. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 56.

In certain embodiments, the soluble ALK6 fusion polypeptide comprises an amino acid sequence according to SEQ ID NO: 60. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 60.

In certain embodiments, the soluble ALK6 fusion polypeptide comprises an amino acid sequence according to SEQ ID NO: 62. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 62.

In certain embodiments, the soluble ALK6 fusion polypeptide comprises an amino acid sequence according to SEQ ID NO: 64. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 64.

In certain embodiments, the soluble ALK6 fusion polypeptide comprises an amino acid sequence according to SEQ ID NO: 66. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 66.

In certain embodiments, the soluble ALK6 fusion polypeptide comprises an amino acid sequence according to SEQ ID NO: 68. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 68.

In certain embodiments, the soluble ALK6 fusion polypeptide comprises an amino acid sequence according to SEQ ID NO: 70. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 70.

In certain embodiments, the soluble ALK6 fusion polypeptide comprises an amino acid sequence according to SEQ ID NO: 72. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 72.

In certain embodiments, the soluble ALK6 fusion polypeptide comprises an amino acid sequence according to SEQ ID NO: 74. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 74.

In certain embodiments, the soluble ALK6 fusion polypeptide comprises an amino acid sequence according to SEQ ID NO: 76. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 76.

In certain embodiments, the soluble ALK6 fusion polypeptide comprises an amino acid sequence according to SEQ ID NO: 78. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 78.

In certain embodiments, the soluble ALK6 fusion polypeptide comprises an amino acid sequence according to SEQ ID NO: 84. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 84.

In certain embodiments, the soluble ALK6 fusion polypeptide comprises an amino acid sequence according to SEQ ID NO: 85. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 85.

In certain embodiments, the soluble ALK6 fusion polypeptide comprises an amino acid sequence according to SEQ ID NO: 87. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 87.

In certain embodiments, the soluble ALK6 fusion polypeptide comprises an amino acid sequence according to SEQ ID NO: 89. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 89.

In certain embodiments, the soluble ALK6 fusion polypeptide comprises an amino acid sequence according to SEQ ID NO: 91. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 91.

In certain embodiments, the soluble ALK6 fusion polypeptide comprises an amino acid sequence according to SEQ ID NO: 93. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 93.

In certain embodiments, the soluble ALK6 fusion polypeptide comprises an amino acid sequence according to SEQ ID NO: 95. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 95.

In certain embodiments, the soluble ALK6 fusion polypeptide comprises an amino acid sequence according to SEQ ID NO: 97. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 97.

In certain embodiments, the soluble ALK6 fusion polypeptide comprises an amino acid sequence according to SEQ ID NO: 99. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 99.

In certain embodiments, the soluble ALK6 fusion polypeptide comprises an amino acid sequence according to SEQ ID NO: 101. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 101.

In certain embodiments, the soluble ALK6 fusion polypeptide comprises an amino acid sequence according to SEQ ID NO: 103. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 103.

In certain embodiments, the soluble ALK6 fusion polypeptide comprises an amino acid sequence according to SEQ ID NO: 105. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 105.

In certain embodiments, the soluble ALK6 fusion polypeptide comprises an amino acid sequence according to SEQ ID NO: 111. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 111.

In certain embodiments, the soluble ALK6 fusion polypeptide comprises an amino acid sequence according to SEQ ID NO: 112. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 112.

In certain embodiments, the soluble ALK6 fusion polypeptide comprises an amino acid sequence according to SEQ ID NO: 114. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 114.

In certain embodiments, the soluble ALK6 fusion polypeptide comprises an amino acid sequence according to SEQ ID NO: 115. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 115.

In certain embodiments, the soluble ALK6 fusion polypeptide comprises an amino acid sequence according to SEQ ID NO: 116. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 116.

In certain embodiments, the soluble ALK6 fusion polypeptide comprises an amino acid sequence according to SEQ ID NO: 117. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 117.

In certain embodiments, the soluble ALK6 fusion polypeptide comprises an amino acid sequence according to SEQ ID NO: 118. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 118.

In certain embodiments, the soluble ALK6 fusion polypeptide comprises an amino acid sequence according to SEQ ID NO: 119. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 119.

In certain embodiments, the soluble ALK6 fusion polypeptide comprises an amino acid sequence according to SEQ ID NO: 120. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 120.

In certain embodiments, the soluble ALK6 fusion polypeptide comprises an amino acid sequence according to SEQ ID NO: 121. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 121.

In certain embodiments, the soluble ALK6 fusion polypeptide comprises an amino acid sequence according to SEQ ID NO: 122. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 122.

In certain embodiments, the soluble ALK6 fusion polypeptide comprises an amino acid sequence according to SEQ ID NO: 123. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 123.

In certain embodiments, the soluble ALK6 fusion polypeptide comprises an amino acid sequence according to SEQ ID NO: 124. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 124.

In certain embodiments, the soluble ALK6 fusion polypeptide comprises an amino acid sequence according to SEQ ID NO: 125. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 125.

In certain embodiments, the soluble ALK6 fusion polypeptide comprises an amino acid sequence according to SEQ ID NO: 126. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 126.

In certain embodiments, the soluble ALK6 fusion polypeptide comprises an amino acid sequence according to SEQ ID NO: 127. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 127.

In certain embodiments, the soluble ALK6 fusion polypeptide comprises an amino acid sequence according to SEQ ID NO: 128. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 128.

In certain embodiments, the soluble ALK6 fusion polypeptide comprises an amino acid sequence according to SEQ ID NO: 129. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 129.

In certain embodiments, the soluble ALK6 fusion polypeptide comprises an amino acid sequence according to SEQ ID NO: 203. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 203.

In certain embodiments, the soluble ALK6 fusion polypeptide comprises an amino acid sequence according to SEQ ID NO: 204. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 204.

In certain embodiments, the soluble ALK6 fusion polypeptide comprises an amino acid sequence according to SEQ ID NO: 205. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 205.

In certain embodiments, the soluble ALK6 fusion polypeptide comprises an amino acid sequence according to SEQ ID NO: 206. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 206.

In certain embodiments, the soluble ALK6 fusion polypeptide comprises an amino acid sequence according to SEQ ID NO: 207. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 207.

In certain embodiments, the soluble ALK6 fusion polypeptide comprises an amino acid sequence according to SEQ ID NO: 208. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 208.

In certain embodiments, the soluble ALK6 fusion polypeptide comprises an amino acid sequence according to SEQ ID NO: 209. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 209.

In certain embodiments, the soluble ALK6 fusion polypeptide comprises an amino acid sequence according to SEQ ID NO: 210. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 210.

In certain embodiments, the soluble ALK6 fusion polypeptide comprises an amino acid sequence according to SEQ ID NO: 211. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 211.

In certain embodiments, the soluble ALK6 fusion polypeptide comprises an amino acid sequence according to SEQ ID NO: 212. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 212.

In certain embodiments, the soluble ALK6 fusion polypeptide comprises an amino acid sequence according to SEQ ID NO: 214. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 214.

In certain embodiments, the soluble ALK6 fusion polypeptide comprises an amino acid sequence according to SEQ ID NO: 215. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 215.

In certain embodiments, the soluble ALK6 fusion polypeptide comprises an amino acid sequence according to SEQ ID NO: 216. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 216.

In certain embodiments, the soluble ALK6 fusion polypeptide comprises an amino acid sequence according to SEQ ID NO: 217. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 217.

In certain embodiments, the soluble ALK6 fusion polypeptide comprises an amino acid sequence according to SEQ ID NO: 219. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 219.

In certain embodiments, the soluble ALK6 fusion polypeptide comprises an amino acid sequence according to SEQ ID NO: 220. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 220.

In certain embodiments, the soluble ALK6 fusion polypeptide comprises an amino acid sequence according to SEQ ID NO: 221. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 221.

In certain embodiments, the soluble ALK6 fusion polypeptide comprises an amino acid sequence according to SEQ ID NO: 222. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 222.

In certain embodiments, the soluble ALK6 fusion polypeptide comprises an amino acid sequence according to SEQ ID NO: 224. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 224.

In certain embodiments, the soluble ALK6 fusion polypeptide comprises an amino acid sequence according to SEQ ID NO: 225. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 225.

In certain embodiments, the soluble ALK6 fusion polypeptide comprises an amino acid sequence according to SEQ ID NO: 226. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 226.

In certain embodiments, the soluble ALK6 fusion polypeptide comprises an amino acid sequence according to SEQ ID NO: 227. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 227.

In certain embodiments, the soluble ALK6 fusion polypeptide comprises an amino acid sequence according to SEQ ID NO: 229. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 229.

In certain embodiments, the soluble ALK6 fusion polypeptide comprises an amino acid sequence according to SEQ ID NO: 230. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 230.

In certain embodiments, the soluble ALK6 fusion polypeptide comprises an amino acid sequence according to SEQ ID NO: 231. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 231.

In certain embodiments, the soluble ALK6 fusion polypeptide comprises an amino acid sequence according to SEQ ID NO: 232. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 232.

In certain embodiments, the soluble ALK6 fusion polypeptide comprises an amino acid sequence according to SEQ ID NO: 234. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 234.

In certain embodiments, the soluble ALK6 fusion polypeptide comprises an amino acid sequence according to SEQ ID NO: 235. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 235.

In certain embodiments, the soluble ALK6 fusion polypeptide comprises an amino acid sequence according to SEQ ID NO: 236. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 236.

In certain embodiments, the soluble ALK6 fusion polypeptide comprises an amino acid sequence according to SEQ ID NO: 237. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 237.

In certain embodiments, the soluble ALK6 fusion polypeptide comprises an amino acid sequence according to SEQ ID NO: 238. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 238.

In certain embodiments, the soluble ALK6 fusion polypeptide comprises an amino acid sequence according to SEQ ID NO: 239. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 239.

In certain embodiments, the soluble ALK6 fusion polypeptide comprises an amino acid sequence according to SEQ ID NO: 240. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 240.

In certain embodiments, the soluble ALK6 fusion polypeptide comprises an amino acid sequence according to SEQ ID NO: 241. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 241.

In certain embodiments, the soluble ALK6 fusion polypeptide comprises an amino acid sequence according to SEQ ID NO: 243. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 243.

In certain embodiments, the soluble ALK6 fusion polypeptide comprises an amino acid sequence according to SEQ ID NO: 244. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 244.

In certain embodiments, the soluble ALK6 fusion polypeptide comprises an amino acid sequence according to SEQ ID NO: 245. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 245.

In certain embodiments, the soluble ALK6 fusion polypeptide comprises an amino acid sequence according to SEQ ID NO: 246. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 246.

In certain embodiments, the soluble ALK6 fusion polypeptide comprises an amino acid sequence according to SEQ ID NO: 248. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 248.

In certain embodiments, the soluble ALK6 fusion polypeptide comprises an amino acid sequence according to SEQ ID NO: 249. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 249.

In certain embodiments, the soluble ALK6 fusion polypeptide comprises an amino acid sequence according to SEQ ID NO: 250. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 250.

In certain embodiments, the soluble ALK6 fusion polypeptide comprises an amino acid sequence according to SEQ ID NO: 251. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 251.

In certain embodiments, the soluble ALK6 fusion polypeptide comprises an amino acid sequence according to SEQ ID NO: 253. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 253.

In certain embodiments, the soluble ALK6 fusion polypeptide comprises an amino acid sequence according to SEQ ID NO: 254. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 254.

In certain embodiments, the soluble ALK6 fusion polypeptide comprises an amino acid sequence according to SEQ ID NO: 255. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 255.

In certain embodiments, the soluble ALK6 fusion polypeptide comprises an amino acid sequence according to SEQ ID NO: 256. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 256.

In certain embodiments, the soluble ALK6 fusion polypeptide comprises an amino acid sequence according to SEQ ID NO: 258. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 258.

In certain embodiments, the soluble ALK6 fusion polypeptide comprises an amino acid sequence according to SEQ ID NO: 259. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 259.

In certain embodiments, the soluble ALK6 fusion polypeptide comprises an amino acid sequence according to SEQ ID NO: 260. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 260.

In certain embodiments, the soluble ALK6 fusion polypeptide comprises an amino acid sequence according to SEQ ID NO: 261. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 261.

In certain embodiments, the soluble ALK6 fusion polypeptide comprises an amino acid sequence according to SEQ ID NO: 263. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 263.

In certain embodiments, the soluble ALK6 fusion polypeptide comprises an amino acid sequence according to SEQ ID NO: 264. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 264.

In certain embodiments, the soluble ALK6 fusion polypeptide comprises an amino acid sequence according to SEQ ID NO: 265. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 265.

In certain embodiments, the soluble ALK6 fusion polypeptide comprises an amino acid sequence according to SEQ ID NO: 266. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 266.

In certain embodiments, the soluble ALK6 fusion polypeptide comprises an amino acid sequence according to SEQ ID NO: 268. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 268.

In certain embodiments, the soluble ALK6 fusion polypeptide comprises an amino acid sequence according to SEQ ID NO: 269. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 269.

In certain embodiments, the soluble ALK6 fusion polypeptide comprises an amino acid sequence according to SEQ ID NO: 270. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 270.

In certain embodiments, the soluble ALK6 fusion polypeptide comprises an amino acid sequence according to SEQ ID NO: 271. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 271.

In certain embodiments, the soluble ALK6 fusion polypeptide comprises an amino acid sequence according to SEQ ID NO: 273. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 273.

In certain embodiments, the soluble ALK6 fusion polypeptide comprises an amino acid sequence according to SEQ ID NO: 274. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 274.

In certain embodiments, the soluble ALK6 fusion polypeptide comprises an amino acid sequence according to SEQ ID NO: 275. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 275.

In certain embodiments, the soluble ALK6 fusion polypeptide comprises an amino acid sequence according to SEQ ID NO: 276. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 276.

In certain embodiments, the soluble ALK6 fusion polypeptide comprises an amino acid sequence according to SEQ ID NO: 278. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 278.

In certain embodiments, the soluble ALK6 fusion polypeptide comprises an amino acid sequence according to SEQ ID NO: 279. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 279.

In certain embodiments, the soluble ALK6 fusion polypeptide comprises an amino acid sequence according to SEQ ID NO: 280. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 280.

In certain embodiments, the soluble ALK6 fusion polypeptide comprises an amino acid sequence according to SEQ ID NO: 281. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 281.

In certain embodiments, the soluble ALK6 fusion polypeptide comprises an amino acid sequence according to SEQ ID NO: 282. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 282.

In certain embodiments, the soluble ALK6 fusion polypeptide comprises an amino acid sequence according to SEQ ID NO: 283. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 283.

In certain embodiments, the soluble ALK6 fusion polypeptide comprises an amino acid sequence according to SEQ ID NO: 284. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 284.

In certain embodiments, the soluble ALK6 fusion polypeptide comprises an amino acid sequence according to SEQ ID NO: 291. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 291.

In certain embodiments, the soluble ALK6 fusion polypeptide comprises an amino acid sequence according to SEQ ID NO: 297. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 297.

In certain embodiments, the soluble ALK6 fusion polypeptide comprises an amino acid sequence according to SEQ ID NO: 303. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 303.

In certain embodiments, the soluble ALK6 fusion polypeptide comprises an amino acid sequence according to SEQ ID NO: 309. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 309.

In certain embodiments, the soluble ALK6 fusion polypeptide comprises an amino acid sequence according to SEQ ID NO: 315. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 315.

In certain embodiments, the soluble ALK6 fusion polypeptide comprises an amino acid sequence according to SEQ ID NO: 321. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 321.

In certain embodiments, the soluble ALK6 fusion polypeptide comprises an amino acid sequence according to SEQ ID NO: 327. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 327.

In certain embodiments, the soluble ALK6 fusion polypeptide comprises an amino acid sequence according to SEQ ID NO: 333. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 333.

In certain embodiments, the soluble ALK6 fusion polypeptide comprises an amino acid sequence according to SEQ ID NO: 339. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 339.

In certain embodiments, the soluble ALK6 fusion polypeptide comprises an amino acid sequence according to SEQ ID NO: 345. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 345.

In certain embodiments, the soluble ALK6 fusion polypeptide comprises an amino acid sequence according to SEQ ID NO: 351. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 351.

In certain embodiments, the soluble ALK6 fusion polypeptide comprises an amino acid sequence according to SEQ ID NO: 357. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 357.

In certain embodiments, the soluble ALK6 fusion polypeptide comprises an amino acid sequence according to SEQ ID NO: 363. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 363.

In certain embodiments, the soluble ALK6 fusion polypeptide comprises an amino acid sequence according to SEQ ID NO: 369. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 369.

In certain embodiments, the soluble ALK6 fusion polypeptide comprises an amino acid sequence according to SEQ ID NO: 375. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 375.

In certain embodiments, the soluble ALK6 fusion polypeptide comprises an amino acid sequence according to SEQ ID NO: 381. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 381.

In certain embodiments, the soluble ALK6 fusion polypeptide comprises an amino acid sequence according to SEQ ID NO: 387. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 387.

In certain embodiments, the soluble ALK6 fusion polypeptide comprises an amino acid sequence according to SEQ ID NO: 393. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 393.

In certain embodiments, the soluble ALK6 fusion polypeptide comprises an amino acid sequence according to SEQ ID NO: 375. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 375.

In certain embodiments, the soluble ALK6 fusion polypeptide comprises an amino acid sequence according to SEQ ID NO: 381. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 381.

In certain embodiments, the soluble ALK6 fusion polypeptide comprises an amino acid sequence according to SEQ ID NO: 387. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 387.

In certain embodiments, the soluble ALK6 fusion polypeptide comprises an amino acid sequence according to SEQ ID NO: 393. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 393.

In certain embodiments, the soluble ALK6 fusion polypeptide comprises an amino acid sequence according to SEQ ID NO: 399. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 399.

In certain embodiments, the soluble ALK6 fusion polypeptide comprises an amino acid sequence according to SEQ ID NO: 405. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 405.

In certain embodiments, the soluble ALK6 fusion polypeptide comprises an amino acid sequence according to SEQ ID NO: 411. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 411.

In certain embodiments, the soluble ALK6 fusion polypeptide comprises an amino acid sequence according to SEQ ID NO: 417. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 417.

In certain embodiments, the soluble ALK6 fusion polypeptide comprises an amino acid sequence according to SEQ ID NO: 423. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 423.

In certain embodiments, the soluble ALK6 fusion polypeptide comprises an amino acid sequence according to SEQ ID NO: 424. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 424.

In certain embodiments, the soluble ALK6 fusion polypeptide comprises an amino acid sequence according to SEQ ID NO: 430. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 430.

In certain embodiments, the soluble ALK6 fusion polypeptide comprises an amino acid sequence according to SEQ ID NO: 436. Therefore ALK6 fusion polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to ALK6 of SEQ ID NO: 436.

In certain embodiments, the soluble ALK6 fusion polypeptides (not including the leader peptide (e.g., SEQ ID NO: 9)) can be selected from the group consisting of: SEQ ID NOs: 46-50, 52-56, 60, 62, 64, 66, 68, 70, 72, 74, 76, 78, 84, 85, 87, 89, 91, 93, 95, 97, 99, 101, 103, 105, 111, 112, 114-129, 203, 214, 219, 224, 229, 234, 238, 243, 248, 253, 258, 263, 268, 273, 278, 282, and 284. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 46-50, 52-56, 60, 62, 64, 66, 68, 70, 72, 74, 76, 78, 84, 85, 87, 89, 91, 93, 95, 97, 99, 101, 103, 105, 111, 112, 114-129, 203, 214, 219, 224, 229, 234, 238, 243, 248, 253, 258, 263, 268, 273, 278, 282, and 284.

In certain embodiments, the soluble ALK6 fusion polypeptides (not including the leader peptide (e.g., SEQ ID NO: 9)) can be selected from the group consisting of: SEQ ID NOs: 291, 297, 303, 309, 315, 321, 327, 333, 339, 345, 351, 357, 363, 369, 375, 381, 387, 393, 399, 405, 411, 417, 423, 424, 430, and 436. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 291, 297, 303, 309, 315, 321, 327, 333, 339, 345, 351, 357, 363, 369, 375, 381, 387, 393, 399, 405, 411, 417, 423, 424, 430, and 436.

In certain embodiments, soluble ALK6 fusion polypeptides (not including the leader peptide (e.g., SEQ ID NO: 9)) can be selected from the group consisting of: SEQ ID NOs: 87, 89, 91, 112, 234, 243 and 284. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOS: 87, 89, 91, 112, 234, 243 and 284.

In certain other embodiments, soluble ALK6 fusion polypeptides (not including the leader peptide (e.g., SEQ ID NO: 9)) can be selected from the group consisting of: SEQ ID NOs: 132-156, 158-189, 191-194, 202, 213, 218, 223, 228, 233, 242, 247, 252, 257, 262, 272, 277 and 283. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 132-156, 158-189, 191-194, 202, 213, 218, 223, 228, 233, 242, 247, 252, 257, 262, 272, 277 and 283.

In certain other embodiments, soluble ALK6 fusion polypeptides (not including the leader peptide (e.g., SEQ ID NO: 9)) can be selected from a sequence according to SEQ ID NOs: 291 or 424. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence of SEQ ID NOs: 291 or 424.

Other fusion proteins may also be derived from this disclosure as would be understood by those of ordinary skill in the art.

### Compositions and Pharmaceutical Formulations

The polypeptides described herein may be combined with one or more pharmaceutically acceptable carriers prior to administration to a host. A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington's: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration of polypeptides and/or fragments thereof to humans or other subjects. Pharmaceutical compositions may also include carriers, thickeners, diluents, buffers, preservatives, surface active agents, adjuvants, immunostimulants, in addition to the immunogenic polypeptide. Pharmaceutical compositions may also include one or more active ingredients such as antimicrobial agents, anti-inflammatory agents and anesthetics. The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of polypeptide and/or fusion protein disclosed herein.

Methods for treating one or more disease conditions in a mammalian host comprising administering to the mammal at least one or more effective doses of one or more binding agents (and/or derivative(s) thereof) described herein are also provided, as disclosed in more detail below. In some embodiments, one or more polypeptides and/or fusion proteins and/or compositions comprising the same may be administered in a dosage amount of about 1 to about 50 mg (of polypeptide or protein)/kg, about 1 to about 30 mg/kg, or about 5 to about 30 mg/kg (e.g., about any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 mg/kg). In certain embodiments, the one or more polypeptides and/or fusion proteins and/or compositions comprising the same may be administered to the mammal (e.g., intradermally, intravenously, orally, rectally) at about 10 mg/kg one or more times. When multiple doses are administered, the doses may comprise about the same or different amount of binding agent in each dose. The doses may also be separated in time from one another by the same or different intervals. For instance, the doses may be separated by about any of 6, 12, 24, 36, 48, 60, 72, 84, or 96 hours, one week, two weeks, three weeks, one month, two months, three months, four months, five months, six months, seven months, eight months, nine months, 10 months, 11 months, 12 months, 1.5 years, 2 years, 3 years, 4 years, 5 years, or any time period before, after, and/or between any of these time periods. In some embodiments, the polypeptides and/or fusion proteins and/or compositions comprising the same may be administered in conjunction with other agents (e.g., anti-infective agents and/or chemotherapeutic agent). Such other agents may be administered about simultaneously with the polypeptides and/or fusion proteins and/or compositions comprising the same, or at a different time and/or frequency. Other embodiments of such methods may also be appropriate as could be readily determined by one of ordinary skill in the art.

In embodiments the pharmaceutic compositions of this disclosure are administered in any way suitable to effectively achieve a desired therapeutic effect in the eye. Thus, methods of administration include without limitation, topical, intraocular (including intravitreal), transdermal, oral, intravenous, subconjunctival, subretinal, or peritoneal routes of administration. In certain embodiments, the pharmaceutical composition comprising a soluble ALK-6 Fc fusion protein is configured to be administered via an intraocular (including intravitreal) injection.

### Methods of Treatment

The present disclosure relates to methods for the treatment of a retinopathy disorder. In embodiments, the retinopathy disorder is a neovascular retinopathy disorder. Provided herein is a method for the treatment of diabetic retinopathy in a human subject in need thereof (alleviating one or more symptoms, or stasis of one or more symptoms) by administering to the human subject a therapeutically effective amount of a composition provided herein. The treatment can result in improving the patient's condition and can be assess by determining if one or more of the following factors has occurred: decreased macular edema, or increased visual acuity. The compounds described herein can also be used in medicaments for the treatment of diabetic retinopathy.

As used herein “patient” and “human subject in need thereof” are used interchangeably. As used herein, “patient” or “human subject in need thereof” is a human who exhibits one or more clinical manifestations and/or symptoms of a disease or disorder described herein. In certain situations, the patient may be asymptomatic and yet still have clinical manifestations of the disease or disorder. In one embodiment, a patient to be treated is a human being.

As used herein, “diabetic retinopathy” refers to changes in the retina due to the micro vascular changes seen in diabetes. The blood vessels that supply oxygen to the retina of the eye are damaged due to long-term high levels of blood sugar (hyperglycemia). The disease generally develops slowly over a period of months but over time, diabetic retinopathy can get worse and cause vision loss. Diabetic retinopathy usually affects both eyes. Diabetic retinopathy progresses from mild non-proliferative abnormalities, characterized by increased vascular permeability, to moderate and severe non-proliferative diabetic retinopathy (NPDR), characterized by vascular closure, to proliferative diabetic retinopathy (PDR), characterized by the growth of new blood vessels on the retina and posterior surface of the vitreous. Macular edema, characterized by retinal thickening from leaky blood vessels, can develop at all stages of retinopathy. Furthermore conditions such as pregnancy, puberty, blood glucose control, hypertension, and cataract surgery can accelerate these changes.

Non-proliferative diabetic retinopathy, proliferative diabetic retinopathy and diabetic maculopathy are the three main types of diabetic retinopathy.

Non-Proliferative Diabetic Retinopathy (NDPR) is considered as the early stage of retinopathy and is the most common seen in diabetics. The tiny blood vessels in the retina are only mildly affected, but may form bulges (micro aneurysms) and connections with each other (intraretinal micro vascular anomalies) and/or leak fluid (edema), protein deposits (exudates) and blood (hemorrhage). Another typical sign of non-proliferative diabetic retinopathy (NPDR) is the presence of puffy white patches on the retina (cotton wool spots). These changes can occur anywhere throughout the retina, including the macula.

There are three stages of non-proliferative diabetic retinopathy which are detailed below:
(1) Mild Non-proliferative Diabetic Retinopathy: At this earliest stage, at least one micro aneurysm may occur. Micro aneurysms are small areas of balloon-like swelling in the retina's blood vessels.
(2) Moderate Non-proliferative Diabetic Retinopathy: As the disease progresses, some blood vessels that nourish the retina are blocked.
(3) Severe Non-proliferative Diabetic Retinopathy: Many more blood vessels are blocked, depriving several areas of the retina of blood supply. These areas of the retina send signals to the body to grow new blood vessels for nourishment.

Non-proliferative diabetic retinopathy should not cause any problems to the patient, as the vision remains normal as long as the macula is not affected. However, as the symptoms of diabetic retinopathy are generally not visible in this stage, it is recommended that regular retinal screening eye tests should be done to monitor the signs of progression to more serious stages of retinopathy.

Proliferative Diabetic Retinopathy (PDR): This stage comes after severe non-proliferative diabetic retinopathy and is characterized by the growth of abnormal new blood vessels in the eye. When the diabetes causes the blood vessels to become blocked, parts of the eye and retina develop ischemia, as they become starved of oxygen and nutrients. The eye tries to respond to this condition, by growing a new blood supply to the oxygen starved areas. Unfortunately, fragile new blood vessels that bleed easily are formed instead. This process is called neo-vascularization. These abnormal new blood vessels grow in the wrong place on the surface of the retina and into the vitreous gel. Vitreous hemorrhage occurs when these new blood vessels bleed into the vitreous cavity. The blood blocks light that enters the eye from reaching the retina. The amount of sight loss can be mild to severe, and depends on how much blood is in the eye. The vision might slowly improve as the hemorrhage gradually clears over several months.

Abnormal new vessels also cause the formation of scar tissue which pulls on the retina and may result in tractional retinal detachment. The retinal detachment can affect any part of the retina. If it affects the macula, the patient might lose his/her central vision and it can be treated only with surgery.

Diabetic Maculopathy: Diabetic maculopathy is the most common cause of visual loss in diabetes. It occurs when the macula becomes affected by the retinopathy changes caused by diabetes. The macula is located at the center of the retina and is important for central vision and for seeing fine details clearly. Therefore, the central vision and ability to see detail will be affected in the patients that develop diabetic maculopathy. For instance, the affected individuals might find it difficult to recognize faces in the distance or to read small prints. The amount of sight loss may be mild to severe. However, even in the worst cases, the peripheral (side) vision that allows the individual to get around at home and outside will remain unaffected.

Diabetic retinopathy (DR) is an ocular disorder characterized by excessive angiogenesis that develops in diabetes due to thickening of capillary basement membranes, and lack of contact between pericytes and endothelial cells of the capillaries. Loss of pericytes increases leakage of the capillaries and leads to breakdown of the blood-retina barrier. Diabetic retinopathy is the result of microvascular retinal changes. Hyperglycemia-induced pericyte death and thickening of the basement membrane lead to incompetence of the vascular walls. These damages change the formation of the blood-retinal barrier and also make the retinal blood vessels become more permeable. Small blood vessels—such as those in the eye—are especially vulnerable to poor blood sugar (blood glucose) control. An over-accumulation of glucose and/or fructose damages the tiny blood vessels in the retina. Macular edema can also develop when the damaged blood vessels leak fluid and lipids onto the macula. These fluids make the macula swell, which blurs vision. This damage also results in a lack of oxygen at the retina.

As the disease progresses, the lack of oxygen in the retina stimulates angiogenesis along the retina and in the clear, gel-like vitreous humor that fills the inside of the eye. Without timely treatment, these new blood vessels can bleed, cloud vision, and destroy the retina. Fibrovascular proliferation can also cause tractional retinal detachment. The new blood vessels can also grow into the angle of the anterior chamber of the eye and cause neovascular glaucoma.

Vision loss from diabetic maculopathy occurs in 2 ways.

Diabetic macular edema (DME) is the swelling and thickening of the macula. This is due to fluid leakage from the retinal blood vessels in the macula. The vision becomes blurry because the structure and function of the macular photoreceptor cells becomes disrupted. Vision loss from macular edema can be controlled with laser and injections into the eyeball.

Macular ischemia occurs when the tiny retinal blood vessels (capillaries) to the macula close up. The vision becomes blurry because the macula does not receive enough blood supply for it to work properly. Unfortunately, there are no effective treatments for macular ischemia. Macular edema is due to leakage of fluid from the retinal blood vessels. Hard exudates are the yellowish deposits seen on the retina. They are caused by leakage of protein material.

The following medical conditions are some of the possible causes of diabetic retinopathy.

Diabetes: Prolonged hyperglycemia (high blood glucose levels) affects the anatomy and function of retinal capillaries. The excess glucose is converted into sorbitol when it is diverted to alternative metabolic pathways. Sorbitol leads to death or dysfunction of the pericytes of the retinal capillaries. This weakens the capillary walls allowing for the formation of micro aneurysms, which are the earliest signs of diabetic retinopathy. The weak capillary walls can also be responsible for increased permeability and the exudates. Due to the predisposition to increased platelet aggregation and adhesion (blood clot formation) as a result of diabetes, the capillary circulation becomes sluggish or even totally impaired by an occlusion. This can also contribute to the development of diabetic retinopathy.

Type 1 and Type 2 diabetes: Individuals diagnosed with type 1 diabetes, are considered insulin-dependent as they require injections or other medications to supply the insulin that the body is unable to produce on its own. Due to lack of insulin the blood sugar is unregulated and levels are too high. Individuals with type 2 diabetes are considered non-insulin-dependent or insulin-resistant. The individuals affected with this type of diabetes, produce enough insulin but the body is unable to make proper use of it. The body then compensates by producing even more insulin, which can cause an accompanying abnormal increase in blood sugar levels. All people with Type I diabetes (juvenile onset) and with Type II diabetes (adult onset) are at risk of developing diabetic retinopathy. However, people with Type 1 diabetes are more likely to cause retinopathy compared to type 2 diabetes.

Diabetes mellitus type 1 and Diabetes mellitus type 2: People with Diabetes mellitus type 1 and type 2 are at increased risk of developing diabetic retinopathy.

Excessive alcohol: Alcohol if used to extreme reduces Vitamin B12 and thiamine levels. However, alcohol consumption alone is not associated with diabetic retinopathy, the consumption of empty calories from alcohol makes adhering to a calorie-restricted diabetic diet very difficult and it is unclear that what effect moderate alcohol has on retinopathy.

Hypertension and other vascular risk factors such as obesity and dyslipidaemia can influence the onset and progression of retinopathy.

High cholesterol: Cholesterol can exacerbate retinopathy by hardening of large artery blood vessels and can cause damage to the small blood vessels of the eye.

Renal disease, as evidenced by proteinuria and elevated urea/creatinine levels, is an excellent predictor of the presence of retinopathy.

Pregnancy: It can exacerbate existing retinopathy though probably not cause it directly. Women with diabetes have a slightly higher risk during pregnancy. It is recommended that all pregnant women with diabetes have dilated eye examinations each trimester to protect their vision.

Kidney impairment: Associated with diabetic retinopathy, though it appears that diabetic retinopathy leads to kidney impairment rather than vice versa.

Chromosome 15q deletion: A rare chromosomal disorder involving deletion of genetic material from the long arm of chromosome 15.

It is thought that intraocular surgery may possibly increase the risk of progression of diabetic retinopathy.

There are often no symptoms in the earliest stages of non-proliferative diabetic retinopathy. The signs and symptoms of diabetic retinopathy are commonly presented as the disease progresses toward advanced or proliferative diabetic retinopathy. The diagnostic signs of diabetic retinopathy include one more of the following: changes in the blood vessels; retinal swelling (macular edema); pale deposits on the retina; damaged nerve tissue; visual appearance of leaking blood vessels; loss of central or peripheral vision; temporary or permanent vision loss; development of a scotoma or shadow in the field of view; spotty, blurry, hazy or double vision; eye pain; near vision problems unrelated to presbyopia; spots or dark strings floating in the vision (floaters); impaired color vision; vision loss; a dark or blind spot in the central vision; poor or reduced night vision; venous dilation and intraretinal micro vascular abnormalities; in the advanced stage of retinopathy tiny blood vessels grow along the retina, in the clear, gel-like vitreous humor that fills the inside of the eye; nerve damage (neuropathy) affecting ocular muscles that control eye movements; involuntary eye movement (nystagmus); fluctuating and progressive deterioration of vision; macular edema; macular ischemia; traction retinal detachment; sudden, severe painless vision loss; increased vascular permeability, leading to edema; endothelial cell proliferation; flashes of light (photopsias) or defects in the field of vision; presence of abnormal blood vessels on the iris (rubeosis), cataract (associated with diabetes) and vitreous cells (blood in the vitreous or pigmented cells if there is a retinal detachment with hole formation); micro aneurysms—physical weakening of the capillary walls which predisposes them to leakages; hard exudates—precipitates of lipoproteins/other proteins leaking from retinal blood vessels; haemorrhages—rupture of weakened capillaries, appearing as small dots/larger blots or ‘flame’ hemorrhages that track along nerve-fiber bundles in superficial retinal layers (the haemorrhage arises from larger and more superficial arterioles); cotton wool spots—build-up of axonal debris due to poor axonal metabolism at the margins of ischaemic infarcts; and neo-vascularization—an attempt (by residual healthy retina) to revascularize hypoxic retinal tissue.

Described herein are soluble ALK6 fusion proteins which bind their BMP ligands and sequester the ligands preventing their binding to endogenous transmembrane receptors and thus inhibiting signal transduction for the treatment of retinopathy.

As a system, the mammalian retina is subject to diseases that affect the balanced interconnection of the neural retina and the vasculature that nourishes it; visual loss occurs when this balance is disturbed. Diseases such as photoreceptor degenerations that primarily affect the neural retina also affect the retinal vasculature. Diseases that are clinically characterized by abnormality in the choroidal or retinal vasculature, such as ROP, also affect the retinal neurons. These conditions all involve hypoxic ischemic disorders of neural tissue. Photoreceptors are specialized cells that have the highest oxygen requirements of any cell in the body, which plays a role in all hypoxic ischemic diseases of the retina.

In normal development, as the rod photoreceptors differentiate and begin to produce rhodopsin (the molecule responsible for the capture of light); their extraordinarily high oxygen demands render the retina hypoxic, driving the growth of the retinal blood vessels. However, in ROP, supplemental oxygen administered for the acute cardiopulmonary care of the prematurely born infant renders the retina hyperoxic, interrupting normal vascular growth and leaving the peripheral retina avascular. Upon cessation of the supplemental oxygen, the peripheral retina becomes hypoxic. Hypoxia instigates a molecular cascade that leads to the formation of the abnormal retinal blood vessels that are clinically used to diagnose ROP. Even though a premature infant is subjected to high ambient oxygen, immature lungs and other medical complications often lead to fluctuations in blood oxygen and, consequently, to episodes of both hypoxia and hyperoxia at the retina which affect the sensitive photoreceptors. The developing neural retina and its vasculature are under cooperative molecular control, and the vascular abnormalities of ROP are related to the function of the neural retina. Recent studies have found that the degree of dysfunction of the rods in ROP helps predict the degree of abnormality observed in the retinal vasculature, but the degree of abnormality observed in the retinal vasculature may not help predict the degree of dysfunction of the rods in ROP. Thus, the rods cause ROP.

As used herein, an “immature retina” refers to a retina of a preterm infant or a retina of similar morphology/function to that of a pre-term infant retina. An immature retina can be characterized by the presence of poorly developed or disorganized blood vessels with or without the presence of scar tissue. In general, a human preterm infant is one born at 37 weeks gestation, or earlier. Conversely, the term “retinal maturity” refers to a retina of a full-term infant or a retina of similar morphology/function to that of a full-term infant.

As used herein, the phrases “reduces rod energy demand” or “suppresses rod energy demand” refer to a reduction in oxygen demand of a rod cell of at least 10%; preferably the reduction of oxygen demand of a rod cell is at least 20%, at least 30%, at least 40%, at least 50%, at least 60%, at least 70%, at least 80%, at least 90% or more. In general, it is preferred that the oxygen demand of a rod cell is maintained below the level necessary to induce pathological angiogenesis (i.e., blood vessel growth) or vascular abnormalities.

As used herein, the term “vascular abnormalities” is used to refer to an abnormal or pathological level of vascular blood vessel growth (e.g., angiogenesis) or morphology (e.g., tortuosity) that does not permit proper development of the retina to “retinal maturity” as that term is used herein. One of skill in the art can titrate the amount of agent administered or the timing of administration to maintain the growth and morphology of blood vessels below that of pathological blood vessel growth as assessed by, for example, Laser Doppler Blood Flow analysis. In an alternative embodiment, the level of tortuosity of retinal blood vessels is used to assess the degree of pathological blood vessel morphology and/or growth. Methods for measuring tortuosity are further described herein.

As used herein, the term “supplemental oxygen” refers to a concentration of oxygen above that of ambient air (i.e., about 20-21%) that is necessary to maintain blood oxygen levels in a subject at a desired level. In general, supplemental oxygen is supplied in a clinical setting to maintain a blood oxygen level of 100% as assessed using, for example, transcutaneous oxygen monitoring. Monitoring blood oxygen levels and altering the level of “supplemental oxygen” to maintain, for example, a 100% blood oxygen level is a standard procedure in a clinical setting (e.g., a neonatal intensive care unit) and is well known to those of skill in the art of medicine.

### Rod Cell Physiology and Metabolism

The rods perform three linked, metabolically demanding processes: generation of the dark current, maintenance of the visual pigment (the visual cycle), and outer segment turnover, all of which ensue concomitant to developmental elongation of the rod outer segments (ROS) and increase of the rhodopsin content of the eye. The signal transduction mechanism of the rods is physiologically unique. In darkness, sodium and other cations intromitted through cyclic guanosine monophosphate (cGMP) gated channels in the ROS are expelled by pumps in the rod inner segment (RIS) so rapidly that a volume equal to the entire cytosol is circulated every half minute (Hagins, et al., Proc Natl Acad Sci USA (1989) 86:1224). The molecular cascade initiated by photon capture by rhodopsin following a flash of light and leading to a reduction of cGMP leads the dark current to decay following the form of a delayed Gaussian that can be described by an intrinsic amplification constant, A (Lamb and EPugh, J Physiol (1992) 449:719; and Pugh and Lamb, Biochem Biophys Acta (1993) 1141:111).

Following photon capture, rhodopsin's chromophore (retinol) undergoes an isomeric change which frees it from opsin and initiates phototransduction. Spent chromophore is passed from the ROS to the retinal pigment epithelium (RPE) where it undergoes a series of transformations before being returned to the ROS through the apical processes of the RPE as retinol again. There it becomes covalently linked to its active-site lysine in opsin, becoming rhodopsin again and completing the visual cycle (R. R. Rando, Chem Rev (2001) 101:1881). The rate-limiting step in the visual cycle mediated by the isomerohydrolase enzyme complex, RPE65 (Moiseyev et al., Proc Natl Acad Sci USA (2005) 102:12413). Other byproducts of photo-transduction in the ROS are expelled through a process of circadian shedding of the ROS tips; each RPE cell phagocytizes thousands of disks shed from 30-50 embedded rods each day (R. W. Young, J Cell Biol (1967) 33:61). Controlled down-regulation of the visual cycle through targeted inhibition of RPE65 activity lowers the flux of retinoids through the ROS/RPE complex; this would render the rods less vulnerable to insult from hyperoxia and hypoxia (Wellard et al., Vis Neurosci (2005) 22:501) by reducing their metabolic demands. It may also slow phagocytosis and thus lengthen the rod outer segments.

### Macular Degeneration

Macular Degeneration refers to the loss of photoreceptors in the portion of the central retina, termed the macula, responsible for high-acuity vision. Degeneration of the macula is associated with abnormal deposition of extracellular matrix components and other debris in the membrane between the retinal pigment epithelium and the vascular choroid. This debris-like material is termed drusen. Drusen is observed with a funduscopic eye examination. Normal eyes may have maculas free of drusen, yet drusen may be abundant in the retinal periphery. The presence of soft drusen in the macula, in the absence of any loss of macular vision, is considered an early stage of AMD.

### Age-Related Macular Degeneration

Age-related Macular Degeneration (AMD) refers to a disease that causes abnormality in the macula of the retina; it is the leading cause of vision loss in Europe and the United States. In Japan, the disease is also steadily increasing because of the aging population. The macula is located in the center of the retina, and the region is densely populated with cone cells among the photoreceptor cells. Rays of light coming from outside are refracted by the cornea and crystalline lens, and then converge on the macula, the central fovea in particular. The ability to read letters depends on the function of this area. In age-related macular degeneration, the macula, which is an important area as described above, degenerates with age and results in visual impairment, mainly in the form of image distortion (anorthopia) and central scotoma.

Central geographic atrophy, the “dry” form of advanced AMD, results from atrophy to the retinal pigment epithelial layer below the retina, which causes vision loss through loss of photoreceptors (rods and cones) in the central part of the eye. Neovascular or exudative AMD, the “wet” form of advanced AMD, causes vision loss due to abnormal blood vessel growth (choroidal neovascularization) in the choriocapillaris, through Bruch's membrane, ultimately leading to blood and protein leakage below the macula. Bleeding, leaking, and scarring from these blood vessels eventually cause irreversible damage to the photoreceptors and rapid vision loss if left untreated. The wet form of age-related macular degeneration is a disease with a poor prognosis, which results in rapid and severe visual impairment. The major pathological condition is choroidal neovascularization.

### Choroidal Neovascularization

Choroidal Neovascularization (CNV) refers to the creation of new blood vessels in the choroid layer of the eye. CNV can occur rapidly in individuals with defects in Bruch's membrane, the innermost layer of the choroid. It is also associated with excessive amounts of vascular endothelial growth factor (VEGF). As well as in wet AMD, CNV can also occur frequently with the rare genetic disease pseudoxanthoma elasticum and rarely with the more common optic disc drusen. CNV has also been associated with extreme myopia or malignant myopic degeneration, where in choroidal neovascularization occurs primarily in the presence of cracks within the retinal (specifically) macular tissue known as lacquer cracks.

CNV can create a sudden deterioration of central vision, noticeable within a few weeks. Other symptoms which can occur include metamorphopsia, and color disturbances. Hemorrhaging of the new blood vessels can accelerate the onset of symptoms of CNV.

CNV can be detected by measuring the Preferential Hyperacuity Perimeter. On the basis of fluorescein angiography, CNV may be described as classic or occult. PHP is a specialized perimeter that applies principles of both static and automated perimetry to detect defects in the visual field. Rather than measuring peripheral visual fields, PHP relies on the concept of hyperacuity to measure subtle differences in the central and paracentral fields. Hyperacuity is the ability to discern a subtle misalignment of an object. Hyperacuity, or Vernier acuity, has a threshold of 3 to 6 seconds of arc in the fovea. Therefore, hyperacuity's threshold is approximately 10 fold lower than that required for optimal resolution of an object, which is 30 to 60 seconds of arc in the fovea.

Choroidal neovascularization (CNV) commonly occurs in macular degeneration in addition to other ocular disorders and is associated with proliferation of choroidal endothelial cells, overproduction of extracellular matrix, and formation of a fibrovascular subretinal membrane. Retinal pigment epithelium cell proliferation and production of angiogenic factors appears to effect choroidal neovascularization.

CNV refers to ectopic growth of choroidal vessels, penetrating through Bruch's membrane and retinal pigment epithelia. In wet age-related macular degeneration, hemorrhage and leakage of plasma components comprising fat from the premature vascular plexus is the direct cause of the rapid functional impairment of the neural retina. CNV is thought to be induced by inflammatory cells mainly comprising macrophages that infiltrate to phagocytose drusen accumulated at the subretinal macular area. Inflammatory cells such as macrophages are also sources of production of angiogenic factors, such as vascular endothelial growth factor (VEGF), and they function to enhance neovascularization at sites of inflammation. This process is called “inflammatory neovascularization”. Meanwhile, drusen comprise advanced glycation end-products (AGE) and amyloid beta, which are substances that stimulate VEGF production; these substances stimulate retinal pigment epithelia that have migrated to engulf drusen, resulting in VEGF secretion, and this is thought to be another possible mechanism by which CNV develops. Diseases involving CNV include myopic choroidal neovascularization and idiopathic choroidal neovascularization as well as age-related macular degeneration. Development of diseases involving CNV can sometimes be ascribed to angioid streaks, injury, uveitis, or such. Tissue damage mainly of the Bruch's membrane and retinal pigment epithelia in the subretinal macular area, and the subsequent inflammation, have been suggested to be involved in the mechanism of CNV onset in these diseases, as well as in age-related macular degeneration.

Provided herein are methods for the treatment of retinopathy in a human subject in need thereof comprising: administering an effective amount of a pharmaceutical composition to a subject in need thereof, wherein the composition comprises a soluble recombinant bone morphogenetic protein receptor type-1B fusion protein comprising an ALK6 ECD polypeptide and a Fc sequence, wherein the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 1; SEQ ID NO: 4; SEQ ID NO: 11, SEQ ID NO: 69, SEQ ID NO: 80; SEQ ID NO: 90; SEQ ID NO: 113; SEQ ID NO: 153; SEQ ID NO: 154; SEQ ID NO: 155, SEQ ID NO: 156 and SEQ ID NO: 286.

The compositions provided herein can be administered once or multiple times depending on the health of the patient, the progression of the disease or condition, and the efficacy of the treatment. Adjustments to therapy and treatments can be made throughout the course of treatment.

Signs and symptoms of diabetic retinopathy include, but are not limited to, one or more of the following: changes in the blood vessels; retinal swelling (macular edema); pale deposits on the retina; damaged nerve tissue; visual appearance of leaking blood vessels; loss of central or peripheral vision; temporary or permanent vision loss; spotty, blurry, hazy or double vision; eye pain; floaters; impaired color vision; vision loss; a dark or blind spot in the central vision; venous dilation and intraretinal micro vascular abnormalities; neuropathy; fluctuating and progressive deterioration of vision; macular edema; macular ischemia; traction retinal detachment; endothelial cell proliferation; photopsias; rubeosis; micro aneurysms; hard exudates; hemorrhages; and cotton wool spots; are the symptoms for diabetic retinopathy.

In one embodiment, treatment of DR with a compound described herein blocks formation of abnormal blood vessels, slows leakage from blood vessels, reduces retinal swelling, prevents retinal detachment, prevents or slows blindness, and/or reduces vision loss.

The compound to be administered in such methods is administered by any suitable means such as those described herein and known in the art.

For the prevention or treatment of disease, the appropriate dosage of compound will depend, in part, on the patient to be treated, the severity and course of the disease, whether the compound is administered for preventive or therapeutic purposes, previous therapy, the patient's clinical history and response to the compound, and the discretion of the attending physician. The compound is suitably administered to the patient at one time or over a series of treatments.

Provided herein are BMP antagonists for use in the treatment of retinopathy. In certain embodiments, are methods for the treatment of age-related macular degeneration, choroidal neovascularization, diabetic retinopathy and/or retinitis pigmentosa. As provided herein the BMP antagonists are soluble ALK6 fusion proteins which bind their BMP ligands and sequester the ligands preventing their binding to endogenous transmembrane receptors and thus inhibiting signal transduction. In embodiments provided herein, are methods for treating a retinopathy disease by administering a soluble ALK6 fusion protein to a patient in need thereof. In embodiments, the soluble ALK6 fusion protein is selected from any of the fusion proteins described herein comprising an ALK6 ECD sequence (e.g., SEQ ID NO: 1). In particular, comprising an ALK6 ECD sequence selected from any of SEQ ID NO: 3, 5, 79, 81, 82, 83, 108, 109, 112, 130, 157 or 190. In certain embodiments, the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 1; SEQ ID NO: 4; SEQ ID NO: 11, SEQ ID NO: 69; SEQ ID NO: 80; SEQ ID NO: 90; SEQ ID NO: 113; SEQ ID NO: 153; SEQ ID NO: 154; SEQ ID NO: 155; SEQ ID NO: 156 and SEQ ID NO: 286. In certain embodiments, the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence selected from SEQ ID NO: 33; SEQ ID NO: 86; SEQ ID NO: 88, SEQ ID NO: 90, SEQ ID NO: 233, SEQ ID NO: 242 and SEQ ID NO: 286. In certain embodiments, soluble ALK6 fusion polypeptides (not including the leader peptide (e.g., SEQ ID NO: 9)) can be selected from the group consisting of: SEQ ID NOs: 87, 89, 91, 112, 234, 243, 291 or 424. For example, the ALK6 fusion polypeptides may comprise an amino acid sequence that is at least 80%, 85%, 90%, 91%, 92%, 93%, 94%, 95%, 96%, 97%, 98%, or 99% identical to an amino acid sequence selected from the group consisting of: SEQ ID NOs: 87, 89, 91, 112, 234, 243, 291 or 424. In particular, the soluble ALK6 fusion polypeptide for use in treating retinopathy disease comprises an amino acid sequence that is at least 80%, 85%, 90%, 91%, 92%, 93%, 94%, 95%, 96%, 97%, 98%, or 99% identical to an amino acid sequence according to SEQ ID NO: 87. In particular, the soluble ALK6 fusion polypeptide for use in treating a retinopathy disease comprises an amino acid sequence that is at least 80%, 85%, 90%, 91%, 92%, 93%, 94%, 95%, 96%, 97%, 98%, or 99% identical to an amino acid sequence according to SEQ ID NO: 291 or 424.

In certain embodiments provided herein are methods in the treatment of age-related macular degeneration, choroidal neovascularization diabetic retinopathy and/or retinitis pigmentosa, comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker sequence and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102, 113, 132-156, 158-189, 191-194, 202, 213, 218, 223, 228, 233, 242, 247, 252, 257, 262, 272, 277, 283 and 286, wherein amino acid position 60 is a glycine (G) residue. In certain embodiments, the ALK6 ECD sequence is selected from SEQ ID NO: 86, 90, 92, 94, 96, 98, 100, 102, 233, 242, 283 and 286. In certain embodiment, the ALK6 ECD sequence comprises at least one point mutation as compared to SEQ ID NO: 1, such as those selected from K20Y, K20F, K20V, H22S, H22G, H23S, H23G, I33T, S35I and V106F. In certain embodiments, the ALK6 ECD sequence comprises an amino acid insertion between position 22 and position 23 of an alanine (A) residue. In certain embodiments, the ALK6 ECD sequence comprises mutations K20Y, H22S, _22′A, H23G and one or more of V16I and L51M. In particular, the soluble recombinant bone morphogenetic protein receptor type-1B fusion protein for use in treating a retinopathy disease binds the ligand BMP10.

In certain embodiments provided herein are methods in the treatment of age-related macular degeneration, choroidal neovascularization diabetic retinopathy and/or retinitis pigmentosa, comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker sequence and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102, 113, 132-156, 158-189, 191-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, 431 and 437 wherein amino acid position 60 is a glycine (G) residue. In certain embodiments, the ALK6 ECD sequence is selected from SEQ ID NO: 86, 90, 92, 94, 96, 98, 100, 102, 233, 242, 283 and 286. In certain embodiment, the ALK6 ECD sequence comprises at least one point mutation as compared to SEQ ID NO: 1, such as those selected from K20Y, K20F, K20V, H22S, H22G, H23S, H23G 133T, S35I and V106F. In certain embodiments, the ALK6 ECD sequence comprises an amino acid insertion between position 22 and position 23 of an alanine (A) residue. In particular, the soluble recombinant bone morphogenetic protein receptor type-1B fusion protein for use in the treatment of age-related macular degeneration, choroidal neovascularization and/or diabetic retinopathy binds the ligand BMP10. In certain embodiments, the ALK6 ECD sequence comprises mutations K20Y, H22S, _22′A, H23G and one or more of V16I and L51M. In certain other embodiments, the soluble recombinant bone morphogenetic protein receptor type-1B fusion protein for use in the treatment of age-related macular degeneration, choroidal neovascularization and/or diabetic retinopathy does not bind the ligand BMP10.

In certain embodiments provided herein are methods in the treatment of age-related macular degeneration, choroidal neovascularization and/or diabetic retinopathy, comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 157, 190 or 437, wherein amino acid position 60 is a glycine (G) residue.

In certain embodiments provided herein are methods in the treatment of age-related macular degeneration, choroidal neovascularization and/or diabetic retinopathy comprising administering an effective amount of a pharmaceutical composition to a subject in need thereof, wherein the composition comprises a soluble recombinant bone morphogenetic protein receptor type-1B fusion protein comprising an ALK6 ECD polypeptide, a linker and a Fc sequence, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from any one of SEQ ID NO: 132 to 156, 158 to 189, 191-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, 431 and 437 wherein amino acid position 60 is a glycine (G) residue.

In certain embodiments provided herein are methods in the treatment of age-related macular degeneration, choroidal neovascularization and/or diabetic retinopathy, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 87, SEQ ID NO: 91, SEQ ID NO: 291 or SEQ ID NO: 424.

In certain embodiments provided herein are methods of treating age-related macular degeneration, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 87.

In certain embodiments provided herein are methods of treating choroidal neovascularization, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 87.

In certain embodiments provided herein are methods of treating diabetic retinopathy, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 87.

In certain embodiments provided herein are methods of treating age-related macular degeneration, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 291.

In certain embodiments provided herein are methods of treating choroidal neovascularization, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 291.

In certain embodiments provided herein are methods of treating diabetic retinopathy, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 291.

In certain embodiments provided herein are methods of treating age-related macular degeneration, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 424.

In certain embodiments provided herein are methods of treating choroidal neovascularization, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 424.

In certain embodiments provided herein are methods of treating diabetic retinopathy, comprising administering an effective amount of a pharmaceutical composition to a subject in need thereof, wherein the composition comprises a soluble recombinant bone morphogenetic protein receptor type-1B fusion protein having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 424.

In embodiments provided herein are methods for treating age-related macular degeneration, choroidal neovascularization and/or diabetic retinopathy with the ALK-6 Fc fusion polypeptides of the disclosure, wherein the ALK6 fusion polypeptide binds BMP10. In embodiments, the methods comprise administering an effective amount of a composition comprising the ALK-6 Fc fusion polypeptide to a human subject in need thereof. In embodiments, the method is effective to alleviate a symptom of or a condition associated with age-related macular degeneration, choroidal neovascularization and/or diabetic retinopathy. In certain embodiments, the method is effective for improving of at least one of the symptoms described herein. Improvement includes, for example, a 2%, 5%, 10%, 15%, 20%, 25%, 30%, 35%, 40%, 45%, 50%, 55%, 60%, 65%, 70%, 75%, 80%, 85%, 90%, 95%, or 100% improvement in one or more signs or symptoms described herein. Compositions can be administered to a patient in a therapeutically effective amount which is effective for producing some desired therapeutic effect, at a reasonable benefit/risk ratio applicable to any medical treatment. For the administration of the present compositions to human patients, the compositions can be formulated by methodology known by one of ordinary skill in the art.

The eye provides a tissue or structure well suited for topical administration of many drugs. Intraocular injection and oral administration can also be effective. In general, an compound is determined to be “therapeutically effective” in the methods described herein if (a) measurable symptom(s) of, for example, vascular abnormalities, are reduced for example by at least 10% compared to the measurement prior to treatment onset, (b) the progression of the disease is halted (e.g., patients do not worsen or the vasculature stops growing pathologically, or (c) symptoms are reduced or even ameliorated, for example, by measuring a reduction in vessel number or tortuosity. Efficacy of treatment can be judged by an ordinarily practitioner or as described herein and known in the art.

As used herein, “improving rod-mediated retinal function” refers to an increase in rod-mediated retinal function of at least 10%, at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 50-fold, at least 100-fold, at least 1000-fold or higher.

“Rod-mediated retinal function” refers to a function of rod cells in a functioning retina and can include such clinical end-points as degree of peripheral vision, low-level light vision, scotopic/“night vision”, and sensitivity to peripheral movement. Rod-mediated retinal function can be assessed in vivo by, for example, electroretinography measurement of rod activation of photo-transduction or deactivation of photo-transduction; recovery of the dark current following photobleaching; measurement of the ERG a-wave or b-wave; speed of recovery to photo-transduction; or rod-mediated response amplitudes. Methods for measuring rod-mediated retinal function are known in the art and/or explained herein in more detail.

Efficacy of treatment can be monitored by the administering clinician. Where the disease or disorder is retinopathy of prematurity, the International Classification of Retinopathy or Prematurity (ICROP) can be applied. The ICROP uses a range of parameters to classify the disease. These parameters include location of the disease into zones (zones 1, 2 and 3), the circumferential extent of the disease based on clock hours 1-12, severity of the disease (stages 1-5), and the presence or absence of “Plus Disease.”

The zones are centered on the optic nerve. Zone 1 is the posterior zone of the retina, defined as the circle with a radius extending from the optic nerve to double the distance to the macula. Zone 2 is an annulus with the inner border defined by zone 1 and the outer border defined by the radius defined as the distance from the optic nerve to the nasal ora serrata. Zone 3 is the residual temporal crescent of the retina.

The circumferential extent of the disease is described in segments as if the top of the eye were 12 on the face of a clock. For example one might report that there is stage 1 disease for 3 clock hours from 4 to 7 o'clock.

The Stages describe the ophthalmoscopic findings at the junction between the vascularized and avascular retina. Stage 1 is a faint demarcation line. Stage 2 is an elevated ridge. Stage 3 is extraretinal fibrovascular tissue. Stage 4 is sub-total retinal detachment. Stage 5 is total retinal detachment.

In addition, “Plus disease” may be present at any stage. “Plus disease” describes a significant level of vascular dilation and tortuosity observed at the posterior retinal vessels. This reflects the increase of blood flow through the retina.

Any improvement on the ICROP relative to pre-treatment classification is considered to be effective treatment. Similarly, where prevention of disease is the goal, treatment is considered effective if one or more signs or symptoms of ROP is (are) less severe in a treated individual relative to the expected course of disease in a similar individual not receiving such treatment. The disease has been known and characterized to an extent that skilled clinicians can often predict the extent of disease that would occur in the absence of treatment, based, for example, on knowledge of earlier patients. The failure to develop or experience a worsening of one or more symptoms of ROP, or, for that matter any other retinal disease or disorder involving abnormal vascularization, can be considered effective prevention of disease in an individual otherwise expected to develop or experience worsening of such disease. Similarly, any improvement relative to expected disease state in the absence of treatment can be considered effective treatment.

As an alternative to the ICROP scale, other clinically accepted markers of retinal disease known to those of skill in the art can also be measured to monitor or determine the efficacy of treatment or prevention of retinal diseases or disorders as described herein. Generally a difference of at least 10% in a marker of retinal disease is considered significant.

Provided herein are methods for reducing or inhibiting vascularization in the eye (e.g., neovascularization) of a patient. Also provided herein is a method for treating an ophthalmic disease or disorder associated with neovascularization in the eye of a patient wherein the ophthalmic disease or disorder associated with neovascularization is retinal neovascularization. Another embodiment provides a method for treating an ophthalmic disease or disorder associated with neovascularization in the eye of a patient wherein the ophthalmic disease or disorder associated with neovascularization is choroidal neovascularization. Another embodiment provides a method for treating an ophthalmic disease or disorder associated with neovascularization in the eye of a patient wherein the ophthalmic disease or disorder associated with neovascularization is selected from sickle cell retinopathy, Eales disease, ocular ischemic syndrome, carotid cavernous fistula, familial exudative vitreoretinopathy, hyperviscosity syndrome, idiopathic occlusive arteriolitis, radiation retinopathy, retinal vein occlusion, retinal artery occlusion, retinal embolism, birdshot retinochoroidopathy, retinal vasculitis, sarcoidosis, toxoplasmosis, uveitis, choroidal melanoma, chronic retinal detachment, incontinentia pigmenti, and retinitis pigmentosa. Another embodiment provides a method for treating an ophthalmic disease or disorder associated with neovascularization in the eye of a patient wherein the ophthalmic disease or disorder associated with neovascularization is wet age-related macular degeneration. Another embodiment provides a method for treating an ophthalmic disease or disorder associated with neovascularization in the eye of a patient wherein the ophthalmic disease or disorder associated with neovascularization is neovascular age-related macular degeneration.

Provided herein is a method for treating neovascular age-related macular degeneration (e.g., wet age-related macular degeneration (AMD)) or choroidal neovascularization (CNV) in a patient by administering to the patient a therapeutically effective amount of a composition provided herein. The compounds described herein can also be used in medicaments for the treatment of macular degeneration (e.g., age-related macular degeneration (AMD)) or choroidal neovascularization (CNV). As provided herein all references to age-related macular degeneration refer to the neovascular or wet stage of the disease.

Provided herein is a method for treating age-related macular degeneration (AMD) in a patient by administering to the patient a therapeutically effective amount of a composition provided herein. The treatment can result in improving the patient's condition and can be assess by determining if one or more of the following factors has occurred: Drusen; pigmentary alterations; eudative changes (e.g., hemorrhages in the eye, hard exudates, subretinal/sub-RPE/intraretinal fluid); atrophy (incipient and geographic); visual acuity drastically decreasing (two levels or more; ex: 20/20 to 20/80); preferential hyperacuity perimetry changes (for wet AMD); blurred vision (those with non-exudative macular degeneration may be asymptomatic or notice a gradual loss of central vision, whereas those with exudative macular degeneration often notice a rapid onset of vision loss); central scotomas (shadows or missing areas of vision); distorted vision (i.e., metamorphopsia; a grid of straight lines appears wavy and parts of the grid may appear blank); trouble discerning colors (specifically dark ones from dark ones and light ones from light ones); slow recovery of visual function after exposure to bright light; and a loss in contrast sensitivity. Described herein are methods of treating or preventing AMD via the administration of the compounds described herein. The compounds described herein can also be used in medicaments for the treatment of AMD. In one embodiment, one or more signs or symptoms of AMD are improved following administration of one of the compounds described herein to a patient. Improvement also encompasses stasis of one or more symptoms such that they do not worsen.

“Treatment” of diseases involving CNV refers to diseases involving CNV, where a symptom caused by an above disease is suppressed or ameliorated. The treatment of diseases involving CNV also refers to suppressing CNV progression and functional impairment of neural retina caused by hemorrhage or leakage of plasma components from abnormal newly generated vessels.

As used herein, “suppressing CNV” refers to suppressing inflammation in the retina (suppressing the growth of inflammatory cells in the retina) and suppressing the production of angiogenic factors by inflammatory cells, in addition to suppressing neovascularization. An inflammation reaction in the retina may be induced by an injury, or by accumulation of metabolic decomposition products, such as drusen.

CNV can be confirmed to be suppressed by detecting the size (volume) of neovascularization using fluorescein fundus angiography or the like. When the volume of neovascularization is reduced after administration of an agent of the present disclosure, CNV is regarded as suppressed. Methods for detecting CNV are not limited to the methods described above, and CNV can be detected by known methods, and also by the methods described in the Examples herein.

As a disease involving CNV progresses, vision is impaired due to image distortion, central scotoma, and such. In such cases of visual impairment, when visual acuity is improved upon administration of a compound described herein, the compound is regarded as useful for patients with such a disease involving CNV. Provided herein is a method for treating choroidal neovascularization The treatment can result in improving the patient's condition and can be assess by determining if visual acuity has increased. Described herein are methods of treating or preventing choroidal neovascularization via the administration of the compounds described herein.

Choroidal neovascularization (CNV) commonly occurs in macular degeneration in addition to other ocular disorders and is associated with proliferation of choroidal endothelial cells, overproduction of extracellular matrix, and formation of a fibrovascular subretinal membrane. Retinal pigment epithelium cell proliferation and production of angiogenic factors appears to effect choroidal neovascularization. Choroidal neovascularization (CNV), the development of abnormal blood vessels beneath the retinal pigment epithelium (RPE) layer of the retina. These vessels break through the Bruch's membrane, disrupting the retinal pigmented epithelium, bleed, and eventually cause macular scarring which results in profound loss of central vision (disciform scarring).

In one embodiment, treatment of CNV with a compound described herein decreases slows or inhibits development of abnormal blood vessels beneath the retinal pigment epithelium layer of the retina, slows or inhibits damage of the Bruch's membrane, and slows or inhibits disruption of the retinal pigmented epithelium and slows or inhibits macular scarring.

Retinal neovascularization develops in numerous retinopathies associated with retinal ischemia, such as sickle cell retinopathy, Eales disease, ocular ischemic syndrome, carotid cavernous fistula, familial exudative vitreoretinopathy, a hyperviscosity syndrome, idiopathic occlusive arteriolitis, radiation retinopathy, retinal vein occlusion, retinal artery occlusion, or retinal embolism. Retinal neovascularization can also occur with inflammatory diseases (such as birdshot retinochoroidopathy, retinal vasculitis, sarcoidosis, toxoplasmosis, or uveitis), or other conditions such as choroidal melanoma, chronic retinal detachment, incontinentia pigmenti, and rarely in retinitis pigmentosa.

A factor common to almost all retinal neovascularization is retinal ischemia, which is thought to release diffusible angiogenic factors (such as VEGF). The neovascularization begins within the retina and then breaches the retinal internal limiting membrane. The new vessels grow on the inner retina and the posterior surface of the vitreous after it has detached (vitreous detachment). Neovascularization may erupt from the surface of the optic disk or the retina. Retinal neovascularization commonly progresses to vitreoretinal neovascularization. Iris neovasularization and neovascular glaucoma often follow retinal neovascularization.

The efficacy of the treatment of the measured by various endpoints commonly used in evaluating intraocular neovascular diseases. For example, vision loss can be assessed. Vision loss can be evaluated by, but not limited to, e.g., measuring by the mean change in best correction visual acuity (BCVA) from baseline to a desired time point (e.g., where the BCVA is based on Early Treatment Diabetic Retinopathy Study (ETDRS) visual acuity chart and assessment at a test distance of 4 meters), measuring the proportion of subjects who lose fewer than 15 letters in visual acuity at a desired time point compared to baseline, measuring the proportion of subjects who gain greater than or equal to 15 letters in visual acuity at a desired time point compared to baseline, measuring the proportion of subjects with a visual-acuity Snellen equivalent of 20/2000 or worse at a desired time point, measuring the NEI Visual Functioning Questionnaire, measuring the size of CNV and amount of leakage of CNV at a desired time point, e.g., by fluorescein angiography, etc. Ocular assessments can be done, e.g., which include, but are not limited to, e.g., performing eye exam, measuring intraocular pressure, assessing visual acuity, measuring slitlamp pressure, assessing intraocular inflammation, etc.

Although embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the embodiments, specific terminology will be resorted to for the sake of clarity.

The skilled artisan will understand that the figures, described above, and example, described below, are for illustration purposes only. Neither the figures nor the examples are intended to limit the scope of the disclosed teachings in any way.

### EXAMPLES

The following examples are provided to illustrate certain disclosed embodiments and are not to be construed as limiting the scope of this disclosure in any way.

### Example 1: Generation of ALK-6 Fc Fusion Proteins

Applicants constructed a soluble ALK6 wt-Fc dimeric complex comprising the extracellular domain (ECD) of human ALK6 (SEQ ID NO: 1) wherein the ECD is fused to an Fc domain with a linker positioned between the extracellular domain and the Fc domain. The individual constructs are referred to as ALK6 wt-Fc fusion polypeptide, the sequence of which is provided below. In addition, the ALK6-Fc fusion polypeptide employs the leader sequence:
SEQ ID NO: 9 MGWSCIILFLVATATGVHS

The ALK6 wt-Fc polypeptide sequence (SEQ ID NO: 49) is shown below:

MGWSCIILFLVATATGVHSKKEDGESTAPTPRPKVLRCKCHHHCPED
SVNNICSTDGYCFTMIEEDDSGLPVVTSGCLGLEGSDFQCRDTPIPH
QRRSIECCTERNECNKDLHPTLPPLKNRDFVDGPIHHRTGGGGSGGG
GSGGGGSGGGGSTHTCPPCPAPELLGGPSVFLFPPKPKDTLMISRTP
EVTCVVVDVSHEDPEVKFNWYVDGVEVHNAKTKPREEQYNSTYRVVS
VLTVLHQDWLNGKEYKCKVSNKALPAPIEKTISKAKGQPREPQVYTL
PPSREEMTKNQVSLTCLVKGFYPSDIAVEWESNGQPENNYKTTPPVL
DSDGSFFLYSKLTVDKSRWQQGNVFSCSVMHEALHNHYTQKSLSLSP
GK

The leader (signal) sequence and linker are underlined. The amino acid sequence of SEQ ID NO: 49 may optionally be provided with lysine (K) removed from the C-terminus.

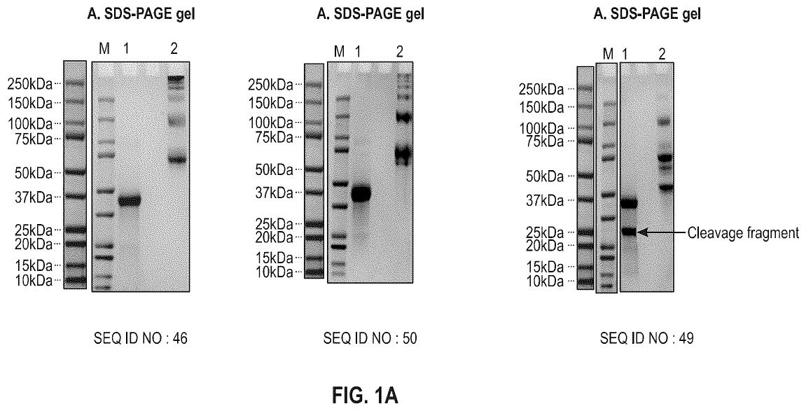

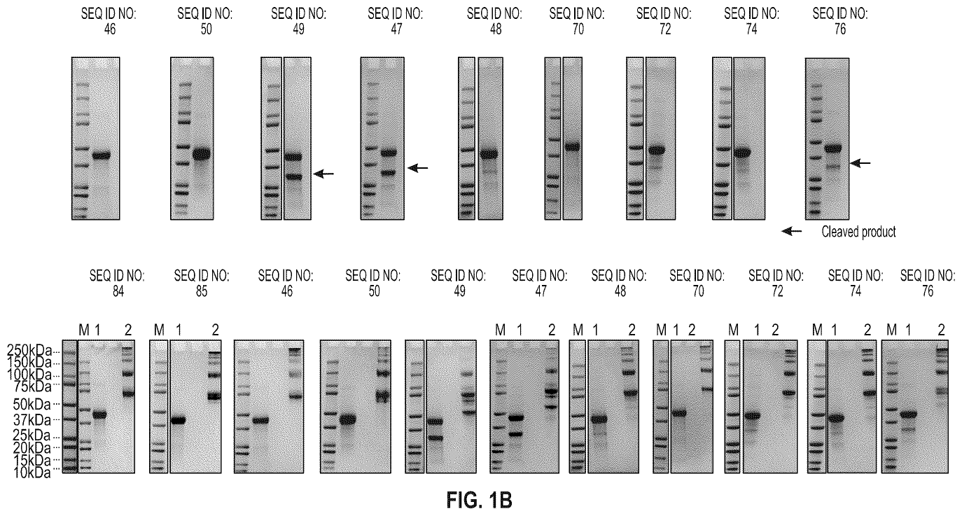

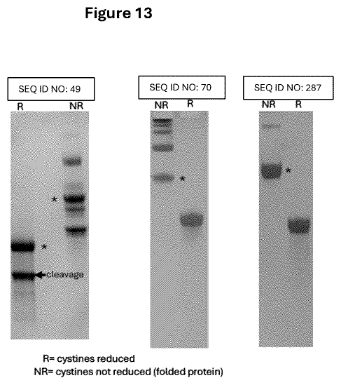

SDS gel analysis of SEQ ID NO: 49 shows a prominent cleavage fragment at about 25 kDa. See FIG. 1 and FIG. 13 (left gel). Based on the fragment size and analysis of the sequence a protease cleavage site in the C-terminus end of the ECD of ALK6 was indicated.

To generate a non-cleavable soluble ALK6 Fc polypeptide, Applicants constructed a soluble ALK-6 Fc fusion polypeptide wherein the C-terminus 14 amino acids (SEQ ID NO: 51; LKNRDFVDGPIHHR) were deleted from the ECD of human wild type ALK6 providing SEQ ID NO: 4. The ECD was fused to an Fc domain with a linker positioned between the ECD and the Fc domain. The individual constructs are referred to as ALK6-Fc fusion polypeptide (SEQ ID NO: 50); the fusion polypeptide sequence of which is provided below.

MGWSCIILFLVATATGVHSKKEDGESTAPTPRPKVLRCKCHHHCPED
SVNNICSTDGYCFTMIEEDDSGLPVVTSGCLGLEGSDFQCRDTPIPH
QRRSIECCTERNECNKDLHPTLPPLTGGGGSGGGGSGGGGSGGGGST
HTCPPCPAPELLGGPSVFLFPPKPKDTLMISRTPEVTCVVVDVSHED
PEVKFNWYVDGVEVHNAKTKPREEQYNSTYRVVSVLTVLHQDWLNGK
EYKCKVSNKALPAPIEKTISKAKGQPREPQVYTLPPSREEMTKNQVS
LTCLVKGFYPSDIAVEWESNGQPENNYKTTPPVLDSDGSFFLYSKLT
VDKSRWQQGNVFSCSVMHEALHNHYTQKSLSLSPGK

The leader (signal) sequence and linker are underlined. The amino acid sequence of SEQ ID NO: 50 may optionally be provided with lysine (K) removed from the C-terminus.

SDS gel analysis of SEQ ID NO: 50 showed no cleave fragment as compared to SEQ ID NO: 49. See FIG. 1.

In another approach to generate a non-cleavable ALK6-Fc fusion polypeptide, Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR). The ALK6 ECD (SEQ ID NO: 11) was fused to a Fc domain with a linker positioned between the ECD and the Fc domain. The individual constructs are referred to as ALK6-Fc fusion polypeptide (SEQ ID NO: 46), the fusion polypeptide sequence of which is provided below.

KKEDGESTAPTPRPKVLRCKCHHHCPEDSVNNICSTDGYCFTMIEED
DSGLPVVTSGCLGLEGSDFQCRDTPIPHQRRSIECCTERNECNKDLH
PTLPPVVIGPFFDGSIRTGGGGSGGGGSGGGGSGGGGSTHTCPPCPA
PELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSN
KALPAPIEKTISKAKGQPREPQVYTLPPSREEMTKNQVSLTCLVKGF
YPSDIAVEWESNGQPENNYKTTPPVLDSDGSFFLYSKLTVDKSRWQQ
GNVFSCSVMHEALHNHYTQKSLSLSPGK

The linker sequence is underlined, and the new C-terminus sequence in bold. The amino acid sequence of SEQ ID NO: 46 may optionally be provided with lysine (K) removed from the C-terminus and/or with a leader sequence.

SDS gel analysis of SEQ ID NO: 46 showed no cleave fragment as compared to SEQ ID NO: 49. See FIG. 1.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D104A introduced into the SEQ ID NO: 1 sequence providing SEQ ID NO: 12. The ECD was fused to a Fc domain with a linker positioned between the ECD and the Fc domain. The individual constructs are referred to as a ALK6-Fc fusion polypeptide (SEQ ID NO: 47), the fusion polypeptide sequence of which is provided below.

KKEDGESTAPTPRPKVLRCKCHHHCPEDSVNNICSTDGYCFTMIEED
DSGLPVVTSGCLGLEGSDFQCRDTPIPHQRRSIECCTERNECNKDLH
PTLPPLKNRAFVDGPIHHRTGGGGSGGGGSGGGGSGGGGSTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVYTLPPSREEMTKNQVSLTCLVK
GFYPSDIAVEWESNGQPENNYKTTPPVLDSDGSFFLYSKLTVDKSRW
QQGNVFSCSVMHEALHNHYTQKSLSLSPGK

The linker sequence and the individual point mutation in the ALK6 ECD are underlined and bold. The amino acid sequence of SEQ ID NO: 47 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D104A introduced into the SEQ ID NO: 1 sequence and seven amino acids deleted from the C-terminus providing SEQ ID NO: 13. The ALK6 ECD was fused to a Fc domain with a linker positioned between the ECD and the Fc domain. The individual constructs are referred to as an ALK6-Fc fusion polypeptide (SEQ ID NO: 48), the fusion polypeptide sequence of which is provided below.

KKEDGESTAPTPRPKVLRCKCHHHCPEDSVNNICSTDGYCFTMIEED
DSGLPVVTSGCLGLEGSDFQCRDTPIPHQRRSIECCTERNECNKDLH
PTLPPLKNRAFV_______TGGGGSGGGGSGGGGSGGGGSTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVYTLPPSREEMTKNQVSLTCLVK
GFYPSDIAVEWESNGQPENNYKTTPPVLDSDGSFFLYSKLTVDKSRW
QQGNVFSCSVMHEALHNHYTQKSLSLSPGK

The linker sequence and the individual point mutation in the ALK6 ECD are underlined and bold. The amino acid sequence of SEQ ID NO: 48 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein seven amino acids were deleted from the C-terminus of ALK-6 ECD of SEQ ID NO: 1 providing SEQ ID NO:69. The ALK6 ECD was fused to a Fc domain with a linker positioned between the ECD and the Fc domain. The individual constructs are referred to as a ALK6-Fc fusion polypeptide (SEQ ID NO: 70), the fusion polypeptide sequence of which is provided below.

KKEDGESTAPTPRPKVLRCKCHHHCPEDSVNNICSTDGYCFTMIEED
DSGLPVVTSGCLGLEGSDFQCRDTPIPHQRRSIECCTERNECNKDLH
PTLPPLKNRDFV_______TGGGGSGGGGSGGGGSGGGGSTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVYTLPPSREEMTKNQVSLTCLVK
GFYPSDIAVEWESNGQPENNYKTTPPVLDSDGSFFLYSKLTVDKSRW
QQGNVFSCSVMHEALHNHYTQKSLSLSPGK

The linker sequence and the deletion mutations in the ALK6 ECD are underlined and bold. The amino acid sequence of SEQ ID NO: 70 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D107A introduced into the SEQ ID NO: 1 sequence providing SEQ ID NO: 71. The ECD was fused to a Fc domain with a linker positioned between the ECD and the Fc domain. The individual constructs are referred to as an ALK6-Fc fusion polypeptide (SEQ ID NO: 72) the fusion polypeptide sequence of which is provided below.

KKEDGESTAPTPRPKVLRCKCHHHCPEDSVNNICSTDGYCFTMIEED
DSGLPVVTSGCLGLEGSDFQCRDTPIPHQRRSIECCTERNECNKDLH
PTLPPLKNRDFVAGPIHHRTGGGGSGGGGSGGGGSGGGGSTHTCPPC
PAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKV
SNKALPAPIEKTISKAKGQPREPQVYTLPPSREEMTKNQVSLTCLVK
GFYPSDIAVEWESNGQPENNYKTTPPVLDSDGSFFLYSKLTVDKSRW
QQGNVFSCSVMHEALHNHYTQKSLSLSPGK

The linker sequence and the point mutation in the ALK6 ECD are underlined and bold. The amino acid sequence of SEQ ID NO: 72 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation D107A introduced into the SEQ ID NO: 1 sequence and two amino acids deleted (H111_ and H112_) from the C-terminus providing SEQ ID NO: 73. The ECD was fused to a Fc domain with a linker positioned between the ECD and the Fc domain. The individual constructs are referred to as ALK6-Fc fusion polypeptide (SEQ ID NO: 74), the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFVAGPI RTGGGGSGGGGSGGGGSGGGGSTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

The linker sequence and the point and deletion mutations in the ALK6 ECD are underlined and bold. The amino acid sequence of SEQ ID NO: 74 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein a point mutation was introduced into the suspected cleavage site with the mutation P109S introduced into the SEQ ID NO: 1 sequence and two amino acids deleted (H112_ and H113_) from the C-terminus providing SEQ ID NO: 75. The ECD was fused to a Fc domain with a linker positioned between the ECD and the Fc domain. The individual constructs are referred to as an ALK6-Fc fusion polypeptide (SEQ ID NO: 76), the fusion polypeptide sequence of which is provided below.

KKEDGESTAPTPRPKVLRCKCHHHCPEDSVNNICSTDGYCFTMIEEDDSG
LPVVTSGCLGLEGSDFQCRDTPIPHQRRSIECCTERNECNKDLHPTLPPL
KNRDFVDGSI RTGGGGSGGGGSGGGGSGGGGSTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

The linker sequence and the point and deletion mutations in the ALK6 ECD are underlined and bold. The amino acid sequence of SEQ ID NO: 76 may optionally be provided with lysine (K) removed from the C-terminus and/or with a leader sequence.

In another approach to generate a non-cleavable ALK6-Fc fusion polypeptide and maintain the ALK6 ligand binding properties, Applicants constructed a soluble ALK-6 Fc fusion polypeptide wherein an additional amino acid was introduced between position 22 and 23 with the mutation _22′A introduced into the SEQ ID NO: 1 sequence and seven amino acids were deleted from the C-terminus of ALK-6 ECD of SEQ ID NO: 1 providing SEQ ID NO:113. The ECD was fused to a Fc domain with a linker positioned between the ECD and the Fc domain. The individual constructs are referred to as an ALK6-Fc fusion polypeptide (SEQ ID NO: 114), the fusion polypeptide sequence of which is provided below.

KKEDGESTAPTPRPKVLRCKCHAHHCPEDSVNNICSTDGYCFTMI
EEDDSGLPVVTSGCLGLEGSDFQCRDTPIPHQRRSIECCTERNECNKDLH
PTLPPLKNRDFV TGGGGSGGGGSGGGGSGGGGSTHTCPPCPAPEA
AGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHN
HYTQKSLSLSPG

The linker sequence and the point and deletion mutations in the ALK6 ECD are underlined and bold as are the two point mutations in the Fc sequence. The amino acid sequence of SEQ ID NO: 114 may optionally be provided with lysine (K) added to the C-terminus and/or with a leader sequence.

### Example 1b: Generation of ALK-6 Fusion Proteins without Aggregation or Cleavage Sites

As described above, SEQ ID NO: 70 (comprising ECD sequence SEQ ID NO: 69) was aggregated on an SDS-PAGE gel but was no longer cleaved as compared to the ALK-6 wt fusion protein (SEQ ID NO: 49).

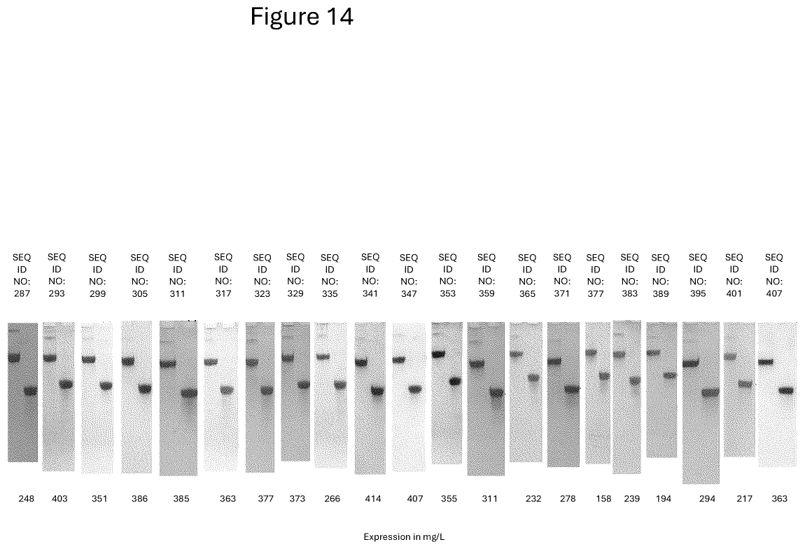

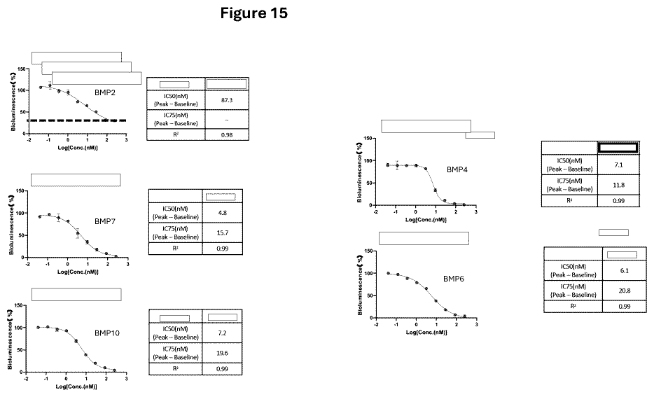

Applicants constructed a number of soluble ALK-6 Fc fusion polypeptides using ECD sequences SEQ ID NO: 69 and subsequently SEQ ID NO: 90 as background sequences introducing one or more point mutations in the modified ECD of ALK6 wherein an amino acid was introduced between position 22 and 23 (SEQ ID NO: 113), referred to herein as Xaa′22 (“SEQ ID NO: 157 variants”, “SEQ ID NO: 190 variants” and “SEQ ID NO: 437 variants”). These variant soluble ALK-6 Fc fusion polypeptides were analyzed on a SDS gel to confirm the lack of aggregation and lack of a cleavage product (See FIGS. 13 and 14) and select variants tested in a cell-based binding assay for ligand binding specificity. See FIG. 15. Note the ALK6 Fc fusion proteins tested in the cell based assays comprised the Fc sequence according to SEQ ID NO: 106, while for purposes of intravitreal dosing in the treatment of a retinopathy disease the ALK6 ECD sequences of this disclosure comprise with the Fc sequence according to SEQ ID NO: 285, or an Fab sequence such as SEQ ID NO: 196 to 201, reducing in vivo half-life and off target systemic effects. The Fc fusion proteins described in this example comprise the Fc sequence according to SEQ ID NO: 285.

In an approach to generate a non-aggregated ALK6-Fc fusion polypeptide and maintain the ALK6 ligand binding properties, Applicants constructed a soluble ALK-6 Fc fusion polypeptide wherein point mutations were introduced at positions 20, 22, 23 and an additional amino acid was introduced between position 22 and 23 with the mutation _22′A introduced into the SEQ ID NO: 69 ECD sequence with seven amino acids deleted from the C-terminus of ALK-6 ECD of SEQ ID NO: 1 providing SEQ ID NO:90. The ECD was fused to a Fc domain with a linker positioned between the ECD and the Fc domain. The individual constructs are referred to as an ALK6-Fc fusion polypeptide (SEQ ID NO: 424), the fusion polypeptide sequence of which is provided below.

KKEDGESTAPTPRPKVLRCYCSAGHCPEDSVNNICSTDGYCFTMIEEDDS
GLPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 424 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein two point mutations (V16I and L51M) were introduced into the SEQ ID NO: 90 sequence with seven amino acids deleted from the C-terminus of ALK-6 ECD of SEQ ID NO: 1 providing SEQ ID NO:286. The ECD was fused to a Fc domain with a linker positioned between the ECD and the Fc domain. The individual constructs are referred to as an ALK6-Fc fusion polypeptide (SEQ ID NO: 291), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 291 may optionally be provided with lysine (K) added to the C-terminus and/or with a leader sequence. The corresponding Fc fusion protein with Fc sequence SEQ ID NO: 106, SEQ ID NO: 287 is no longer aggregated or cleaved. See FIG. 13, right gel.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, E27D, and L51M) were introduced into the SEQ ID NO: 90 sequence with seven amino acids deleted from the C-terminus of ALK-6 ECD of SEQ ID NO: 1 providing SEQ ID NO:292. The ECD was fused to a Fc domain with a linker positioned between the ECD and the Fc domain. The individual constructs are referred to as an ALK6-Fc fusion polypeptide (SEQ ID NO: 297), the fusion polypeptide sequence of which is provided below.

KKEDGESTAPTPRPKILRCYCSAGHCPDDSVNNICSTDGYCFTMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 297 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, V30I, and L51M) were introduced into the SEQ ID NO: 90 sequence with seven amino acids deleted from the C-terminus of ALK-6 ECD of SEQ ID NO: 1 providing SEQ ID NO:298. The ECD was fused to a Fc domain with a linker positioned between the ECD and the Fc domain. The individual constructs are referred to as an ALK6-Fc fusion polypeptide (SEQ ID NO: 303), the fusion polypeptide sequence of which is provided below.

KKEDGESTAPTPRPKILRCYCSAGHCPEDSINNICSTDGYCFTMIEEDDS
GMPVVTSGCLGLEGSDFQCRDTPIPHQRRSIECCTERNECNKDLHPTLPP
LKNRDFV TGGGGSGGGGSGGGGGGGGSTHTCPPCPAPEAAGGPSV
FLFPPKPKDTLMASRTPEVTCVVVDVSHEDPEVKFNWYVDGVEVHNAKTK
PREEQYNSTYRVVSVLTVLAQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

The linker sequence and the point and deletion mutations in the ALK6 ECD are underlined and bold as are the two point mutations in the Fc sequence. The amino acid sequence of SEQ ID NO: 303 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, I33T, and L51M) were introduced into the SEQ ID NO: 90 sequence with seven amino acids deleted from the C-terminus of ALK-6 ECD of SEQ ID NO: 1 providing SEQ ID NO:304. The ECD was fused to a Fc domain with a linker positioned between the ECD and the Fc domain. The individual constructs are referred to as an ALK6-Fc fusion polypeptide (SEQ ID NO: 309), the fusion polypeptide sequence of which is provided below.

KKEDGESTAPTPRPKILRCYCSAGHCPEDSVNNTCSTDGYCFTMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 309 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, S35I, and L51M) were introduced into the SEQ ID NO: 90 sequence with seven amino acids deleted from the C-terminus of ALK-6 ECD of SEQ ID NO: 1 providing SEQ ID NO:310. The ECD was fused to a Fc domain with a linker positioned between the ECD and the Fc domain. The individual constructs are referred to as an ALK6-Fc fusion polypeptide (SEQ ID NO: 315), the fusion polypeptide sequence of which is provided below.

KKEDGESTAPTPRPKILRCYCSAGHCPEDSVNNICITDGYCFTMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 315 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, D37N, and L51M) were introduced into the SEQ ID NO: 90 sequence with seven amino acids deleted from the C-terminus of ALK-6 ECD of SEQ ID NO: 1 providing SEQ ID NO:316. The ECD was fused to a Fc domain with a linker positioned between the ECD and the Fc domain. The individual constructs are referred to as an ALK6-Fc fusion polypeptide (SEQ ID NO: 321), the fusion polypeptide sequence of which is provided below.

KKEDGESTAPTPRPKILRCYCSAGHCPEDSVNNICSTNGYCFTMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 321 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, Y39H, and L51M) were introduced into the SEQ ID NO: 90 sequence with seven amino acids deleted from the C-terminus of ALK-6 ECD of SEQ ID NO: 1 providing SEQ ID NO:322. The ECD was fused to a Fc domain with a linker positioned between the ECD and the Fc domain. The individual constructs are referred to as an ALK6-Fc fusion polypeptide (SEQ ID NO: 327), the fusion polypeptide sequence of which is provided below.

KKEDGESTAPTPRPKILRCYCSAGHCPEDSVNNICSTDGHCFTMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 327 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, T42A, and L51M) were introduced into the SEQ ID NO: 90 sequence with seven amino acids deleted from the C-terminus of ALK-6 ECD of SEQ ID NO: 1 providing SEQ ID NO:328. The ECD was fused to a Fc domain with a linker positioned between the ECD and the Fc domain. The individual constructs are referred to as an ALK6-Fc fusion polypeptide (SEQ ID NO: 333), the fusion polypeptide sequence of which is provided below.

KKEDGESTAPTPRPKILRCYCSAGHCPEDSVNNICSTDGYCFAMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 333 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, M43I, and L51M) were introduced into the SEQ ID NO: 90 sequence with seven amino acids deleted from the C-terminus of ALK-6 ECD of SEQ ID NO: 1 providing SEQ ID NO:334. The ECD was fused to a Fc domain with a linker positioned between the ECD and the Fc domain. The individual constructs are referred to as an ALK6-Fc fusion polypeptide (SEQ ID NO: 339), the fusion polypeptide sequence of which is provided below.

KKEDGESTAPTPRPKILRCYCSAGHCPEDSVNNICSTDGYCFTIIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 339 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, M43I, and L51M) were introduced into the SEQ ID NO: 90 sequence with seven amino acids deleted from the C-terminus of ALK-6 ECD of SEQ ID NO: 1 providing SEQ ID NO:340. The ECD was fused to a Fc domain with a linker positioned between the ECD and the Fc domain. The individual constructs are referred to as an ALK6-Fc fusion polypeptide (SEQ ID NO: 345), the fusion polypeptide sequence of which is provided below.

KKEDGESTAPTPRPKILRCYCSAGHCPEDSVNNICSTDGYCFTMIEEDDS
GMPVVASGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 345 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and L59M) were introduced into the SEQ ID NO: 90 sequence with seven amino acids deleted from the C-terminus of ALK-6 ECD of SEQ ID NO: 1 providing SEQ ID NO:346. The ECD was fused to a Fc domain with a linker positioned between the ECD and the Fc domain. The individual constructs are referred to as an ALK6-Fc fusion polypeptide (SEQ ID NO: 351), the fusion polypeptide sequence of which is provided below.

KKEDGESTAPTPRPKILRCYCSAGHCPEDSVNNICSTDGYCFTMIEEDDS
GMPVVTSGCMGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 351 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and G60K) were introduced into the SEQ ID NO: 90 sequence with seven amino acids deleted from the C-terminus of ALK-6 ECD of SEQ ID NO: 1 providing SEQ ID NO:352. The ECD was fused to a Fc domain with a linker positioned between the ECD and the Fc domain. The individual constructs are referred to as an ALK6-Fc fusion polypeptide (SEQ ID NO: 357), the fusion polypeptide sequence of which is provided below.

KKEDGESTAPTPRPKILRCYCSAGHCPEDSVNNICSTDGYCFTMIEEDDS
GMPVVTSGCLKLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 357 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and L61Y) were introduced into the SEQ ID NO: 90 sequence with seven amino acids deleted from the C-terminus of ALK-6 ECD of SEQ ID NO: 1 providing SEQ ID NO:358. The ECD was fused to a Fc domain with a linker positioned between the ECD and the Fc domain. The individual constructs are referred to as an ALK6-Fc fusion polypeptide (SEQ ID NO: 363), the fusion polypeptide sequence of which is provided below.

KKEDGESTAPTPRPKILRCYCSAGHCPEDSVNNICSTDGYCFTMIEEDDS
GMPVVTSGCLGYEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 363 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and R68K) were introduced into the SEQ ID NO: 90 sequence with seven amino acids deleted from the C-terminus of ALK-6 ECD of SEQ ID NO: 1 providing SEQ ID NO:364. The ECD was fused to a Fc domain with a linker positioned between the ECD and the Fc domain. The individual constructs are referred to as an ALK6-Fc fusion polypeptide (SEQ ID NO: 369), the fusion polypeptide sequence of which is provided below.

KKEDGESTAPTPRPKILRCYCSAGHCPEDSVNNICSTDGYCFTMIEEDDS
GMPVVTSGCLGLEGSDFQCKDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 369 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and T71S) were introduced into the SEQ ID NO: 90 sequence with seven amino acids deleted from the C-terminus of ALK-6 ECD of SEQ ID NO: 1 providing SEQ ID NO:370. The ECD was fused to a Fc domain with a linker positioned between the ECD and the Fc domain. The individual constructs are referred to as an ALK6-Fc fusion polypeptide (SEQ ID NO: 375), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDSPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 375 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and I73K) were introduced into the SEQ ID NO: 90 sequence with seven amino acids deleted from the C-terminus of ALK-6 ECD of SEQ ID NO: 1 providing SEQ ID NO:376. The ECD was fused to a Fc domain with a linker positioned between the ECD and the Fc domain. The individual constructs are referred to as an ALK6-Fc fusion polypeptide (SEQ ID NO: 381), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDTPKPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 381 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and P74A) were introduced into the SEQ ID NO: 90 sequence with seven amino acids deleted from the C-terminus of ALK-6 ECD of SEQ ID NO: 1 providing SEQ ID NO:382. The ECD was fused to a Fc domain with a linker positioned between the ECD and the Fc domain. The individual constructs are referred to as an ALK6-Fc fusion polypeptide (SEQ ID NO: 387), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDTPIAHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 387 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and H75Q) were introduced into the SEQ ID NO: 90 sequence with seven amino acids deleted from the C-terminus of ALK-6 ECD of SEQ ID NO: 1 providing SEQ ID NO:388. The ECD was fused to a Fc domain with a linker positioned between the ECD and the Fc domain. The individual constructs are referred to as an ALK6-Fc fusion polypeptide (SEQ ID NO: 393), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDTPIPQQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 393 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and Q76L) were introduced into the SEQ ID NO: 90 sequence with seven amino acids deleted from the C-terminus of ALK-6 ECD of SEQ ID NO: 1 providing SEQ ID NO:394. The ECD was fused to a Fc domain with a linker positioned between the ECD and the Fc domain. The individual constructs are referred to as an ALK6-Fc fusion polypeptide (SEQ ID NO: 399), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDTPIPHLRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 399 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein three point mutations (V16I, L51M and S78T) were introduced into the SEQ ID NO: 90 sequence with seven amino acids deleted from the C-terminus of ALK-6 ECD of SEQ ID NO: 1 providing SEQ ID NO:400. The ECD was fused to a Fc domain with a linker positioned between the ECD and the Fc domain. The individual constructs are referred to as an ALK6-Fc fusion polypeptide (SEQ ID NO: 405), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDTPIPHQRRTIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 405 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein two point mutations (V16I and L51M) were introduced into the SEQ ID NO: 440 sequence with thirty amino acids deleted from the C-terminus of ALK-3 ECD and SEQ ID NO: 438 added providing SEQ ID NO:406. The ECD was fused to a Fc domain with a linker positioned between the ECD and the Fc domain. The individual constructs are referred to as an ALK6-Fc fusion polypeptide (SEQ ID NO: 411), the fusion polypeptide sequence of which is provided below.

KKEDGESTAPTPRPKILRCYCSAGHCPEDSVNNICSTDGYCFTMIEEDDS
GMPVVTSGCLGLEGSDFQCRDTPIPHQRRSIECCTNLCNQYLQPTLPPVV
IGPFFDGSIR TGGGGSGGGGSGGGGSGGGGSTHTCPPCPAPEAAG
GPSVFLFPPKPKDTLMASRTPEVTCVVVDVSHEDPEVKFNWYVDGVEVHN
AKTKPREEQYNSTYRVVSVLTVLAQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPG

The linker sequence and the point and deletion mutations in the ALK6 ECD are underlined and bold as are the two point mutations in the Fc sequence. The amino acid sequence of SEQ ID NO: 411 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein one point mutation (V16I) was introduced into the SEQ ID NO: 90 sequence with seven amino acids deleted from the C-terminus of ALK-6 ECD of SEQ ID NO: 1 providing SEQ ID NO:412. The ECD was fused to a Fc domain with a linker positioned between the ECD and the Fc domain. The individual constructs are referred to as an ALK6-Fc fusion polypeptide (SEQ ID NO: 417), the fusion polypeptide sequence of which is provided below.

KKEDGESTAPTPRPKILRCYCSAGHCPEDSVNNICSTDGYCFTMIEEDDS
GLPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 417 may optionally be provided with lysine (K) added to the C-terminus and/or with a leader sequence.

In another approach to generate a non-aggregated ALK6-Fc fusion polypeptide and maintain the ALK6 ligand binding properties without generating cleavage products, Applicants constructed a soluble ALK-6 Fc fusion polypeptide wherein one point mutation (L51M) was introduced into the SEQ ID NO: 90 sequence with seven amino acids deleted from the C-terminus of ALK-6 ECD of SEQ ID NO: 1 providing SEQ ID NO:418. The ECD was fused to a Fc domain with a linker positioned between the ECD and the Fc domain. The individual constructs are referred to as an ALK6-Fc fusion polypeptide (SEQ ID NO: 423), the fusion polypeptide sequence of which is provided below.

KKEDGESTAPTPRPKVLRCYCSAGHCPEDSVNNICSTDGYCFTMIEEDDS
GMPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPS
VFLFPPKPKDTLMASRTPEVTCVVVDVSHEDPEVKFNWYVDGVEVHNAKT
KPREEQYNSTYRVVSVLTVLAQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPG

The linker sequence and the point and deletion mutations in the ALK6 ECD are underlined and bold as are the two point mutations in the Fc sequence. The amino acid sequence of SEQ ID NO: 423 may optionally be provided with lysine (K) added to the C-terminus and/or with a leader sequence.

### Example 2: Generation of Alternative ALK-6 Fc Fusion Proteins

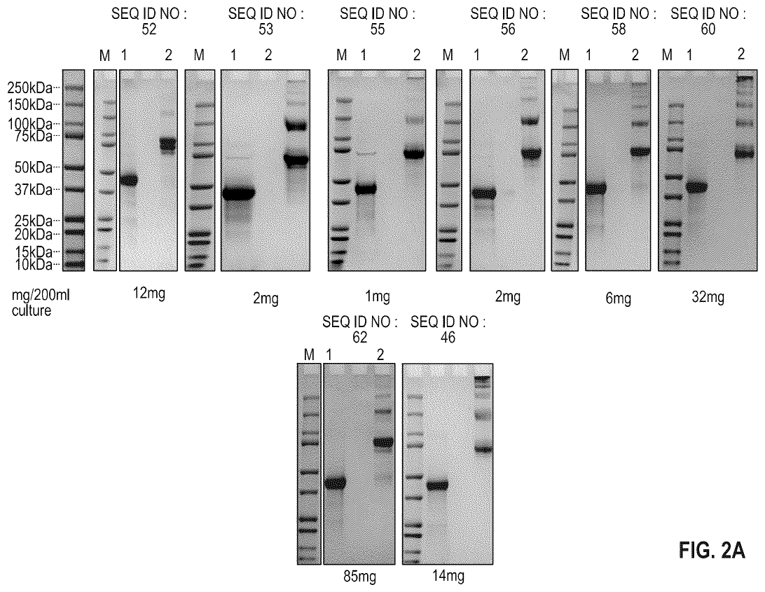

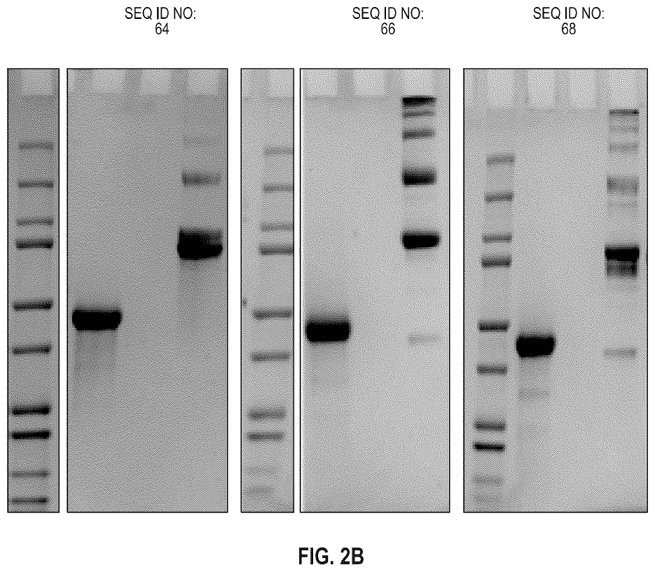

Applicants constructed a number of soluble ALK-6 Fc fusion polypeptides using SEQ ID NO: 46 as a background sequence introducing one or more point mutations in the modified ECD of ALK6 (“SEQ ID NO:11 variants”). These variant soluble ALK-6 Fc fusion polypeptides were analyzed on a SDS gel to confirm the lack of a cleavage product and select variants tested in a Biacore-based binding assay for ligand binding specificity. See FIGS. 2 and 3.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation H23G was introduced into the modified ECD of ALK6 providing SEQ ID NO:20. The ECD was fused to a Fc domain with a linker positioned between the ECD and the Fc domain. The individual constructs are referred to as an ALK6-Fc fusion polypeptide (SEQ ID NO: 52), the fusion polypeptide sequence of which is provided below

KKEDGESTAPTPRPKVLRCKCHGHCPEDSVNNICSTDGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutation in the ALK6 ECD are underlined and bold. The amino acid sequence of SEQ ID NO: 52 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation D37N was introduced into the modified ECD of ALK6 providing SEQ ID NO:26. The ECD was fused to a Fc domain with a linker positioned between the ECD and the Fc domain. The individual constructs are referred to as ALK6-Fc fusion polypeptide (SEQ ID NO: 53), the fusion polypeptide sequence of which is provided below.

KKEDGESTAPTPRPKVLRCKCHHHCPEDSVNNICSTNGYCFTMIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutation in the ALK6 ECD are underlined and bold. The amino acid sequence of SEQ ID NO: 53 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations T42A and M43I were introduced into the modified ECD of ALK6 providing SEQ ID NO:28. The ECD was fused to a Fc domain with a linker positioned between the ECD and the Fc domain. The individual constructs are referred to as an ALK6-Fc fusion polypeptide (SEQ ID NO: 54), the fusion polypeptide sequence of which is provided below.

KKEDGESTAPTPRPKVLRCKCHHHCPEDSVNNICSTDGYCFAIIEEDDS
GLPVVTSGCLGLEGSDFQCRDTPIPHQRRSIECCTERNECNKDLHPTLP
PVVIGPFFDGSIRTGGGGSGGGGSGGGGSGGGGSTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPGK

The linker sequence and the individual point mutations in the ALK6 ECD are underlined and bold. The amino acid sequence of SEQ ID NO: 54 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations L59M, G60K and L61Y were introduced into the modified ECD of ALK6 providing SEQ ID NO:33. The ECD was fused to a Fc domain with a linker positioned between the ECD and the Fc domain. The individual constructs are referred to as ALK6-Fc fusion polypeptide (SEQ ID NO: 55), the fusion polypeptide sequence of which is provided below.

KKEDGESTAPTPRPKVLRCKCHHHCPEDSVNNICSTDGYCFTMIEEDDSG
LPVVTSGCMKYEGSDFQCRDTPIPHQRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutations in the ALK6 ECD are underlined and bold. The amino acid sequence of SEQ ID NO: 55 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations G60K, I73K, P74A, H75Q and Q76L were introduced into the modified ECD of ALK6 providing SEQ ID NO:41. The ECD was fused to a Fc domain with a linker positioned between the ECD and the Fc domain. The individual constructs are referred to as ALK6-Fc fusion polypeptide (SEQ ID NO: 56), the fusion polypeptide sequence of which is provided below.

KKEDGESTAPTPRPKVLRCKCHHHCPEDSVNNICSTDGYCFTMIEEDDSG
LPVVTSGCLKLEGSDFQCRDTPKAQLRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutations in the ALK6 ECD are underlined and bold. The amino acid sequence of SEQ ID NO: 56 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation H22S was introduced into the modified ECD of ALK6 providing SEQ ID NO:57. The ECD was fused to a Fc domain with a linker positioned between the ECD and the Fc domain. The individual constructs are referred to as ALK6-Fc fusion polypeptide (SEQ ID NO: 58), the fusion polypeptide sequence of which is provided below.

KKEDGESTAPTPRPKVLRCKCSHHCPEDSVNNICSTDGYCFTMIEEDDSG
LPVVTSGCLGLEGSDFQCRDTPIPHQRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutations in the ALK6 ECD are underlined and bold. The amino acid sequence of SEQ ID NO: 58 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations H22S and H23G were introduced into the modified ECD of ALK6 providing SEQ ID NO:59. The ECD was fused to a Fc domain with a linker positioned between the ECD and the Fc domain. The individual constructs are referred to as ALK6-Fc fusion polypeptide (SEQ ID NO: 60), the fusion polypeptide sequence of which is provided below.

KKEDGESTAPTPRPKVLRCKCSGHCPEDSVNNICSTDGYCFTMIEEDDSG
LPVVTSGCLGLEGSDFQCRDTPIPHQRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutations in the ALK6 ECD are underlined and bold. The amino acid sequence of SEQ ID NO: 60 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations K20Y, H22S and H23G were introduced into the modified ECD of ALK6 providing SEQ ID NO:61. The ECD was fused to a Fc domain with a linker positioned between the ECD and the Fc domain. The individual constructs are referred to as an ALK6-Fc fusion polypeptide (SEQ ID NO: 62), the fusion polypeptide sequence of which is provided below.

KKEDGESTAPTPRPKVLRCYCSGHCPEDSVNNICSTDGYCFTMIEEDDSG
LPVVTSGCLGLEGSDFQCRDTPIPHQRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutations in the ALK6 ECD are underlined and bold. The amino acid sequence of SEQ ID NO: 62 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations K20Y and H23G were introduced into the modified ECD of ALK6 providing SEQ ID NO:63. The ECD was fused to a Fc domain with a linker positioned between the ECD and the Fc domain. The individual constructs are referred to as an ALK6-Fc fusion polypeptide (SEQ ID NO: 64), the fusion polypeptide sequence of which is provided below.

KKEDGESTAPTPRPKVLRCYCHGHCPEDSVNNICSTDGYCFTMIEEDDSG
LPVVTSGCLGLEGSDFQCRDTPIPHQRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutations in the ALK6 ECD are underlined and bold. The amino acid sequence of SEQ ID NO: 64 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutation K20Y was introduced into the modified ECD of ALK6 providing SEQ ID NO:65. The ECD was fused to a Fc domain with a linker positioned between the ECD and the Fc domain. The individual constructs are referred to as an ALK6-Fc fusion polypeptide (SEQ ID NO: 66), the fusion polypeptide sequence of which is provided below.

KKEDGESTAPTPRPKVLRCYCHHHCPEDSVNNICSTDGYCFTMIEEDDSG
LPVVTSGCLGLEGSDFQCRDTPIPHQRRSIECCTERNECNKDLHPTLPPV
VIGPFFDGSIRTGGGGSGGGGSGGGGSGGGGSTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPGK

The linker sequence and the individual point mutations in the ALK6 ECD are underlined and bold. The amino acid sequence of SEQ ID NO: 66 may optionally be provided with lysine (K) removed from the C-terminus and/or with a leader sequence.

Applicants constructed a soluble ALK-6 Fc fusion polypeptide wherein the 14 deleted amino acids from the C-terminus of SEQ ID NO: 4 were replaced with SEQ ID NO: 2 (VVIGPFFDGSIR) and wherein the point mutations K20Y, H22S, H23G, and G60L was introduced into the modified ECD of ALK6 providing SEQ ID NO:88. The ECD was fused to a Fc domain with a linker positioned between the ECD and the Fc domain. The individual constructs are referred to as an ALK6-Fc fusion polypeptide (SEQ ID NO: 88), the fusion polypeptide sequence of which is provided below.

KKEDGESTAPTPRPKVLRCYCSGHCPEDSVNNICSTDGYCFTMIEEDDSG
LPVVTSGCLKLEGSDFQCRDTPIPHQRRSIECCTERNECNKDLHPTLPPV
VIGPFFDGSIRTGGGGSGGGGSGGGGSGGGGSTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

The linker sequence and the individual point mutations in the ALK6 ECD and Fc sequence are underlined and bold. The amino acid sequence of SEQ ID NO: 89 may optionally be provided with lysine (K) removed (as shown) from the C-terminus, or added and/or with a leader sequence.

Applicants further constructed a soluble ALK-6 Fc fusion polypeptide using SEQ ID NO: 73 as a background sequence introducing three point mutations in the modified ECD of ALK6 (SEQ ID NO:1 variant). The variant soluble ALK-6 Fc fusion polypeptide was analyzed on a SDS gel to confirm the lack of a cleavage product. See FIG. 2.

Applicants constructed a soluble ALK-6 Fc fusion polypeptide wherein the point mutation D107A was introduced and the last two histidine amino acids (H111_and H112_) were deleted from the C terminus of SEQ ID NO: 1 and wherein the point mutations K20Y, H22S and H23G were introduced into the modified ECD of ALK6 providing SEQ ID NO:67. The ECD was fused to a Fc domain with a linker positioned between the ECD and the Fc domain. The individual constructs are referred to as an ALK6-Fc fusion polypeptide (SEQ ID NO: 68), the fusion polypeptide sequence of which is provided below.

KKEDGESTAPTPRPKVLRCYCSGHCPEDSVNNICSTDGYCFTMIEEDDSG
LPVVTSGCLGLEGSDFQCRDTPIPHQRRSIECCTERNECNKDLHPTLPPL
KNRDFVAGPI_RTGGGGSGGGGSGGGGSGGGGSTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

The linker sequence and the individual point mutations in the ALK6 ECD are underlined and bold. The amino acid sequence of SEQ ID NO: 68 may optionally be provided with lysine (K) removed from the C-terminus and/or with a leader sequence.

Applicants further constructed a soluble ALK-6 Fc fusion polypeptide using SEQ ID NO: 69 as a background sequence introducing point mutations in the modified ECD of ALK6 (SEQ ID NO: 1 variant). The variant soluble ALK-6 Fc fusion polypeptide was analyzed on a SDS gel to confirm the lack of a cleavage product and select variants tested in a Biacore-based binding assay for ligand binding specificity. Data not shown.

Applicants constructed a soluble ALK-6 Fc fusion polypeptide wherein seven amino acids from the C-terminus of SEQ ID NO: 1 were deleted and wherein the point mutations K20Y, H22S and H23G were introduced into the modified ECD of ALK6 providing SEQ ID NO:86. The ECD was fused to a Fc domain with a linker positioned between the ECD and the Fc domain. The individual constructs are referred to as an ALK6-Fc fusion polypeptide (SEQ ID NO: 87), the fusion polypeptide sequence of which is provided below.

KKEDGESTAPTPRPKVLRCYCSGHCPEDSVNNICSTDGYCFTMIEEDDS
GLPVVTSGCLGLEGSDFQCRDTPIPHQRRSIECCTERNECNKDLHPTLP
PLKNRDFV TGGGGSGGGGSGGGGSGGGGSTHTCPPCPAPEAAGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALH
NHYTQKSLSLSPG

The linker sequence and the individual point mutations in the ALK6 ECD and Fc sequence are underlined and bold. The amino acid sequence of SEQ ID NO: 87 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein an amino acid Arginine was added between positions 22 and 23 (_22′A) and the point mutations K20Y, H22S and H23G were introduced into the modified ECD of ALK6 providing SEQ ID NO:90. The ECD was fused to a Fc domain with a linker positioned between the ECD and the Fc domain. The individual constructs are referred to as an ALK6-Fc fusion polypeptide (SEQ ID NO: 91), the fusion polypeptide sequence of which is provided below.

KKEDGESTAPTPRPKVLRCYCSAGHCPEDSVNNICSTDGYCFTMIEEDDS
GLPVVTSGCLGLEGSDFQCRDTPIPHQRRSIECCTERNECNKDLHPTLPP
LKNRDFV TGGGGSGGGGSGGGGSGGGGSTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

The linker sequence and the individual point mutations in the ALK6 ECD and Fc sequence are underlined and bold. The amino acid sequence of SEQ ID NO: 91 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20Y, H22S, H23G and D37N were introduced into the modified ECD of ALK6 providing SEQ ID NO:92. The ECD was fused to a Fc domain with a linker positioned between the ECD and the Fc domain. The individual constructs are referred to as an ALK6-Fc fusion polypeptide (SEQ ID NO: 93), the fusion polypeptide sequence of which is provided below.

KKEDGESTAPTPRPKVLRCYCSGHCPEDSVNNICSTNGYCFTMIEEDDSG
LPVVTSGCLGLEGSDFQCRDTPIPHQRRSIECCTERNECNKDLHPTLPPL
KNRDFV TGGGGSGGGGSGGGGSGGGGSTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

The linker sequence and the individual point mutations in the ALK6 ECD and Fc sequence are underlined and bold. The amino acid sequence of SEQ ID NO: 93 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein point mutations were introduced to reduce aggregation, improve folding and expression while maintaining ALK-6 ECD amino acid residues that interact directly with their ligand. See FIG. 2C.

Applicants constructed a soluble ALK-6 Fc fusion polypeptide wherein seven amino acids from the C-terminus of SEQ ID NO: 1 were deleted and wherein the point mutation 133T was introduced into the modified ECD of ALK6 providing SEQ ID NO:136. The ECD was fused to a Fc domain with a linker positioned between the ECD and the Fc domain. The individual constructs are referred to as an ALK6-Fc fusion polypeptide (SEQ ID NO: 282), the fusion polypeptide sequence of which is provided below.

KKEDGESTAPTPRPKVLRCKCHHHCPEDSVNNTCSTDGYCFTMIEEDDSG
LPVVTSGCLGLEGSDFQCRDTPIPHQRRSIECCTERNECNKDLHPTLPPL
KNRDFV TGGGGSGGGGSGGGGSGGGGSTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

The linker sequence and the individual point mutations in the ALK6 ECD and Fc sequence are underlined and bold. The amino acid sequence of SEQ ID NO: 282 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations H22S, V30I, I33T, and S35I were introduced into the modified ECD of ALK6 providing SEQ ID NO:202. The ECD was fused to a Fc domain with a linker positioned between the ECD and the Fc domain. The individual constructs are referred to as an ALK6-Fc fusion polypeptide (SEQ ID NO: 203), the fusion polypeptide sequence of which is provided below.

KKEDGESTAPTPRPKVLRCKCSHHCPEDSINNTCITDGYCFTMIEEDDSG
LPVVTSGCLGLEGSDFQCRDTPIPHQRRSIECCTERNECNKDLHPTLPPL
KNRDFV TGGGGSGGGGSGGGGSGGGGSTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

The linker sequence and the individual point mutations in the ALK6 ECD and Fc sequence are underlined and bold. The amino acid sequence of SEQ ID NO: 203 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations V30I, I33T, and S35I were introduced into the modified ECD of ALK6 providing SEQ ID NO:213. The ECD was fused to a Fc domain with a linker positioned between the ECD and the Fc domain. The individual constructs are referred to as an ALK6-Fc fusion polypeptide (SEQ ID NO: 214), the fusion polypeptide sequence of which is provided below.

KKEDGESTAPTPRPKVLRCKCHHHCPEDSINNTCITDGYCFTMIEEDDSG
LPVVTSGCLGLEGSDFQCRDTPIPHQRRSIECCTERNECNKDLHPTLPPL
KNRDFV TGGGGSGGGGSGGGGSGGGGSTHTCPPCPAPEAAGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG

The linker sequence and the individual point mutations in the ALK6 ECD and Fc sequence are underlined and bold. The amino acid sequence of SEQ ID NO: 214 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutation H22I, I33T, and S35I was introduced into the modified ECD of ALK6 providing SEQ ID NO:218. The ECD was fused to a Fc domain with a linker positioned between the ECD and the Fc domain. The individual constructs are referred to as an ALK6-Fc fusion polypeptide (SEQ ID NO: 219), the fusion polypeptide sequence of which is provided below.

KKEDGESTAPTPRPKVLRCKCSHHCPEDSVNNTCITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 219 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutation H22S, V30I, and I33T was introduced into the modified ECD of ALK6 providing SEQ ID NO:223. The ECD was fused to a Fc domain with a linker positioned between the ECD and the Fc domain. The individual constructs are referred to as an ALK6-Fc fusion polypeptide (SEQ ID NO: 224), the fusion polypeptide sequence of which is provided below.

KKEDGESTAPTPRPKVLRCKCSHHCPEDSINNTCSTDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 224 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutation H22S and I33T was introduced into the modified ECD of ALK6 providing SEQ ID NO:228. The ECD was fused to a Fc domain with a linker positioned between the ECD and the Fc domain. The individual constructs are referred to as an ALK6-Fc fusion polypeptide (SEQ ID NO: 229), the fusion polypeptide sequence of which is provided below.

KKEDGESTAPTPRPKVLRCKCSHHCPEDSVNNTCSTDGYCFTMIEEDDSGLPVVTTGGG
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 229 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20Y, V30I, I33T, and S35I were introduced into the modified ECD of ALK6 providing SEQ ID NO:277. The ECD was fused to a Fc domain with a linker positioned between the ECD and the Fc domain. The individual constructs are referred to as an ALK6-Fc fusion polypeptide (SEQ ID NO: 278), the fusion polypeptide sequence of which is provided below.

KKEDGESTAPTPRPKVLRCYCHHHCPEDSINNTCITDGYCFTMIEEDDSGLPVVT
SGCLGLEGSDFQCRDTPIPHQRRSIECCTERNECNKDLHPTLPPLKNRDFV____TGG
GGSGGGGSGGGGSGGGGSTHTCPPCPAPEAAGGPSVFLFPPKPKDTLMISRTPEVTCV
VVDVSHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNH
YTQKSLSLSPG

The linker sequence and the individual point mutations in the ALK6 ECD and Fc sequence are underlined and bold. The amino acid sequence of SEQ ID NO: 27 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations I33T and S35I were introduced into the modified ECD of ALK6 providing SEQ ID NO:233. The ECD was fused to a Fc domain with a linker positioned between the ECD and the Fc domain. The individual constructs are referred to as an ALK6-Fc fusion polypeptide (SEQ ID NO: 234), the fusion polypeptide sequence of which is provided below.

KKEDGESTAPTPRPKVLRCKCHHHCPEDSVNNTCITDGYCFTMIEEDDSGLPVTS
GCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVV
VDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 234 may optionally be provided with lysine (K) added or removed from the C-terminus and/or with a leader sequence. This fusion polypeptide, with the ALK-6 ECD point mutation on both sides of the cystine residue, created a glycosylation site, and dramatically improved folding and expression as compared to SEQ ID NO: 69 and SEQ ID NO: 70. See FIG. 2C.

Applicants constructed a soluble ALK-6 Fc fusion polypeptide wherein seven amino acids from the C-terminus of SEQ ID NO: 1 were deleted and wherein the point mutation S35I was introduced into the modified ECD of ALK6 providing SEQ ID NO:137. The ECD was fused to a Fc domain with a linker positioned between the ECD and the Fc domain. The individual constructs are referred to as an ALK6-Fc fusion polypeptide (SEQ ID NO: 238), the fusion polypeptide sequence of which is provided below.

KKEDGESTAPTPRPKVLRCKCHHHCPEDSVNNICITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 238 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20F, I33T, and S35I was introduced into the modified ECD of ALK6 providing SEQ ID NO:242. The ECD was fused to a Fc domain with a linker positioned between the ECD and the Fc domain. The individual constructs are referred to as an ALK6-Fc fusion polypeptide (SEQ ID NO: 243), the fusion polypeptide sequence of which is provided below.

KKEDGESTAPTPRPKVLRCFCHHHCPEDSVNNTCITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 243 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations H22S and S35I was introduced into the modified ECD of ALK6 providing SEQ ID NO:247. The ECD was fused to a Fc domain with a linker positioned between the ECD and the Fc domain. The individual constructs are referred to as an ALK6-Fc fusion polypeptide (SEQ ID NO: 248), the fusion polypeptide sequence of which is provided below.

KKEDGESTAPTPRPKVLRCKCSHHCPEDSVNNICITDGYCFTMIEEDDSGLPVVTS
GCLGLEGSDFQCRDTPIPHQRRSIECCTERNECNKDLHPTLPPLKNRDFV____TGGGG
SGGGGSGGGGSGGGGSTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMHEALHNHYTQK
SLSLSPG

The linker sequence and the individual point mutations in the ALK6 ECD and Fc sequence are underlined and bold. The amino acid sequence of SEQ ID NO: 248 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20F, H22S, I33T, and S35I was introduced into the modified ECD of ALK6 providing SEQ ID NO:252. The ECD was fused to a Fc domain with a linker positioned between the ECD and the Fc domain. The individual constructs are referred to as an ALK6-Fc fusion polypeptide (SEQ ID NO: 253), the fusion polypeptide sequence of which is provided below.

KKEDGESTAPTPRPKVLRCFCSHHCPEDSVNNTCITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 253 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20I, I33T, and S35I was introduced into the modified ECD of ALK6 providing SEQ ID NO:257. The ECD was fused to a Fc domain with a linker positioned between the ECD and the Fc domain. The individual constructs are referred to as an ALK6-Fc fusion polypeptide (SEQ ID NO: 258), the fusion polypeptide sequence of which is provided below.

KKEDGESTAPTPRPKVLRCICHHHCPEDSVNNTCITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 258 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20A, I33T, and S35I was introduced into the modified ECD of ALK6 providing SEQ ID NO:262. The ECD was fused to a Fc domain with a linker positioned between the ECD and the Fc domain. The individual constructs are referred to as an ALK6-Fc fusion polypeptide (SEQ ID NO: 263), the fusion polypeptide sequence of which is provided below.

KKEDGESTAPTPRPKVLRCACHHHCPEDSVNNTCITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 263 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20T, I33T, and S35I was introduced into the modified ECD of ALK6 providing SEQ ID NO:267. The ECD was fused to a Fc domain with a linker positioned between the ECD and the Fc domain. The individual constructs are referred to as an ALK6-Fc fusion polypeptide (SEQ ID NO: 268), the fusion polypeptide sequence of which is provided below.

KKEDGESTAPTPRPKVLRCTCHHHCPEDSVNNTCITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 268 may optionally be provided with lysine (K) added or removed from the C-terminus and/or with a leader sequence.

Applicants constructed a soluble ALK-6 Fc fusion polypeptide wherein seven amino acids from the C-terminus of SEQ ID NO: 1 were deleted and wherein the point mutations K20V, I33T, and S35I was introduced into the modified ECD of ALK6 providing SEQ ID NO:272. The ECD was fused to a Fc domain with a linker positioned between the ECD and the Fc domain. The individual constructs are referred to as an ALK6-Fc fusion polypeptide (SEQ ID NO: 273), the fusion polypeptide sequence of which is provided below.

KKEDGESTAPTPRPKVLRCVCHHHCPEDSVNNTCITDGYCFTMIEEDDSGLPVVT
SGCLGLEGSDFQCRDTPIPHQRRSIECCTERNECNKDLHPTLPPLKNRDFV____TGGG
GSGGGGSGGGGSGGGGSTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYT
QKSLSLSPG

The linker sequence and the individual point mutations in the ALK6 ECD and Fc sequence are underlined and bold. The amino acid sequence of SEQ ID NO: 273 may optionally be provided with lysine (K) added or removed from the C-terminus and/or with a leader sequence.

Applicants constructed a soluble ALK-6 Fc fusion polypeptide wherein the 29 deleted amino acids from the C-terminus of SEQ ID NO: 439 were replaced with SEQ ID NO: 438 (TNLCNQYLQPTLPPVVIGPFFDGSIR) and providing SEQ ID NO:425. The ECD was fused to a Fc domain with a linker positioned between the ECD and the Fc domain. The individual constructs are referred to as an ALK6-Fc fusion polypeptide (SEQ ID NO: 430), the fusion polypeptide sequence of which is provided below.

KKEDGESTAPTPRPKVLRCKCHHHCPEDSVNNICSTDGYCFTMIEEDDSGLPVVT
SGCLGLEGSDFQCRDTPIPHQRRSIECCTTNLCNQYLQPTLPPVVIGPFFDGSIRTGGG
GSGGGGSGGGGSGGGGSTHTCPPCPAPEAAGGPSVFLFPPKPKDTLMASRTPEVTCVV
VDVSHEDPEVKFNWYVDGVEVHNAKTKPREEQYNSTYRVVSVLTVLAQDWLNGKEY
KCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYT
QKSLSLSPG

The linker sequence and the ALK3 C terminus sequence in the ALK6 ECD and Fc sequence are underlined and bold. The amino acid sequence of SEQ ID NO: 430 may optionally be provided with lysine (K) added or removed from the C-terminus and/or with a leader sequence.

Applicants constructed a soluble ALK-6 Fc fusion polypeptide wherein the 29 deleted amino acids from the C-terminus of SEQ ID NO: 439 were replaced with SEQ ID NO: 438 (TNLCNQYLQPTLPPVVIGPFFDGSIR) wherein two point mutations (V16I and L51M) were introduced into the ALK6 ECD sequence and providing SEQ ID NO:431. The ECD was fused to a Fc domain with a linker positioned between the ECD and the Fc domain. The individual constructs are referred to as an ALK6-Fc fusion polypeptide (SEQ ID NO: 436), the fusion polypeptide sequence of which is provided below.

KKEDGESTAPTPRPKILRCKCHHHCPEDSVNNICSTDGYCFTMIEEDDSGMPVVT
SGCLGLEGSDFQCRDTPIPHQRRSIECCTTNLCNQYLQPTLPPVVIGPFFDGSIRTGGG
GSGGGGSGGGGSGGGGSTHTCPPCPAPEAAGGPSVFLFPPKPKDTLMASRTPEVTCVV
VDVSHEDPEVKFNWYVDGVEVHNAKTKPREEQYNSTYRVVSVLTVLAQDWLNGKEY
KCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYT
QKSLSLSPG

The linker sequence and the ALK3 C terminus sequence in the ECD and Fc sequence are underlined and bold. The amino acid sequence of SEQ ID NO: 436 may optionally be provided with lysine (K) added or removed from the C-terminus and/or with a leader sequence.

### Example 3: In Vitro Screening to Identify ALK-6 Fc Fusion Protein with Binding to BMP4 but not BMP10

A Biacore™-based binding assay was used to compare ligand binding selectivity of the ALK6 variant-Fc dimeric complex (e.g., SEQ ID NO: 46) described above with that of ALK6 wt-Fc complexes (SEQ ID NO: 49). The Fc dimeric complexes were independently captured onto the system using an anti-Fc antibody. Ligands were injected and allowed to flow over the captured receptor protein. Results are summarized in the table below and provided in FIGS. 3A and 3B.

TABLE 1
Ligand Binding Profile of ALK6 variant-Fc dimeric complex

| BMP4 Positive Binding | BMP10 Positive Binding |
|---|---|
| ALK6wt-Fc SEQ ID NO: 49 | ALK6wt-Fc SEQ ID NO: 49 |
| SEQ ID NO: 46 | SEQ ID NO: 46 |
| SEQ ID NO: 47 | SEQ ID NO: 47 |
| SEQ ID NO: 48 | SEQ ID NO: 48 |
| SEQ ID NO: 52 | SEQ ID NO: 52 |
| SEQ ID NO: 53 | SEQ ID NO: 53 |
| SEQ ID NO: 55 | No binding |

Ligand off-rate is a particularly significant parameter to evaluate for ligand traps. Soluble receptor-Fc proteins administered in vivo are in constant competition with native receptors for ligands. When endogenous ligands of the TGF□ superfamily typically bind to cognate receptors at the cell surface, a multi-step signal transduction process is triggered that is relatively slow on a molecular time scale. Native receptors dissociate from ligand slowly in part because significant time is required to generate an intracellular signal from a ligand binding event. For a soluble receptor-Fc protein to compete effectively for ligand, the off-rate for its complex with the ligand needs to be similar to, or slower than, the off-rate for a ligand complex with native receptor. Ligand binding is a dynamic process and some fraction of ligands will always be in unbound form, so it is important therapeutically for a dose of receptor-Fe protein to capture target ligand for as long as possible. One way to shift the binding equilibrium in favor of more captured ligand is to increase the concentration (dose level) of inhibitor, however this can generate off-target effects that reduce tolerability and safety. A preferable approach is to use an inhibitor with a slower ligand off-rate (longer capture time) combined with ligand binding selectivity to achieve an effective level of ligand antagonism at a lower concentration of inhibitor.

### Example 4: Cell Based Reporter Gene Assay

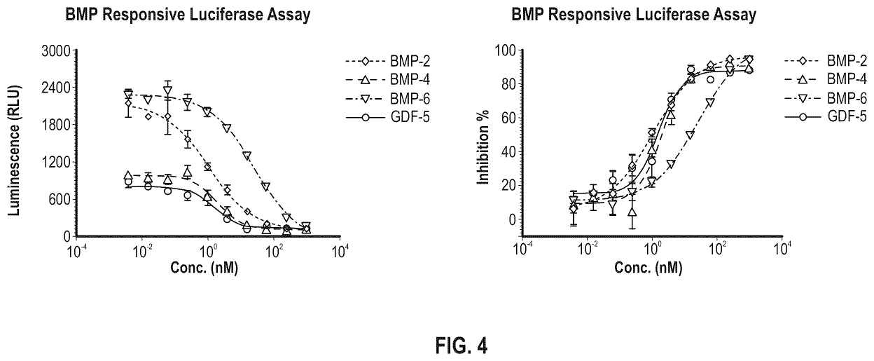

20,000 cells/well in 96-well plate were inoculated with the activation concentrations of 4 ligands (BMP-2; BMP-4; BMP-6 and GDF-5) set at their respective EC50 values (1.076 nM; 1.995 nM; 25.01 nM and 1.647 nM). The concentration of the inhibitor (SEQ ID NO: 87) was 1000 nM, with 4-fold dilution, 10 concentration points, and incubation at 37° C. for 3 hours. SEQ ID NO: 87 effectively inhibited the luciferase signals activated by the four ligands demonstrating binding consistent with Biacore data for SEQ ID NO 87. See FIG. 4.

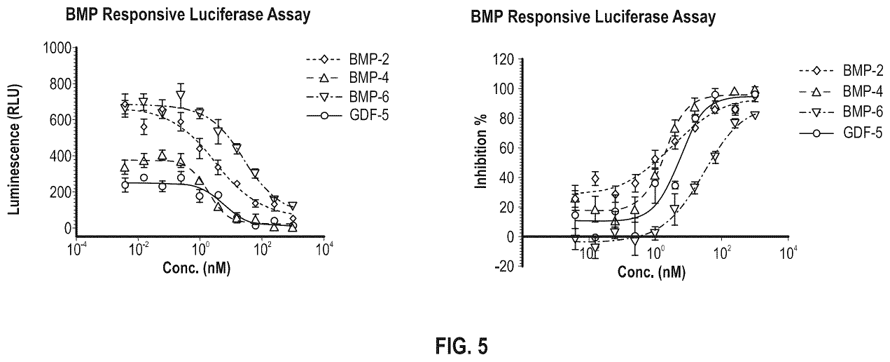

20,000 cells/well in 96-well plate were inoculated with the activation concentrations of 4 ligands (BMP-2; BMP-4; BMP-6 and GDF-5) set at their respective EC50 values (3.286 nM; 1.951 nM; 29.1 nM and 5.51 nM). The concentration of the inhibitor (SEQ ID NO: 70) was 1000 nM, with 4-fold dilution, 10 concentration points, and incubation at 37° C. for 3 hours. SEQ ID NO: 70 effectively inhibited the luciferase signals activated by the four ligands demonstrating binding consistent with Biacore data for SEQ ID NO 70. See FIG. 5.

In a separate experiment, a cell-based reporter line was used to quantitate the inhibitory activity of proteins against BMP signaling in a cell-based format. An HEK-293 cell line with a BRE-luciferase reporter was used to determine the effects of an ALK6 Fc fusion protein comprising ALK6 ECD sequence SEQ ID NO: 286 (e.g. SEQ ID NO: 291) on the ability of BMP2, BMP4, BMP6, BMP7 and BMP10 to produce a luciferase signal. Cells were seeded at 5*104 cells/well (96 well plate), in 100 μL of DMEM+0.1% FBS with 2 μg/mL Puromycin. Overnight cell viability was >95%. The ALK6 Fc fusion protein comprising ALK6 ECD sequence SEQ ID NO: 286 was first added to the cells at increasing concentrations and incubated for 15 minutes prior to adding either BMP2, BMP4, BMP6, BMP7 or BMP10 at a final concentration of 125 ng/ml. Cells were incubated at @ 37° C., 5% CO₂, for 18 hr. After 18 hr, 70 μL of ONE-Glo Luciferase Assay System was added to each well, and incubated at RT for 10 min. All samples were performed in duplicate. Luciferase readouts used a microplate reader detector and dose-response curves were generated to determine IC₅₀ and IC75 values for SEQ ID NO: 286. See FIG. 15.

In embodiments provided herein are ALK6 ECD Fc fusion proteins of this disclosure designed to remove cleavage sites and aggregation (See Examples 1 and 2) but retain a BMP ligand binding profile similar to ALK6 ECD wildtype (wt) binding, including one or more of BMP10, BMP7, BMP4 and/or BMP6. SEQ ID NO: 49 represents ALK6 ECD wt sequence (e.g. SEQ ID NO: 1) fused to a linker and an Fc sequence and used as a comparator. Applicants surprisingly found that introducing the point mutations K20Y; H22S; _22′A (inserted between aa22 and aa23) H23G in the ALK6 ECD while also removing seven amino acid positions from the C-terminus removed cleavage of the ALK6 Fc fusion protein. See FIG. 3B wherein SEQ ID NO: 91 binds BMP2, BMP6 and BMP7. Surprisingly, this binding was retained when the additional mutations of V16I; L51M were introduced in SEQ ID NO: 90, (e.g. SEQ ID NO: 286) but removed aggregation of the Fc fusion protein. See FIG. 13.

In certain embodiments provided herein are ALK6 ECD Fc fusion proteins of this disclosure designed to remove cleavage sites and aggregation (See Examples 1 and 2) with decreased binding to certain BMP ligands but remain active in disease models. For example SEQ ID NO: 286 demonstrated reduced binding to BMP2 as compared to wt. See FIG. 17.

### Example 5: Accepted Animal Models of Diabetic Retinopathy

Mice, rats, hamsters, dogs, cats, and monkeys are some of the common animal models that are used for studying Diabetic Retinopathy.

Animal experiments have been pivotal in the understanding of the pathogenesis of retinopathy since systematic structural, functional and biochemical studies cannot be undertaken in human subjects. Animal experiments are of immense importance in an attempt to develop adjuvant treatment strategies. Characteristic retinal lesions in diabetes have successfully been reproduced in experimental diabetic or galactose-fed animals. See Diabetic Retinopathy: a comprehensive update on in vivo, in vitro and ex vivo experimental models, Sadikan et al. BMC Ophthalmology (2023) 23:421.

Data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration arrange that includes the IC50 (i.e., the concentration of the test compound which achieves a half-maximal inhibition) as determined in cell culture. Levels in plasma can be measured, for example, by high performance liquid chromatography. Such information can be used to more accurately determine useful doses in humans.

Some of the common animal models for studying Diabetes Retinopathy along with the source and relevant text are detailed below:

### Mice

Protocols which may be used to test compounds for efficacy of treatment in mice include those described in, for example, Diabetic Retinopathy by Elia Duh, Springer, Humana Press, 2009; Kern et al. (Arch Ophthalmol. 1996; 114(8):986-990); Feit-Leichman et al. (Investigative Ophthalmology & Visual Science, 46(11): 4281-4287 November 2005).

### Rats

Protocols which may be used to test compounds for efficacy of treatment in rats include those described in, for example, Diabetic Retinopathy by Elia Duh, Springer, Humana Press, 2009; Sima et al. (Current Eye Research, 1985, Vol. 4(10) Pages 1087-1092); Kato et al. (Journal of Diabetes and Its Complications, Volume 17(6): 374-379, November 2003); Sima et al. (Metabolism, 32(7, Suppl. 1): 136-140, July 1983); Lu et al. (Journal of Ophthalmology, 47(1): 28-35, 2003); and Deng et al. (International Journal of Diabetes, vol. 6 (issue 1), 1998).

### Hamsters and Other Rodents

Protocols which may be used to test compounds for efficacy of treatment in hamsters and other rodents include those described in, for example, Diabetic Retinopathy by Elia Duh, Springer, Humana Press, 2009.

### Dogs

Protocols which may be used to test compounds for efficacy of treatment in dogs include those described in, for example, Diabetic Retinopathy by Elia Duh, Springer, Humana Press, 2009; Engerman et al. (Arch Ophthalmol. 1995; 113(3):355-358); and Kador et al. (Arch Ophthalmol. 1990; 108(9):1301-1309).

### Cats

Protocols which may be used to test compounds for efficacy of treatment in cats include those described in, for example, Diabetic Retinopathy by Elia Duh, Springer, Humana Press, 2009; Mansour et al. (Investigative Ophthalmology & Visual Science, Vol. 31, No. 3, March 1990); and Henson and O'Brien (ILAR Journal Volume 47(3): 234-242).

### Monkeys/Primates

Protocols which may be used to test compounds for efficacy of treatment in monkeys and primates include those described in, for example, Kim et al. (Invest Ophthalmol Vis Sci. 2004; 45:4543-4553); Akimba: A Novel Murine Model for Diabetic Retinopathy (www.Bio-link.com); and Diabetic Retinopathy by Elia Duh, Springer, Humana Press, 2009.

### Example 6: Use of ALK6 Fc Fusion Proteins in an Age-Related Macular Degeneration Animal Model

C57BL/6J, male, 7-8 weeks old will be split into groups of six animals each, and all will have CNV induced using a 532 nm laser to make three laser spots per eye for both eyes at RPE/Bruch's membrane. One group will be treated with vehicle (PBS) and the other groups will each be treated with an ALK-6 Fc fusion polypeptide of this disclosure (e.g., ALK-6 ECD sequence of SEQ ID NO: 86; SEQ ID NO: 88; SEQ ID NO:69; SEQ ID NO:90; SEQ ID NO: 140; SEQ ID NO: 158; SEQ ID NO: 159; SEQ ID NO: 188; SEQ ID NO: 189, SEQ ID NO: 191 to 194 and SEQ ID NO: 286) via subcutaneous injection on day 1, 3 and 6 at a dose of 10 mg/kg or by direct intravitreal injection in to the eye of 10-50 micrograms/eye). Detailed ocular observation is carried out using slit lamp or Ophthalmoscope, abnormal conditions will be recorded (such as massive hemorrhage, retinal detachment, inflammation, turbid anterior). Following i.v. injection of sodium fluorescein, fundus fluorescent angiography is recorded. Leakage at lesion site is graded (I~IV). After final examinations, eyes are removed and fixed for flatmount staining. Immunofluorescence staining of isolectin B4 on RPE/Choroid flatmount is carried out for lesion area quantification.

Provided herein is a method for treating AMD and/or CNV comprising administering an effective amount of the soluble ALK6 Fc fusion polypeptide of this disclosure to a subject in need thereof.

### Example 7: Use of ALK6 Ligand Traps in an Diabetic Macular Edema Animal Model

C57BL/6J, male, 6-8 weeks old will be split into groups of six animals each, and all will have streptozocin (STZ) induced diabetes mellitus. One group will be treated with vehicle (PBS) and the other groups will each be treated with an ALK-6 Fc fusion polypeptide of this disclosure (e.g., ALK-6 ECD sequence of SEQ ID NO: 86; SEQ ID NO: 88; SEQ ID NO:69; SEQ ID NO: 90; SEQ ID NO: 140; SEQ ID NO: 158; SEQ ID NO: 159; SEQ ID NO: 188; SEQ ID NO: 189 and SEQ ID NO: 191 to 194 and SEQ ID NO: 286) via subcutaneous injection on day 1, 3 and 6 at a dose of 10 mg/kg or by direct intravitreal injection in to the eye of 10-50 micrograms/eye).

Six- to 8-week old C57BL/6J, are marked, weighed, and their baseline blood glucose levels determined before STZ injection. Mice receive daily intraperitoneal injections of 45 mg/kg body weight STZ dissolved in sterile sodium citrate dehydrate buffer, pH 4.5, for 5 consecutive days. For nondiabetic controls, mice receive citrate buffer injections. Tail blood glucose is monitored every week for ≤8 weeks after the final STZ injection. Mice were considered diabetic if their nonfasted blood glucose levels reached 17 mmol/L (300 mg/dL).

Six or seven weeks post STZ treatment, 1 group will be treated subcutaneously with vehicle (PBS) and another group will be treated subcutaneously with PBS twice a week or intravitreally at week 7 twice a week. Two other groups will each be treated subcutaneously twice a week beginning at week 6 or 7 or intravitreally twice a week at week 6 or 7 with an ALK-6 Fc fusion polypeptide of this disclosure (e.g., ALK-6 ECD sequence of SEQ ID NO: 86; SEQ ID NO: 88; SEQ ID NO:69; SEQ ID NO: 90; SEQ ID NO: 140; SEQ ID NO: 158; SEQ ID NO: 159; SEQ ID NO: 188; SEQ ID NO: 189; SEQ ID NO: 191 to 194 and SEQ ID NO: 286) via subcutaneous injection on day 1, 3 and 6 at a dose of 10 mg/kg or by direct intravitreal injection in to the eye of 10-50 micrograms/eye) on day 3 and or day 7.

At week 8 post STZ treatment, eyes will be assessed for diabetes induced permeability using an In Vivo Miles Assay for Vessel Permeability (diabetic macular edema). At week 8 post STZ treatment Mice are injected intravenously with Evans blue dye and 2 hours later were perfused with 9 mL of PBS. Immediately after, eyes were enucleated and fixed in 4% paraformaldehyde, and retinas are microsurgically removed and placed in formamide for 24 hours at 65 ÅãC for Evans blue extraction. Retinas were then dried and weighed on cotton wool paper. Absorbance maximum at 620 nm is read with a spectrophotometer. The concentration of dye in the extracts is calculated from a standard curve of Evans blue in formamide. Retinal vascular permeability will be normalized taking into consideration circulating Evans blue plasma concentration, retinal dry weight, and time of Evans blue circulation and calculated.

### Example 8: Use of ALK6 Ligand Traps in a Non-Proliferative Diabetic Retinopathy (NPDR) Animal Model

Diabetes will be induced in 6-week-old male C57Bl/6J mice via streptozotocin (STZ) injection in experiment Week 0 (W0) to recapitulate vascular pathologies characteristic of human non-proliferative diabetic retinopathy (NPDR). Animals will be individually identified and weighed, and their baseline fasting blood glucose (FBG) levels will be determined. After a 4-hour fast, animals will receive daily IP injection(s) of 50 mg/kg STZ in 10 mM citrate buffer (pH 4.5) for 5 consecutive days. Animals with FBG levels ≥300 mg/dL 10 days after the first STZ injection will be deemed diabetic. Body weight and FBG will be assessed every other week for the duration of the experiment.

Treatments will be administered via twice weekly (BIW) subcutaneous (SC) injections in W6, W7, and W8 (total of 6 doses per mouse) or via intravitreal (IVIT) injection in W8, one day prior to quantitative fluorescein angiography (qFA). qFA assessment will be performed in W8 after the final SC treatment. Mice will be euthanized in W8 after qFA assessment for collection of retinal tissue or analysis of vascular permeability via EBD extravasation.

Retinal vascular permeability will be assessed via quantitative fluorescein angiography (qFA). Animals will be anesthetized with an IP injection of ketamine/xylazine. Sodium fluorescein will be administered via IP injection at a dose of 10% fluorescein at 10 μL/g of body weight. Fluorescent fundus images will be captured for one eye of each animal with a Micron IV imaging system at the time of capillary filling with fluorescein, and again two minutes later. ImageJ software will be used by a masked observer to quantify the fluorescence intensity for each eye using the “integrated density” function. The difference in integrated density between the two times post-injection will be reported as a readout of vascular leakage.

Retinal vascular permeability will be assessed using Evans blue dye (EBD) extravasation. Mice will be injected via tail vein with 4 μL/g of 30 mg/mL EBD dissolved in 0.9% saline. Ninety minutes after tail vein injection, mice will be deeply anesthetized with an IP injection of ketamine/xylazine. Blood will be drawn from the left ventricle prior to perfusion for later determination of EBD concentration in plasma. Next, 0.9% saline will be perfused through the left ventricle. After perfusion, retinas will be dissected, weighed, and incubated in 100% formamide for 18 hours at 70 C to extract EBD into formamide. The following day, samples will be centrifuged at 100,000×g for 1 hour and the formamide supernatant will be removed for analysis. Extravasation of EBD from both retinas and plasma will be measured using a plate reader at A620, and relative EBD extravasation will be calculated by the following formula: [Retinal Evans Blue (ng/ml)/retina mass (g)] [Evans Blue plasma concentration (ng/ml)×circulation time (h)].

### Example 9: Systemic Dosing of ALK6-Fc Variants Reduces Inner Retinal Barrier Neurovascular Lesions in a Spontaneous CNV Mouse Model

Spontaneous choroidal neovascularization (sCNV) in mice, particularly in mutants like JR5558 or Vldlr−/−, provides a, chronic, non-laser model that replicates key features of wet age-related macular degeneration (AMD), including persistent, self-perpetuating vascular growth from the choroid. Unlike acute laser-induced models, these mice exhibit long-lasting lesions, retinal edema, and neuronal dysfunction, making them ideal for studying disease progression and long-term therapeutic interventions. A total 24 JR5558 mice were used in this study, which are a spontaneous mutant strain where neovascularization arises specifically from the choroid, leading to persistent, non-self-limiting lesions for 3+ months.

Before treatment, a detailed ocular examination was performed using a slit lamp to select mice with no abnormalities in the anterior segment of both eyes for FFA (Fundus Fluorescein Angiography) detection. 30 animals were selected and ranked from the smallest to the largest leakage area observed in FFA. These animals were then allocated to 3 treatment groups (G1-G3, 8 mice per group) in sequential group order with reverse leakage area rank, with final grouping refined by the number of leakage spots. Subsequently, a statistical analysis was conducted on the leakage area values of each group to ensure no significant differences (p>0.05).

TABLE 2

| | | | | | | | | Animal |
|---|---|---|---|---|---|---|---|---|
| Group | Induction | TA | Route | Concentration | | Dosage | | quantity |
| 1 | JR5558 | PBS | SC | — | | 15 | mg/kg | 8 |
| | | | injection | | | | | |
| 2 | JR5558 | ALK6-Fc | SC | 4 | mg/mL | 15 | mg/kg | 8 |
| | | fusion | injection | | | | | |
| | | comprising | | | | | | |
| | | SEQ ID NO: | | | | | | |
| | | 286 | | | | | | |
| 3 | JR5558 | ALK6-Fc | SC | 9.49 | mg/mL | 15 | mg/kg | 8 |

| | |
|---|---|
| fusion | injection |
| comprising | |
| SEQ ID NO: | |
| 87 | |

Group 1-3: Day-0, subcutaneous, twice a week, for 3 weeks, 15 mg/kg.

Time points: Day0, Day 7 and Day 21

In the study, the first dosing day is defined as Day 0/DO; the day before the first dosing day is defined as Day-1/D-1; the week before dosing is defined as Week-1/W-1; the first dosing week is defined as Week 1/W1 and so on.

Detailed ocular observation was carried out (Before treatment, Day14, Day21) using slit lamp or ophthalmoscope, abnormal conditions were recorded (such as massive hemorrhage, retinal detachment, inflammation, turbid anterior).

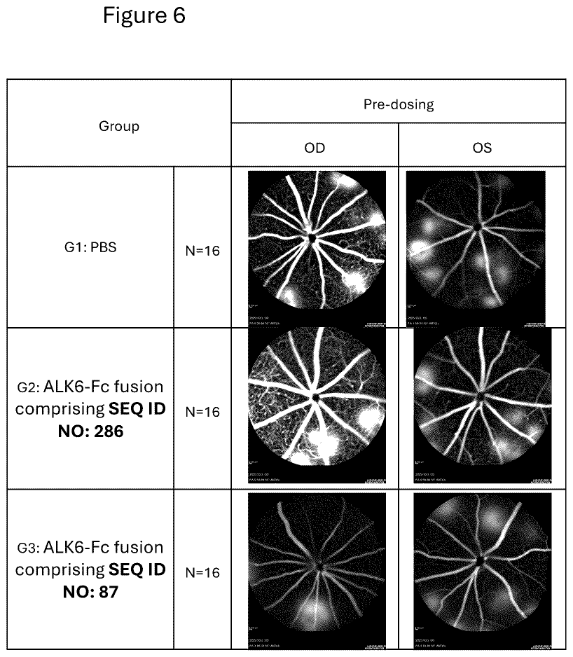

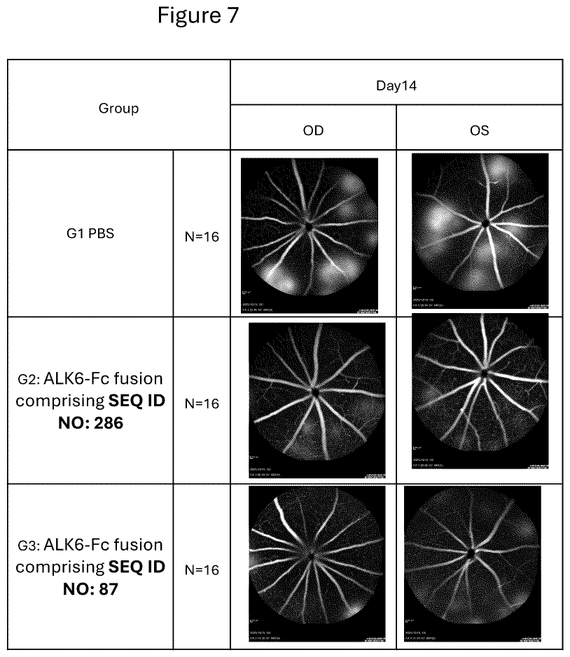

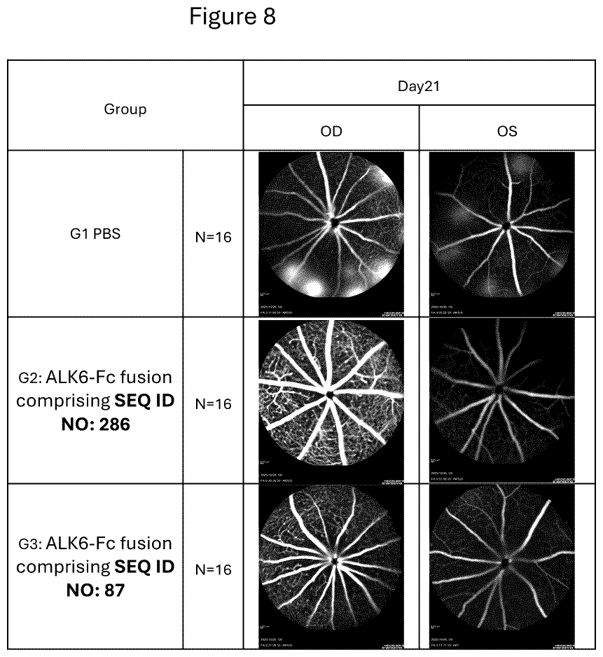

Early-phase (within 1.5 minutes) and late-phase (after 3 minutes) fundus fluorescein angiography images were captured before treatment, Day14 and Day21 after performing fluorescein sodium angiography. The number of leakage lesions across the entire fundus image were counted and the area of the leakage regions were measured. Representative images of FFA and result analysis are shown in FIG. 6 (pretreatment) FIG. 7 (Day 14) and FIG. 8 (Day 21), and Tables 3 and 4 below.

TABLE 3
Number of Leakage Lesions
The number of leakage lesions - Mean ± SEM

| Group | | Pre-dosing | Day 14 | Day 21 |
|---|---|---|---|---|
| G1 PBS | N = 16 | 11.25 ± 0.72 | 14.75 ± 1.39 | 13.38 ± 1.04 |
| G2 SEQ ID | N = 16 | 10.44 ± 1.45 | 7.88 ± 1.15 | 4.19 ± 0.82 |
| NO: 286 | | | | |
| G3 SEQ ID | N = 16 | 11.88 ± 1.47 | 8.63 ± 1.05 | 4.56 ± 0.70 |
| NO: 87 | | | | |

TABLE 4
Number of Leakage Lesions
The area of leakage lesions (μm²)- Mean ± SEM

| Group | | Pre-dosing | Day 14 | Day 21 |
|---|---|---|---|---|
| G1 PBS | N = 16 | 13.62 ± 2.15 | 22.06 ± 1.81 | 12.04 ± 1.09 |
| G2 SEQ ID | N = 16 | 13.66 ± 1.76 | 7.50 ± 1.07 | 5.08 ± 1.04 |
| NO: 286 | | | | |
| G3 SEQ ID | N = 16 | 13.76 ± 1.74 | 9.76 ± 1.64 | 4.73 ± 0.93 |
| NO: 87 | | | | |

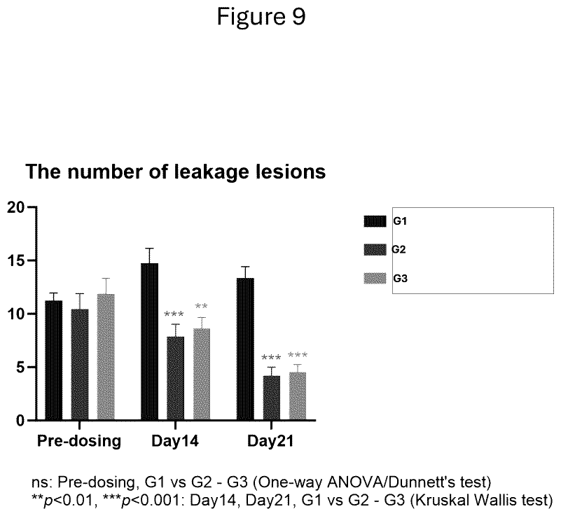

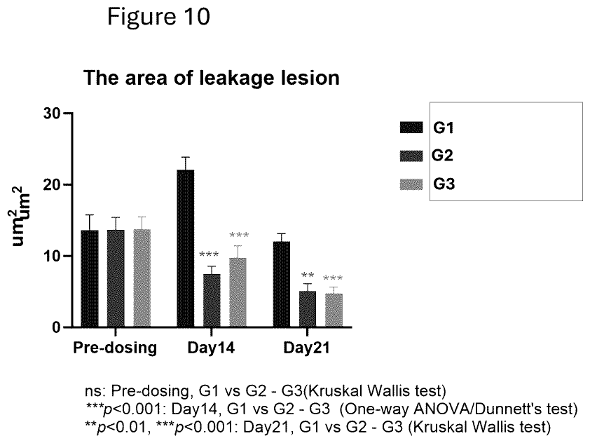

Prior to treatment, no significant differences were observed in either the number of leakage lesions or the total leakage area of G2 (ALK6-Fc fusion comprising SEQ ID NO: 286) and G3 (ALK6-Fc fusion comprising SEQ ID NO: 87) compared to G1 (PBS), but on Day 14 and Day21, the number of leakage lesions and leakage area were significantly decreased in G2 (ALK6-Fc fusion comprising SEQ ID NO: 286) and G3 (ALK6-Fc fusion comprising SEQ ID NO: 87) compared to G1 (PBS). See FIGS. 9 and 10.

The infrared images and OCT scanning of animals were performed and the whole retinal thickness and outer nuclear layer thickness were measured. The animals were anesthetized with Zoletil (25-50 mg/kg, i.p.)+Xylazine (5 mg/kg, i.p.) before examination.

Day21, the infrared images and OCT scanning of animals were performed and the whole retinal thickness and outer nuclear layer thickness were measured. G2 (ALK6-Fc fusion comprising SEQ ID NO: 286) did not show significant differences in retinal thickness at any distance from the optic center compared to G1 (PBS), but G3 (ALK6-Fc fusion comprising SEQ ID NO: 87) showed a significant decrease in retinal thickness at 1500 μm from the optic center compared to G1 (PBS). Compared with G1 (PBS), there was also no significant difference in ONL thickness of G3 (ALK6-Fc fusion comprising SEQ ID NO: 87) at any distance from the optic center, but the ONL thickness of G2 (ALK6-Fc fusion comprising SEQ ID NO: 286) was significantly increased at 500 μm and 1000 μm from the optic center.

This study evaluated the efficacy of ALK6-Fc fusion comprising SEQ ID NO: 286 and ALK6-Fc fusion comprising SEQ ID NO: 87 in spontaneous CNV mouse models by using fundus fluorescent angiograph and OCT. In this study, no ocular abnormalities were observed. Subcutaneous injections of ALK6-Fc fusion comprising SEQ ID NO: 286 and ALK6-Fc fusion comprising SEQ ID NO: 87 did not cause significant weight changes in any animals during all treatment periods. Both treatments significantly decreased the number of leakage lesions and leakage area. Both ALK6-Fc fusion comprising SEQ ID NO: 286 and ALK6-Fc fusion comprising SEQ ID NO: 87 demonstrated significant therapeutic effects in spontaneous CNV mouse models.

### Example 10: Intravitreal Dosing of ALK6-Fc Variants Reduces Inner Retinal Barrier Neurovascular Lesions in a Spontaneous CNV Mouse Model

This study was performed similar to Example 9 except for the change in route of administration and as described below.

TABLE 5
Grouping and dosing regiment

| | | | | | | | Animal |
|---|---|---|---|---|---|---|---|
| Group | Induction | TA | Route | Concentration | | Dosage | quantity |
| 1 | JR5558 | PBS | IVT injection | — | | 2 μL/eye | 8 |
| 2 | JR5558 | ALK6-Fc | IVT injection | 26.3 | mg/mL | 52.6 μg/2 | 8 |
| | | fusion | | | | μL/eye | |
| | | comprising | | | | | |
| | | SEQ ID | | | | | |
| | | NO: 286 | | | | | |
| 3 | JR5558 | EYLEA | IVT injection | 5.5 | mg/mL | 11 μg/2 | 8 |

μL/eye

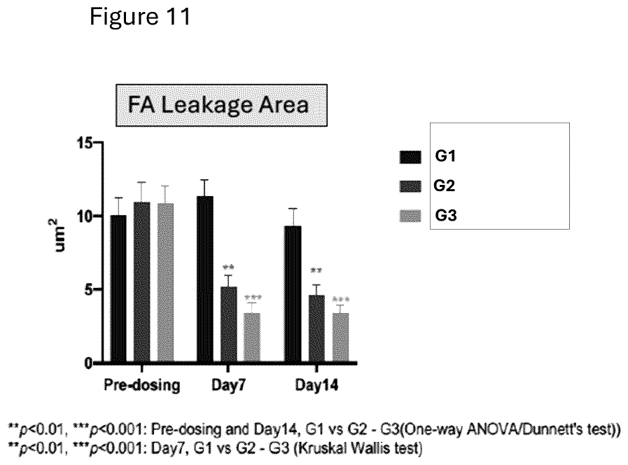

Group 1-3: Day0, single IVT injection, 2 μL/eye. See FIG. 11.

### Example 11: Intravitreal Dosing of ALK6-Fc Variants Effectively Reduces Vascular Leakage in a Rabbit Model of Glia Cell Injury

The rabbit model of glia cell injury is a valuable tool for studying retinal disease mechanisms and testing new treatments for retinopathies (e.g., diabetic retinopathy, retinal detachment, and glaucoma). The rabbit model of glia cell injury is used to study how glial cells in the eye, particularly in the retina, respond to treatments for retinopathy disease. In this model, injury or stress is induced in the rabbit retina—often through mechanical, ischemic, or chemical means—to mimic conditions seen in human retinal vascular diseases. After injury, retinal glial cells (mainly Müller cells and astrocytes) become reactive, proliferate, and may release inflammatory mediators. The rabbit model allows for the evaluation of potential treatments aimed at reducing glial cell activation, inflammation, and scarring in the retina.

In this study, eight (8) male rabbits (3-6 months old) are used and glia cell injury induced with intravitreal injection of DL-2-aminoadipic acid (DL-AAA) into unilateral eyes to induce the persistent retinal neovascularization (PRNV) model.

Around 8 weeks after DL-AAA induction, induced animals were screened by FFA (Fundus Fluorescein Angiography) to evaluate retinal vascular leakage and neovascularization. The animals with successfully induced eyes without serious inflammation and hemorrhage were randomly grouped to receive treatments.

TABLE 6

| Group | Induction | TA | Route | Concentration | Dosage | Eye |
|---|---|---|---|---|---|---|
| 1 | DL-AAA | ALK6 Fc fusion | IVT injection, | 26.3 mg/mL | 20 μL/eye | 8 |
| | | proteins | once | | | |
| | | comprising SEQ | | | | |
| | | ID NO: 286 | | | | |

Group 1: Day0, single IVT injection for model eye, 20 mL/eye. In the study, the first dosing day was defined as Day 0/DO; the day before the first dosing day was defined as Day-1/D-1; the week before dosing was defined as Week-1/W-1; the first dosing week was defined as Week 1/W1 and so on.

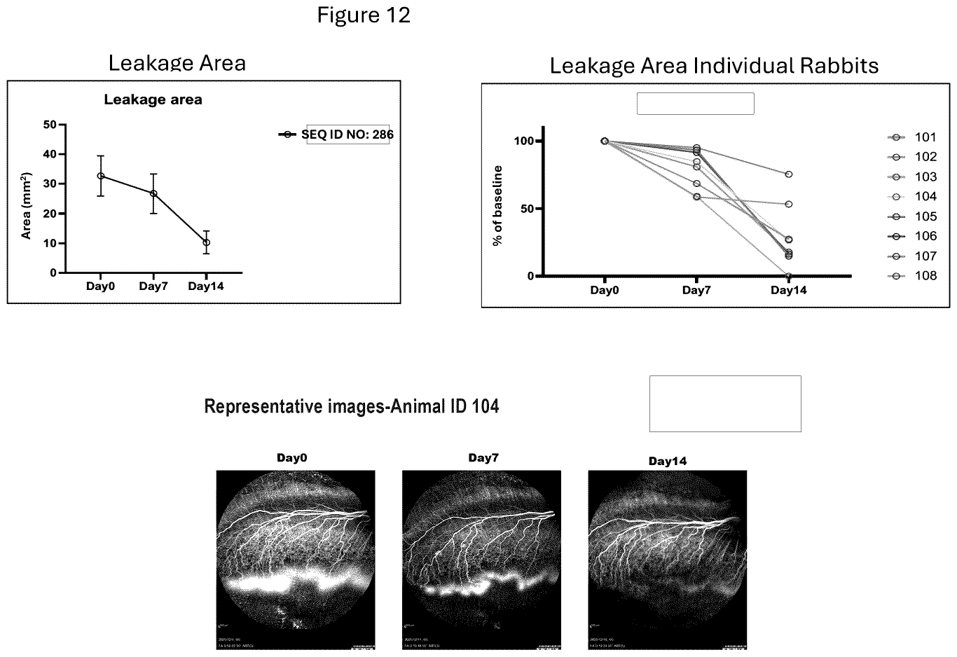

Day0 (Before treatment), Day7, Day 14, FFA images of the early stage (within 1.5 minutes) and the late stage (after 3 minutes) were collected respectively after the injection of sodium fluorescein injection. The retinal leakage and neovascularization were graded using a 6-part grading system, and the leakage area quantified. See FIG. 12.

Provided herein in embodiments is a method for treating AMD comprising administering an effective amount of the soluble ALK6 ECD Fc fusion polypeptide of this disclosure to a human subject in need thereof. In these embodiments, the soluble ALK-6 Fc fusion polypeptide comprises an ALK-6 ECD sequence having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 437. In certain embodiments, the ALK-6 ECD Fc fusion protein for treatment of AMD comprises an ALK-6 ECD sequence having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 286. For methods of treating AMD in a human subject the ALK6 Fc fusion polypeptide comprises a sequence having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD Fc fusion protein sequence of SEQ ID NO: 291.

SEQUENCE LISTING TABLE

| SEQ | | |
|---|---|---|
| ID | | |
| No | Description | Amino acid sequence |
| 1 | Wild type ALK-6 | KKEDGESTAPTPRPKVLRCKCHHHCPEDSVNNICSTDGYCFTMI |
| | ECD Sequence | EEDDSGLPVVTSGCLGLEGSDFQCRDTPIPHQRRSIECCTERNEC |
| | | NKDLHPTLPPLKNRDFVDGPIHHR |
| 2 | C terminus ALK 3 | VVIGPFFDGSIR |
| | ECD sequence | |
| 3 | Wild type ALK 6 ECD | Lys₁-Lys₂-Glu₃-Asp₄-Gly₅-Glu₆-Ser₇-Thr₈-Ala₉-Pro₁₀- |
| | background with | Thr₁₁-Pro₁₂-Arg₁₃-Pro₁₄-Xaa₁₅-Val₁₆-Leu₁₇-Arg₁₈- |
| | point mutations with | Cys₁₉-Xaa₂₀-Cys₂₁-Xaa₂₂-Xaa₂₃-His₂₄-Cys₂₅-Pro₂₆-Xaa₂₇- |
| | ALK6 or ALK3 C | Asp₂₈-Xaa₂₉-Xaa₃₀-Asn₃₁-Asn₃₂-Xaa₃₃-Cys₃₄-Xaa₃₅- |
| | terminus sequence | Thr₃₆-Xaa₃₇-Gly₃₈-Xaa₃₉-Cys₄₀-Phe₄₁-Xaa₄₂-Xaa₄₃-Ile₄₄- |
| | | Glu₄₅-Glu₄₆-Asp₄₇-Asp₄₈-Xaa₄₉-Gly₅₀-Xaa₅₁-Xaa₅₂-Xaa₅₃- |
| | | Xaa₅₄-Xaa₅₅-Ser₅₆-Gly₅₇-Cys₅₈-Xaa₅₉-Xaa₆₀-Xaa₆₁- |
| | | Glu₆₂-Gly₆₃-Ser₆₄-Asp₆₅-Phe₆₆-Gln₆₇-Cys₆₈-Xaa₆₉-Asp₇₀- |
| | | Xaa₇₁-Pro₇₂-Xaa₇₃-Xaa₇₄-Xaa₇₅-Xaa₇₆-Arg₇₇-Arg₇₈- |
| | | Xaa₇₉-Ile₈₀-Glu₈₁-Cys₈₂-Cys₈₃-Xaa₈₄-Xaa₈₅-Xaa₈₆-Xaa₈₇- |
| | | Xaa₈₈-Cys₈₉-Asn₉₀-Xaa₉₁-Xaa₉₂-Leu₉₃-Xaa₉₄-Pro₉₅-Thr₉₆- |
| | | Leu₉₇-Pro₉₈-Pro₉₉-Xaa₁₀₀-Xaa₁₀₁-Xaa₁₀₂-Xaa₁₀₃-Xaa₁₀₄- |
| | | Xaa₁₀₅-Xaa₁₀₆-Xaa₁₀₇-Xaa₁₀₈-Xaa₁₀₉-Xaa₁₁₀-Xaa₁₁₁-Xaa₁₁₂- |
| | | Xaa₁₁₃ |
| | | wherein: Xaa₁₅ is K or P; Xaa₂₀ is V, F, K or Y; Xaa₂₂ is G, H or S; |
| | | Xaa₂₃ is S, H or G; Xaa₂₇ is E or D; Xaa₂₉ is S or A; Xaa₃₀ is V or I; |
| | | Xaa₃₃ is I or T; Xaa₃₅ is S or I; Xaa₃₇ is D or N; Xaa₃₉ is Y or H; |
| | | Xaa₄₂ is T or A; Xaa₄₃ is M or I; Xaa₄₉ is S or Q; Xaa₅₁ is L or E; |
| | | Xaa₅₂ is P or T; Xaa₅₃ is V or T; Xaa₅₄ is V or L; Xaa₅₅ is T or A; |
| | | Xaa₅₉ is L or M Xaa₆₀ is G or K; Xaa₆₁ is L or Y; Xaa₆₉ is R or K; |
| | | Xaa₇₁ is T or S; Xaa₇₃ is I or K; Xaa₇₄ is P or A; Xaa₇₅ is H or Q; |
| | | Xaa₇₆ is Q or L; Xaa₇₉ is S or T; Xaa₈₄ is T or R; Xaa₈₅ is absent, T |
| | | or E; Xaa₈₆ is R or N; Xaa₈₇ is N or L; Xaa₈₈ is absent, E or L; Xaa₉₁ |
| | | is K or Q; Xaa₉₂ is D or L; Xaa₉₄ is H or Q; Xaa₁₀₀ is absent, V or L; |
| | | Xaa₁₀₁ is absent, V or K; Xaa₁₀₂ is absent, I or N; Xaa₁₀₃ is absent, G |
| | | or R; Xaa₁₀₄ is absent, P, D or A; Xaa₁₀₅ is absent or F; Xaa₁₀₆ is |
| | | absent, F or V; Xaa₁₀₇ is absent or D; Xaa₁₀₈ is absent or G; Xaa₁₀₉ is |
| | | absent, S or P; Xaa₁₁₀ is absent or I; Xaa₁₁₁ is absent, R or H; Xaa₁₁₂ |
| | | is absent or H; and Xaa₁₁₃ is absent or R |
| 4 | Wild type ALK-6 with | KKEDGESTAPTPRPKVLRCKCHHHCPEDSVNNICSTDGYCFT |
| | C terminus truncated | MIEEDDSGLPVVTSGCLGLEGSDFQCRDTPIPHQRRSIECCTE |
| | (13 aa deleted) | RNECNKDLHPTLPPL |
| 5 | Wild type ALK-6 | Lys₁-Lys₂-Glu₃-Asp₄-Gly₅-Glu₆-Ser₇-Thr₈-Ala₉-Pro₁₀- |
| | ECD with C terminus | Thr₁₁-Pro₁₂-Arg₁₃-Pro₁₄-Xaa₁₅-Val₁₆-Leu₁₇-Arg₁₈- |
| | truncated background | Cys₁₉-Xaa₂₀-Cys₂₁-Xaa₂₂-Xaa₂₃-His₂₄-Cys₂₅-Pro₂₆-Xaa₂₇- |
| | with point mutations | Asp₂₈-Xaa₂₉-Xaa₃₀-Asn₃₁-Asn₃₂-Xaa₃₃-Cys₃₄-Xaa₃₅- |
| | | Thr₃₆-Xaa₃₇-Gly₃₈-Xaa₃₉-Cys₄₀-Phe₄₁-Xaa₄₂-Xaa₄₃-Ile₄₄- |
| | | Glu₄₅-Glu₄₆-Asp₄₇-Asp₄₈-Xaa₄₉-Gly₅₀-Xaa₅₁-Xaa₅₂-Xaa₅₃- |
| | | Xaa₅₄-Xaa₅₅-Ser₅₆-Gly₅₇-Cys₅₈-Xaa₅₉-Xaa₆₀-Xaa₆₁- |
| | | Glu₆₂-Gly₆₃-Ser₆₄-Asp₆₅-Phe₆₆-Gln₆₇-Cys₆₈-Xaa₆₉-Asp₇₀- |
| | | Xaa₇₁-Pro₇₂-Xaa₇₃-Xaa₇₄-Xaa₇₅-Xaa₇₆-Arg₇₇-Arg₇₈- |
| | | Xaa₇₉-Ile₈₀-Glu₈₁-Cys₈₂-Cys₈₃-Xaa₈₄-Xaa₈₅-Xaa₈₆-Xaa₈₇- |
| | | Xaa₈₈-Cys₈₉-Asn₉₀-Xaa₉₁-Xaa₉₂-Leu₉₃-Xaa₉₄-Pro₉₅-Thr₉₆- |
| | | Leu₉₇-Pro₉₈-Pro₉₉-Leu₁₀₀ |
| | | wherein Xaa₁₅ is K or P; Xaa₂₀ is V, F, K or Y; Xaa₂₂ is G, H or S; |
| | | Xaa₂₃ is S, H or G; Xaa₂₇ is E or D; Xaa₂₉ is S or A; Xaa₃₀ is V or I; |
| | | Xaa₃₃ is I or T; Xaa₃₅ is S or I; Xaa₃₇ is D or N; Xaa₃₉ is Y or H; Xaa₄₂ |
| | | is T or A; Xaa₄₃ is M or I; Xaa₄₉ is S or Q; Xaas₁ is L or E; Xaa₅₂ is P |
| | | or T; Xaa₅₃ is V or T; Xaa₅₄ is V or L; Xaa₅₅ is T or A; Xaa₅₉ is L or M; |
| | | Xaa₆₀ is G or K; Xaa₆₁ is L or Y; Xaa₆₉ is R or K; Xaa₇₁ is T or S; Xaa₇₃ |
| | | is I or K; Xaa₇₄ is P or A; Xaa₇₅ is H or Q; Xaa₇₆ is Q or L; Xaa₇₉ is S |
| | | or T; Xaa₈₄ is T or R; Xaa₈₅ is absent, T or E; Xaa₈₆ is R or N; Xaa₈₇ |
| | | is N or L; Xaa₈₈ is absent, E or L; Xaa₉₁ is K or Q; Xaa₉₂ is D or L; |
| | | and, Xaa₉₄ is H or Q |
| 6 | Linker sequence | TGGGGSGGGGSGGGGSGGGGS |
| 7 | Linker sequence | TGGG |
| 8 | Linker sequence | EAAAKEAAAKEAAAKEAAAK |
| 9 | Leader sequence | MGWSCIILFLVATATGVHS |
| 10 | Fc sequence | THTCPPCPAPELLGGPSVFLFPPKPKDTLMISRTPEVTCVVVDVS |
| | | HEDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQ |
| | | KSLSLSPGK |
| 11 | Truncated ALK6 | KKEDGESTAPTPRPKVLRCKCHHHCPEDSVNNICSTDGYCFTMI |
| | ECD (14 aa deleted) | EEDDSGLPVVTSGCLGLEGSDFQCRDTPIPHQRRSIECCTERNEC |
| | with ALK3 C | NKDLHPTLPPVVIGPFFDGSIR |
| | terminus sequence | |
| 12 | SEQ ID NO: 1 D104A | KKEDGESTAPTPRPKVLRCKCHHHCPEDSVNNICSTDGYCFTMI |
| | Variant | EEDDSGLPVVTSGCLGLEGSDFQCRDTPIPHQRRSIECCTERNEC |
| | | NKDLHPTLPPLKNRAFVDGPIHHR |
| 13 | Wild type ALK 6 ECD | KKEDGESTAPTPRPKVLRCKCHHHCPEDSVNNICSTDGYCFTMI |
| | with D104A mutation | EEDDSGLPVVTSGCLGLEGSDFQCRDTPIPHQRRSIECCTERNEC |
| | and C terminus | NKDLHPTLPPLKNRAFV____ |
| | truncated | |
| 14 | SEQ ID NO: 1 D104A | KKEDGESTAPTPRPKVLRCKCHHHCPEDSVNNICSTDGYCFTMI |
| | and E107 deletion | EEDDSGLPVVTSGCLGLEGSDFQCRDTPIPHQRRSIECCTERNEC |
| | Variant | NKDLHPTLPPLKNRAFV___GPIHHR |
| 15 | SEQ ID NO: 11 | KKEDGESTAPTPRPKVLRCKCHGHCPEDSVNNICSTNGYCFTIIE |
| | Variant with 6 point | EDDSGLPVVTSGCMGYEGSDFQCRDTPKAHLRRSIECCTERNE |
| | mutations | CNKDLHPTLPPVVIGPFFDGSIR |
| | (underlined) | |
| 16 | SEQ ID NO: 11 E85 | KKEDGESTAPTPRPKVLRCKCHHHCPEDSVNNICSTDGYCFTMI |
| | Deletion Variant | EEDDSGLPVVTSGCLGLEGSDFQCRDTPIPHQRRSIECCT_RNEC |
| | | NKDLHPTLPPVVIGPFFDGSIR |
| 17 | SEQ ID NO: 11 K15P | KKEDGESTAPTPRPPVLRCKCHHHCPEDSVNNICSTDGYCFTMI |
| | Variant | EEDDSGLPVVTSGCLGLEGSDFQCRDTPIPHQRRSIECCTERNEC |
| | | NKDLHPTLPPVVIGPFFDGSIR |
| 18 | SEQ ID NO: 11 K20Y | KKEDGESTAPTPRPKVLRCYCHHHCPEDSVNNICSTDGYCFTMI |
| | Variant | EEDDSGLPVVTSGCLGLEGSDFQCRDTPIPHQRRSIECCTERNEC |
| | | NKDLHPTLPPVVIGPFFDGSIR |
| 19 | SEQ ID NO: 11 H22S | KKEDGESTAPTPRPKVLRCKCSHHCPEDSVNNICSTDGYCFTMI |
| | Variant | EEDDSGLPVVTSGCLGLEGSDFQCRDTPIPHQRRSIECCTERNEC |
| | | NKDLHPTLPPVVIGPFFDGSIR |
| 20 | SEQ ID NO: 11 H23G | KKEDGESTAPTPRPKVLRCKCHGHCPEDSVNNICSTDGYCFTMI |
| | Variant | EEDDSGLPVVTSGCLGLEGSDFQCRDTPIPHQRRSIECCTERNEC |
| | | NKDLHPTLPPVVIGPFFDGSIR |
| 21 | SEQ ID NO: 11 K20Y, | KKEDGESTAPTPRPKVLRCYCSGHCPEDSVNNICSTDGYCFTMI |
| | H22S and H23G | EEDDSGLPVVTSGCLGLEGSDFQCRDTPIPHQRRSIECCTERNEC |
| | Variant | NKDLHPTLPPVVIGPFFDGSIR |
| 22 | SEQ ID NO: 11 E27D | KKEDGESTAPTPRPKVLRCKCHHHCPDDSVNNICSTDGYCFTMI |
| | Variant | EEDDSGLPVVTSGCLGLEGSDFQCRDTPIPHQRRSIECCTERNEC |
| | | NKDLHPTLPPVVIGPFFDGSIR |
| 23 | SEQ ID NO: 11 V30I | KKEDGESTAPTPRPKVLRCKCHHHCPEDSINNICSTDGYCFTMIE |
| | Variant | EDDSGLPVVTSGCLGLEGSDFQCRDTPIPHQRRSIECCTERNECN |
| | | KDLHPTLPPVVIGPFFDGSIR |
| 24 | SEQ ID NO: 11 I33T | KKEDGESTAPTPRPKVLRCKCHHHCPEDSVNNTCSTDGYCFTM |
| | Variant | IEEDDSGLPVVTSGCLGLEGSDFQCRDTPIPHQRRSIECCTERNE |
| | | CNKDLHPTLPPVVIGPFFDGSIR |
| 25 | SEQ ID NO: 11 S35I | KKEDGESTAPTPRPKVLRCKCHHHCPEDSVNNICITDGYCFTMI |
| | Variant | EEDDSGLPVVTSGCLGLEGSDFQCRDTPIPHQRRSIECCTERNEC |
| | | NKDLHPTLPPVVIGPFFDGSIR |
| 26 | SEQ ID NO: 11 D37N | KKEDGESTAPTPRPKVLRCKCHHHCPEDSVNNICSTNGYCFTMI |
| | Variant | EEDDSGLPVVTSGCLGLEGSDFQCRDTPIPHQRRSIECCTERNEC |
| | | NKDLHPTLPPVVIGPFFDGSIR |
| 27 | SEQ ID NO: 11 Y39H | KKEDGESTAPTPRPKVLRCKCHHHCPEDSVNNICSTDGHCFTMI |
| | Variant | EEDDSGLPVVTSGCLGLEGSDFQCRDTPIPHQRRSIECCTERNEC |
| | | NKDLHPTLPPVVIGPFFDGSIR |
| 28 | SEQ ID NO: 11 T42A | KKEDGESTAPTPRPKVLRCKCHHHCPEDSVNNICSTDGYCFAIIE |
| | and M43I Variant | EDDSGLPVVTSGCLGLEGSDFQCRDTPIPHQRRSIECCTERNECN |
| | | KDLHPTLPPVVIGPFFDGSIR |
| 29 | SEQ ID NO: 11 T42A | KKEDGESTAPTPRPKVLRCKCHHHCPEDSVNNICSTDGYCFAMI |
| | Variant | EEDDSGLPVVTSGCLGLEGSDFQCRDTPIPHQRRSIECCTERNEC |
| | | NKDLHPTLPPVVIGPFFDGSIR |
| 30 | SEQ ID NO: 11 M43I | KKEDGESTAPTPRPKVLRCKCHHHCPEDSVNNICSTDGYCFTIIE |
| | Variant | EDDSGLPVVTSGCLGLEGSDFQCRDTPIPHQRRSIECCTERNECN |
| | | KDLHPTLPPVVIGPFFDGSIR |
| 31 | SEQ ID NO: 11 S49Q | KKEDGESTAPTPRPKVLRCKCHHHCPEDSVNNICSTDGYCFTMI |
| | Variant | EEDDQGLPVVTSGCLGLEGSDFQCRDTPIPHQRRSIECCTERNEC |
| | | NKDLHPTLPPVVIGPFFDGSIR |
| 32 | SEQ ID NO: 11 S49Q, | KKEDGESTAPTPRPKVLRCKCHHHCPEDSVNNICSTDGYCFTMI |
| | L51E, P52T, V53T, | EEDDQGETTLASGCLGLEGSDFQCRDTPIPHQRRSIECCTERNE |
| | V54L, T55A Variant | CNKDLHPTLPPVVIGPFFDGSIR |
| 33 | SEQ ID NO: 11 | KKEDGESTAPTPRPKVLRCKCHHHCPEDSVNNICSTDGYCFTMI |
| | L59M, G60K and | EEDDSGLPVVTSGCMKYEGSDFQCRDTPIPHQRRSIECCTERNE |
| | L61Y Variant | CNKDLHPTLPPVVIGPFFDGSIR |
| 34 | SEQ ID NO: 11 L59M | KKEDGESTAPTPRPKVLRCKCHHHCPEDSVNNICSTDGYCFTMI |
| | Variant | EEDDSGLPVVTSGCMGLEGSDFQCRDTPIPHQRRSIECCTERNE |
| | | CNKDLHPTLPPVVIGPFFDGSIR |
| 35 | SEQ ID NO: 11 G60K | KKEDGESTAPTPRPKVLRCKCHHHCPEDSVNNICSTDGYCFTMI |
| | Variant | EEDDSGLPVVTSGCLKLEGSDFQCRDTPIPHQRRSIECCTERNEC |
| | | NKDLHPTLPPVVIGPFFDGSIR |
| 36 | SEQ ID NO: 11 L61Y | KKEDGESTAPTPRPKVLRCKCHHHCPEDSVNNICSTDGYCFTMI |
| | Variant | EEDDSGLPVVTSGCLGYEGSDFQCRDTPIPHQRRSIECCTERNEC |
| | | NKDLHPTLPPVVIGPFFDGSIR |
| 37 | SEQ ID NO: 11 I73K, | KKEDGESTAPTPRPKVLRCKCHHHCPEDSVNNICSTDGYCFTMI |
| | P74A, H75Q, Q76L | EEDDSGLPVVTSGCLGLEGSDFQCRDTPKAQLRRSIECCTERNE |
| | Variant | CNKDLHPTLPPVVIGPFFDGSIR |
| 38 | SEQ ID NO: 11 I73K | KKEDGESTAPTPRPKVLRCKCHHHCPEDSVNNICSTDGYCFTMI |
| | and P74A Variant | EEDDSGLPVVTSGCLGLEGSDFQCRDTPKAHQRRSIECCTERNE |
| | | CNKDLHPTLPPVVIGPFFDGSIR |
| 39 | SEQ ID NO: 11 H75Q | KKEDGESTAPTPRPKVLRCKCHHHCPEDSVNNICSTDGYCFTMI |
| | and Q76L Variant | EEDDSGLPVVTSGCLGLEGSDFQCRDTPIPQLRRSIECCTERNEC |
| | | NKDLHPTLPPVVIGPFFDGSIR |
| 40 | SEQ ID NO: 11 T84R | KKEDGESTAPTPRPKVLRCKCHHHCPEDSVNNICSTDGYCFTMI |
| | and E88L Variant | EEDDSGLPVVTSGCLGLEGSDFQCRDTPIPHQRRSIECCRERNLC |
| | | NKDLHPTLPPVVIGPFFDGSIR |
| 41 | SEQ ID NO: 11 G60K, | KKEDGESTAPTPRPKVLRCKCHHHCPEDSVNNICSTDGYCFTMI |
| | I73K, P74A, H75Q | EEDDSGLPVVTSGCLKLEGSDFQCRDTPKAQLRRSIECCTERNE |
| | and Q76L Variant | CNKDLHPTLPPVVIGPFFDGSIR |
| 42 | SEQ ID NO: 11 T84R, | KKEDGESTAPTPRPKVLRCKCHHHCPEDSVNNICSTDGYCFTMI |
| | E85T, R86N, N87L, | EEDDSGLPVVTSGCLGLEGSDFQCRDTPIPHQRRSIECCRTNL_C |
| | E88_, K91Q, D92Y | NQYLQPTLPPVVIGPFFDGSIR |
| | and H94Q Variant | |
| 43 | SEQ ID NO: 11 E85 | KKEDGESTAPTPRPKVLRCKCHHHCPEDSVNNICSTDGYCFTMI |
| | Variant | EEDDSGLPVVTSGCLGLEGSDFQCRDTPIPHQRRSIECCT_RNEC |
| | | NKDLHPTLPPVVIGPFFDGSIR |
| 44 | SEQ ID NO: 11 | KKEDGESTAPTPRPKVLRCKCSGHCPDDAVNNTCITNGYCFAII |
| | Variant | EEDDSGLPVVASGCMKYEGSDFQCKDSPKAQLRRTIECCTERN |
| | H22S, H23G, E27D, | ECNKDLHPTLPPVVIGPFFDGSIR |
| | S29A, I33T, S35I, | |
| | D37N, T43A, M43I, | |
| | T55A, L59M, G60K, | |
| | L61Y, R69K, T71S, | |
| | I73K, P74A, H75Q, | |
| | Q76L, S79T | |
| 45 | SEQ ID NO: 11 | KKEDGESTAPTPRPKVLRCKCHGHCPEDSVNNICSTNGYCFTIIE |
| | Variant H23G, D37N, | EDDSGLPVVTSGCMKYEGSDFQCRDTPKAHLRRSIECCTERNE |
| | M43I, L59M, G60K, | CNKDLHPTLPPVVIGPFFDGSIR |
| | L61Y, I73K, P74A, | |
| | Q76L | |
| 46 | SEQ ID NO: 46 | KKEDGESTAPTPRPKVLRCKCHHHCPEDSVNNICSTDGYCFTMI |
| | | EEDDSGLPVVTSGCLGLEGSDFQCRDTPIPHQRRSIECCTERNEC |
| | | NKDLHPTLPPVVIGPFFDGSIRTGGGGSGGGGSGGGGSGGGG |
| | | STHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 47 | SEQ ID NO: 1 D104A | KKEDGESTAPTPRPKVLRCKCHHHCPEDSVNNICSTDGYCFTMI |
| | Variant | EEDDSGLPVVTSGCLGLEGSDFQCRDTPIPHQRRSIECCTERNEC |
| | | NKDLHPTLPPLKNRAFVDGPIHHRTGGGGSGGGGSGGGGSGG |
| | | GGSTHTCPPCPAPELLGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 48 | SEQ ID NO: 1 D104A | KKEDGESTAPTPRPKVLRCKCHHHCPEDSVNNICSTDGYCFTMI |
| | and C terminus 7 aa | EEDDSGLPVVTSGCLGLEGSDFQCRDTPIPHQRRSIECCTERNEC |
| | deletion Variant | NKDLHPTLPPLKNRAFV____TGGGGSGGGGSGGGGSGGG |
| | | GSTHTCPPCPAPELLGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 49 | Wild type ALK-6 ECD | MGWSCIILFLVATATGVHSKKEDGESTAPTPRPKVLRCKCHH |
| | Sequence (SEQ ID NO: | HCPEDSVNNICSTDGYCFTMIEEDDSGLPVVTSGCLGLEGSD |
| | 1) with | FQCRDTPIPHQRRSIECCTERNECNKDLHPTLPPLKNRDFVDG |
| | Leader sequence (SEQ | PIHHRTGGGGSGGGGSGGGGSGGGGSTHTCPPCPAPELLG |
| | ID NO: 9) and linker | GPSVFLFPPKPKDTLMISRTPEVTCVVVDVSHEDPEVKFNWY |
| | sequence (SEQ ID No: | VDGVEVHNAKTKPREEQYNSTYRVVSVLTVLHQDWLNGKE |
| | 6) are underlined | YKCKVSNKALPAPIEKTISKAKGQPREPQVYTLPPSREEMTK |
| | followed by Fc-domain | NQVSLTCLVKGFYPSDIAVEWESNGQPENNYKTTPPVLDSD |
| | sequence (SEQ ID NO: | GSFFLYSKLTVDKSRWQQGNVFSCSVMHEALHNHYTQKSLS |
| | 10) | LSPGK |
| 50 | ALK-6 ECD Sequence | MGWSCIILFLVATATGVHSKKEDGESTAPTPRPKVLRCKCHHH |
| | with C-terminus 14 | CPEDSVNNICSTDGYCFTMIEEDDSGLPVVTSGCLGLEGSDFQC |
| | amino acids deleted | RDTPIPHQRRSIECCTERNECNKDLHPTLPPLTGGGGSGGGGSG |
| | (SEQ ID NO: 4) with | GGGSGGGGSTHTCPPCPAPELLGGPSVFLFPPKPKDTLMISRTP |
| | Leader sequence (SEQ | EVTCVVVDVSHEDPEVKFNWYVDGVEVHNAKTKPREEQYNST |
| | ID NO: 9) and linker | YRVVSVLTVLHQDWLNGKEYKCKVSNKALPAPIEKTISKAKGQ |
| | sequence (SEQ ID No: | PREPQVYTLPPSREEMTKNQVSLTCLVKGFYPSDIAVEWESNGQ |
| | 6) underlined followed | PENNYKTTPPVLDSDGSFFLYSKLTVDKSRWQQGNVFSCSVMH |
| | by Fc-domain sequence | EALHNHYTQKSLSLSPGK |
| | (SEQ ID NO: 10) | |
| 51 | C-terminus 13 amino | LKNRDFVDGPIHHR |
| | acids from the ECD of | |
| | human wild type ALK6 | |
| | (SEQ ID NO: 4) | |
| 52 | SEQ ID NO: 52 | KKEDGESTAPTPRPKVLRCKCHGHCPEDSVNNICSTDGYCFTMI |
| | | EEDDSGLPVVTSGCLGLEGSDFQCRDTPIPHQRRSIECCTERNEC |
| | | NKDLHPTLPPVVIGPFFDGSIRTGGGGSGGGGSGGGGSGGGG |
| | | STHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 53 | SEQ ID NO: 53 | KKEDGESTAPTPRPKVLRCKCHHHCPEDSVNNICSTNGYCFT |
| | | MIEEDDSGLPVVTSGCLGLEGSDFQCRDTPIPHQRRSIECCTE |
| | | RNECNKDLHPTLPPVVIGPFFDGSIRTGGGGSGGGGSGGG |
| | | GSGGGGSTHTCPPCPAPELLGGPSVFLFPPKPKDTLMISRTPE |
| | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| 54 | SEQ ID NO: 54 | KKEDGESTAPTPRPKVLRCKCHHHCPEDSVNNICSTDGYCFAIIE |
| | | EDDSGLPVVTSGCLGLEGSDFQCRDTPIPHQRRSIECCTERNECN |
| | | KDLHPTLPPVVIGPFFDGSIRTGGGGSGGGGSGGGGSGGGGS |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQ |
| | | KSLSLSPGK |
| 55 | SEQ ID NO: 55 | KKEDGESTAPTPRPKVLRCKCHHHCPEDSVNNICSTDGYCFTMI |
| | | EEDDSGLPVVTSGCMKYEGSDFQCRDTPIPHQRRSIECCTERNE |
| | | CNKDLHPTLPPVVIGPFFDGSIRTGGGGSGGGGSGGGGSGGG |
| | | GSTHTCPPCPAPELLGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 56 | SEQ ID NO: 56 | KKEDGESTAPTPRPKVLRCKCHHHCPEDSVNNICSTDGYCFTMI |
| | | EEDDSGLPVVTSGCLKLEGSDFQCRDTPKAQLRRSIECCTERNE |
| | | CNKDLHPTLPPVVIGPFFDGSIRTGGGGSGGGGSGGGGSGGG |
| | | GSTHTCPPCPAPELLGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 57 | SEQ ID NO: 11 Variant | KKEDGESTAPTPRPKVLRCKCSHHCPEDSVNNICSTDGYCFTMI |
| | H22S | EEDDSGLPVVTSGCLGLEGSDFQCRDTPIPHQRRSIECCTERNEC |
| | | NKDLHPTLPPVVIGPFFDGSIR |
| 58 | SEQ ID NO: 58 | KKEDGESTAPTPRPKVLRCKCSHHCPEDSVNNICSTDGYCFTMI |
| | | EEDDSGLPVVTSGCLGLEGSDFQCRDTPIPHQRRSIECCTERNEC |
| | | NKDLHPTLPPVVIGPFFDGSIRTGGGGSGGGGSGGGGSGGGG |
| | | STHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 59 | SEQ ID NO: 11 Variant | KKEDGESTAPTPRPKVLRCKCSGHCPEDSVNNICSTDGYCFTMI |
| | H22S and H23G | EEDDSGLPVVTSGCLGLEGSDFQCRDTPIPHQRRSIECCTERNEC |
| | | NKDLHPTLPPVVIGPFFDGSIR |
| 60 | SEQ ID NO: 60 | KKEDGESTAPTPRPKVLRCKCSGHCPEDSVNNICSTDGYCFTMI |
| | | EEDDSGLPVVTSGCLGLEGSDFQCRDTPIPHQRRSIECCTERNEC |
| | | NKDLHPTLPPVVIGPFFDGSIRTGGGGSGGGGSGGGGSGGGG |
| | | STHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 61 | SEQ ID NO: 11 Variant | KKEDGESTAPTPRPKVLRCYCSGHCPEDSVNNICSTDGYCFTMI |
| | K20Y, H22S and H23G | EEDDSGLPVVTSGCLGLEGSDFQCRDTPIPHQRRSIECCTERNEC |
| | | NKDLHPTLPPVVIGPFFDGSIR |
| 62 | SEQ ID NO: 62 | KKEDGESTAPTPRPKVLRCYCSGHCPEDSVNNICSTDGYCFTMI |
| | | EEDDSGLPVVTSGCLGLEGSDFQCRDTPIPHQRRSIECCTERNEC |
| | | NKDLHPTLPPVVIGPFFDGSIRTGGGGSGGGGSGGGGSGGGG |
| | | STHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 63 | SEQ ID NO: 11 Variant | KKEDGESTAPTPRPKVLRCYCHGHCPEDSVNNICSTDGYCFTMI |
| | K20Y and H23G | EEDDSGLPVVTSGCLGLEGSDFQCRDTPIPHQRRSIECCTERNEC |
| | | NKDLHPTLPPVVIGPFFDGSIR |
| 64 | SEQ ID NO: 64 | KKEDGESTAPTPRPKVLRCYCHGHCPEDSVNNICSTDGYCFTMI |
| | | EEDDSGLPVVTSGCLGLEGSDFQCRDTPIPHQRRSIECCTERNEC |
| | | NKDLHPTLPPVVIGPFFDGSIRTGGGGSGGGGSGGGGSGGGG |
| | | STHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 65 | SEQ ID NO: 11 Variant | KKEDGESTAPTPRPKVLRCYCHHHCPEDSVNNICSTDGYCFTMI |
| | K20Y | EEDDSGLPVVTSGCLGLEGSDFQCRDTPIPHQRRSIECCTERNEC |
| | | NKDLHPTLPPVVIGPFFDGSIR |
| 66 | SEQ ID NO: 66 | KKEDGESTAPTPRPKVLRCYCHHHCPEDSVNNICSTDGYCFTMI |
| | | EEDDSGLPVVTSGCLGLEGSDFQCRDTPIPHQRRSIECCTERNEC |
| | | NKDLHPTLPPVVIGPFFDGSIRTGGGGSGGGGSGGGGSGGGG |
| | | STHTCPPCPAPELLGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPGK |
| 67 | SEQ ID NO: 1 K20Y, | KKEDGESTAPTPRPKVLRCYCSGHCPEDSVNNICSTDGYCFTMI |
| | H22S, H23G, D107A, | EEDDSGLPVVTSGCLGLEGSDFQCRDTPIPHQRRSIECCTERNEC |
| | H111_, and H112_ | NKDLHPTLPPLKNRDFVAGPI_R |
| | Variant | |
| 68 | SEQ ID NO: 68 | KKEDGESTAPTPRPKVLRCYCSGHCPEDSVNNICSTDGYCFTMI |
| | | EEDDSGLPVVTSGCLGLEGSDFQCRDTPIPHQRRSIECCTERNEC |
| | | NKDLHPTLPPLKNRDFVAGPI_RTGGGGSGGGGSGGGGSGG |
| | | GGSTHTCPPCPAPELLGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 69 | SEQ ID NO: 1 Variant | KKEDGESTAPTPRPKVLRCKCHHHCPEDSVNNICSTDGYCFTMI |
| | C-terminus seven | EEDDSGLPVVTSGCLGLEGSDFQCRDTPIPHQRRSIECCTERNEC |
| | amino acid deletion | NKDLHPTLPPLKNRDFV___ |
| | Wild type ALK-6 with | |
| | C terminus truncated | |
| | (7 aa deleted) | |
| 70 | SEQ ID NO: 70 | KKEDGESTAPTPRPKVLRCKCHHHCPEDSVNNICSTDGYCFTMI |
| | | EEDDSGLPVVTSGCLGLEGSDFQCRDTPIPHQRRSIECCTERNEC |
| | | NKDLHPTLPPLKNRDFV____TGGGGSGGGGSGGGGSGGG |
| | | GSTHTCPPCPAPELLGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPGK |
| 71 | SEQ ID NO: 1 D107A | KKEDGESTAPTPRPKVLRCKCHHHCPEDSVNNICSTDGYCFTMI |
| | Variant | EEDDSGLPVVTSGCLGLEGSDFQCRDTPIPHQRRSIECCTERNEC |
| | | NKDLHPTLPPLKNRDFVAGPIHHR |
| 72 | SEQ ID NO: 72 | KKEDGESTAPTPRPKVLRCKCHHHCPEDSVNNICSTDGYCFTMI |
| | | EEDDSGLPVVTSGCLGLEGSDFQCRDTPIPHQRRSIECCTERNEC |
| | | NKDLHPTLPPLKNRDFVAGPIHHRTGGGGGGGGSGGGGSGG |
| | | GGSTHTCPPCPAPELLGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 73 | SEQ ID NO: 1 D107A, | KKEDGESTAPTPRPKVLRCKCHHHCPEDSVNNICSTDGYCFTMI |
| | H111_and H112_ | EEDDSGLPVVTSGCLGLEGSDFQCRDTPIPHQRRSIECCTERNEC |
| | Variant | NKDLHPTLPPLKNRDFVAGPI_R |
| 74 | SEQ ID NO: 74 | KKEDGESTAPTPRPKVLRCKCHHHCPEDSVNNICSTDGYCFTMI |
| | | EEDDSGLPVVTSGCLGLEGSDFQCRDTPIPHQRRSIECCTERNEC |
| | | NKDLHPTLPPLKNRDFVAGPI_RTGGGGSGGGGSGGGGSGG |
| | | GGSTHTCPPCPAPELLGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 75 | SEQ ID NO: 1 P109S, | KKEDGESTAPTPRPKVLRCKCHHHCPEDSVNNICSTDGYCFTMI |
| | H111_ and H112_ | EEDDSGLPVVTSGCLGLEGSDFQCRDTPIPHQRRSIECCTERNEC |
| | Variant | NKDLHPTLPPLKNRDFVDGSI_R |
| 76 | SEQ ID NO: 76 | KKEDGESTAPTPRPKVLRCKCHHHCPEDSVNNICSTDGYCFTMI |
| | | EEDDSGLPVVTSGCLGLEGSDFQCRDTPIPHQRRSIECCTERNEC |
| | | NKDLHPTLPPLKNRDFVDGSI_RTGGGGSGGGGSGGGGSGG |
| | | GGSTHTCPPCPAPELLGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 77 | SEQ ID NO: 1 P109S, | KKEDGESTAPTPRPKVLRCKCHHHCPEDSVNNICSTDGYCFTMI |
| | H111_ and H112_ | EEDDSGLPVVTSGCLGLEGSDFQCRDTPIPHQRRSIECCTERNEC |
| | Variant | NKDLHPTLPPLKNRDFFDGPI_R |
| 78 | SEQ ID NO: 78 | KKEDGESTAPTPRPKVLRCKCHHHCPEDSVNNICSTDGYCFTMI |
| | | EEDDSGLPVVTSGCLGLEGSDFQCRDTPIPHQRRSIECCTERNEC |
| | | NKDLHPTLPPLKNRDFFDGPI_RTGGGGSGGGGSGGGGSGG |
| | | GGSTHTCPPCPAPELLGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPGK |
| 79 | Truncated ALK6 | Lys₁-Lys₂-Glu₃-Asp₄-Gly₅-Glu₆-Ser₇-Thr₈-Ala₉ |
| | extracellular domain | Pro₁₀-Thr₁₁-Pro₁₂-Arg₁₃-Pro₁₄-Xaa₁₅-Val₁₆-Leu₁₇- |
| | (ECD) combined with | Arg₁₈-Cys₁₉-Xaa₂₀-Cys₂₁-Xaa₂₂-Xaa₂₃-His₂₄-Cys₂₅- |
| | an ALK3 C terminus | Pro₂₆-Xaa₂₇-Asp₂₈-Xaa₂₉-Xaa₃₀-Asn₃₁-Asn₃₂-Xaa₃₃- |
| | sequence (from SEQ ID | Cys₃₄-Xaa₃₅-Thr₃₆-Xaa₃₇-Gly₃₈-Xaa₃₉-Cys₄₀-Phe₄₁- |
| | NO: 11) | Xaa₄₂-Xaa₄₃-Ile₄₄-Glu₄₅-Glu₄₆-Asp₄₇-Asp₄₈-Xaa₄₉- |
| | | Gly₅₀-Xaa₅₁-Xaa₅₂-Xaa₅₃-Xaa₅₄-Xaa₅₅-Ser₅₆-Gly₅₇- |
| | | Cys₅₈-Xaa₅₉-Xaa₆₀-Xaa₆₁-Glu₆₂-Gly₆₃-Ser₆₄-Asp₆₅- |
| | | Phe₆₆-Gln₆₇-Cys₆₈-Xaa₆₉-Asp₇₀-Xaa₇₁-Pro₇₂-Xaa₇₃- |
| | | Xaa₇₄-Xaa₇₅-Xaa₇₆-Arg₇₇-Arg₇₈-Xaa₇₉-Ile₈₀-Glu₈₁- |
| | | Cys₈₂-Cys₈₃-Xaa₈₄-Xaa₈₅-Xaa₈₆-Xaa₈₇-Xaa₈₈-Cys₈₉- |
| | | Asn₉₀-Xaa₉₁-Xaa₉₂-Leu₉₃-Xaa₉₄-Pro₉₅-Thr₉₆-Leu₉₇- |
| | | Pro₉₈-Pro₉₉-Val₁₀₀-Val₁₀₁-Ile₁₀₂-Gly₁₀₃-Pro₁₀₄-Phe₁₀₅- |
| | | Phe₁₀₆-Asp₁₀₇-Gly₁₀₈-Ser₁₀₉-Ile₁₁₀-Arg₁₁₁; |
| | | wherein Xaa₁₅ is K or P; Xaa₂₀ is V, F, K or Y; Xaa₂₂ is G, H or S; |
| | | Xaa₂₃ is S, H or G; Xaa₂₇ is E or D; Xaa₂₉ is S or A; Xaa₃₀ is V or I; |
| | | Xaa₃₃ is I or T; Xaa₃₅ is S or I; Xaa₃₇ is D or N; Xaa₃₉ is Y or H; Xaa₄₂ |
| | | is T or A; Xaa₄₃ is M or I; Xaa₄₉ is S or Q; Xaas₁ is L or E; Xaa₅₂ is |
| | | P or T; Xaa₅₃ is V or T; Xaa₅₄ is V or L; Xaa₅₅ is T or A; Xaa₅₉ is L or |
| | | M; Xaa₆₀ is G or K; Xaa₆₁ is L or Y Xaa₆₉ is R or K; Xaa₇₁ is T or S; |
| | | Xaa₇₃ is I or K; Xaa₇₄ is P or A; Xaa₇₅ is H or Q; Xaa₇₆ is Q or L; Xaa₇₉ |
| | | is S or T; Xaa₈₄ is T or R; Xaa₈₅ is absent, T or E; Xaa₈₆ is R or N; |
| | | Xaa₈₇ is N or L; Xaa₈₈ is absent, E or L; Xaa₉₁ is K or Q; Xaa₉₂ is D or |
| | | L; and, Xaa₉₄ is H or Q |
| 80 | SEQ ID NO: 1 H111_ | KKEDGESTAPTPRPKVLRCKCHHHCPEDSVNNICSTDGYCFTMI |
| | and H112_Variant | EEDDSGLPVVTSGCLGLEGSDFQCRDTPIPHQRRSIECCTERNEC |
| | | NKDLHPTLPPLKNRDFVDGPI_R |
| 81 | Truncated (7 amino | Lys₁-Lys₂-Glu₃-Asp₄-Gly₅-Glu₆-Ser₇-Thr₈-Ala₉- |
| | acids) ALK6 | Pro₁₀-Thr₁₁-Pro₁₂-Arg₁₃-Pro₁₄-Xaa₁₅-Val₁₆-Leu₁₇- |
| | extracellular domain | Arg₁₈-Cys₁₉-Xaa₂₀-Cys₂₁-Xaa₂₂-Xaa₂₃-His₂₄-Cys₂₅- |
| | (ECD) (From SEQ ID | Pro₂₆-Xaa₂₇-Asp₂₈-Xaa₂₉-Xaa₃₀-Asn₃₁-Asn₃₂-Xaa₃₃- |
| | NO: 1 and 69) | Cys₃₄-Xaa₃₅-Thr₃₆-Xaa₃₇-Gly₃₈-Xaa₃₉-Cys₄₀-Phe₄₁- |
| | | Xaa₄₂-Xaa₄₃-Ile₄₄-Glu₄₅-Glu₄₆-Asp₄₇-Asp₄₈-Xaa₄₉- |
| | | Gly₅₀-Xaa₅₁-Xaa₅₂-Xaa₅₃-Xaa₅₄-Xaa₅₅-Ser₅₆-Gly₅₇- |
| | | Cys₅₈-Xaa₅₉-Xaa₆₀-Xaa₆₁-Glu₆₂-Gly₆₃-Ser₆₄-Asp₆₅- |
| | | Phe₆₆-Gln₆₇-Cys₆₈-Xaa₆₉-Asp₇₀-Xaa₇₁-Pro₇₂-Xaa₇₃- |
| | | Xaa₇₄-Xaa₇₅-Xaa₇₆-Arg₇₇-Arg₇₈-Xaa₇₉-Ile₈₀-Glu₈₁- |
| | | Cys₈₂-Cys₈₃-Xaa₈₄-Xaa₈₅-Xaa₈₆-Xaa₈₇-Xaa₈₈-Cys₈₉- |
| | | Asn₉₀-Xaa₉₁-Xaa₉₂-Leu₉₃-Xaa₉₄-Pro₉₅-Thr₉₆-Leu₉₇- |
| | | Pro₉₈-Pro₉₉-Leu₁₀₀-Lys₁₀₁-Asn₁₀₂-Arg₁₀₃-Asp₁₀₄- |
| | | Phe₁₀₅-Val₁₀₆-Xaa₁₀₇-Xaa₁₀₈-Xaa₁₀₉-Xaa₁₁₀-Xaa₁₁₁- |
| | | Xaa₁₁₂-Xaa₁₁₃; |
| | | wherein Xaa₁₅ is K or P; Xaa₂₀ is V, F, K or Y; Xaa₂₂ is G, |
| | | H or S; Xaa₂₃ is S, H or G; Xaa₂₇ is E or D; Xaa₂₉ is S or A; |
| | | Xaa₃₀ is V or I; Xaa₃₃ is I or T; Xaa₃₅ is S or I; Xaa₃₇ is D or |
| | | N; Xaa₃₉ is Y or H; Xaa₄₂ is T or A; Xaa₄₃ is M or I; Xaa₄₉ is |
| | | S or Q; Xaa₅₁ is L or E; Xaa₅₂ is P or T; Xaa₅₃ is V or T; |
| | | Xaa₅₄ is V or L; Xaa₅₅ is T or A; Xaa₅₉ is L or M; Xaa₆₀ is G |
| | | or K; Xaa₆₁ is L or Y; Xaa₆₉ is R or K; Xaa₇₁ is Tor S; Xaa₇₃ |
| | | is I or K; Xaa₇₄ is P or A; Xaa₇₅ is H or Q; Xaa₇₆ is Q or L; |
| | | Xaa₇₉ is S or T; Xaa₈₄ is T or R; Xaa₈₅ is absent, T or E; Xaa₈₆ |
| | | is R or N; Xaa₈₇ is N or L; Xaa₈₈ is absent, E or L; Xaa₉₁ is |
| | | K or Q; Xaa₉₂ is D or L; Xaa₉₄ is H or Q; Xaa₁₀₇ is absent; |
| | | Xaa₁₀₈ is absent; Xaa₁₀₉ is absent; Xaa₁₁₀ is absent; Xaa₁₁₁ is |
| | | absent; Xaa₁₁₂ is absent; and, Xaa₁₁₃ is absent |
| 82 | Truncated (2 amino | Lys₁-Lys₂-Glu₃-Asp₄-Gly₅-Glu₆-Ser₇-Thr₈-Ala₉- |
| | acids) ALK6 | Pro₁₀-Thr₁₁-Pro₁₂-Arg₁₃-Pro₁₄-Xaa₁₅-Val₁₆-Leu₁₇- |
| | extracellular domain | Arg₁₈-Cys₁₉-Xaa₂₀-Cys₂₁-Xaa₂₂-Xaa₂₃-His₂₄-Cys₂₅- |
| | (ECD)(From SEQ ID | Pro₂₆-Xaa₂₇-Asp₂₈-Xaa₂₉-Xaa₃₀-Asn₃₁-Asn₃₂-Xaa₃₃- |
| | NO: 1 and 80) | Cys₃₄-Xaa₃₅-Thr₃₆-Xaa₃₇-Gly₃₈-Xaa₃₉-Cys₄₀-Phe₄₁- |
| | | Xaa₄₂-Xaa₄₃-Ile₄₄-Glu₄₅-Glu₄₆-Asp₄₇-Asp₄₈-Xaa₄₉- |
| | | Gly₅₀-Xaa₅₁-Xaa₅₂-Xaa₅₃-Xaa₅₄-Xaa₅₅-Ser₅₆-Gly₅₇- |
| | | Cys₅₈-Xaas₉-Xaa₆₀-Xaa₆₁-Glu₆₂-Gly₆₃-Ser₆₄-Asp₆₅- |
| | | Phe₆₆-Gln₆₇-Cys₆₈-Xaa₆₉-Asp₇₀-Xaa₇₁-Pro₇₂-Xaa₇₃- |
| | | Xaa₇₄-Xaa₇₅-Xaa₇₆-Arg₇₇-Arg₇₈-Xaa₇₉-Ile₈₀-Glu₈₁- |
| | | Cys₈₂-Cys₈₃-Xaa₈₄-Xaa₈₅-Xaa₈₆-Xaa₈₇-Xaa₈₈-Cys₈₉- |
| | | Asn₉₀-Xaa₉₁-Xaa₉₂-Leu₉₃-Xaa₉₄-Pro₉₅-Thr₉₆-Leu₉₇- |
| | | Pro₉₈-Pro₉₉-Leu₁₀₀-Lys₁₀₁-Asn₁₀₂-Arg₁₀₃-Asp₁₀₄- |
| | | Phe₁₀₅-Val₁₀₆-Xaa₁₀₇-Gly₁₀₈-Xaa₁₀₉-Ile₁₁₀-Xaa₁₁₁- |
| | | Xaa₁₁₂-Arg₁₁₃; |
| | | wherein Xaa₁₅ is K or P; Xaa₂₀ is V, F, K or Y; Xaa₂₂ is G, H |
| | | or S; Xaa₂₃ is S, H or G; Xaa₂₇ is E or D; Xaa₂₉ is S or A; |
| | | Xaa₃₀ is V or I; Xaa₃₃ is I or T; Xaa₃₅ is S or I; Xaa₃₇ is D or |
| | | N; Xaa₃₉ is Y or H; Xaa₄₂ is T or A; Xaa₄₃ is M or I; Xaa₄₉ is |
| | | S or Q; Xaa₅₁ is L or E; Xaa₅₂ is P or T; Xaa₅₃ is V or T; Xaa₅₄ |
| | | is V or L; Xaa₅₅ is T or A; Xaa₅₉ is L or M; Xaa₆₀ is G or K; |
| | | Xaa₆₁ is L or Y; Xaa₆₉ is R or K; Xaa₇₁ is T or S; Xaa₇₃ is I or |
| | | K; Xaa₇₄ is P or A; Xaa₇₅ is H or Q; Xaa₇₆ is Q or L; Xaa₇₉ is |
| | | S or T; Xaa₈₄ is T or R; Xaa₈₅ is absent, T or E; Xaa₈₆ is R or |
| | | N; Xaa₈₇ is N or L; Xaa₈₈ is absent, E or L; Xaa₉₁ is K or Q; |
| | | Xaa₉₂ is D or L; Xaa₉₄ is H or Q; Xaa₁₀₇ is D or A; Xaa₁₀₉ is |
| | | P or S; Xaa₁₁₁ is absent; and, Xaa₁₂ is absent; |
| 83 | ALK6 extracellular | Lys₁-Lys₂-Glu₃-Asp₄-Gly₅-Glu₆-Ser₇-Thr₈-Ala₉-Pro₁₀- |
| | domain (ECD)with | Thr₁₁-Pro₁₂-Arg₁₃-Pro₁₄-Xaa₁₅-Val₁₆-Leu₁₇-Arg₁₈-Cys₁₉- |
| | D104A and D107 | Xaa₂₀-Cys₂₁-Xaa₂₂-Xaa₂₃-His₂₄-Cys₂₅-Pro₂₆-Xaa₂₇-Asp₂₈- |
| | (From SEQ ID NO: 1) | Xaa₂₉-Xaa₃₀-Asn₃₁-Asn₃₂-Xaa₃₃-Cys₃₄-Xaa₃₅-Thr₃₆-Xaa₃₇- |
| | | Gly₃₈-Xaa₃₉-Cys₄₀-Phe₄₁-Xaa₄₂-Xaa₄₃-Ile₄₄-Glu₄₅-Glu₄₆- |
| | | Asp₄₇-Asp₄₈-Xaa₄₉-Gly₅₀-Xaa₅₁-Xaa₅₂-Xaa₅₃-Xaa₅₄-Xaa₅₅- |
| | | Ser₅₆-Gly₅₇-Cys₅₈-Xaa₅₉-Xaa₆₀-Xaa₆₁-Glu₆₂-Gly₆₃-Ser₆₄- |
| | | Asp₆₅-Phe₆₆-Gln₆₇-Cys₆₈-Xaa₆₉-Asp₇₀-Xaa₇₁-Pro₇₂-Xaa₇₃- |
| | | Xaa₇₄-Xaa₇₅-Xaa₇₆-Arg₇₇-Arg₇₈-Xaa₇₉-Ile₈₀-Glu₈₁-Cys₈₂- |
| | | Cys₈₃-Xaa₈₄-Xaa₈₅-Xaa₈₆-Xaa₈₇-Xaa₈₈-Cys₈₉-Asn₉₀-Xaa₉₁- |
| | | Xaa₉₂-Leu₉₃-Xaa₉₄-Pro₉₅-Thr₉₆-Leu₉₇-Pro₉₈-Pro₉₉-Leu₁₀₀- |
| | | Lys₁₀₁-Asn₁₀₂-Arg₁₀₃-Xaa₁₀₄-Phe₁₀₅-Val₁₀₆-Xaa₁₀₇-Gly₁₀₈- |
| | | Xaa₁₀₉-Ile₁₁₀-His₁₁₁-His₁₁₂-Arg₁₁₃ |
| | | wherein Xaa₁₅ is K or P; Xaa₂₀ is V, F, K or Y; Xaa₂₂ is G, H |
| | | or S; Xaa₂₃ is S, H or G; Xaa₂₇ is E or D; Xaa₂₉ is S or A; |
| | | Xaa₃₀ is V or I; Xaa₃₃ is I or T; Xaa₃₅ is S or I; Xaa₃₇ is D or |
| | | N; Xaa₃₉ is Y or H; Xaa₄₂ is T or A; Xaa₄₃ is M or I; Xaa₄₉ is |
| | | S or Q; Xaa₅₁ is L or E; Xaa₅₂ is P or T; Xaa₅₃ is V or T; Xaa₅₄ |
| | | is V or L; Xaas₅ is T or A; Xaa₅₉ is L or M; Xaa₆₀ is G or K; |
| | | Xaa₆₁ is L or Y; Xaa₆₉ is R or K; Xaa₇₁ is T or S; Xaa₇₃ is I or |
| | | K; Xaa₇₄ is P or A; Xaa₇₅ is H or Q; Xaa₇₆ is Q or L; Xaa₇₉ is |
| | | S or T; Xaa₈₄ is T or R; Xaa₈₅ is absent, T or E; Xaa₈₆ is R or |
| | | N; Xaa₈₇ is N or L; Xaa₈₈ is absent, E or L; Xaa₉₁ is K or Q; |
| | | Xaa₉₂ is D or L; Xaa₉₄ is H or Q; Xaa₁₀₄ is D or A; Xaa₁₀₇ is |
| | | absent or D; and Xaa₁₀₉ is P or S. |
| 84 | ECD and Fc same as | KKEDGESTAPTPRPKVLRCKCHHHCPEDSVNNICSTDGYCFTMI |
| | SEQ ID NO: 46; with | EEDDSGLPVVTSGCLGLEGSDFQCRDTPIPHQRRSIECCTERNEC |
| | Alpha helix linker | NKDLHPTLPPVVIGPFFDGSIREAAAKEAAAKEAAAKEAAAK |
| | | THTCPPCPAPELLGGPSVFLFPPKPKDTLMISRTPEVTCVVVDVS |
| | | HEDPEVKFNWYVDGVEVHNAKTKPREEQYNSTYRVVSVLTVL |
| | | HQDWLNGKEYKCKVSNKALPAPIEKTISKAKGQPREPQVYTLP |
| | | PSREEMTKNQVSLTCLVKGFYPSDIAVEWESNGQPENNYKTTPP |
| | | VLDSDGSFFLYSKLTVDKSRWQQGNVFSCSVMHEALHNHYTQ |
| | | KSLSLSPGK |
| 85 | ECD and Fc same as | KKEDGESTAPTPRPKVLRCKCHHHCPEDSVNNICSTDGYCFTMI |
| | SEQ ID NO: 46; with | EEDDSGLPVVTSGCLGLEGSDFQCRDTPIPHQRRSIECCTERNEC |
| | short linker | NKDLHPTLPPVVIGPFFDGSIRTGGGTHTCPPCPAPELLGGPSV |
| | | FLFPPKPKDTLMISRTPEVTCVVVDVSHEDPEVKFNWYVDGVE |
| | | VHNAKTKPREEQYNSTYRVVSVLTVLHQDWLNGKEYKCKVSN |
| | | KALPAPIEKTISKAKGQPREPQVYTLPPSREEMTKNQVSLTCLV |
| | | KGFYPSDIAVEWESNGQPENNYKTTPPVLDSDGSFFLYSKLTVD |
| | | KSRWQQGNVFSCSVMHEALHNHYTQKSLSLSPGK |
| 86 | SEQ ID NO: 69 and | KKEDGESTAPTPRPKVLRCYCSGHCPEDSVNNICSTDGYCFTMI |
| | 81 variant ECD; | EEDDSGLPVVTSGCLGLEGSDFQCRDTPIPHQRRSIECCTERNEC |
| | K20Y; H22S; H23G; | NKDLHPTLPPLKNRDFV____ |
| | and C-terminus seven | |
| | amino acid deletion | |
| 87 | SEQ ID NO: 87 | KKEDGESTAPTPRPKVLRCYCSGHCPEDSVNNICSTDGYCFTMI |
| | | EEDDSGLPVVTSGCLGLEGSDFQCRDTPIPHQRRSIECCTERNEC |
| | | NKDLHPTLPPLKNRDFV____TGGGGSGGGGSGGGGSGGG |
| | | GSTHTCPPCPAPEAAGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 88 | SEQ ID NO: 11 | KKEDGESTAPTPRPKVLRCYCSGHCPEDSVNNICSTDGYCFTMI |
| | variant ECD; K20Y; | EEDDSGLPVVTSGCLKLEGSDFQCRDTPIPHQRRSIECCTERNEC |
| | H22S; H23G; G60K | NKDLHPTLPPVVIGPFFDGSIR |
| | and with ALK3 C | |
| | terminus sequence | |
| 89 | SEQ ID NO: 89 | KKEDGESTAPTPRPKVLRCYCSGHCPEDSVNNICSTDGYCFTMI |
| | | EEDDSGLPVVTSGCLKLEGSDFQCRDTPIPHQRRSIECCTERNEC |
| | | NKDLHPTLPPVVIGPFFDGSIRTGGGGSGGGGSGGGGSGGGG |
| | | STHTCPPCPAPEAAGGPSVFLFPPKPKDTLMISRTPEVTCVVVDV |
| | | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPG |
| 90 | SEQ ID NO: 69 and | KKEDGESTAPTPRPKVLRCYCSAGHCPEDSVNNICSTDGYCFTM |
| | 81 Variant ECD; | IEEDDSGLPVVTSGCLGLEGSDFQCRDTPIPHQRRSIECCTERNE |
| | K20Y; H22S; _22′A | CNKDLHPTLPPLKNRDFV____ |
| | (inserted between | |
| | aa22 and aa23) H23G; | |
| | and C-terminus seven | |
| | amino acid deletion | |
| 91 | SEQ ID NO: 91 | KKEDGESTAPTPRPKVLRCYCSAGHCPEDSVNNICSTDGYCFTM |
| | | IEEDDSGLPVVTSGCLGLEGSDFQCRDTPIPHQRRSIECCTERNE |
| | | CNKDLHPTLPPLKNRDFV____TGGGGSGGGGSGGGGSGG |
| | | GGSTHTCPPCPAPEAAGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 92 | SEQ ID NO: 69 and | KKEDGESTAPTPRPKVLRCYCSGHCPEDSVNNICSTNGYCFTMI |
| | 81 Variant; ECD | EEDDSGLPVVTSGCLGLEGSDFQCRDTPIPHQRRSIECCTERNEC |
| | K20Y; H22S; H23G; | NKDLHPTLPPLKNRDFV____ |
| | D37N; and C- | |
| | terminus seven amino | |
| | acid deletion | |
| 93 | SEQ ID NO: 93 | KKEDGESTAPTPRPKVLRCYCSGHCPEDSVNNICSTNGYCFTMI |
| | | EEDDSGLPVVTSGCLGLEGSDFQCRDTPIPHQRRSIECCTERNEC |
| | | NKDLHPTLPPLKNRDFV____TGGGGSGGGGSGGGGSGGG |
| | | GSTHTCPPCPAPEAAGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 94 | SEQ ID NO: 69 and | KKEDGESTAPTPRPKVLRCVCSGHCPEDSVNNICSTDGYCFTMI |
| | 81 variant ECD; | EEDDSGLPVVTSGCLGLEGSDFQCRDTPIPHQRRSIECCTERNEC |
| | K20V; H22S; H23G; | NKDLHPTLPPLKNRDFV____ |
| | and C-terminus seven | |
| | amino acid deletion | |
| 95 | SEQ ID NO: 95 | KKEDGESTAPTPRPKVLRCVCSGHCPEDSVNNICSTDGYCFTMI |
| | | EEDDSGLPVVTSGCLGLEGSDFQCRDTPIPHQRRSIECCTERNEC |
| | | NKDLHPTLPPLKNRDFV____TGGGGSGGGGSGGGGSGGG |
| | | GSTHTCPPCPAPEAAGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 96 | SEQ ID NO: 69 and | KKEDGESTAPTPRPKVLRCFCSGHCPEDSVNNICSTDGYCFTMI |
| | 81 variant ECD; | EEDDSGLPVVTSGCLGLEGSDFQCRDTPIPHQRRSIECCTERNEC |
| | K20F; H22S; H23G; | NKDLHPTLPPLKNRDFV____ |
| | and C-terminus seven | |
| | amino acid deletion | |
| 97 | SEQ ID NO: 97 | KKEDGESTAPTPRPKVLRCFCSGHCPEDSVNNICSTDGYCFTMI |
| | | EEDDSGLPVVTSGCLGLEGSDFQCRDTPIPHQRRSIECCTERNEC |
| | | NKDLHPTLPPLKNRDFV____TGGGGSGGGGSGGGGSGGG |
| | | GSTHTCPPCPAPEAAGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 98 | SEQ ID NO: 69 and | KKEDGESTAPTPRPKVLRCYCGSHCPEDSVNNICSTDGYCFTMI |
| | 81 variant ECD; | EEDDSGLPVVTSGCLGLEGSDFQCRDTPIPHQRRSIECCTERNEC |
| | K20Y; H22G; H23S; | NKDLHPTLPPLKNRDFV____ |
| | and C-terminus seven | |
| | amino acid deletion | |
| 99 | SEQ ID NO: 99 | KKEDGESTAPTPRPKVLRCYCGSHCPEDSVNNICSTDGYCFTMI |
| | | EEDDSGLPVVTSGCLGLEGSDFQCRDTPIPHQRRSIECCTERNEC |
| | | NKDLHPTLPPLKNRDFV____TGGGGSGGGGSGGGGSGGG |
| | | GSTHTCPPCPAPEAAGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 100 | SEQ ID NO: 69 and | KKEDGESTAPTPRPKVLRCFCGSHCPEDSVNNICSTDGYCFTMI |
| | 81 variant ECD; | EEDDSGLPVVTSGCLGLEGSDFQCRDTPIPHQRRSIECCTERNEC |
| | K20F; H22G; H23S; | NKDLHPTLPPLKNRDFV____ |
| | and C-terminus seven | |
| | amino acid deletion | |
| 101 | SEQ ID NO: 101 | KKEDGESTAPTPRPKVLRCFCGSHCPEDSVNNICSTDGYCFTMI |
| | | EEDDSGLPVVTSGCLGLEGSDFQCRDTPIPHQRRSIECCTERNEC |
| | | NKDLHPTLPPLKNRDFV____TGGGGSGGGGSGGGGSGGG |
| | | GSTHTCPPCPAPEAAGGPSVFLFPPKPKDTLMISRTPEVTCVVVD |
| | | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 102 | SEQ ID NO: 69 and | KKEDGESTAPTPRPKVLRCYCGASHCPEDSVNNICSTDGYCFTM |
| | 81 Variant ECD; | IEEDDSGLPVVTSGCLGLEGSDFQCRDTPIPHQRRSIECCTERNE |
| | K20Y; H22G; _22′A | CNKDLHPTLPPLKNRDFV____ |
| | (inserted between | |
| | aa22 and aa23) H23S; | |
| | and C-terminus seven | |
| | amino acid deletion | |
| 103 | SEQ ID NO: 103 | KKEDGESTAPTPRPKVLRCYCGASHCPEDSVNNICSTDGYCFTM |
| | | IEEDDSGLPVVTSGCLGLEGSDFQCRDTPIPHQRRSIECCTERNE |
| | | CNKDLHPTLPPLKNRDFV____TGGGGSGGGGSGGGGSGG |
| | | GGSTHTCPPCPAPEAAGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 104 | SEQ ID NO: 69 and | KKEDGESTAPTPRPKVLRCYCSAGHCPEDSVNNICSTDGYCFTM |
| | 81 Variant ECD; | IEEDDSGLPVVTSGCLKLEGSDFQCRDTPIPHQRRSIECCTERNE |
| | K20Y; H22S; _22′A | CNKDLHPTLPPLKNRDFV____ |
| | (inserted between | |
| | aa22 and aa23) H23G; | |
| | G60K; and C- | |
| | terminus seven amino | |
| | acid deletion | |
| 105 | SEQ ID NO: 105 | KKEDGESTAPTPRPKVLRCYCSAGHCPEDSVNNICSTDGYCFTM |
| | | IEEDDSGLPVVTSGCLKLEGSDFQCRDTPIPHQRRSIECCTERNE |
| | | CNKDLHPTLPPLKNRDFV____TGGGGSGGGGSGGGGSGG |
| | | GGSTHTCPPCPAPEAAGGPSVFLFPPKPKDTLMISRTPEVTCVVV |
| | | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 106 | Human Fc Sequence | THTCPPCPAPEAAGGPSVFLFPPKPKDTLMISRTPEVTCVVVDVS |
| | with LALA mutation | HEDPEVKFNWYVDGVEVHNAKTKPREEQYNSTYRVVSVLTVL |
| | and(w/o terminal | HQDWLNGKEYKCKVSNKALPAPIEKTISKAKGQPREPQVYTLP |
| | lysine) | PSREEMTKNQVSLTCLVKGFYPSDIAVEWESNGQPENNYKTTPP |
| | | VLDSDGSFFLYSKLTVDKSRWQQGNVFSCSVMHEALHNHYTQ |
| | | KSLSLSPG |
| 107 | Human Fc Sequence | THTCPPCPAPEAAGGPSVFLFPPKPKDTLMISRTPEVTCVVVDVS |
| | with LALA mutation | HEDPEVKFNWYVDGVEVHNAKTKPREEQYNSTYRVVSVLTVL |
| | and(w/ terminal | HQDWLNGKEYKCKVSNKALPAPIEKTISKAKGQPREPQVYTLP |
| | lysine) | PSREEMTKNQVSLTCLVKGFYPSDIAVEWESNGQPENNYKTTPP |
| | | VLDSDGSFFLYSKLTVDKSRWQQGNVFSCSVMHEALHNHYTQ |
| | | KSLSLSPGK |
| 108 | SEQ ID NO: 81 with | Lys₁-Lys₂-Glu₃-Asp₄-Gly₅-Glu₆-Ser₇-Thr₈-Ala₉- |
| | addition of aaA | Pro₁₀-Thr₁₁-Pro₁₂-Arg₁₃-Pro₁₄-Xaa₁₅-Xaa₁₆-Leu₁₇- |
| | between 22 and 23 | Arg₁₈-Cys₁₉-Xaa₂₀-Cys₂₁-Xaa₂₂-Xaa′₂₂-Xaa₂₃-His₂₄- |
| | | Cys₂₅-Pro₂₆-Xaa₂₇-Asp₂₈-Xaa₂₉-Xaa₃₀-Asn₃₁-Asn₃₂- |
| | | Xaa₃₃-Cys₃₄-Xaa₃₅-Thr₃₆-Xaa₃₇-Gly₃₈-Xaa₃₉-Cys₄₀- |
| | | Phe₄₁-Xaa₄₂-Xaa₄₃-Ile₄₄-Glu₄₅-Glu₄₆-Asp₄₇-Asp₄₈- |
| | | Xaa₄₉-Gly₅₀-Xaa₅₁-Xaa₅₂-Xaa₅₃-Xaa₅₄-Xaa₅₅-Ser₅₆- |
| | | Gly₅₇-Cys₅₈-Xaa₅₉-Xaa₆₀-Xaa₆₁-Glu₆₂-Gly₆₃-Ser₆₄- |
| | | Asp₆₅-Phe₆₆-Gln₆₇-Cys₆₈-Xaa₆₉-Asp₇₀-Xaa₇₁-Pro₇₂- |
| | | Xaa₇₃-Xaa₇₄-Xaa₇₅-Xaa₇₆-Arg₇₇-Arg₇₈-Xaa₇₉-Ile₈₀- |
| | | Glu₈₁-Cys₈₂-Cys₈₃-Xaa₈₄-Xaa₈₅-Xaa₈₆-Xaa₈₇-Xaa₈₈- |
| | | Cys₈₉-Asn₉₀-Xaa₉₁-Xaa₉₂-Leu₉₃-Xaa₉₄-Pro₉₅-Thr₉₆- |
| | | Leu₉₇-Pro₉₈-Pro₉₉-Leu₁₀₀-Lys₁₀₁-Asn₁₀₂-Arg₁₀₃- |
| | | Asp₁₀₄-Phe₁₀₅-Val₁₀₆-Xaa₁₀₇-Xaa₁₀₈-Xaa₁₀₉-Xaa₁₁₀- |
| | | Xaa₁₁₁-Xaa₁₁₂-Xaa₁₁₃; |
| | | wherein Xaa₁₅ is K or P; Xaa₁₆ is V or I; Xaa₂₀ is V, F, K or Y; Xaa₂₂ |
| | | is G, H or S; Xaa′₂₂ is absent or A; Xaa₂₃ is S, H or G; Xaa₂₇ is E or |
| | | D; Xaa₂₉ is S or A; Xaa₃₀ is V or I; Xaa₃₃ is I or T; Xaa₃₅ is S or I; |
| | | Xaa₃₇ is D or N; Xaa₃₉ is Y or H; Xaa₄₂ is T or A; Xaa₄₃ is M or I; Xaa₄₉ |
| | | is S or Q; Xaa₅₁ is M, L or E; Xaa₅₂ is P or T; Xaa₅₃ is V or T; Xaa₅₄ |
| | | is V or L; Xaa₅₅ is T or A; Xaa₅₉ is L or M; Xaa₆₀ is G or K; Xaa₆₁ is |
| | | L or Y; Xaa₆₉ is R or K; Xaa₇₁ is T or S; Xaa₇₃ is I or K; Xaa₇₄ is P |
| | | or A; Xaa₇₅ is H or Q; Xaa₇₆ is Q or L; Xaa₇₉ is S or T; Xaa8₄ is T or |
| | | R; Xaa₈₅ is absent, T or E; Xaa₈₆ is R or N; Xaa₈₇ is N or L; Xaa₈₈ is |
| | | absent, E or L; Xaa₉₁ is K or Q; Xaa₉₂ is D or L; Xaa₉₄ is H or Q; |
| | | Xaa₁₀₇ is absent; Xaa₁₀₈ is absent; Xaa₁₀₉ is absent; Xaa₁₁₀ is absent; |
| | | Xaa₁₁₁ is absent; Xaa₁₁₂ is absent; and, Xaa₁₁₃ is absent |
| 109 | SEQ ID NO: 79 with | Lys₁-Lys₂-Glu₃-Asp₄-Gly₅-Glu₆-Ser₇-Thr₈-Ala₉- |
| | addition of aaA | Pro₁₀-Thr₁₁-Pro₁₂-Arg₁₃-Pro₁₄-Xaa₁₅-Xaa₁₆-Leu₁₇- |
| | between 22 and 23 | Arg₁₈-Cys₁₉-Xaa₂₀-Cys₂₁-Xaa₂₂-Xaa′₂₂-Xaa₂₃-His₂₄- |
| | | Cys₂₅-Pro₂₆-Xaa₂₇-Asp₂₈-Xaa₂₉-Xaa₃₀-Asn₃₁-Asn₃₂- |
| | | Xaa₃₃-Cys₃₄-Xaa₃₅-Thr₃₆-Xaa₃₇-Gly₃₈-Xaa₃₉-Cys₄₀- |
| | | Phe₄₁-Xaa₄₂-Xaa₄₃-Ile₄₄-Glu₄₅-Glu₄₆-Asp₄₇-Asp₄₈- |
| | | Xaa₄₉-Gly₅₀-Xaa₅₁-Xaa₅₂-Xaa₅₃-Xaa₅₄-Xaa₅₅-Ser₅₆- |
| | | Gly₅₇-Cys₅₈-Xaa₅₉-Xaa₆₀-Xaa₆₁-Glu₆₂-Gly₆₃-Ser₆₄- |
| | | Asp₆₅-Phe₆₆-Gln₆₇-Cys₆₈-Xaa₆₉-Asp₇₀-Xaa₇-Pro₇₂- |
| | | Xaa₇₃-Xaa₇₄-Xaa₇₅-Xaa₇₆-Arg₇₇-Arg₇₈-Xaa₇₉-Ile₈₀- |
| | | Glu₈₁-Cys₈₂-Cys₈₃-Xaa₈₄-Xaa₈₅-Xaa₈₆-Xaa₈₇-Xaa₈₈- |
| | | Cys₈₉-Asn₉₀-Xaa₉₁-Xaa₉₂-Leu₉₃-Xaa₉₄-Pro₉₅-Thr₉₆- |
| | | Leu₉₇-Pro₉₈-Pro₉₉-Val₁₀₀-Val₁₀₁-Ile₁₀₂-Gly₁₀₃-Pro₁₀₄- |
| | | Phe₁₀₅-Phe₁₀₆-Asp₁₀₇-Gly₁₀₈-Ser₁₀₉-Ile₁₁₀-Arg₁₁₁; |
| | | wherein Xaa₁₅ is Kor P; Xaa₁₆ is V or I; Xaa₂₀ is V, F, K or Y; Xaa₂₂ is |
| | | G, H or S; Xaa′₂₂ is absent or A-Xaa₂₃ is S, H or G; Xaa₂₇ is E or D; |
| | | Xaa₂₉ is S or A; Xaa₃₀ is V or I; Xaa₃₃ is I or T; Xaa₃₅ is S or I; Xaa₃₇ |
| | | is D or N; Xaa₃₉ is Y or H; Xaa₄₂ is T or A; Xaa₄₃ is M or I; Xaa₄₉ is S |
| | | or Q; Xaa₅₁ is L or E; Xaa₅₂ is P or T; Xaa₅₃ is V or T; Xaa₅₄ is V or L; |
| | | Xaa₅₅ is T or A; Xaa₅₉ is L or M; Xaa₆₀ is G or K; Xaa₆₁ is L or Y Xaa₆₉ |
| | | is R or K; Xaa₇₁ is T or S; Xaa₇₃ is I or K; Xaa₇₄ is P or A; Xaa₇₅ is H |
| | | or Q; Xaa₇₆ is Q or L; Xaa₇₉ is S or T; Xaa₈₄ is T or R; Xaa₈₅ is absent, |
| | | T or E; Xaa₈₆ is R or N; Xaa₈₇ is N or L; Xaa₈₈ is absent, E or L; Xaa₉₁ |
| | | is K or Q; Xaa₉₂ is D or L; and, Xaa₉₄ is H or Q |
| 110 | SEQ ID NO: 69 | KKEDGESTAPTPRPKVLRCYCSAGHCPEDSVNNICST |
| | Variant ECD; K20Y; | DGYCFTMIEEDDSGLPVVTSGCMKYEGSDFQCRDTPI |
| | H22S; 22aA (inserted | PHQRRSIECCTERNECNKDLHPTLPPLKNRDFV____ |
| | between aa22 and | _ |
| | aa23) H23G; L59M, | |
| | G60K and L61Y and | |
| | C-terminus seven | |
| | amino acid deletion | |
| 111 | SEQ ID NO: 111 | KKEDGESTAPTPRPKVLRCYCSAGHCPEDSVNNICST |
| | | DGYCFTMIEEDDSGLPVVTSGCMKYEGSDFQCRDTPI |
| | | PHQRRSIECCTERNECNKDLHPTLPPLKNRDFV___ |
| | | _TGGGGSGGGGSGGGGSGGGGSTHTCPPCPAPEAA |
| | | GGPSVFLFPPKPKDTLMISRTPEVTCVVVDVSHEDPE |
| | | VKFNWYVDGVEVHNAKTKPREEQYNSTYRVVSVLT |
| | | VLHQDWLNGKEYKCKVSNKALPAPIEKTISKAKGQP |
| | | REPQVYTLPPSREEMTKNQVSLTCLVKGFYPSDIAVE |
| | | WESNGQPENNYKTTPPVLDSDGSFFLYSKLTVDKSR |
| | | WQQGNVFSCSVMHEALHNHYTQKSLSLSPG |
| 112 | SEQ ID NO: 33 + | KKEDGESTAPTPRPKVLRCKCHHHCPEDSVNNICSTD |
| | SEQ ID NO: 6 + SEQ | GYCFTMIEEDDSGLPVVTSGCMKYEGSDFQCRDTPIP |
| | ID NO: 106 | HQRRSIECCTERNECNKDLHPTLPPVVIGPFFDGSIRT |
| | | GGGGSGGGGSGGGGSGGGGSTHTCPPCPAPEAAG |
| | | GPSVFLFPPKPKDTLMISRTPEVTCVVVDVSHEDPEV |
| | | KFNWYVDGVEVHNAKTKPREEQYNSTYRVVSVLTV |
| | | LHQDWLNGKEYKCKVSNKALPAPIEKTISKAKGQPR |
| | | EPQVYTLPPSREEMTKNQVSLTCLVKGFYPSDIAVEW |
| | | ESNGQPENNYKTTPPVLDSDGSFFLYSKLTVDKSRW |
| | | QQGNVFSCSVMHEALHNHYTQKSLSLSPG |
| 113 | SEQ ID NO: 69 | KKEDGESTAPTPRPKVLRCKCHAHHCPEDSVNNICSTDGYCFT |
| | background with aaA | MIEEDDSGLPVVTSGCLGLEGSDFQCRDTPIPHQRRSIECCTERN |
| | inserted between aa22 | ECNKDLHPTLPPLKNRDFV____ |
| | and aa23 | |
| 114 | ALK6-Fc fusion | KKEDGESTAPTPRPKVLRCKCHAHHCPEDSVNNICSTDGYCFT |
| | polypeptide | MIEEDDSGLPVVTSGCLGLEGSDFQCRDTPIPHQRRSIECCTERN |
| | _22′A | ECNKDLHPTLPPLKNRDFV____TGGGGSGGGGSGGGGSG |
| | | GGGSTHTCPPCPAPEAAGGPSVFLFPPKPKDTLMISRTPEVTCV |
| | | VVDVSHEDPEVKFNWYVDGVEVHNAKTKPREEQYNSTYRVVS |
| | | VLTVLHQDWLNGKEYKCKVSNKALPAPIEKTISKAKGQPREPQ |
| | | VYTLPPSREEMTKNQVSLTCLVKGFYPSDIAVEWESNGQPENN |
| | | YKTTPPVLDSDGSFFLYSKLTVDKSRWQQGNVFSCSVMHEALH |
| | | NHYTQKSLSLSPG |
| 115 | Alk6-Fc fusion | KKEDGESTAPTPRPKVLRCYCSGSCPEDSVNNICSTDGYCFT |
| | polypeptide | MIEEDDSGLPVVTSGCLGLEGSDFQCRDTPIPHQRRSIECCTE |
| | K20Y, H22S, H23G, | RNECNKDLHPTLPPLKNRDFV____TGGGGSGGGGSGG |
| | H24S (i.e., Xaa₂₀ is | GGSGGGGSTHTCPPCPAPEAAGGPSVFLFPPKPKDTLMISRT |
| | Y, Xaa₂₂ is S Xaa₂₃ is | PEVTCVVVDVSHEDPEVKFNWYVDGVEVHNAKTKPREEQY |
| | G, Xaa₂₄ is S) | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 116 | Alk6-Fc fusion | KKEDGESTAPTPRPKVLRCYCGAGHCPEDSVNNICSTDGYC |
| | polypeptide | FTMIEEDDSGLPVVTSGCLGLEGSDFQCRDTPIPHQRRSIECC |
| | K20Y, H22G, _22′A | TERNECNKDLHPTLPPLKNRDFV____TGGGGSGGGGSG |
| | (inserted between | GGGSGGGGSTHTCPPCPAPEAAGGPSVFLFPPKPKDTLMISR |
| | aa22 and aa23), H23G | TPEVTCVVVDVSHEDPEVKFNWYVDGVEVHNAKTKPREEQ |
| | (i.e., Xaa₂₀ is Y, | YNSTYRVVSVLTVLHQDWLNGKEYKCKVSNKALPAPIEKTI |
| | Xaa₂₂ is G _aa_{22′} is A, | SKAKGQPREPQVYTLPPSREEMTKNQVSLTCLVKGFYPSDIA |
| | Xaa₂₃ is G) | VEWESNGQPENNYKTTPPVLDSDGSFFLYSKLTVDKSRWQQ |
| | | GNVFSCSVMHEALHNHYTQKSLSLSPG |
| 117 | Alk6-Fc fusion | KKEDGESTAPTPRPKVLRCYCGGGHCPEDSVNNICSTDGYC |
| | polypeptide | FTMIEEDDSGLPVVTSGCLGLEGSDFQCRDTPIPHQRRSIECC |
| | K20Y, H22G, 22′G | TERNECNKDLHPTLPPLKNRDFV____TGGGGSGGGGSG |
| | (inserted between | GGGSGGGGSTHTCPPCPAPEAAGGPSVFLFPPKPKDTLMISR |
| | aa22 and aa23), H23G | TPEVTCVVVDVSHEDPEVKFNWYVDGVEVHNAKTKPREEQ |
| | (i.e., Xaa₂₀ is Y, | YNSTYRVVSVLTVLHQDWLNGKEYKCKVSNKALPAPIEKTI |
| | Xaa₂₂ is G _aa_{22′} is G, | SKAKGQPREPQVYTLPPSREEMTKNQVSLTCLVKGFYPSDIA |
| | Xaa₂₃ is G) | VEWESNGQPENNYKTTPPVLDSDGSFFLYSKLTVDKSRWQQ |
| | | GNVFSCSVMHEALHNHYTQKSLSLSPG |
| 118 | ALK6-Fc fusion | KKEDGESTAPTPRPKVLRCVCEGLSCPEDSVNNICSTDGYCF |
| | polypeptide | TMIEEDDSGLPVVTSGCLGLEGSDFQCRDTPIPHQRRSIECCT |
| | K20V, H22E, 22′G | ERNECNKDLHPTLPPLKNRDFV____TGGGGSGGGGSGG |
| | (inserted between | GGSGGGGSTHTCPPCPAPEAAGGPSVFLFPPKPKDTLMISRT |
| | aa22 and aa23), H23L, | PEVTCVVVDVSHEDPEVKFNWYVDGVEVHNAKTKPREEQY |
| | and H24S (i.e., Xaa₂₀ | NSTYRVVSVLTVLHQDWLNGKEYKCKVSNKALPAPIEKTIS |
| | is V, Xaa₂₂ is E _aa₂₂′ | KAKGQPREPQVYTLPPSREEMTKNQVSLTCLVKGFYPSDIAV |
| | is G, Xaa₂₃ is L, Xaa₂₄ | EWESNGQPENNYKTTPPVLDSDGSFFLYSKLTVDKSRWQQG |
| | is S) | NVFSCSVMHEALHNHYTQKSLSLSPG |
| 119 | ALK6-Fc fusion | KKEDGESTAPTPRPKVLRCVCEGLSHCPEDSVNNICSTDGYC |
| | polypeptide | FTMIEEDDSGLPVVTSGCLGLEGSDFQCRDTPIPHQRRSIECC |
| | K20V, H22E, 22′G | TERNECNKDLHPTLPPLKNRDFV____TGGGGSGGGGSG |
| | (inserted between | GGGSGGGGSTHTCPPCPAPEAAGGPSVFLFPPKPKDTLMISR |
| | aa22 and aa23), _22”L | TPEVTCVVVDVSHEDPEVKFNWYVDGVEVHNAKTKPREEQ |
| | (inserted between _22′ | YNSTYRVVSVLTVLHQDWLNGKEYKCKVSNKALPAPIEKTI |
| | and aa23)H23S (i.e., | SKAKGQPREPQVYTLPPSREEMTKNQVSLTCLVKGFYPSDIA |
| | Xaa₂₀ is V, Xaa₂₂ is E | VEWESNGQPENNYKTTPPVLDSDGSFFLYSKLTVDKSRWQQ |
| | _aa₂₂′ is G,_aa_{22″} is | GNVFSCSVMHEALHNHYTQKSLSLSPG |
| | L, Xaa₂₃ is S) | |
| 120 | ALK6-Fc fusion | KKEDGESTAPTPRPKVLRCTCHHHCPEDSVNNICSTDGYCFT |
| | polypeptide | MIEEDDSGLPVVTSGCLGLEGSDFQCRDTPIPHQRRSIECCTE |
| | K20T (i.e., Xaa₂₀ | RNECNKDLHPTLPPLKNRDFV____TGGGGGGGGSGG |
| | is T) | GGSGGGGSTHTCPPCPAPEAAGGPSVFLFPPKPKDTLMISRT |
| | | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 121 | ALK6-Fc fusion | KKEDGESTAPTPRPKVLRCVCHHHCPEDSVNNICSTDGYCFT |
| | polypeptide | MIEEDDSGLPVVTSGCLGLEGSDFQCRDTPIPHQRRSIECCTE |
| | K20V (i.e., Xaa₂₀ | RNECNKDLHPTLPPLKNRDFV____TGGGGSGGGGSGG |
| | is V) | GGSGGGGSTHTCPPCPAPEAAGGPSVFLFPPKPKDTLMISRT |
| | | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 122 | ALK6-Fc fusion | KKEDGESTAPTPRPKVLRCFCHHHCPEDSVNNICSTDGYCFT |
| | polypeptide | MIEEDDSGLPVVTSGCLGLEGSDFQCRDTPIPHQRRSIECCTE |
| | K20F (i.e., Xaa₂₀ | RNECNKDLHPTLPPLKNRDFV____TGGGGSGGGGSGG |
| | is F) | GGSGGGGSTHTCPPCPAPEAAGGPSVFLFPPKPKDTLMISRT |
| | | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 123 | ALK6-Fc fusion | KKEDGESTAPTPRPKVLRCACHHHCPEDSVNNICSTDGYCFT |
| | polypeptide | MIEEDDSGLPVVTSGCLGLEGSDFQCRDTPIPHQRRSIECCTE |
| | K20A (i.e., Xaa₂₀ | RNECNKDLHPTLPPLKNRDFV____TGGGGSGGGGSGG |
| | is A) | GGSGGGGSTHTCPPCPAPEAAGGPSVFLFPPKPKDTLMISRT |
| | | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 124 | ALK6-Fc fusion | KKEDGESTAPTPRPKVLRCVCGSHCPEDSVNNICSTDGYCFT |
| | polypeptide | MIEEDDSGLPVVTSGCLGLEGSDFQCRDTPIPHQRRSIECCTE |
| | K20V, H22G, H23S | RNECNKDLHPTLPPLKNRDFV____TGGGGSGGGGSGG |
| | (i.e., Xaa₂₀ is V, | GGSGGGGSTHTCPPCPAPEAAGGPSVFLFPPKPKDTLMISRT |
| | Xaa₂₂ is G, Xaa₂₃ is S) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 125 | ALK6-Fc fusion | KKEDGESTAPTPRPKVLRCFCGSHCPEDSVNNICSTDGYCFT |
| | polypeptide | MIEEDDSGLPVVTSGCLGLEGSDFQCRDTPIPHQRRSIECCTE |
| | K20F, H22G, H23S | RNECNKDLHPTLPPLKNRDFV____TGGGGGGGGSGG |
| | (i.e., Xaa₂₀ is F, | GGSGGGGSTHTCPPCPAPEAAGGPSVFLFPPKPKDTLMISRT |
| | Xaa₂₂ is G, Xaa₂₃ is S) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 126 | ALK6-Fc fusion | KKEDGESTAPTPRPKVLRCACGSHCPEDSVNNICSTDGYCFT |
| | polypeptide | MIEEDDSGLPVVTSGCLGLEGSDFQCRDTPIPHQRRSIECCTE |
| | K20A, H22G, H23S | RNECNKDLHPTLPPLKNRDFV____TGGGGSGGGGSGG |
| | (i.e., Xaa₂₀ is A, | GGSGGGGSTHTCPPCPAPEAAGGPSVFLFPPKPKDTLMISRT |
| | Xaa₂₂ is G, Xaa₂₃ is S) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 127 | ALK6-Fc fusion | KKEDGESTAPTPRPKVLRCYCGSLHCPEDSVNNICSTDGYCF |
| | polypeptide | TMIEEDDSGLPVVTSGCLGLEGSDFQCRDTPIPHQRRSIECCT |
| | K20Y, H22G, 22′S | ERNECNKDLHPTLPPLKNRDFV____TGGGGSGGGGSGG |
| | (inserted between | GGSGGGGSTHTCPPCPAPEAAGGPSVFLFPPKPKDTLMISRT |
| | aa22 and aa23), | PEVTCVVVDVSHEDPEVKFNWYVDGVEVHNAKTKPREEQY |
| | H23L(i.e., Xaa₂₀ is Y, | NSTYRVVSVLTVLHQDWLNGKEYKCKVSNKALPAPIEKTIS |
| | Xaa₂₂ is G _aa₂₂′ is S, | KAKGQPREPQVYTLPPSREEMTKNQVSLTCLVKGFYPSDIAV |
| | Xaa₂₃ is L) | EWESNGQPENNYKTTPPVLDSDGSFFLYSKLTVDKSRWQQG |
| | | NVFSCSVMHEALHNHYTQKSLSLSPG |
| 128 | ALK6-Fc fusion | KKEDGESTAPTPRPKVLRCYCGSGHCPEDSVNNICSTDGYCF |
| | polypeptide | TMIEEDDSGLPVVTSGCLGLEGSDFQCRDTPIPHQRRSIECCT |
| | K20Y, H22G, _22′S | ERNECNKDLHPTLPPLKNRDFV____TGGGGSGGGGSGG |
| | (inserted between | GGSGGGGSTHTCPPCPAPEAAGGPSVFLFPPKPKDTLMISRT |
| | aa22 and aa23), | PEVTCVVVDVSHEDPEVKFNWYVDGVEVHNAKTKPREEQY |
| | H23G(i.e., Xaa₂₀ is Y, | NSTYRVVSVLTVLHQDWLNGKEYKCKVSNKALPAPIEKTIS |
| | Xaa₂₂ is G _aa₂₂′ is S, | KAKGQPREPQVYTLPPSREEMTKNQVSLTCLVKGFYPSDIAV |
| | Xaa₂₃ is G) | EWESNGQPENNYKTTPPVLDSDGSFFLYSKLTVDKSRWQQG |
| | | NVFSCSVMHEALHNHYTQKSLSLSPG |
| 129 | ALK6-Fc fusion | KKEDGESTAPTPRPKVLRCYCSGHCPEDSVNNICSTDGYCFT |
| | polypeptide | MIEEDDSGLPVVTSGCLGLEGSDFQCRDTPIPHQRRSIECCTE |
| | K20Y, H22S, _H23G | RNECNKDLHPTLPPLKNRDFV____TGGGGSGGGGSGG |
| | (i.e., Xaa₂₀ is Y, | GGSGGGGSTHTCPPCPAPEAAGGPSVFLFPPKPKDTLMISRT |
| | Xaa₂₂ is S, _Xaa₂₃ is | PEVTCVVVDVSHEDPEVKFNWYVDGVEVHNAKTKPREEQY |
| | G) | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 130 | SEQ ID NO: 108 with | Lys₁-Lys₂-Glu₃-Asp₄-Glys-Glu₆-Ser₇-Thr₈-Ala₉- |
| | addition of aaA | Pro₁₀-Thr₁₁-Pro₁₂-Arg₁₃-Pro₁₄-Lys₁₅-Xaa₁₆-Leu₁₇- |
| | between ′22 and 23 | Arg₁₈-Cys₁₉-Xaa₂₀-Cys₂₁-Xaa₂₂-Xaa′₂₂- Xaa”₂₂-Xaa₂₃- |
| | | Xaa′₂₃-Xaa₂₄-Cys₂₅-Pro₂₆-Xaa₂₇-Asp₂₈-Xaa₂₉- |
| | | Xaa₃₀-Asn₃₁-Asn₃₂-Xaa₃₃-Cys₃₄-Xaa₃₅-Thr₃₆-Xaa₃₇- |
| | | Gly₃₈-Xaa₃₉-Cys₄₀-Phe₄₁-Xaa₄₂-Xaa₄₃-Ile₄₄-Glu₄₅- |
| | | Glu₄₆-Asp₄₇-Asp₄₈-Xaa₄₉-Gly₅₀-Xaa₅₁-Xaa₅₂-Xaa₅₃- |
| | | Xaa₅₄-Xaa₅₅-Ser₅₆-Gly₅₇-Cys₅₈-Xaa₅₉-Xaa₆₀-Xaa₆₁- |
| | | Glu₆₂-Gly₆₃-Ser₆₄-Asp₆₅-Phe₆₆-Gln₆₇-Cys₆₈-Xaa₆₉- |
| | | Asp₇₀-Xaa₇₁-Pro₇₂-Xaa₇₃-Xaa₇₄-Xaa₇₅-Xaa₇₆-Arg₇₇- |
| | | Arg₇₈-Xaa₇₉ -Ile₈₀-Glu₈₁-Cys₈₂-Cys₈₃-Xaa₈₄-Xaa₈₅- |
| | | Xaa₈₆-Xaa₈₇-Xaa₈₈-Cys₈₉-Asn₉₀-Xaa₉₁-Xaa₉₂-Leu₉₃- |
| | | Xaa₉₄-Pro₉₅-Thr₉₆-Leu₉₇-Pro₉₈-Pro₉₉-Leu₁₀₀-Lys₁₀₁- |
| | | Asn₁₀₂-Arg₁₀₃-Asp₁₀₄-Phe₁₀₅-Val₁₀₆-Xaa₁₀₇-Xaa₁₀₈- |
| | | Xaa₁₀₉-Xaa₁₁₀-Xaa₁₁₁-Xaa₁₁₂-Xaa₁₁₃; |
| | | wherein Xaa₁₆ is V or I; Xaa₂₀ is V, F, K, Y, T, or A; Xaa₂₂ is G, E, |
| | | H or S; Xaa′₂₂ is absent, G, S, or A; Xaa”₂₂ is absent or S; Xaa₂₃ is |
| | | S, H, G, or L; Xaa′₂₃ is absent or L; Xaa₂₄ is H or S; Xaa₂₇ is E or D; |
| | | Xaa₂₉ is S or A; Xaa₃₀ is V or I; Xaa₃₃ is I or T; Xaa₃₅ is S or I; Xaa₃₇ |
| | | is D or N; Xaa₃₉ is Y or H; Xaa₄₂ is T or A; Xaa₄₃ is M or I; Xaa₄₉ is |
| | | S or Q; Xaa₅₁ is M, L or E; Xaa₅₂ is P or T; Xaa₅₃ is V or T; Xaa₅₄ is |
| | | V or L; Xaa₅₅ is T or A; Xaa₅₉ is L or M; Xaa₆₀ is G or K; Xaa₆₁ is L |
| | | or Y; Xaa₆₉ is R or K; Xaa₇₁ is T or S; Xaa₇₂ is I or K; Xaa₇₄ is P or |
| | | A; Xaa₇₅ is H or Q; Xaa₇₆ is Q or L; Xaa₇₉ is S or T; Xaa₈₄ is T or R; |
| | | Xaa₈₅ is absent, T or E; Xaa₈₆ is R or N; Xaa₈₇ is N or L; Xaa₈₈ is |
| | | absent, E or L; Xaa₉₁ is K or Q; Xaa₉₂ is D or L; Xaa₉₄ is H or Q; |
| | | Xaa₁₀₇ is absent; Xaa₁₀₈ is absent; Xaa₁₀₉ is absent; Xaa₁₁₀ is absent; |
| | | Xaa₁₁₁ is absent; Xaa₁₁₂ is absent; and, Xaa₁₁₃ is absent |
| 131 | ALK3 ECD + liker | QNLDSMLHGTGMKSDSDQKKSENGVTLAPEDTLPFL |
| | +Fc | KCYCSGHCPDDAINNTCITNGHCFAIIEEDDQGETTLA |
| | | SGCMKYEGSDFQCKDSPKAQLRRTIECCRTNLCNQY |
| | | LQPTLPPVVIGPFFDGSIRTGGGGSGGGGSGGGGSG |
| | | GGGSTHTCPPCPAPELLGGPSVFLFPPKPKDTLMISRT |
| | | PEVTCVVVDVSHEDPEVKFNWYVDGVEVHNAKTKP |
| | | REEQYNSTYRVVSVLTVLHQDWLNGKEYKCKVSNK |
| | | ALPAPIEKTISKAKGQPREPQVYTLPPSREEMTKNQVS |
| | | LTCLVKGFYPSDIAVEWESNGQPENNYKTTPPVLDSD |
| | | GSFFLYSKLTVDKSRWQQGNVFSCSVMHEALHNHY |
| | | TQKSLSLSPGK |
| 132 | SEQ ID NO: 69 and | KKEDGESTAPTPRPKVLRCKCHHHCPEDSVNNICSTD |
| | 157 variant ECD + | GYCFTMIEEDDSGLPVVTSGCLGLEGSDFQCRDTPIP |
| | D104A | HQRRSIECCTERNECNKDLHPTLPPLKNRAFV____ |
| 133 | SEQ ID NO: 69 and | KKEDGESTAPTPRPPVLRCKCHHHCPEDSVNNICSTDGYCFTMI |
| | 157 variant ECD + | EEDDSGLPVVTSGCLGLEGSDFQCRDTPIPHQRRSIECCTERNEC |
| | K15P | NKDLHPTLPPLKNRDFV____ |
| 134 | SEQ ID NO: 69, 81 | KKEDGESTAPTPRPKVLRCKCHHHCPEDSVNNICSTDGYCFTMI |
| | and 157 variant + | EEDDSGLPVVTSGCLGLEGSDFQCRDTPIPHQRRSIECCT_RNEC |
| | E85 | NKDLHPTLPPLKNRDFV____ |
| 135 | SEQ ID NO: 69, 81 | KKEDGESTAPTPRPKVLRCKCHHHCPEDSINNICSTDGYCFTMIE |
| | and 157 variant + | EDDSGLPVVTSGCLGLEGSDFQCRDTPIPHQRRSIECCTERNECN |
| | V30I | KDLHPTLPPLKNRDFV____ |
| 136 | SEQ ID NO: 69, 81 | KKEDGESTAPTPRPKVLRCKCHHHCPEDSVNNTCSTDGYCFTM |
| | and 157 variant + | IEEDDSGLPVVTSGCLGLEGSDFQCRDTPIPHQRRSIECCTERNE |
| | I33T | CNKDLHPTLPPLKNRDFV____ |
| 137 | SEQ ID NO: 69, 81 | KKEDGESTAPTPRPKVLRCKCHHHCPEDSVNNICITDGYCFTMI |
| | and 157 variant + | EEDDSGLPVVTSGCLGLEGSDFQCRDTPIPHQRRSIECCTERNEC |
| | S35I | NKDLHPTLPPLKNRDFV____ |
| 138 | SEQ ID NO: 69, 81 | KKEDGESTAPTPRPKVLRCKCHHHCPEDSVNNICSTDGHCFTMI |
| | and 157 variant + | EEDDSGLPVVTSGCLGLEGSDFQCRDTPIPHQRRSIECCTERNEC |
| | Y39H | NKDLHPTLPPLKNRDFV____ |
| 139 | SEQ ID NO: 69, 81 | KKEDGESTAPTPRPKVLRCKCHHHCPEDSVNNICSTDGYCFTMI |
| | and 157 variant + | EEDDQGLPVVTSGCLGLEGSDFQCRDTPIPHQRRSIECCTERNEC |
| | S49Q | NKDLHPTLPPLKNRDFV____ |
| 140 | SEQ ID NO: 69, 81 | KKEDGESTAPTPRPKVLRCKCHHHCPEDSVNNICSTDGYCFTMI |
| | and 157 variant + | EEDDSGLPVVTSGCLKLEGSDFQCRDTPIPHQRRSIECCTERNEC |
| | G60K | NKDLHPTLPPLKNRDFV____ |
| 141 | SEQ ID NO: 69, 81 | KKEDGESTAPTPRPKVLRCKCHHHCPEDSVNNICSTDGYCFTMI |
| | and 157 variant + | EEDDSGLPVVTSGCMKYEGSDFQCRDTPIPHQRRSIECCTERNE |
| | L59M, G60K and | CNKDLHPTLPPLKNRDFV____ |
| | L61Y | |
| 142 | SEQ ID NO: 69, 81 | KKEDGESTAPTPRPKVLRCKCHHHCPEDSVNNICSTDGYCFTMI |
| | and 157 variant + | EEDDSGLPVVTSGCLGLEGSDFQCRDTPIPHQRRSIECCRERNEC |
| | T84R | NKDLHPTLPPLKNRDFV____ |
| 143 | SEQ ID NO: 69, 81 | KKEDGESTAPTPRPKVLRCKCHHHCPEDSVNNICSTDGYCFTMI |
| | and 157 variant + | EEDDSGLPVVTSGCLGLEGSDFQCRDTPIPHQRRSIECCTENNEC |
| | R86N | NKDLHPTLPPLKNRDFV____ |
| 144 | SEQ ID NO: 69, 81 | KKEDGESTAPTPRPKVLRCKCHHHCPEDSVNNICSTDGYCFTMI |
| | and 157 variant + | EEDDSGLPVVTSGCLGLEGSDFQCRDTPIPHQRRSIECCTERLEC |
| | N87L | NKDLHPTLPPLKNRDFV____ |
| 145 | SEQ ID NO: 69, 81 | KKEDGESTAPTPRPKVLRCKCHHHCPEDSVNNICSTDGYCFTMI |
| | and 157 variant + | EEDDSGLPVVTSGCLGLEGSDFQCRDTPIPHQRRSIECCTTRNEC |
| | E85T | NKDLHPTLPPLKNRDFV____ |
| 146 | SEQ ID NO: 69, 157 | KKEDGESTAPTPRPKVLRCKCHHHCPEDSVNNICSTDGYCFTMI |
| | and 190 variant + | EEDDSGLPVVTSGCLGLEGSDFQCRDTPIPHQRRSIECCTERNEC |
| | D92Y | NKYLHPTLPPLKNRDFV____ |
| 147 | SEQ ID NO: 69,81 | KKEDGESTAPTPRPKVLRCKCHHHCPEDSVNNICSTDGYCFTMI |
| | and 157 variant + | EEDDSGLPVVTSGCLGLEGSDFQCRDTPIPHQRRSIECCTERN_C |
| | E88 | NKDLHPTLPPLKNRDFV____ |
| 148 | SEQ ID NO: 69 157 | KKEDGESTAPTPRPKVLRCKCHHHCPEDSVNNICSTDGYCFTMI |
| | and 190 variant + | EEDDSGLPVVTSGCLGLEGSDFQCRDTPIPHQRRSIECCRTNL_C |
| | T84R, E85T, R86N, | NQYLQPTLPPLKNRDFV____ |
| | N87L, E88_, K91Q, | |
| | D92Y and H94Q | |
| 149 | SEQ ID NO: 69, 81 | KKEDGESTAPTPRPKVLRCKCSGHCPEDSVNNICSTDGYCFTMI |
| | and 157 variant + | EEDDSGLPVVTSGCLGLEGSDFQCRDTPIPHQRRSIECCTERNEC |
| | H22S and H23G | NKDLHPTLPPLKNRDFV____ |
| 150 | SEQ ID NO: 69, 157 | KKEDGESTAPTPRPKVLRCVCEGLSCPEDSVNNICSTDGYCFTM |
| | and 190 variant + | IEEDDSGLPVVTSGCLGLEGSDFQCRDTPIPHQRRSIECCTERNE |
| | K20V, H22E, H23G, | CNKDLHPTLPPLKNRDFV____ |
| | 23′L, H24S | |
| 151 | SEQ ID NO: 69, 157 | KKEDGESTAPTPRPKVLRCVCEGLHCPEDSVNNICSTDGYCFT |
| | and 190 variant + | MIEEDDSGLPVVTSGCLGLEGSDFQCRDTPIPHQRRSIECCTERN |
| | K20V, H22E, H23G, | ECNKDLHPTLPPLKNRDFV____ |
| | 23′L | |
| 152 | SEQ ID NO: 69, 157 | KKEDGESTAPTPRPKVLRCVCEGHSCPEDSVNNICSTDGYCFTM |
| | and 190 variant + | IEEDDSGLPVVTSGCLGLEGSDFQCRDTPIPHQRRSIECCTERNE |
| | K20V, H22E, H23G, | CNKDLHPTLPPLKNRDFV____ |
| | 23′H, H24S | |
| 153 | Wild type ALK-6 with | ____TPRPKVLRCKCHHHCPEDSVNNICSTDGYCFTMIEE |
| | N terminus truncated | DDSGLPVVTSGCLGLEGSDFQCRDTPIPHQRRSIECCTERNECNK |
| | (10 aa deleted) | DLHPTLPPLKNRDFVDGPIHHR |
| 154 | Wild type ALK-6 with | _____PKVLRCKCHHHCPEDSVNNICSTDGYCFTMIEED |
| | N terminus truncated | DSGLPVVTSGCLGLEGSDFQCRDTPIPHQRRSIECCTERNECNKD |
| | (13 aa deleted) | LHPTLPPLKNRDFVDGPIHHR |
| 155 | Wild type ALK-6 with | ______LRCKCHHHCPEDSVNNICSTDGYCFTMIEEDD |
| | N terminus truncated | SGLPVVTSGCLGLEGSDFQCRDTPIPHQRRSIECCTERNECNKDL |
| | (16 aa deleted) | HPTLPPLKNRDFVDGPIHHR |
| 156 | Truncated ALK6 | KKEDGESTAPTPRPKVLRCKCHHHCPEDSVNNICSTDGYCFTMI |
| | ECD (30 aa deleted) | EEDDSGLPVVTSGCLGLEGSDFQCRDTPIPHQRRSIECCRTNLC |
| | with ALK3 C | NQYLQPTLPPVVIG |
| | terminus sequence | |
| 157 | | Xaa₁-Xaa₂-Xaa₃-Xaa₄-Xaas-Xaa₆-Xaa₇-Xaas-Xaa₉- |
| | | Xaa₁₀-Xaa₁₁-Xaa₁₂-Xaa₁₃-Xaa₁₄-Xaa₁₅-Xaa₁₆-Leu₁₇- |
| | | Arg₁₈-Cys₁₉-Xaa₂₀-Cys₂₁-Xaa₂₂-Xaa′₂₂-Xaa”₂₂-Xaa₂₃- |
| | | Xaa′₂₃-Xaa₂₄-Cys₂₅-Pro₂₆-Xaa₂₇-Asp₂₈-Xaa₂₉-Xaa₃₀- |
| | | Asn₃₁-Asn₃₂-Xaa₃₃-Cys₃₄-Xaa₃₅-Thr₃₆-Xaa₃₇-Gly₃₈-Xaa₃₉- |
| | | Cys₄₀-Phe₄₁-Xaa₄₂-Xaa₄₃-Ile₄₄-Glu₄₅-Glu₄₆-Asp₄₇- |
| | | Asp₄₈-Xaa₄₉-Gly₅₀-Xaa₅₁-Xaa₅₂-Xaa₅₃-Xaa₅₄-Xaa₅₅-Ser₅₆- |
| | | Gly₅₇-Cys₅₈-Xaa₅₉-Xaa₆₀-Xaa₆₁-Glu₆₂-Gly₆₃-Ser₆₄-Asp₆₅- |
| | | Phe₆₆-Gln₆₇-Cys₆₈-Xaa₆₉-Asp₇₀-Xaa₇₁-Pro₇₂-Xaa₇₃- |
| | | Xaa₇₄-Xaa₇₅-Xaa₇₆-Arg₇₇-Arg₇₈-Xaa₇₉-Ile₈₀-Glu₈₁-Cys₈₂- |
| | | Cys₈₃-Xaa₈₄-Xaa₈₅-Xaa₈₆-Xaa₈₇-Xaa₈₈-Cys₈₉-Asn₉₀-Xaa₉₁- |
| | | Xaa₉₂-Leu₉₃-Xaa₉₄-Pro₉₅-Thr₉₆-Leu₉₇-Pro₉₈-Pro₉₉- |
| | | Xaa₁₀₀-Xaa₁₀₁-Xaa₁₀₂-Xaa₁₀₃-Xaa₁₀₄-Xaa₁₀₅-Xaa₁₀₆-Xaa₁₀₇- |
| | | Xaa₁₀₈-Xaa₁₀₉-Xaan₁₀-Xaa₁₁₁-Xaa₁₁₂-Xaa₁₁₃ |
| | | wherein: Xaa₁ is K or absent; Xaa₂ is K or absent; Xaa₃ is E or absent; |
| | | Xaa₄ is D or absent; Xaa₅ is G or absent, Xaa₆ is E or absent; Xaa₇ is |
| | | S or absent; Xaa₈ is T or absent; Xaa₉ is A or absent; Xaa₁₀ is P or |
| | | absent; Xaa₁₁ is T or absent; Xaa₁₂ is P or absent; Xaa₁₃ is R or |
| | | absent; Xaa₁₄ is P or absent, Xaa₁₅ is K, P or absent; Xaa₁₆ is V or |
| | | absence; Xaa₂₀ is V, F, K, A, R, N, D, C, E, Q, G, H, I, L, M, P, S, |
| | | T, W, or Y; Xaa₂₂ is G, H, V, F, K, A, R, N, D, C, E, Q, I, L, M, P, |
| | | T, W, Y or S; Xaa′₂₂ is absent, G, S, or A; Xaa”₂₂ is absent or S; |
| | | Xaa₂₃ is S, H, V, F, K, A, R, N, D, C, E, Q, I, L, M, P, T, W, Y or |
| | | G; Xaa′₂₃ is absent, L or H; Xaa₂₄ is V, F, K, A, R, N, D, C, E, Q, G, |
| | | H, I, L, M, P, S, T, W, or Y; Xaa₂₇ is E or D; Xaa₂₉ is S, G or A; |
| | | Xaa₃₀ is V, A, G or I; Xaa₃₃ is I, S or T; Xaa₃₅ is S, T, G, A or I; |
| | | Xaa₃₇ is D, A, G or N; Xaa₃₉ is Y, A, G or H; Xaa₄₂ is T or A; Xaa₄₃ is |
| | | M or I; Xaa₄₉ is S, A, G or Q; Xaa₅₁ is L, A, G or E; Xaa₅₂ is P or T; |
| | | Xaa₅₃ is V, A, G or T; Xaa₅₄ is V, A, G or L; Xaa₅₅ is T, G or A; |
| | | Xaa₅₉ is L, M, V, F, K, A, R, N, D, C, E, Q, G, H, I, P, S, T, W, or |
| | | Y; Xaa₆₀ is G, K, V, F, A, R, N, D, C, E, Q, H, I, L, M, P, S, T, W, |
| | | or Y; Xaa₆₁ is L, Y, V, F, K, A, R, N, D, C, E, Q, G, H, I, M, P, S, |
| | | T, or W; Xaa₆₉ is R or K; Xaa₇₁ is T or S; Xaa₇₃ is I or K; Xaa₇₄ is |
| | | P or A; Xaa₇₅ is H or Q; Xaa₇₆ is Q or L; Xaa₇₇ is S or T; Xaa₈₄ is |
| | | T, R or absent; Xaa85 is absent, T or E; Xaa8₆ is R, N or absent; |
| | | Xaa₈₇ is N, L or absent; Xaa₈₈ is absent, E or L; Xaa₉₁ is K, Q or |
| | | absent; Xaa₉₂ is D, L, Y or absent; Xaa₉₄ is H, Q or absent; Xaa₁₀₀ |
| | | is absent, V or L; Xaa₁₀₁ is absent, V or K; Xaa₁₀₂ is absent, I or N; |
| | | Xaa₁₀₃ is absent, G or R; Xaa₁₀₄ is absent, P, D or A; Xaa₁₀₅ is absent |
| | | or F; Xaa₁₀₆ is absent, F, W, Y, N, Q, K, H, D, G, E or V; Xaa₁₀₇ is |
| | | absent, L, M, V, F, K, A, R, N, C, E, Q, G, H, I, P, S, T, W, or Y |
| | | or D; Xaa₁₀₈ is absent, R, T, Q, L, K, W or G; Xaa₁₀₉ is absent, S or |
| | | P; Xaa₁₁₀ is absent or I; Xaa₁₁₁ is absent, R or H; Xaa₁₁₂ is absent or |
| | | H; and Xaa₁₁₃ is absent or R |
| 158 | SEQ ID NO: 69 and | KKEDGESTAPTPRPKVLRCKCHHHCPEDSINNICITDGYCFTMIE |
| | 190 variant + V30I, | EDDQGLPVVTSGCLGLEGSDFQCRDTPIPHQRRSIECCTERNEC |
| | S35I, and S49Q | NKDLHPTLPPLKNRDFV____ |
| 159 | SEQ ID NO: 69 and | KKEDGESTAPTPRPKVLRCKCSHHCPEDSVNNICSTDGYCFTMI |
| | 190 variant + H22S | EEDDSGLPVVTSGCLGLEGSDFQCRDTPIPHQRRSIECCTERNEC |
| | | NKDLHPTLPPLKNRDFV____ |
| 160 | SEQ ID NO: 69 and | KKEDGESTAPTPRPKVLRCKCHHHCPEDSVNNICTTDGYCFTMI |
| | 190 variant + S35T | EEDDSGLPVVTSGCLGLEGSDFQCRDTPIPHQRRSIECCTERNEC |
| | | NKDLHPTLPPLKNRDFV____ |
| 161 | SEQ ID NO: 69 and | KKEDGESTAPTPRPKVLRCKCHHHCPEDSVNNSCSTDGYCFTMI |
| | 190 variant + 133S | EEDDSGLPVVTSGCLGLEGSDFQCRDTPIPHQRRSIECCTERNEC |
| | | NKDLHPTLPPLKNRDFV____ |
| 162 | SEQ ID NO: 69 and | KKEDGESTAPTPRPKVLRCKCHHHCPEDGVNNICSTDGYCFTMI |
| | 190 variant + S29G | EEDDSGLPVVTSGCLGLEGSDFQCRDTPIPHQRRSIECCTERNEC |
| | | NKDLHPTLPPLKNRDFV____ |
| 163 | SEQ ID NO: 69 and | KKEDGESTAPTPRPKVLRCKCHHHCPEDSGNNICSTDGYCFTMI |
| | 190 variant + V30G | EEDDSGLPVVTSGCLGLEGSDFQCRDTPIPHQRRSIECCTERNEC |
| | | NKDLHPTLPPLKNRDFV____ |
| 164 | SEQ ID NO: 69 and | KKEDGESTAPTPRPKVLRCKCHHHCPEDSANNICSTDGYCFTMI |
| | 190 variant + V30A | EEDDSGLPVVTSGCLGLEGSDFQCRDTPIPHQRRSIECCTERNEC |
| | | NKDLHPTLPPLKNRDFV____ |
| 165 | SEQ ID NO: 69 and | KKEDGESTAPTPRPKVLRCKCHHHCPEDSVNNICGTDGYCFTMI |
| | 190 variant + S35G | EEDDSGLPVVTSGCLGLEGSDFQCRDTPIPHQRRSIECCTERNEC |
| | | NKDLHPTLPPLKNRDFV____ |
| 166 | SEQ ID NO: 69 and | KKEDGESTAPTPRPKVLRCKCHHHCPEDSVNNICATDGYCFTMI |
| | 190 variant + S35A | EEDDSGLPVVTSGCLGLEGSDFQCRDTPIPHQRRSIECCTERNEC |
| | | NKDLHPTLPPLKNRDFV____ |
| 167 | SEQ ID NO: 69 and | KKEDGESTAPTPRPKVLRCKCHHHCPEDSVNNICSTAGYCFTMI |
| | 190 variant + S37A | EEDDSGLPVVTSGCLGLEGSDFQCRDTPIPHQRRSIECCTERNEC |
| | | NKDLHPTLPPLKNRDFV____ |
| 168 | SEQ ID NO: 69 and | KKEDGESTAPTPRPKVLRCKCHHHCPEDSVNNICSTGGYCFTMI |
| | 190 variant + S37G | EEDDSGLPVVTSGCLGLEGSDFQCRDTPIPHQRRSIECCTERNEC |
| | | NKDLHPTLPPLKNRDFV____ |
| 169 | SEQ ID NO: 69 and | KKEDGESTAPTPRPKVLRCKCHHHCPEDSVNNICSTDGACFTMI |
| | 190 variant + Y39A | EEDDSGLPVVTSGCLGLEGSDFQCRDTPIPHQRRSIECCTERNEC |
| | | NKDLHPTLPPLKNRDFV____ |
| 170 | SEQ ID NO: 69 and | KKEDGESTAPTPRPKVLRCKCHHHCPEDSVNNICSTDGGCFTMI |
| | 190 variant + Y39G | EEDDSGLPVVTSGCLGLEGSDFQCRDTPIPHQRRSIECCTERNEC |
| | | NKDLHPTLPPLKNRDFV____ |
| 171 | SEQ ID NO: 69 and | KKEDGESTAPTPRPKVLRCKCHHHCPEDSVNNICSTDGYCFTMI |
| | 190 variant + S49A | EEDDAGLPVVTSGCLGLEGSDFQCRDTPIPHQRRSIECCTERNEC |
| | | NKDLHPTLPPLKNRDFV____ |
| 172 | SEQ ID NO: 69 and | KKEDGESTAPTPRPKVLRCKCHHHCPEDSVNNICSTDGYCFTMI |
| | 190 variant + S49G | EEDDGGLPVVTSGCLGLEGSDFQCRDTPIPHQRRSIECCTERNEC |
| | | NKDLHPTLPPLKNRDFV____ |
| 173 | SEQ ID NO: 69 and | KKEDGESTAPTPRPKVLRCKCHHHCPEDSVNNICSTDGYCFTMI |
| | 190 variant + L51E | EEDDSGEPVVTSGCLGLEGSDFQCRDTPIPHQRRSIECCTERNEC |
| | | NKDLHPTLPPLKNRDFV____ |
| 174 | SEQ ID NO: 69 and | KKEDGESTAPTPRPKVLRCKCHHHCPEDSVNNICSTDGYCFTMI |
| | 190 variant + L51A | EEDDSGAPVVTSGCLGLEGSDFQCRDTPIPHQRRSIECCTERNEC |
| | | NKDLHPTLPPLKNRDFV____ |
| 175 | SEQ ID NO: 69 and | KKEDGESTAPTPRPKVLRCKCHHHCPEDSVNNICSTDGYCFTMI |
| | 190 variant + L51G | EEDDSGGPVVTSGCLGLEGSDFQCRDTPIPHQRRSIECCTERNEC |
| | | NKDLHPTLPPLKNRDFV____ |
| 176 | SEQ ID NO: 69 and | KKEDGESTAPTPRPKVLRCKCHHHCPEDSVNNICSTDGYCFTMI |
| | 190 variant + V53T | EEDDSGLPTVTSGCLGLEGSDFQCRDTPIPHQRRSIECCTERNEC |
| | | NKDLHPTLPPLKNRDFV____ |
| 177 | SEQ ID NO: 69 and | KKEDGESTAPTPRPKVLRCKCHHHCPEDSVNNICSTDGYCFTMI |
| | 190 variant + V53A | EEDDSGLPAVTSGCLGLEGSDFQCRDTPIPHQRRSIECCTERNEC |
| | | NKDLHPTLPPLKNRDFV____ |
| 178 | SEQ ID NO: 69 and | KKEDGESTAPTPRPKVLRCKCHHHCPEDSVNNICSTDGYCFTMI |
| | 190 variant + V53G | EEDDSGLPGVTSGCLGLEGSDFQCRDTPIPHQRRSIECCTERNEC |
| | | NKDLHPTLPPLKNRDFV____ |
| 179 | SEQ ID NO: 69 and | KKEDGESTAPTPRPKVLRCKCHHHCPEDSVNNICSTDGYCFTMI |
| | 190 variant + V54T | EEDDSGLPVTTSGCLGLEGSDFQCRDTPIPHQRRSIECCTERNEC |
| | | NKDLHPTLPPLKNRDFV____ |
| 180 | SEQ ID NO: 69 and | KKEDGESTAPTPRPKVLRCKCHHHCPEDSVNNICSTDGYCFTMI |
| | 190 variant + V54A | EEDDSGLPVATSGCLGLEGSDFQCRDTPIPHQRRSIECCTERNEC |
| | | NKDLHPTLPPLKNRDFV____ |
| 181 | SEQ ID NO: 69 and | KKEDGESTAPTPRPKVLRCKCHHHCPEDSVNNICSTDGYCFTMI |
| | 190 variant + V54G | EEDDSGLPVGTSGCLGLEGSDFQCRDTPIPHQRRSIECCTERNEC |
| | | NKDLHPTLPPLKNRDFV____ |
| 182 | SEQ ID NO: 69 and | KKEDGESTAPTPRPKVLRCKCHHHCPEDSVNNICSTDGYCFTMI |
| | 190 variant + T55A | EEDDSGLPVVASGCLGLEGSDFQCRDTPIPHQRRSIECCTERNEC |
| | | NKDLHPTLPPLKNRDFV____ |
| 183 | SEQ ID NO: 69 and | KKEDGESTAPTPRPKVLRCKCHHHCPEDSVNNICSTDGYCFTMI |
| | 190 variant + T55G | EEDDSGLPVVGSGCLGLEGSDFQCRDTPIPHQRRSIECCTERNEC |
| | | NKDLHPTLPPLKNRDFV____ |
| 184 | SEQ ID NO: 69 and | KKEDGESTAPTPRPKVLRCKCHHHCPEDSVNNICSTDGYCFTMI |
| | 190 variant + L59M | EEDDSGLPVVTSGCMGLEGSDFQCRDTPIPHQRRSIECCTERNE |
| | | CNKDLHPTLPPLKNRDFV____ |
| 185 | SEQ ID NO: 69 and | KKEDGESTAPTPRPKVLRCKCHHHCPEDSVNNICSTDGYCFTMI |
| | 190 variant + L59A | EEDDSGLPVVTSGCAGLEGSDFQCRDTPIPHQRRSIECCTERNEC |
| | | NKDLHPTLPPLKNRDFV____ |
| 186 | SEQ ID NO: 69 and | KKEDGESTAPTPRPKVLRCKCHHHCPEDSVNNICSTDGYCFTMI |
| | 190 variant ECD + | EEDDSGLPVVTSGCGGLEGSDFQCRDTPIPHQRRSIECCTERNEC |
| | L59G | NKDLHPTLPPLKNRDFV____ |
| 187 | SEQ ID NO: 69 and | KKEDGESTAPTPRPKVLRCYCSHHCPEDSVNNICSTDGYCFTMI |
| | 190 variant ECD; | EEDDSGLPVVTSGCLGLEGSDFQCRDTPIPHQRRSIECCTERNEC |
| | K20Y; H22S; and C- | NKDLHPTLPPLKNRDFV____ |
| | terminus seven amino | |
| | acid deletion | |
| 188 | SEQ ID NO: 69 and | KKEDGESTAPTPRPKVLRCYCSHHCPEDSVNNTCSTDGYCFTMI |
| | 190 variant ECD; | EEDDSGLPVVTSGCLGLEGSDFQCRDTPIPHQRRSIECCTERNEC |
| | K20Y; H22S; 133T | NKDLHPTLPPLKNRDFV____ |
| | and C-terminus seven | |
| | amino acid deletion | |
| 189 | SEQ ID NO: 69 and | KKEDGESTAPTPRPKVLRCKCSHHCPEDSVNNSCSTDGYCFTMI |
| | 190 variant ECD; | EEDDSGLPVVTSGCLGLEGSDFQCRDTPIPHQRRSIECCTERNEC |
| | H22S; I33S and C- | NKDLHPTLPPLKNRDFV____ |
| | terminus seven amino | |
| | acid deletion | |
| 190 | | Lys₁-Lys₂-Glu₃-Asp₄-Gly₅-Glu₆-Ser₇-Thr₈-Ala₉- |
| | | Pro₁₀-Thr₁₁-Pro₁₂-Arg₁₃-Pro₁₄-Xaa₁₅-Xaa₁₆-Leu₁₇- |
| | | Arg₁₈-Cys₁₉-Xaa₂₀-Cys₂₁-Xaa₂₂-Xaa′₂₂-Xaa”₂₂- |
| | | Xaa₂₃-Xaa′₂₃-Xaa₂₄-Cys₂₅-Pro₂₆-Xaa₂₇-Asp₂₈-Xaa₂₉- |
| | | Xaa₃₀-Asn₃₁-Asn₃₂-Xaa₃₃-Cys₃₄-Xaa₃₅-Thr₃₆-Xaa₃₇- |
| | | Gly₃₈-Xaa₃₉-Cys₄₀-Phe₄₁-Xaa₄₂-Xaa₄₃-Ile₄₄-Glu₄₅- |
| | | Glu₄₆-Asp₄₇-Asp₄₈-Xaa₄₉-Gly₅₀-Xaa₅₁-Xaa₅₂-Xaa₅₃- |
| | | Xaa₅₄-Xaa₅₅-Ser₅₆-Gly₅₇-Cys₅₈-Xaa₅₉-Xaa₆₀-Xaa₆₁- |
| | | Glu₆₂-Gly₆₃-Ser₆₄-Asp₆₅-Phe₆₆-Gln₆₇-Cys₆₈-Xaa₆₉- |
| | | Asp₇₀-Xaa₇₁-Pro₇₂-Xaa₇₃-Xaa₇₄-Xaa₇₅-Xaa₇₆-Arg₇₇- |
| | | Arg₇₈-Xaa₇₉-Ile₈₀-Glu₈₁-Cys₈₂-Cys₈₃-Xaas₄-Xaa₈₅- |
| | | Xaa₈₆-Xaa₈₇-Xaa₈₈-Cys₈₉-Asn₉₀-Xaa₉₁-Xaa₉₂-Leu₉₃- |
| | | Xaa₉₄-Pro₉₅-Thr₉₆-Leu₉₇-Pro₉₈-Pro₉₉-Leu₁₀₀-Lys₁₀₁- |
| | | Asn₁₀₂-Arg₁₀₃-Xaa₁₀₄-Phe₁₀₅-Val₁₀₆-Xaa₁₀₇-Xaa₁₀₈- |
| | | Xaa₁₀₉-Xaa₁₁₀-Xaa₁₁₁-Xaa₁₁₂-Xaa₁₁₃; |
| | | wherein Xaa₁₅ is K or P; Xaa₁₆ is V or I; Xaa₂₀ is V, F, K, A, R, N, D, |
| | | C, E, Q, G, H, I, L, M, P, S, T, W, or Y; Xaa₂₂ is G, H, V, F, K, A, |
| | | R, N, D, C, E, Q, I, L, M, P, T, W, Y or S; Xaa′₂₂ is absent, G, S, |
| | | or A; Xaa”₂₂ is absent or S; Xaa₂₃ is S, H, V, F, K, A, R, N, D, C, |
| | | E, Q, I, L, M, P, T, W, Y or G; Xaa′₂₃ is absent, L or H; Xaa₂₄ is |
| | | V, F, K, A, R, N, D, C, E, Q, G, H, I, L, M, P, S, T, W, or Y; |
| | | Xaa₂₇ is E or D; Xaa₂₉ is S, G or A; Xaa₃₀ is V, A, G or I; Xaa₃₃ is |
| | | I, S or T; Xaa₃₅ is S, T, G, A or I; Xaa₃₇ is D, A, G or N; Xaa₃₉ is Y, |
| | | A, G or H; Xaa₄₂ is T or A; Xaa₄₃ is M or I; Xaa₄₉ is S, A, G or Q; |
| | | Xaa₅₁ is L, A, G or E; Xaa₅₂ is P or T; Xaa₅₃ is V, A, G or T; Xaa₅₄ is |
| | | V, A, G or L; Xaa₅₅ is T, G or A; Xaa₅₉ is L, M, V, F, K, A, R, N, D, |
| | | C, E, Q, G, H, I, P, S, T, W, or Y; Xaa₆₀ is G, K, V, F, A, R, N, D, |
| | | C, E, Q, H, I, L, M, P, S, T, W, or Y; Xaa₆₁ is L, Y, V, F, K, A, R, |
| | | N, D, C, E, Q, G, H, I, M, P, S, T, or W; Xaa₆₉ is R or K; Xaa₇₁ is |
| | | T or S; Xaa₇₃ is I or K; Xaa₇₄ is P or A; Xaa₇₅ is H or Q; Xaa₇₆ is |
| | | Q or L; Xaa₇₉ is S or T; Xaa₈₄ is T, R or absent; Xaa₈₅ is absent, T |
| | | or E; Xaa₈₆ is absent, R or N; Xaa₈₇ is N or L; Xaa₈₈ is absent, E or |
| | | L; Xaa₉₁ is absent, K or Q; Xaa₉₂ is absent, D, Y or L; Xaa₉₄ is |
| | | absent, H or Q; Xaa₁₀₄ is D or A; Xaa₁₀₇ is absent; Xaa₁₀₈ is absent; |
| | | Xaa₁₀₉ is absent; Xaa₁₁₀ is absent; Xaa₁₁₁ is absent; Xaa₁₁₂ is absent; |
| | | and, Xaa₁₁₃ is absent |
| 191 | SEQ ID NO: 69 and | KKEDGESTAPTPRPKVLRCKCHHHCPEDSINNICITD |
| | 190 variant + V30I, | GYCFTMIEEDDQGLPVVTSGCLKLEGSDFQCRDTPIP |
| | S35I, S49Q and G60K | HQRRSIECCTERNECNKDLHPTLPPLKNRDFV____ |
| 192 | SEQ ID NO: 69 and | KKEDGESTAPTPRPKVLRCKCSHHCPEDSVNNICSTD |
| | 190 variant + H22S | GYCFTMIEEDDSGLPVVTSGCLKLEGSDFQCRDTPIP |
| | and G60K | HQRRSIECCTERNECNKDLHPTLPPLKNRDFV____ |
| 193 | SEQ ID NO: 69 and | KKEDGESTAPTPRPKVLRCYCSHHCPEDSVNNTCSTD |
| | 190 variant ECD; | GYCFTMIEEDDSGLPVVTSGCLKLEGSDFQCRDTPIP |
| | K20Y; H22S; I33T, | HQRRSIECCTERNECNKDLHPTLPPLKNRDFV____ |
| | G60K and C-terminus | |
| | seven amino acid | |
| | deletion | |
| 194 | SEQ ID NO: 69 and | KKEDGESTAPTPRPKVLRCKCSHHCPEDSVNNSCSTD |
| | 190 variant ECD; | GYCFTMIEEDDSGLPVVTSGCLKLEGSDFQCRDTPIP |
| | H22S; I33S, G60K | HQRRSIECCTERNECNKDLHPTLPPLKNRDFV____ |
| | and C-terminus seven | |
| | amino acid deletion | |
| 195 | ALK3 C terminal | RTNLCNQYLQPTLPPVVIG |
| | sequence | |
| 196 | Fab | EPKSCDKTHTCPPCPAPELLGGP |
| 197 | Fab | ERKCCVECPPCPAPPVAGP |
| 198 | Fab | ESKYGPPCPSCPAPEFLGGP |
| 199 | Fab | THTCPPCPAPEAAGGP |
| 200 | Fab | THTCPPCKCPEAAGGP |
| 201 | Fab | PTIKPCPPCKCPAP |
| 202 | SEQ ID NO: 202 | KKEDGESTAPTPRPKVLRCKCSHHCPEDSINNTCITDGYCFTMIE |
| | H22S, V30I, I33T, and | EDDSGLPVVTSGCLGLEGSDFQCRDTPIPHQRRSIECCTERNECN |
| | S35I | KDLHPTLPPLKNRDFV____ |
| 203 | SEQ ID NO: 202 with | KKEDGESTAPTPRPKVLRCKCSHHCPEDSINNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | H22S, V30I, I33T, and | TYRVVSVLTVLHQDWLNGKEYKCKVSNKALPAPIEKTISKA |
| | S35I | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 204 | SEQ ID NO: 202 with | KKEDGESTAPTPRPKVLRCKCSHHCPEDSINNTCITDGYCFTMIE |
| | linker sequence (SEQ | EDDSGLPVVTSGCLGLEGSDFQCRDTPIPHQRRSIECCTERNECN |
| | ID NO: 6), and Fab | KDLHPTLPPLKNRDFV____TGGGGSGGGGSGGGGSGGGGS |
| | (SEQ ID NO: 199) | THTCPPCPAPEAAGGP |
| | H22S, V30I, I33T, and | |
| | S35I | |
| 205 | SEQ ID NO: 202 with | KKEDGESTAPTPRPKVLRCKCSHHCPEDSINNTCITDGYCFTMIE |
| | linker sequence (SEQ | EDDSGLPVVTSGCLGLEGSDFQCRDTPIPHQRRSIECCTERNECN |
| | ID NO: 6), and Fab | KDLHPTLPPLKNRDFV____TGGGGSGGGGSGGGGSGGGGS |
| | (SEQ ID NO: 200) | THTCPPCKCPEAAGGP |
| | H22S, V30I, I33T, and | |
| | S35I | |
| 206 | SEQ ID NO: 202 with | KKEDGESTAPTPRPKVLRCKCSHHCPEDSINNTCITDGYCFTMIE |
| | linker sequence (SEQ | EDDSGLPVVTSGCLGLEGSDFQCRDTPIPHQRRSIECCTERNECN |
| | ID NO: 6), and Fab | KDLHPTLPPLKNRDFV____TGGGGSGGGGSGGGGSGGGGSP |
| | (SEQ ID NO: 201) | TIKPCPPCKCPAP |
| | H22S, V30I, I33T, and | |
| | S35I | |
| 207 | SEQ ID NO: 86 with | KKEDGESTAPTPRPKVLRCYCSGHCPEDSVNNICSTDGYCFTMI |
| | linker sequence (SEQ | EEDDSGLPVVTSGCLGLEGSDFQCRDTPIPHQRRSIECCTERNEC |
| | ID NO: 6), and Fab | NKDLHPTLPPLKNRDFV____TGGGGSGGGGSGGGGSGGGG |
| | (SEQ ID NO: 199) | STHTCPPCPAPEAAGGP |
| | K20Y, H22S, H23G | |
| 208 | SEQ ID NO: 86 with | KKEDGESTAPTPRPKVLRCYCSGHCPEDSVNNICSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | K20Y, H22S, H23G | |
| 209 | SEQ ID NO: 86 with | KKEDGESTAPTPRPKVLRCYCSGHCPEDSVNNICSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | K20Y, H22S, H23G | |
| 210 | SEQ ID NO: 90 with | KKEDGESTAPTPRPKVLRCYCSAGHCPEDSVNNICSTDGYCF |
| | linker sequence (SEQ | TMIEEDDSGLPVVTSGCLGLEGSDFQCRDTPIPHQRRSIECCT |
| | ID NO: 6), and Fab | ERNECNKDLHPTLPPLKNRDFV____TGGGGSGGGGSGG |
| | (SEQ ID NO: 199) | GGSGGGGSTHTCPPCPAPEAAGGP |
| | K20Y, H22S, _22′A, | |
| | H23G | |
| 211 | SEQ ID NO: 90 with | KKEDGESTAPTPRPKVLRCYCSAGHCPEDSVNNICSTDGYCF |
| | linker sequence (SEQ | TMIEEDDSGLPVVTSGCLGLEGSDFQCRDTPIPHQRRSIECCT |
| | ID NO: 6), and Fab | ERNECNKDLHPTLPPLKNRDFV____TGGGGSGGGGSGG |
| | (SEQ ID NO: 200) | GGSGGGGSTHTCPPCKCPEAAGGP |
| | K20Y, H22S, _22′A, | |
| | H23G | |
| 212 | SEQ ID NO: 90 with | KKEDGESTAPTPRPKVLRCYCSAGHCPEDSVNNICSTDGYCF |
| | linker sequence (SEQ | TMIEEDDSGLPVVTSGCLGLEGSDFQCRDTPIPHQRRSIECCT |
| | ID NO: 6), and Fab | ERNECNKDLHPTLPPLKNRDFV____TGGGGSGGGGSGG |
| | (SEQ ID NO: 201) | GGSGGGGSPTIKPCPPCKCPAP |
| | K20Y, H22S, _22′A, | |
| | H23G | |
| 213 | SEQ ID NO: 213 | KKEDGESTAPTPRPKVLRCKCHHHCPEDSINNTCITDGYCFT |
| | V30I, I33T, S35I | MIEEDDSGLPVVTSGCLGLEGSDFQCRDTPIPHQRRSIECCTE |
| | | RNECNKDLHPTLPPLKNRDFV____ |
| 214 | SEQ ID NO: 213 with | KKEDGESTAPTPRPKVLRCKCHHHCPEDSINNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | V30I, I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 215 | SEQ ID NO: 213 with | KKEDGESTAPTPRPKVLRCKCHHHCPEDSINNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | V30I, I33T, S35I | |
| 216 | SEQ ID NO: 213 with | KKEDGESTAPTPRPKVLRCKCHHHCPEDSINNTCITDGY CFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | V30I, I33T, S35I | |
| 217 | SEQ ID NO: 213 with | KKEDGESTAPTPRPKVLRCKCHHHCPEDSINNTCITDGY CFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | V30I, I33T, S35I | |
| 218 | SEQ ID NO: 218 | KKEDGESTAPTPRPKVLRCKCSHHCPEDSVNNTCITDGYCFT |
| | H22S, I33T, S35I | MIEEDDSGLPVVTSGCLGLEGSDFQCRDTPIPHQRRSIECCTE |
| | | RNECNKDLHPTLPPLKNRDFV____ |
| 219 | SEQ ID NO: 218 with | KKEDGESTAPTPRPKVLRCKCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | H22S, I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 220 | SEQ ID NO: 218 with | KKEDGESTAPTPRPKVLRCKCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | H22S, I33T, S35I | |
| 221 | SEQ ID NO: 218 with | KKEDGESTAPTPRPKVLRCKCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | H22S, I33T, S35I | |
| 222 | SEQ ID NO: 218 with | KKEDGESTAPTPRPKVLRCKCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | H22S, I33T, S35I | |
| 223 | SEQ ID NO: 223 | KKEDGESTAPTPRPKVLRCKCSHHCPEDSINNTCSTDGYCFT |
| | H22S, V30I, I33T | MIEEDDSGLPVVTSGCLGLEGSDFQCRDTPIPHQRRSIECCTE |
| | | RNECNKDLHPTLPPLKNRDFV____ |
| 224 | SEQ ID NO: 223 with | KKEDGESTAPTPRPKVLRCKCSHHCPEDSINNTCSTDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV___TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | H22S, V30I, I33T | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 225 | SEQ ID NO: 223 with | KKEDGESTAPTPRPKVLRCKCSHHCPEDSINNTCSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | H22S, V301, I33T | |
| 226 | SEQ ID NO: 223 with | KKEDGESTAPTPRPKVLRCKCSHHCPEDSINNTCSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | H22S, V30I, I33T | |
| 227 | SEQ ID NO: 223 with | KKEDGESTAPTPRPKVLRCKCSHHCPEDSINNTCSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | H22S, V30I, I33T | |
| 228 | SEQ ID NO: 228 | KKEDGESTAPTPRPKVLRCKCSHHCPEDSVNNTCSTDGYCFT |
| | H22S, I33T | MIEEDDSGLPVVTSGCLGLEGSDFQCRDTPIPHQRRSIECCTE |
| | | RNECNKDLHPTLPPLKNRDFV____ |
| 229 | SEQ ID NO: 228 with | KKEDGESTAPTPRPKVLRCKCSHHCPEDSVNNTCSTDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | H22S, I33T | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 230 | SEQ ID NO: 228 with | KKEDGESTAPTPRPKVLRCKCSHHCPEDSVNNTCSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | H22S, I33T | |
| 231 | SEQ ID NO: 228 with | KKEDGESTAPTPRPKVLRCKCSHHCPEDSVNNTCSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | H22S, I33T | |
| 232 | SEQ ID NO: 228 with | KKEDGESTAPTPRPKVLRCKCSHHCPEDSVNNTCSTDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | H22S, I33T | |
| 233 | SEQ ID NO: 233 | KKEDGESTAPTPRPKVLRCKCHHHCPEDSVNNTCITDGYCFT |
| | I33T, S35I | MIEEDDSGLPVVTSGCLGLEGSDFQCRDTPIPHQRRSIECCTE |
| | | RNECNKDLHPTLPPLKNRDFV____ |
| 234 | SEQ ID NO: 233 with | KKEDGESTAPTPRPKVLRCKCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 235 | SEQ ID NO: 233 with | KKEDGESTAPTPRPKVLRCKCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | I33T, S35I | |
| 236 | SEQ ID NO: 233 with | KKEDGESTAPTPRPKVLRCKCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | I33T, S35I | |
| 237 | SEQ ID NO: 233 with | KKEDGESTAPTPRPKVLRCKCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | I33T, S35I | |
| 238 | SEQ ID NO: 137 with | KKEDGESTAPTPRPKVLRCKCHHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 239 | SEQ ID NO: 137 with | KKEDGESTAPTPRPKVLRCKCHHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | S35I | |
| 240 | SEQ ID NO: 137 with | KKEDGESTAPTPRPKVLRCKCHHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | S35I | |
| 241 | SEQ ID NO: 137 with | KKEDGESTAPTPRPKVLRCKCHHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | S35I | |
| 242 | SEQ ID NO: 242 | KKEDGESTAPTPRPKVLRCFCHHHCPEDSVNNTCITDGYCFT |
| | K20F, I33T, S35I | MIEEDDSGLPVVTSGCLGLEGSDFQCRDTPIPHQRRSIECCTE |
| | | RNECNKDLHPTLPPLKNRDFV____ |
| 243 | SEQ ID NO: 242 with | KKEDGESTAPTPRPKVLRCFCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | K20F, I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 244 | SEQ ID NO: 242 with | KKEDGESTAPTPRPKVLRCFCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | K20F, I33T, S35I | |
| 245 | SEQ ID NO: 242 with | KKEDGESTAPTPRPKVLRCFCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | K20F, I33T, S35I | |
| 246 | SEQ ID NO: 242 with | KKEDGESTAPTPRPKVLRCFCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | K20F, I33T, S35I | |
| 247 | SEQ ID NO: 247 | KKEDGESTAPTPRPKVLRCKCSHHCPEDSVNNICITDGYCFT |
| | H22S, S35I | MIEEDDSGLPVVTSGCLGLEGSDFQCRDTPIPHQRRSIECCTE |
| | | RNECNKDLHPTLPPLKNRDFV____ |
| 248 | SEQ ID NO: 247 with | KKEDGESTAPTPRPKVLRCKCSHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | H22S, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 249 | SEQ ID NO: 247 with | KKEDGESTAPTPRPKVLRCKCSHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | H22S, S35I | |
| 250 | SEQ ID NO: 247 with | KKEDGESTAPTPRPKVLRCKCSHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | H22S, S35I | |
| 251 | SEQ ID NO: 247 with | KKEDGESTAPTPRPKVLRCKCSHHCPEDSVNNICITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | H22S, S35I | |
| 252 | SEQ ID NO: 252 | KKEDGESTAPTPRPKVLRCFCSHHCPEDSVNNICITDGYCFT |
| | K20F, H22S, I33T, | MIEEDDSGLPVVTSGCLGLEGSDFQCRDTPIPHQRRSIECCTE |
| | S35I | RNECNKDLHPTLPPLKNRDFV____ |
| 253 | SEQ ID NO: 252 with | KKEDGESTAPTPRPKVLRCFCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | K20F, H22S, I33T, | TYRVVSVLTVLHQDWLNGKEYKCKVSNKALPAPIEKTISKA |
| | S35I | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 254 | SEQ ID NO: 252 with | KKEDGESTAPTPRPKVLRCFCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | K20F, H22S, I33T, | |
| | S35I | |
| 255 | SEQ ID NO: 252 with | KKEDGESTAPTPRPKVLRCFCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | K20F, H22S, I33T, | |
| | S35I | |
| 256 | SEQ ID NO: 252 with | KKEDGESTAPTPRPKVLRCFCSHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | K20F, H22S, I33T, | |
| | S35I | |
| 257 | SEQ ID NO: 257 | KKEDGESTAPTPRPKVLRCICHHHCPEDSVNNTCITDGYCFT |
| | K201, I33T, S35I | MIEEDDSGLPVVTSGCLGLEGSDFQCRDTPIPHQRRSIECCTE |
| | | RNECNKDLHPTLPPLKNRDFV____ |
| 258 | SEQ ID NO: 257 with | KKEDGESTAPTPRPKVLRCICHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | K201, I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 259 | SEQ ID NO: 257 with | KKEDGESTAPTPRPKVLRCICHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | K201, I33T, S35I | |
| 260 | SEQ ID NO: 257 with | KKEDGESTAPTPRPKVLRCICHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | K201, I33T, S35I | |
| 261 | SEQ ID NO: 257 with | KKEDGESTAPTPRPKVLRCICHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | |
| | (SEQ ID NO: 201) | |
| | K20I, I33T, S35I | RNECNKDLHPTLPPLKNRDFV____TGGGGSGGGGSGGG |
| | | GSGGGGSPTIKPCPPCKCPAP |
| 262 | SEQ ID NO: 262 | KKEDGESTAPTPRPKVLRCACHHHCPEDSVNNTCITDGYCFT |
| | K20A, I33T, S35I | MIEEDDSGLPVVTSGCLGLEGSDFQCRDTPIPHQRRSIECCTE |
| | | RNECNKDLHPTLPPLKNRDFV____ |
| 263 | SEQ ID NO: 262 with | KKEDGESTAPTPRPKVLRCACHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | K20A, I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 264 | SEQ ID NO: 262 with | KKEDGESTAPTPRPKVLRCACHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | K20A, I33T, S35I | |
| 265 | SEQ ID NO: 262 with | KKEDGESTAPTPRPKVLRCACHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | K20A, I33T, S35I | |
| 266 | SEQ ID NO: 262 with | KKEDGESTAPTPRPKVLRCACHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | K20A, I33T, S35I | |
| 267 | SEQ ID NO: 267 | KKEDGESTAPTPRPKVLRCTCHHHCPEDSVNNTCITDGYCFT |
| | K20T, I33T, S35I | MIEEDDSGLPVVTSGCLGLEGSDFQCRDTPIPHQRRSIECCTE |
| | | RNECNKDLHPTLPPLKNRDFV____ |
| 268 | SEQ ID NO: 267 with | KKEDGESTAPTPRPKVLRCTCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | K20T, I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 269 | SEQ ID NO: 267 with | KKEDGESTAPTPRPKVLRCTCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | K20T, I33T, S35I | |
| 270 | SEQ ID NO: 267 with | KKEDGESTAPTPRPKVLRCTCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | K20T, I33T, S35I | |
| 271 | SEQ ID NO: 267 with | KKEDGESTAPTPRPKVLRCTCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | K20T, I33T, S35I | |
| 272 | SEQ ID NO: 272 | KKEDGESTAPTPRPKVLRCVCHHHCPEDSVNNTCITDGYCFT |
| | K20V, I33T, S35I | MIEEDDSGLPVVTSGCLGLEGSDFQCRDTPIPHQRRSIECCTE |
| | | RNECNKDLHPTLPPLKNRDFV____ |
| 273 | SEQ ID NO: 272 with | KKEDGESTAPTPRPKVLRCVCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fc- | RNECNKDLHPTLPPLKNRDFV____TGGGGSGGGGSGGG |
| | domain (SEQ ID NO: | GSGGGGSTHTCPPCPAPEAAGGPSVFLFPPKPKDTLMISRTPE |
| | 106) | VTCVVVDVSHEDPEVKFNWYVDGVEVHNAKTKPREEQYNS |
| | K20V, I33T, S35I | TYRVVSVLTVLHQDWLNGKEYKCKVSNKALPAPIEKTISKA |
| | | KGQPREPQVYTLPPSREEMTKNQVSLTCLVKGFYPSDIAVE |
| | | WESNGQPENNYKTTPPVLDSDGSFFLYSKLTVDKSRWQQGN |
| | | VFSCSVMHEALHNHYTQKSLSLSPG |
| 274 | SEQ ID NO: 272 with | KKEDGESTAPTPRPKVLRCVCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 199) | GSGGGGSTHTCPPCPAPEAAGGP |
| | K20V, I33T, S35I | |
| 275 | SEQ ID NO: 272 with | KKEDGESTAPTPRPKVLRCVCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 200) | GSGGGGSTHTCPPCKCPEAAGGP |
| | K20V, I33T, S35I | |
| 276 | SEQ ID NO: 272 with | KKEDGESTAPTPRPKVLRCVCHHHCPEDSVNNTCITDGYCFT |
| | linker sequence (SEQ | MIEEDDSGLPVVTSGCLGLEGSDFQCRDTPIPHQRRSIECCTE |
| | ID NO: 6), and Fab | RNECNKDLHPTLPPLKNRDFV____TGGGGSGGGGSGGG |
| | (SEQ ID NO: 201) | GSGGGGSPTIKPCPPCKCPAP |
| | K20V, I33T, S35I | |
| 277 | SEQ ID NO: 277 | KKEDGESTAPTPRPKVLRCYCHHHCPEDSINNTCITDGYCFT |
| | K20Y, V30I, I33T, | MIEEDDSGLPVVTSGCLGLEGSDFQCRDTPIPHQRRSIECCTE |
| | S35I | RNECNKDLHPTLPPLKNRDFV____ |
| 278 | SEQ ID NO: 277 | KKEDGESTAPTPRPKVLRCYCHHHCPEDSINNTCITDGYCFT |
| | with linker sequence | MIEEDDSGLPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fc-domain (SEQ ID | GGSGGGGSTHTCPPCPAPEAAGGPSVFLFPPKPKDTLMISRT |
| | NO: 106) | PEVTCVVVDVSHEDPEVKFNWYVDGVEVHNAKTKPREEQY |
| | K20Y, V30I, I33T, | NSTYRVVSVLTVLHQDWLNGKEYKCKVSNKALPAPIEKTIS |
| | S35I | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 279 | SEQ ID NO: 277 | KKEDGESTAPTPRPKVLRCYCHHHCPEDSINNTCITDGYCFT |
| | with linker sequence | MIEEDDSGLPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSTHTCPPCPAPEAAGGP |
| | 199) | |
| | K20Y, V30I, I33T, | |
| | S35I | |
| 280 | SEQ ID NO: 277 | KKEDGESTAPTPRPKVLRCYCHHHCPEDSINNTCITDGYCFT |
| | with linker sequence | MIEEDDSGLPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSTHTCPPCKCPEAAGGP |
| | 200) | |
| | K20Y, V30I, I33T, | |
| | S35I | |
| 281 | SEQ ID NO: 277 | KKEDGESTAPTPRPKVLRCYCHHHCPEDSINNTCITDGYCFT |
| | with linker sequence | MIEEDDSGLPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSPTIKPCPPCKCPAP |
| | 201) | |
| | K20Y, V30I, I33T, | |
| | S35I | |
| 282 | SEQ ID NO: 136 | KKEDGESTAPTPRPKVLRCKCHHHCPEDSVNNTCSTDGYCF |
| | with linker sequence | TMIEEDDSGLPVVTSGCLGLEGSDFQCRDTPIPHQRRSIECCT |
| | (SEQ ID NO: 6) and | ERNECNKDLHPTLPPLKNRDFV____TGGGGSGGGGSGG |
| | Fc-domain (SEQ ID | GGSGGGGSTHTCPPCPAPEAAGGPSVFLFPPKPKDTLMISRT |
| | NO: 106) | PEVTCVVVDVSHEDPEVKFNWYVDGVEVHNAKTKPREEQY |
| | I33T | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 283 | SEQ ID NO: 283 full | KKEDGESTAPTPRPKVLRCKCHHHCPEDSVNNTCITDGYCFT |
| | length ALK6 (SEQ | MIEEDDSGLPVVTSGCLGLEGSDFQCRDTPIPHQRRSIECCTE |
| | ID NO: 1) with point | RNECNKDLHPTLPPLKNRDFFDGPIHHR |
| | mutation V106F | |
| | I33T, S35I, V106F | |
| 284 | SEQ ID NO: 283 | KKEDGESTAPTPRPKVLRCKCHHHCPEDSVNNTCITDGYCFT |
| | with linker sequence | MIEEDDSGLPVVTSGCLGLEGSDFQCRDTPIPHQRRSIECCTE |
| | (SEQ ID NO: 6) and | RNECNKDLHPTLPPLKNRDFFDGPIHHRTGGGGSGGGGSG |
| | Fc-domain (SEQ ID | GGGSGGGGSTHTCPPCPAPEAAGGPSVFLFPPKPKDTLMISR |
| | NO: 106) | TPEVTCVVVDVSHEDPEVKFNWYVDGVEVHNAKTKPREEQ |
| | I33T, S35I, V106F | YNSTYRVVSVLTVLHQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPG |
| 285 | SEQ ID NO: 106 | THTCPPCPAPEAAGGPSVFLFPPKPKDTLMASRTPEVTCVVV |
| | Human Fc IVT | DVSHEDPEVKFNWYVDGVEVHNAKTKPREEQYNSTYRVVS |
| | domain with point | VLTVLAQDWLNGKEYKCKVSNKALPAPIEKTISKAKGQPRE |
| | mutations I31A and | PQVYTLPPSREEMTKNQVSLTCLVKGFYPSDIAVEWESNGQP |
| | H88A | ENNYKTTPPVLDSDGSFFLYSKLTVDKSRWQQGNVFSCSVM |
| | | HEALHNHYTQKSLSLSPG |
| 286 | SEQ ID NO: 90 | KKEDGESTAPTPRPKILRCYCSAGHCPEDSVNNICSTDGYCF |
| | Variant ECD with C | TMIEEDDSGMPVVTSGCLGLEGSDFQCRDTPIPHQRRSIECCT |
| | terminus seven | ERNECNKDLHPTLPPLKNRDFV |
| | amino acid deletion | |
| | and point mutations | |
| | V16I and L51M | |
| 287 | SEQ ID NO: 286 | KKEDGESTAPTPRPKILRCYCSAGHCPEDSVNNICSTDGYCF |
| | with linker sequence | TMIEEDDSGMPVVTSGCLGLEGSDFQCRDTPIPHQRRSIECCT |
| | (SEQ ID NO: 6) and | ERNECNKDLHPTLPPLKNRDFV____TGGGGSGGGGSGG |
| | Human Fc sequence | GGSGGGGSTHTCPPCPAPEAAGGPSVFLFPPKPKDTLMISRT |
| | (SEQ ID NO: 106) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 288 | SEQ ID NO: 286 | KKEDGESTAPTPRPKILRCYCSAGHCPEDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSTHTCPPCPAPEAAGGP |
| | 199) | |
| 289 | SEQ ID NO: 286 | KKEDGESTAPTPRPKILRCYCSAGHCPEDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSTHTCPPCKCPEAAGGP |
| | 200) | |
| 290 | SEQ ID NO: 286 | KKEDGESTAPTPRPKILRCYCSAGHCPEDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSPTIKPCPPCKCPAP |
| | 201) | |
| 291 | SEQ ID NO: 286 | KKEDGESTAPTPRPKILRCYCSAGHCPEDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | IVT Fab (SEQ ID | GGSGGGGSTHTCPPCPAPEAAGGPSVFLFPPKPKDTLMASR |
| | NO: 285) | TPEVTCVVVDVSHEDPEVKFNWYVDGVEVHNAKTKPREEQ |
| | | YNSTYRVVSVLTVLAQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPG |
| 292 | SEQ ID NO: 90 | KKEDGESTAPTPRPKILRCYCSAGHCPDDSVNNICSTDGYCF |
| | Variant ECD with | TMIEEDDSGMPVVTSGCLGLEGSDFQCRDTPIPHQRRSIECCT |
| | point mutations | ERNECNKDLHPTLPPLKNRDFV____ |
| | V16I, E27D, and | |
| | L51M | |
| 293 | SEQ ID NO: 292 | KKEDGESTAPTPRPKILRCYCSAGHCPDDSVNNICSTDGYCF |
| | with linker sequence | TMIEEDDSGMPVVTSGCLGLEGSDFQCRDTPIPHQRRSIECCT |
| | (SEQ ID NO: 6) and | ERNECNKDLHPTLPPLKNRDFV____TGGGGSGGGGSGG |
| | Human Fc sequence | GGSGGGGSTHTCPPCPAPEAAGGPSVFLFPPKPKDTLMISRT |
| | (SEQ ID NO: 106) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 294 | SEQ ID NO: 292 | KKEDGESTAPTPRPKILRCYCSAGHCPDDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSTHTCPPCPAPEAAGGP |
| | 199) | |
| 295 | SEQ ID NO: 292 | KKEDGESTAPTPRPKILRCYCSAGHCPDDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSTHTCPPCKCPEAAGGP |
| | 200) | |
| 296 | SEQ ID NO: 292 | KKEDGESTAPTPRPKILRCYCSAGHCPDDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSPTIKPCPPCKCPAP |
| | 201) | |
| 297 | SEQ ID NO: 292 | KKEDGESTAPTPRPKILRCYCSAGHCPDDSVNNICSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSTHTCPPCPAPEAAGGPSVFLFPPKPKDTLMASR |
| | 285) | TPEVTCVVVDVSHEDPEVKFNWYVDGVEVHNAKTKPREEQ |
| | | YNSTYRVVSVLTVLAQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPG |
| 298 | SEQ ID NO: 90 | KKEDGESTAPTPRPKILRCYCSAGHCPEDSINNICSTDGYCFT |
| | Variant ECD with | MIEEDDSGMPVVTSGCLGLEGSDFQCRDTPIPHQRRSIECCTE |
| | point mutations | RNECNKDLHPTLPPLKNRDFV____ |
| | V16I, V30I, and | |
| | L51M | |
| 299 | SEQ ID NO: 298 | KKEDGESTAPTPRPKILRCYCSAGHCPEDSINNICSTDGYCFT |
| | with linker sequence | MIEEDDSGMPVVTSGCLGLEGSDFQCRDTPIPHQRRSIECCTE |
| | (SEQ ID NO: 6) and | RNECNKDLHPTLPPLKNRDFV____TGGGGSGGGGSGG |
| | Human Fc sequence | GGSGGGGSTHTCPPCPAPEAAGGPSVFLFPPKPKDTLMISRT |
| | (SEQ ID NO: 106) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 300 | SEQ ID NO: 298 | KKEDGESTAPTPRPKILRCYCSAGHCPEDSINNICSTDGYCFT |
| | with linker sequence | MIEEDDSGMPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSTHTCPPCPAPEAAGGP |
| | 199) | |
| 301 | SEQ ID NO: 298 | KKEDGESTAPTPRPKILRCYCSAGHCPEDSINNICSTDGYCFT |
| | with linker sequence | MIEEDDSGMPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSTHTCPPCKCPEAAGGP |
| | 200) | |
| 302 | SEQ ID NO: 298 | KKEDGESTAPTPRPKILRCYCSAGHCPEDSINNICSTDGYCFT |
| | with linker sequence | MIEEDDSGMPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSPTIKPCPPCKCPAP |
| | 201) | |
| 303 | SEQ ID NO: 298 | KKEDGESTAPTPRPKILRCYCSAGHCPEDSINNICSTDGYCFT |
| | with linker sequence | MIEEDDSGMPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSTHTCPPCPAPEAAGGPSVFLFPPKPKDTLMASR |
| | 285) | TPEVTCVVVDVSHEDPEVKFNWYVDGVEVHNAKTKPREEQ |
| | | YNSTYRVVSVLTVLAQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPG |
| 304 | SEQ ID NO: 90 | KKEDGESTAPTPRPKILRCYCSAGHCPEDSVNNTCSTDGYCF |
| | Variant ECD with | TMIEEDDSGMPVVTSGCLGLEGSDFQCRDTPIPHQRRSIECCT |
| | point mutations | ERNECNKDLHPTLPPLKNRDFV____ |
| | V161, I33T, and | |
| | L51M | |
| 305 | SEQ ID NO: 304 | KKEDGESTAPTPRPKILRCYCSAGHCPEDSVNNTCSTDGYCF |
| | with linker sequence | TMIEEDDSGMPVVTSGCLGLEGSDFQCRDTPIPHQRRSIECCT |
| | (SEQ ID NO: 6) and | ERNECNKDLHPTLPPLKNRDFV____TGGGGSGGGGSGG |
| | Human Fc sequence | GGSGGGGSTHTCPPCPAPEAAGGPSVFLFPPKPKDTLMISRT |
| | (SEQ ID NO: 106) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 306 | SEQ ID NO: 304 | KKEDGESTAPTPRPKILRCYCSAGHCPEDSVNNTCSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSTHTCPPCPAPEAAGGP |
| | 199) | |
| 307 | SEQ ID NO: 304 | KKEDGESTAPTPRPKILRCYCSAGHCPEDSVNNTCSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSTHTCPPCKCPEAAGGP |
| | 200) | |
| 308 | SEQ ID NO: 304 | KKEDGESTAPTPRPKILRCYCSAGHCPEDSVNNTCSTDGYCF |
| | with linker sequence | TMIEEDDSGMPVVTSGCLGLEGSDFQCRDTPIPHQRRSIECCT |
| | (SEQ ID NO: 6), and | ERNECNKDLHPTLPPLKNRDFV____TGGGGSGGGGSGG |
| | Fab (SEQ ID NO: | GGSGGGGSPTIKPCPPCKCPAP |
| | 201) | |
| 309 | SEQ ID NO: 304 with | KKEDGESTAPTPRPKILRCYCSAGHCPEDSVNNTCSTDGYCF |
| | linker sequence (SEQ | TMIEEDDSGMPVVTSGCLGLEGSDFQCRDTPIPHQRRSIECCT |
| | ID NO: 6), and Fab | ERNECNKDLHPTLPPLKNRDFV____TGGGGSGGGGSGG |
| | (SEQ ID NO: 285) | GGSGGGGSTHTCPPCPAPEAAGGPSVFLFPPKPKDTLMASR |
| | | TPEVTCVVVDVSHEDPEVKFNWYVDGVEVHNAKTKPREEQ |
| | | YNSTYRVVSVLTVLAQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPG |
| 310 | SEQ ID NO: 90 | KKEDGESTAPTPRPKILRCYCSAGHCPEDSVNNICITDGYCFT |
| | Variant ECD with | MIEEDDSGMPVVTSGCLGLEGSDFQCRDTPIPHQRRSIECCTE |
| | point mutations | RNECNKDLHPTLPPLKNRDFV____ |
| | V16I, S35I, and | |
| | L51M | |
| 311 | SEQ ID NO: 310 | KKEDGESTAPTPRPKILRCYCSAGHCPEDSVNNICITDGYCFT |
| | with linker sequence | MIEEDDSGMPVVTSGCLGLEGSDFQCRDTPIPHQRRSIECCTE |
| | (SEQ ID NO:6) and | RNECNKDLHPTLPPLKNRDFV____TGGGGSGGGGSGG |
| | Human Fc sequence | GGSGGGGSTHTCPPCPAPEAAGGPSVFLFPPKPKDTLMISRT |
| | (SEQ ID NO: 106) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 312 | SEQ ID NO: 310 | KKEDGESTAPTPRPKILRCYCSAGHCPEDSVNNICITDGYCFT |
| | with linker sequence | MIEEDDSGMPVVTSGCLGLEGSDFQCRDTPIPHQRRSIECCTE |
| | (SEQ ID NO:6), and | RNECNKDLHPTLPPLKNRDFV____TGGGGSGGGGSGG |
| | Fab (SEQ ID NO: | GGSGGGGSTHTCPPCPAPEAAGGP |
| | 199) | |
| 313 | SEQ ID NO: 310 | KKEDGESTAPTPRPKILRCYCSAGHCPEDSVNNICITDGYCFT |
| | with linker sequence | MIEEDDSGMPVVTSGCLGLEGSDFQCRDTPIPHQRRSIECCTE |
| | (SEQ ID NO:6), and | RNECNKDLHPTLPPLKNRDFV____TGGGGSGGGGSGG |
| | Fab (SEQ ID NO: | GGSGGGGSTHTCPPCKCPEAAGGP |
| | 200) | |
| 314 | SEQ ID NO: 310 | KKEDGESTAPTPRPKILRCYCSAGHCPEDSVNNICITDGYCFT |
| | with linker sequence | MIEEDDSGMPVVTSGCLGLEGSDFQCRDTPIPHQRRSIECCTE |
| | (SEQ ID NO:6), and | RNECNKDLHPTLPPLKNRDFV____TGGGGSGGGGSGG |
| | Fab (SEQ ID NO: | GGSGGGGSPTIKPCPPCKCPAP |
| | 201) | |
| 315 | SEQ ID NO: 310 | KKEDGESTAPTPRPKILRCYCSAGHCPEDSVNNICITDGYCFT |
| | with linker sequence | MIEEDDSGMPVVTSGCLGLEGSDFQCRDTPIPHQRRSIECCTE |
| | (SEQ ID NO: 6), and | RNECNKDLHPTLPPLKNRDFV____TGGGGSGGGGSGG |
| | Fab (SEQ ID NO: | GGSGGGGSTHTCPPCPAPEAAGGPSVFLFPPKPKDTLMASR |
| | 285) | TPEVTCVVVDVSHEDPEVKFNWYVDGVEVHNAKTKPREEQ |
| | | YNSTYRVVSVLTVLAQDWLNGKEYKCKVSNKALPAPIEKTI |
| | | SKAKGQPREPQVYTLPPSREEMTKNQVSLTCLVKGFYPSDIA |
| | | VEWESNGQPENNYKTTPPVLDSDGSFFLYSKLTVDKSRWQQ |
| | | GNVFSCSVMHEALHNHYTQKSLSLSPG |
| 316 | SEQ ID NO: 90 | KKEDGESTAPTPRPKILRCYCSAGHCPEDSVNNICSTNGYCF |
| | Variant ECD with | TMIEEDDSGMPVVTSGCLGLEGSDFQCRDTPIPHQRRSIECCT |
| | point mutations | ERNECNKDLHPTLPPLKNRDFV____ |
| | V16I, D37N, and | |
| | L51M | |
| 317 | SEQ ID NO: 316 | KKEDGESTAPTPRPKILRCYCSAGHCPEDSVNNICSTNGYCF |
| | with linker sequence | TMIEEDDSGMPVVTSGCLGLEGSDFQCRDTPIPHQRRSIECCT |
| | (SEQ ID NO: 6) and | ERNECNKDLHPTLPPLKNRDFV____TGGGGSGGGGSGG |
| | Human Fc sequence | GGSGGGGSTHTCPPCPAPEAAGGPSVFLFPPKPKDTLMISRT |
| | (SEQ ID NO: 106) | PEVTCVVVDVSHEDPEVKFNWYVDGVEVHNAKTKPREEQY |
| | | NSTYRVVSVLTVLHQDWLNGKEYKCKVSNKALPAPIEKTIS |
| | | KAKGQPREPQVYTLPPSREEMTKNQVSLTCLVKGFYPSDIAV |
| | | EWESNGQPENNYKTTPPVLDSDGSFFLYSKLTVDKSRWQQG |
| | | NVFSCSVMHEALHNHYTQKSLSLSPG |
| 318 | SEQ ID NO: 316 | KKEDGESTAPTPRPKILRCYCSAGHCPEDSVNNICSTNGYCF |
| | with linker sequence | TMIEEDDSGMPVVTSGCLGLEGSDFQCRDTPIPHQRRSIECCT |
| | (SEQ ID NO: 6), with | ERNECNKDLHPTLPPLKNRDFV____TGGGGSGGGGSGG |
| | Fab (SEQ ID NO: | GGSGGGGSTHTCPPCPAPEAAGGP |
| | 199) | |
| 319 | SEQ ID NO: 316 | KKEDGESTAPTPRPKILRCYCSAGHCPEDSVNNICSTNGYCF |
| | with linker sequence | TMIEEDDSGMPVVTSGCLGLEGSDFQCRDTPIPHQRRSIECCT |
| | (SEQ ID NO: 6), with | ERNECNKDLHPTLPPLKNRDFV____TGGGGSGGGGSGG |
| | Fab (SEQ ID NO: | GGSGGGGSTHTCPPCKCPEAAGGP |
| | 200) | |
| 320 | SEQ ID NO: 316 | KKEDGESTAPTPRPKILRCYCSAGHCPEDSVNNICSTNGYCFTMI |
| | with linker sequence | EEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 321 | SEQ ID NO: 316 | KKEDGESTAPTPRPKILRCYCSAGHCPEDSVNNICSTNGYCFTMI |
| | with linker sequence | EEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 322 | SEQ ID NO: 90 | KKEDGESTAPTPRPKILRCYCSAGHCPEDSVNNICSTDGHCFTM |
| | Variant ECD with | IEEDDSGMPVVTSGCLGLEGSDFQCRDTPIPHQRRSIECCTERNE |
| | point mutations | CNKDLHPTLPPLKNRDFV____ |
| | V16I, Y39H, and | |
| | L51M | |
| 323 | SEQ ID NO: 322 | KKEDGESTAPTPRPKILRCYCSAGHCPEDSVNNICSTDGHCFTM |
| | with linker sequence | IEEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 324 | SEQ ID NO: 322 | KKEDGESTAPTPRPKILRCYCSAGHCPEDSVNNICSTDGHCFTM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGGGGGSGG |
| | Fab (SEQ ID NO: | GGSTHTCPPCPAPEAAGGP |
| | 199) | |
| 325 | SEQ ID NO: 322 | KKEDGESTAPTPRPKILRCYCSAGHCPEDSVNNICSTDGHCFTM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 326 | SEQ ID NO: 322 | KKEDGESTAPTPRPKILRCYCSAGHCPEDSVNNICSTDGHCFTM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 327 | SEQ ID NO: 322 | KKEDGESTAPTPRPKILRCYCSAGHCPEDSVNNICSTDGHCFTM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 328 | SEQ ID NO: 90 | KKEDGESTAPTPRPKILRCYCSAGHCPEDSVNNICSTDGYCFAM |
| | Variant ECD with | IEEDDSGMPVVTSGCLGLEGSDFQCRDTPIPHQRRSIECCTERNE |
| | point mutations | CNKDLHPTLPPLKNRDFV |
| | V16I, T42A, and | |
| | L51M | |
| 329 | SEQ ID NO: 328 | KKEDGESTAPTPRPKILRCYCSAGHCPEDSVNNICSTDGYCFAM |
| | with linker sequence | IEEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 330 | SEQ ID NO: 328 | KKEDGESTAPTPRPKILRCYCSAGHCPEDSVNNICSTDGYCFAM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 331 | SEQ ID NO: 328 | KKEDGESTAPTPRPKILRCYCSAGHCPEDSVNNICSTDGYCFAM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 332 | SEQ ID NO: 328 | KKEDGESTAPTPRPKILRCYCSAGHCPEDSVNNICSTDGYCFAM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 333 | SEQ ID NO: 328 | KKEDGESTAPTPRPKILRCYCSAGHCPEDSVNNICSTDGYCFAM |
| | with linker sequence | IEEDDSGMPVVTSGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 334 | SEQ ID NO: 90 | KKEDGESTAPTPRPKILRCYCSAGHCPEDSVNNICSTDGYCFTII |
| | Variant ECD with | EEDDSGMPVVTSGCLGLEGSDFQCRDTPIPHQRRSIECCTERNE |
| | point mutations | CNKDLHPTLPPLKNRDFV |
| | V16I, M43I, and | |
| | L51M | |
| 335 | SEQ ID NO: 334 | KKEDGESTAPTPRPKILRCYCSAGHCPEDSVNNICSTDGYCFTII |
| | with linker sequence | EEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 336 | SEQ ID NO: 334 with | KKEDGESTAPTPRPKILRCYCSAGHCPEDSVNNICSTDGYCFTII |
| | linker sequence (SEQ | EEDDSGMPVVTSGCLGLEGSDFQCRDTPIPHQRRSIECCTERNE |
| | ID NO: 6), with Fab | CNKDLHPTLPPLKNRDFV____TGGGGSGGGGSGGGGSGG |
| | (SEQ ID NO: 199) | GGSTHTCPPCPAPEAAGGP |
| 337 | SEQ ID NO: 334 with | KKEDGESTAPTPRPKILRCYCSAGHCPEDSVNNICSTDGYCFTII |
| | linker sequence (SEQ | EEDDSGMPVVTSGCLGLEGSDFQCRDTPIPHQRRSIECCTERNE |
| | | CNKDLHPTLPPLKNRDFV____TGGGGSGGGGSGGGGSGG |
| | ID NO: 6), with Fab | GGSTHTCPPCKCPEAAGGP |
| | (SEQ ID NO: 200) | |
| 338 | SEQ ID NO: 334 with | KKEDGESTAPTPRPKILRCYCSAGHCPEDSVNNICSTDGYCFTII |
| | linker sequence (SEQ | EEDDSGMPVVTSGCLGLEGSDFQCRDTPIPHQRRSIECCTERNE |
| | ID NO: 6), with Fab | CNKDLHPTLPPLKNRDFV____TGGGGSGGGGSGGGGSGG |
| | (SEQ ID NO: 201) | GGSPTIKPCPPCKCPAP |
| 339 | SEQ ID NO: 334 with | KKEDGESTAPTPRPKILRCYCSAGHCPEDSVNNICSTDGYCFTII |
| | linker sequence (SEQ | EEDDSGMPVVTSGCLGLEGSDFQCRDTPIPHQRRSIECCTERNE |
| | ID NO: 6), with Fab | CNKDLHPTLPPLKNRDFV____TGGGGSGGGGSGGGGSGG |
| | (SEQ ID NO: 285) | GGSTHTCPPCPAPEAAGGPSVFLFPPKPKDTLMASRTPEVTCVV |
| | | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 340 | SEQ ID NO: 90 | KKEDGESTAPTPRPKILRCYCSAGHCPEDSVNNICSTDGYCFTMI |
| | Variant ECD with | EEDDSGMPVVASGCLGLEGSDFQCRDTPIPHQRRSIECCTERNE |
| | point mutations | CNKDLHPTLPPLKNRDFV____ |
| | V16I, L51M, and | |
| | T55A | |
| 341 | SEQ ID NO: 340 | KKEDGESTAPTPRPKILRCYCSAGHCPEDSVNNICSTDGYCFTMI |
| | with linker sequence | EEDDSGMPVVASGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 342 | SEQ ID NO: 340 | KKEDGESTAPTPRPKILRCYCSAGHCPEDSVNNICSTDGYCFTMI |
| | with linker sequence | EEDDSGMPVVASGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 343 | SEQ ID NO: 340 | KKEDGESTAPTPRPKILRCYCSAGHCPEDSVNNICSTDGYCFTMI |
| | with linker sequence | EEDDSGMPVVASGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 344 | SEQ ID NO: 340 | KKEDGESTAPTPRPKILRCYCSAGHCPEDSVNNICSTDGYCFTMI |
| | with linker sequence | EEDDSGMPVVASGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 345 | SEQ ID NO: 340 | KKEDGESTAPTPRPKILRCYCSAGHCPEDSVNNICSTDGYCFTMI |
| | with linker sequence | EEDDSGMPVVASGCLGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 346 | SEQ ID NO: 90 | KKEDGESTAPTPRPKILRCYCSAGHCPEDSVNNICSTDGYCFTMI |
| | Variant ECD with | EEDDSGMPVVTSGCMGLEGSDFQCRDTPIPHQRRSIECCTERNE |
| | point mutations | CNKDLHPTLPPLKNRDFV |
| | V16I, L51M, and | |
| | L59M | |
| 347 | SEQ ID NO: 346 | KKEDGESTAPTPRPKILRCYCSAGHCPEDSVNNICSTDGYCFTMI |
| | with linker sequence | EEDDSGMPVVTSGCMGLEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 348 | SEQ ID NO: 346 | KKEDGESTAPTPRPKILRCYCSAGHCPEDSVNNICSTDGYCFTMI |
| | with linker sequence | EEDDSGMPVVTSGCMGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 349 | SEQ ID NO: 346 | KKEDGESTAPTPRPKILRCYCSAGHCPEDSVNNICSTDGYCFTMI |
| | with linker sequence | EEDDSGMPVVTSGCMGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 350 | SEQ ID NO: 346 | KKEDGESTAPTPRPKILRCYCSAGHCPEDSVNNICSTDGYCFTMI |
| | with linker sequence | EEDDSGMPVVTSGCMGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 351 | SEQ ID NO: 346 | KKEDGESTAPTPRPKILRCYCSAGHCPEDSVNNICSTDGYCFTMI |
| | with linker sequence | EEDDSGMPVVTSGCMGLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 352 | SEQ ID NO: 90 | KKEDGESTAPTPRPKILRCYCSAGHCPEDSVNNICSTDGYCFTMI |
| | Variant ECD with | EEDDSGMPVVTSGCLKLEGSDFQCRDTPIPHQRRSIECCTERNE |
| | point mutations | CNKDLHPTLPPLKNRDFV____ |
| | V16I, L51M, and | |
| | G60K | |
| 353 | SEQ ID NO: 352 | KKEDGESTAPTPRPKILRCYCSAGHCPEDSVNNICSTDGYCFTMI |
| | with linker sequence | EEDDSGMPVVTSGCLKLEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 354 | SEQ ID NO: 352 | KKEDGESTAPTPRPKILRCYCSAGHCPEDSVNNICSTDGYCFTMI |
| | with linker sequence | EEDDSGMPVVTSGCLKLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 355 | SEQ ID NO: 352 | KKEDGESTAPTPRPKILRCYCSAGHCPEDSVNNICSTDGYCFTMI |
| | with linker sequence | EEDDSGMPVVTSGCLKLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 356 | SEQ ID NO: 352 | KKEDGESTAPTPRPKILRCYCSAGHCPEDSVNNICSTDGYCFTMI |
| | with linker sequence | EEDDSGMPVVTSGCLKLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 357 | SEQ ID NO: 352 | KKEDGESTAPTPRPKILRCYCSAGHCPEDSVNNICSTDGYCFTMI |
| | with linker sequence | EEDDSGMPVVTSGCLKLEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 358 | SEQ ID NO: 90 | KKEDGESTAPTPRPKILRCYCSAGHCPEDSVNNICSTDGYCFTMI |
| | Variant ECD with | EEDDSGMPVVTSGCLGYEGSDFQCRDTPIPHQRRSIECCTERNE |
| | point mutations | CNKDLHPTLPPLKNRDFV____ |
| | V16I, L51M, and | |
| | L61Y | |
| 359 | SEQ ID NO: 358 | KKEDGESTAPTPRPKILRCYCSAGHCPEDSVNNICSTDGYCFTMI |
| | with linker sequence | EEDDSGMPVVTSGCLGYEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 360 | SEQ ID NO: 358 | KKEDGESTAPTPRPKILRCYCSAGHCPEDSVNNICSTDGYCFTMI |
| | with linker sequence | EEDDSGMPVVTSGCLGYEGSDFQCRDTPIPHQRRSIECCTERNE |
| | (SEQ ID NO: 6), with | CNKDLHPTLPPLKNRDFV TGGGGSGGGGSGGGGSGG |
| | Fab (SEQ ID NO: | GGSTHTCPPCPAPEAAGGP |
| | 199) | |
| 361 | SEQ ID NO: 358 | KKEDGESTAPTPRPKILRCYCSAGHCPEDSVNNICSTDGYCFTMI |
| | with linker sequence | EEDDSGMPVVTSGCLGYEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 362 | SEQ ID NO: 358 | KKEDGESTAPTPRPKILRCYCSAGHCPEDSVNNICSTDGYCFTMI |
| | with linker sequence | EEDDSGMPVVTSGCLGYEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 363 | SEQ ID NO: 358 | KKEDGESTAPTPRPKILRCYCSAGHCPEDSVNNICSTDGYCFTMI |
| | with linker sequence | EEDDSGMPVVTSGCLGYEGSDFQCRDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 364 | SEQ ID NO: 90 | KKEDGESTAPTPRPKILRCYCSAGHCPEDSVNNICSTDGYCFTMI |
| | Variant ECD with | EEDDSGMPVVTSGCLGLEGSDFQCKDTPIPHQRRSIECCTERNE |
| | point mutations | CNKDLHPTLPPLKNRDFV |
| | V16I, L51M, and | |
| | R68K | |
| 365 | SEQ ID NO: 364 | KKEDGESTAPTPRPKILRCYCSAGHCPEDSVNNICSTDGYCFTMI |
| | with linker sequence | EEDDSGMPVVTSGCLGLEGSDFQCKDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 366 | SEQ ID NO: 364 | KKEDGESTAPTPRPKILRCYCSAGHCPEDSVNNICSTDGYCFTMI |
| | with linker sequence | EEDDSGMPVVTSGCLGLEGSDFQCKDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 367 | SEQ ID NO: 364 | KKEDGESTAPTPRPKILRCYCSAGHCPEDSVNNICSTDGYCFTMI |
| | with linker sequence | EEDDSGMPVVTSGCLGLEGSDFQCKDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 368 | SEQ ID NO: 364 | KKEDGESTAPTPRPKILRCYCSAGHCPEDSVNNICSTDGYCFTMI |
| | with linker sequence | EEDDSGMPVVTSGCLGLEGSDFQCKDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 369 | SEQ ID NO: 364 | KKEDGESTAPTPRPKILRCYCSAGHCPEDSVNNICSTDGYCFTMI |
| | with linker sequence | EEDDSGMPVVTSGCLGLEGSDFQCKDTPIPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 370 | SEQ ID NO: 90 | KKEDGESTAPTPRPKILRCYCSAGHCPEDSVNNICSTDGYCFTMI |
| | Variant ECD with | EEDDSGMPVVTSGCLGLEGSDFQCRDSPIPHQRRSIECCTERNEC |
| | point mutations | NKDLHPTLPPLKNRDFV |
| | V16I, L51M, and | |
| | T71S | |
| 371 | SEQ ID NO: 371 | KKEDGESTAPTPRPKILRCYCSAGHCPEDSVNNICSTDGYCFTMI |
| | with linker sequence | EEDDSGMPVVTSGCLGLEGSDFQCRDSPIPHQRRSIECCTERNEC |
| | (SEQ ID NO: 6) and | NKDLHPTLPPLKNRDFV____TGGGGSGGGGSGGGGSGGG |
| | Human Fc sequence | GSTHTCPPCPAPEAAGGPSVFLFPPKPKDTLMISRTPEVTCVVVD |
| | (SEQ ID NO: 106) | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 372 | SEQ ID NO: 371 | KKEDGESTAPTPRPKILRCYCSAGHCPEDSVNNICSTDGYCFTMI |
| | with linker sequence | EEDDSGMPVVTSGCLGLEGSDFQCRDSPIPHQRRSIECCTERNEC |
| | (SEQ ID NO: 6), with | NKDLHPTLPPLKNRDFV____TGGGGSGGGGSGGGGSGGG |
| | Fab (SEQ ID NO: | GSTHTCPPCPAPEAAGGP |
| | 199) | |
| 373 | SEQ ID NO: 371 | KKEDGESTAPTPRPKILRCYCSAGHCPEDSVNNICSTDGYCFTMI |
| | with linker sequence | EEDDSGMPVVTSGCLGLEGSDFQCRDSPIPHQRRSIECCTERNEC |
| | (SEQ ID NO: 6), with | NKDLHPTLPPLKNRDFV____TGGGGSGGGGSGGGGSGGG |
| | Fab (SEQ ID NO: | GSTHTCPPCKCPEAAGGP |
| | 200) | |
| 374 | SEQ ID NO: 371 | KKEDGESTAPTPRPKILRCYCSAGHCPEDSVNNICSTDGYCFTMI |
| | with linker sequence | EEDDSGMPVVTSGCLGLEGSDFQCRDSPIPHQRRSIECCTERNEC |
| | (SEQ ID NO: 6), with | NKDLHPTLPPLKNRDFV____TGGGGSGGGGSGGGGSGGG |
| | Fab (SEQ ID NO: | GSPTIKPCPPCKCPAP |
| | 201) | |
| 375 | SEQ ID NO: 371 | KKEDGESTAPTPRPKILRCYCSAGHCPEDSVNNICSTDGYCFTMI |
| | with linker sequence | EEDDSGMPVVTSGCLGLEGSDFQCRDSPIPHQRRSIECCTERNEC |
| | (SEQ ID NO: 6), with | NKDLHPTLPPLKNRDFV____TGGGGSGGGGSGGGGSGGG |
| | Fab (SEQ ID NO: | GSTHTCPPCPAPEAAGGPSVFLFPPKPKDTLMASRTPEVTCVVV |
| | 285) | DVSHEDPEVKFNWYVDGVEVHNAKTKPREEQYNSTYRVVSVL |
| | | TVLAQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 376 | SEQ ID NO: 90 | KKEDGESTAPTPRPKILRCYCSAGHCPEDSVNNICSTDGYCFTMI |
| | Variant ECD with | EEDDSGMPVVTSGCLGLEGSDFQCRDTPKPHQRRSIECCTERNE |
| | point mutations | CNKDLHPTLPPLKNRDFV____ |
| | V16I, L51M, and | |
| | I73K | |
| 377 | SEQ ID NO: 377 | KKEDGESTAPTPRPKILRCYCSAGHCPEDSVNNICSTDGYCFTMI |
| | with linker sequence | EEDDSGMPVVTSGCLGLEGSDFQCRDTPKPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 378 | SEQ ID NO: 377 | KKEDGESTAPTPRPKILRCYCSAGHCPEDSVNNICSTDGYCFTMI |
| | with linker sequence | EEDDSGMPVVTSGCLGLEGSDFQCRDTPKPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 379 | SEQ ID NO: 377 | KKEDGESTAPTPRPKILRCYCSAGHCPEDSVNNICSTDGYCFTMI |
| | with linker sequence | EEDDSGMPVVTSGCLGLEGSDFQCRDTPKPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 380 | SEQ ID NO: 377 | KKEDGESTAPTPRPKILRCYCSAGHCPEDSVNNICSTDGYCFTMI |
| | with linker sequence | EEDDSGMPVVTSGCLGLEGSDFQCRDTPKPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 381 | SEQ ID NO: 377 | KKEDGESTAPTPRPKILRCYCSAGHCPEDSVNNICSTDGYCFTMI |
| | with linker sequence | EEDDSGMPVVTSGCLGLEGSDFQCRDTPKPHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 382 | SEQ ID NO: 90 | KKEDGESTAPTPRPKILRCYCSAGHCPEDSVNNICSTDGYCFTMI |
| | Variant ECD with | EEDDSGMPVVTSGCLGLEGSDFQCRDTPIAHQRRSIECCTERNE |
| | point mutations | CNKDLHPTLPPLKNRDFV____ |
| | V16I, L51M, and | |
| | P74A | |
| 383 | SEQ ID NO: 382 | KKEDGESTAPTPRPKILRCYCSAGHCPEDSVNNICSTDGYCFTMI |
| | with linker sequence | EEDDSGMPVVTSGCLGLEGSDFQCRDTPIAHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 384 | SEQ ID NO: 382 | KKEDGESTAPTPRPKILRCYCSAGHCPEDSVNNICSTDGYCFTMI |
| | with linker sequence | EEDDSGMPVVTSGCLGLEGSDFQCRDTPIAHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 385 | SEQ ID NO: 382 | KKEDGESTAPTPRPKILRCYCSAGHCPEDSVNNICSTDGYCFTMI |
| | with linker sequence | EEDDSGMPVVTSGCLGLEGSDFQCRDTPIAHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 386 | SEQ ID NO: 382 | KKEDGESTAPTPRPKILRCYCSAGHCPEDSVNNICSTDGYCFTMI |
| | with linker sequence | EEDDSGMPVVTSGCLGLEGSDFQCRDTPIAHQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 387 | SEQ ID NO: 382 | KKEDGESTAPTPRPKILRCYCSAGHCPEDSVNNICSTDGYCFTMI |
| | with linker sequence | EEDDSGMPVVTSGCLGLEGSDFQCRDTPIAHQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 388 | SEQ ID NO: 90 | KKEDGESTAPTPRPKILRCYCSAGHCPEDSVNNICSTDGYCFTMI |
| | Variant ECD with | EEDDSGMPVVTSGCLGLEGSDFQCRDTPIPQQRRSIECCTERNE |
| | point mutations | CNKDLHPTLPPLKNRDFV____ |
| | V16I, L51M, and | |
| | H75Q | |
| 389 | SEQ ID NO: 388 | KKEDGESTAPTPRPKILRCYCSAGHCPEDSVNNICSTDGYCFTMI |
| | with linker sequence | EEDDSGMPVVTSGCLGLEGSDFQCRDTPIPQQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 206) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 390 | SEQ ID NO: 388 | KKEDGESTAPTPRPKILRCYCSAGHCPEDSVNNICSTDGYCFTMI |
| | with linker sequence | EEDDSGMPVVTSGCLGLEGSDFQCRDTPIPQQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 391 | SEQ ID NO: 388 | KKEDGESTAPTPRPKILRCYCSAGHCPEDSVNNICSTDGYCFTMI |
| | with linker sequence | EEDDSGMPVVTSGCLGLEGSDFQCRDTPIPQQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 392 | SEQ ID NO: 388 | KKEDGESTAPTPRPKILRCYCSAGHCPEDSVNNICSTDGYCFTMI |
| | with linker sequence | EEDDSGMPVVTSGCLGLEGSDFQCRDTPIPQQRRSIECCTERNE |
| | (SEQ ID NO: 6), with | CNKDLHPTLPPLKNRDFV____TGGGGSGGGGSGGGGSGG |
| | Fab (SEQ ID NO: | GGSPTIKPCPPCKCPAP |
| | 201) | |
| 393 | SEQ ID NO: 388 | KKEDGESTAPTPRPKILRCYCSAGHCPEDSVNNICSTDGYCFTMI |
| | with linker sequence | EEDDSGMPVVTSGCLGLEGSDFQCRDTPIPQQRRSIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 394 | SEQ ID NO: 90 | KKEDGESTAPTPRPKILRCYCSAGHCPEDSVNNICSTDGYCFTMI |
| | Variant ECD with | EEDDSGMPVVTSGCLGLEGSDFQCRDTPIPHLRRSIECCTERNEC |
| | point mutations | NKDLHPTLPPLKNRDFV____ |
| | V16I, L51M, and | |
| | Q76L | |
| 395 | SEQ ID NO: 394 | KKEDGESTAPTPRPKILRCYCSAGHCPEDSVNNICSTDGYCFTMI |
| | with linker sequence | EEDDSGMPVVTSGCLGLEGSDFQCRDTPIPHLRRSIECCTERNEC |
| | (SEQ ID NO: 6) and | NKDLHPTLPPLKNRDFV____TGGGGSGGGGSGGGGSGGG |
| | Human Fc sequence | GSTHTCPPCPAPEAAGGPSVFLFPPKPKDTLMISRTPEVTCVVVD |
| | (SEQ ID NO: 106) | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 396 | SEQ ID NO: 394 | KKEDGESTAPTPRPKILRCYCSAGHCPEDSVNNICSTDGYCFTMI |
| | with linker sequence | EEDDSGMPVVTSGCLGLEGSDFQCRDTPIPHLRRSIECCTERNEC |
| | (SEQ ID NO: 6), with | NKDLHPTLPPLKNRDFV____TGGGGSGGGGSGGGGSGGG |
| | Fab (SEQ ID NO: | GSTHTCPPCPAPEAAGGP |
| | 199) | |
| 397 | SEQ ID NO: 394 | KKEDGESTAPTPRPKILRCYCSAGHCPEDSVNNICSTDGYCFTMI |
| | with linker sequence | EEDDSGMPVVTSGCLGLEGSDFQCRDTPIPHLRRSIECCTERNEC |
| | (SEQ ID NO: 6), with | NKDLHPTLPPLKNRDFV____TGGGGSGGGGSGGGGSGGG |
| | Fab (SEQ ID NO: | GSTHTCPPCKCPEAAGGP |
| | 200) | |
| 398 | SEQ ID NO: 394 | KKEDGESTAPTPRPKILRCYCSAGHCPEDSVNNICSTDGYCFTMI |
| | with linker sequence | EEDDSGMPVVTSGCLGLEGSDFQCRDTPIPHLRRSIECCTERNEC |
| | (SEQ ID NO: 6), with | NKDLHPTLPPLKNRDFV____TGGGGSGGGGSGGGGSGGG |
| | Fab (SEQ ID NO: | GSPTIKPCPPCKCPAP |
| | 201) | |
| 399 | SEQ ID NO: 394 | KKEDGESTAPTPRPKILRCYCSAGHCPEDSVNNICSTDGYCFTMI |
| | with linker sequence | EEDDSGMPVVTSGCLGLEGSDFQCRDTPIPHLRRSIECCTERNEC |
| | (SEQ ID NO: 6), with | NKDLHPTLPPLKNRDFV____TGGGGSGGGGSGGGGSGGG |
| | Fab (SEQ ID NO: | GSTHTCPPCPAPEAAGGPSVFLFPPKPKDTLMASRTPEVTCVVV |
| | 289) | DVSHEDPEVKFNWYVDGVEVHNAKTKPREEQYNSTYRVVSVL |
| | | TVLAQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 400 | SEQ ID NO: 90 | KKEDGESTAPTPRPKILRCYCSAGHCPEDSVNNICSTDGYCFTMI |
| | Variant ECD with | EEDDSGMPVVTSGCLGLEGSDFQCRDTPIPHQRRTIECCTERNE |
| | point mutations | CNKDLHPTLPPLKNRDFV____ |
| | V16I, L51M, and | |
| | S78T | |
| 401 | SEQ ID NO: 400 | KKEDGESTAPTPRPKILRCYCSAGHCPEDSVNNICSTDGYCFTMI |
| | with linker sequence | EEDDSGMPVVTSGCLGLEGSDFQCRDTPIPHQRRTIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 402 | SEQ ID NO: 400 | KKEDGESTAPTPRPKILRCYCSAGHCPEDSVNNICSTDGYCFTMI |
| | with linker sequence | EEDDSGMPVVTSGCLGLEGSDFQCRDTPIPHQRRTIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGP |
| | 199) | |
| 403 | SEQ ID NO: 400 | KKEDGESTAPTPRPKILRCYCSAGHCPEDSVNNICSTDGYCFTMI |
| | with linker sequence | EEDDSGMPVVTSGCLGLEGSDFQCRDTPIPHQRRTIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCKCPEAAGGP |
| | 200) | |
| 404 | SEQ ID NO: 400 | KKEDGESTAPTPRPKILRCYCSAGHCPEDSVNNICSTDGYCFTMI |
| | with linker sequence | EEDDSGMPVVTSGCLGLEGSDFQCRDTPIPHQRRTIECCTERNE |
| | (SEQ ID NO: 6), with | CNKDLHPTLPPLKNRDFV____TGGGGGGGGSGGGGSGG |
| | Fab (SEQ ID NO: | GGSPTIKPCPPCKCPAP |
| | 201) | |
| 405 | SEQ ID NO: 400 | KKEDGESTAPTPRPKILRCYCSAGHCPEDSVNNICSTDGYCFTMI |
| | with linker sequence | EEDDSGMPVVTSGCLGLEGSDFQCRDTPIPHQRRTIECCTERNE |
| | (SEQ ID NO: 6), with | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 406 | SEQ ID NO: 440 | KKEDGESTAPTPRPKILRCYCSAGHCPEDSVNNICSTDGYCFTMI |
| | Variant ECD with | EEDDSGMPVVTSGCLGLEGSDFQCRDTPIPHQRRSIECCTTNLC |
| | point mutations | NOYLQPTLPPVVIGPFFDGSIR |
| | (V16I; L51M) and | |
| | ALK3 C-Terminus | |
| 407 | SEQ ID NO: 406 | KKEDGESTAPTPRPKILRCYCSAGHCPEDSVNNICSTDGYCFTMI |
| | with linker sequence | EEDDSGMPVVTSGCLGLEGSDFQCRDTPIPHQRRSIECCTTNLC |
| | (SEQ ID NO: 6) and | NQYLQPTLPPVVIGPFFDGSIRTGGGGSGGGGSGGGGSGGG |
| | Human Fc sequence | GSTHTCPPCPAPEAAGGPSVFLFPPKPKDTLMISRTPEVTCVVVD |
| | (SEQ ID NO: 106) | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 408 | SEQ ID NO: 406 | KKEDGESTAPTPRPKILRCYCSAGHCPEDSVNNICSTDGYCFTMI |
| | with linker sequence | EEDDSGMPVVTSGCLGLEGSDFQCRDTPIPHQRRSIECCTTNLC |
| | (SEQ ID NO: 6), with | NQYLQPTLPPVVIGPFFDGSIRTGGGGSGGGGSGGGGSGGG |
| | Fab (SEQ ID NO: | GSTHTCPPCPAPEAAGGP |
| | 199) | |
| 409 | SEQ ID NO: 406 | KKEDGESTAPTPRPKILRCYCSAGHCPEDSVNNICSTDGYCFTMI |
| | with linker sequence | EEDDSGMPVVTSGCLGLEGSDFQCRDTPIPHQRRSIECCTTNLC |
| | (SEQ ID NO: 6), with | NQYLQPTLPPVVIGPFFDGSIRTGGGGSGGGGSGGGGSGGG |
| | Fab (SEQ ID NO: | GSTHTCPPCKCPEAAGGP |
| | 200) | |
| 410 | SEQ ID NO: 406 | KKEDGESTAPTPRPKILRCYCSAGHCPEDSVNNICSTDGYCFTMI |
| | with linker sequence | EEDDSGMPVVTSGCLGLEGSDFQCRDTPIPHQRRSIECCTTNLC |
| | (SEQ ID NO: 6), with | NQYLQPTLPPVVIGPFFDGSIRTGGGGSGGGGSGGGGSGGG |
| | Fab (SEQ ID NO: | GSPTIKPCPPCKCPAP |
| | 201) | |
| 411 | SEQ ID NO: 406 | KKEDGESTAPTPRPKILRCYCSAGHCPEDSVNNICSTDGYCFTMI |
| | with linker sequence | EEDDSGMPVVTSGCLGLEGSDFQCRDTPIPHQRRSIECCTTNLC |
| | (SEQ ID NO: 6), with | NQYLQPTLPPVVIGPFFDGSIRTGGGGSGGGGSGGGGSGGG |
| | Fab (SEQ ID NO: | GSTHTCPPCPAPEAAGGPSVFLFPPKPKDTLMASRTPEVTCVVV |
| | 285) | DVSHEDPEVKFNWYVDGVEVHNAKTKPREEQYNSTYRVVSVL |
| | | TVLAQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 412 | SEQ ID NO: 90 | KKEDGESTAPTPRPKILRCYCSAGHCPEDSVNNICSTDGYCFTMI |
| | Variant ECD with | EEDDSGLPVVTSGCLGLEGSDFQCRDTPIPHQRRSIECCTERNEC |
| | point mutation | NKDLHPTLPPLKNRDFV |
| | (V16I) and c- | |
| | terminus seven | |
| | amino acid deletion | |
| 413 | SEQ ID NO: 412 | KKEDGESTAPTPRPKILRCYCSAGHCPEDSVNNICSTDGYCFTMI |
| | with linker sequence | EEDDSGLPVVTSGCLGLEGSDFQCRDTPIPHQRRSIECCTERNEC |
| | (SEQ ID NO: 6) and | NKDLHPTLPPLKNRDFV____TGGGGSGGGGSGGGGSGGG |
| | Human Fc sequence | GSTHTCPPCPAPEAAGGPSVFLFPPKPKDTLMISRTPEVTCVVVD |
| | (SEQ ID NO: 106) | VSHEDPEVKFNWYVDGVEVHNAKTKPREEQYNSTYRVVSVLT |
| | | VLHQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 414 | SEQ ID NO: 412 | KKEDGESTAPTPRPKILRCYCSAGHCPEDSVNNICSTDGYCFTMI |
| | with linker sequence | EEDDSGLPVVTSGCLGLEGSDFQCRDTPIPHQRRSIECCTERNEC |
| | (SEQ ID NO: 6) and | NKDLHPTLPPLKNRDFV____TGGGGSGGGGSGGGGSGGG |
| | Fab (SEQ ID | GSTHTCPPCPAPEAAGGP |
| | NO: 199) | |
| 415 | SEQ ID NO: 412 | KKEDGESTAPTPRPKILRCYCSAGHCPEDSVNNICSTDGYCFTMI |
| | with linker sequence | EEDDSGLPVVTSGCLGLEGSDFQCRDTPIPHQRRSIECCTERNEC |
| | (SEQ ID NO: 6) and | NKDLHPTLPPLKNRDFV____TGGGGSGGGGSGGGGSGGG |
| | Fab (SEQ ID NO: | GSTHTCPPCKCPEAAGGP |
| | 200) | |
| 416 | SEQ ID NO: 412 | KKEDGESTAPTPRPKILRCYCSAGHCPEDSVNNICSTDGYCFTMI |
| | with linker sequence | EEDDSGLPVVTSGCLGLEGSDFQCRDTPIPHQRRSIECCTERNEC |
| | (SEQ ID NO: 6) and | NKDLHPTLPPLKNRDFV____TGGGGSGGGGSGGGGSGGG |
| | Fab (SEQ ID NO: | GSPTIKPCPPCKCPAP |
| | 201) | |
| 417 | SEQ ID NO: 412 | KKEDGESTAPTPRPKILRCYCSAGHCPEDSVNNICSTDGYCFTMI |
| | with linker sequence | EEDDSGLPVVTSGCLGLEGSDFQCRDTPIPHQRRSIECCTERNEC |
| | (SEQ ID NO: 6) and | NKDLHPTLPPLKNRDFV____TGGGGSGGGGSGGGGSGGG |
| | Fab (SEQ ID NO: | GSTHTCPPCPAPEAAGGPSVFLFPPKPKDTLMASRTPEVTCVVV |
| | 285) | DVSHEDPEVKFNWYVDGVEVHNAKTKPREEQYNSTYRVVSVL |
| | | TVLAQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 418 | SEQ ID NO: 90 | KKEDGESTAPTPRPKVLRCYCSAGHCPEDSVNNICSTDGYCFTM |
| | Variant ECD with | IEEDDSGMPVVTSGCLGLEGSDFQCRDTPIPHQRRSIECCTERNE |
| | point mutation | CNKDLHPTLPPLKNRDFV____ |
| | (L51M) and c- | |
| | terminus seven | |
| | amino acid deletion | |
| 419 | SEQ ID NO: 412 | KKEDGESTAPTPRPKVLRCYCSAGHCPEDSVNNICSTDGYCFTM |
| | with linker sequence | IEEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Human Fc sequence | GGSTHTCPPCPAPEAAGGPSVFLFPPKPKDTLMISRTPEVTCVVV |
| | (SEQ ID NO: 106) | DVSHEDPEVKFNWYVDGVEVHNAKTKPREEQYNSTYRVVSVL |
| | | TVLHQDWLNGKEYKCKVSNKALPAPIEKTISKAKGQPREPQVY |
| | | TLPPSREEMTKNQVSLTCLVKGFYPSDIAVEWESNGQPENNYKT |
| | | TPPVLDSDGSFFLYSKLTVDKSRWQQGNVFSCSVMHEALHNHY |
| | | TQKSLSLSPG |
| 420 | SEQ ID NO: 412 | KKEDGESTAPTPRPKVLRCYCSAGHCPEDSVNNICSTDGYCFTM |
| | with linker sequence | IEEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Fab (SEQ ID | GGSTHTCPPCPAPEAAGGP |
| | NO: 199) | |
| 421 | SEQ ID NO: 412 | KKEDGESTAPTPRPKVLRCYCSAGHCPEDSVNNICSTDGYCFTM |
| | with linker sequence | IEEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Fab (SEQ ID NO: | GGSTHTCPPCKCPEAAGGP |
| | 200) | |
| 422 | SEQ ID NO: 412 | KKEDGESTAPTPRPKVLRCYCSAGHCPEDSVNNICSTDGYCFTM |
| | with linker sequence | IEEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Fab (SEQ ID NO: | GGSPTIKPCPPCKCPAP |
| | 201) | |
| 423 | SEQ ID NO: 412 | KKEDGESTAPTPRPKVLRCYCSAGHCPEDSVNNICSTDGYCFTM |
| | with linker sequence | IEEDDSGMPVVTSGCLGLEGSDFQCRDTPIPHQRRSIECCTERNE |
| | (SEQ ID NO: 6) and | CNKDLHPTLPPLKNRDFV____TGGGGSGGGGSGGGGSGG |
| | Fab (SEQ ID NO: | GGSTHTCPPCPAPEAAGGPSVFLFPPKPKDTLMASRTPEVTCVV |
| | 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 424 | SEQ ID NO: 90 with | KKEDGESTAPTPRPKVLRCYCSAGHCPEDSVNNICSTDGYCFTM |
| | linker sequence (SEQ | IEEDDSGLPVVTSGCLGLEGSDFQCRDTPIPHQRRSIECCTERNE |
| | ID NO: 6) and Fc | CNKDLHPTLPPLKNRDFV____TGGGGSGGGGSGGGGSGG |
| | sequence (SEQ ID | GGSTHTCPPCPAPEAAGGPSVFLFPPKPKDTLMASRTPEVTCVV |
| | NO: 285) | VDVSHEDPEVKFNWYVDGVEVHNAKTKPREEQYNSTYRVVSV |
| | | LTVLAQDWLNGKEYKCKVSNKALPAPIEKTISKAKGQPREPQV |
| | | YTLPPSREEMTKNQVSLTCLVKGFYPSDIAVEWESNGQPENNY |
| | | KTTPPVLDSDGSFFLYSKLTVDKSRWQQGNVFSCSVMHEALHN |
| | | HYTQKSLSLSPG |
| 425 | Truncated ALK6 | KKEDGESTAPTPRPKVLRCKCHHHCPEDSVNNICSTDGYCFTMI |
| | ECD (with C | EEDDSGLPVVTSGCLGLEGSDFQCRDTPIPHQRRSIECCTTNLCN |
| | terminus 29 aa | QYLOPTLPPVVIGPFFDGSIR |
| | deletion and ALK3 C | |
| | terminus sequence) | |
| 426 | SEQ ID NO: 425 | KKEDGESTAPTPRPKVLRCKCHHHCPEDSVNNICSTDGYCFTMI |
| | with linker sequence | EEDDSGLPVVTSGCLGLEGSDFQCRDTPIPHQRRSIECCTTNLCN |
| | (SEQ ID NO: 6) and | QYLQPTLPPVVIGPFFDGSIRTGGGGSGGGGSGGGGSGGGG |
| | Human Fc sequence | STHTCPPCPAPEAAGGPSVFLFPPKPKDTLMISRTPEVTCVVVDV |
| | (SEQ ID NO: 106) | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPG |
| 427 | SEQ ID NO: 425 | KKEDGESTAPTPRPKVLRCKCHHHCPEDSVNNICSTDGYCFTMI |
| | with linker sequence | EEDDSGLPVVTSGCLGLEGSDFQCRDTPIPHQRRSIECCTTNLCN |
| | (SEQ ID NO: 6) and | QYLQPTLPPVVIGPFFDGSIRTGGGGSGGGGSGGGGSGGGG |
| | Fab (SEQ ID NO: | STHTCPPCPAPEAAGGP |
| | 199) | |
| 428 | SEQ ID NO: 425 | KKEDGESTAPTPRPKVLRCKCHHHCPEDSVNNICSTDGYCFTMI |
| | with linker sequence | EEDDSGLPVVTSGCLGLEGSDFQCRDTPIPHQRRSIECCTTNLCN |
| | (SEQ ID NO: 6) and | QYLQPTLPPVVIGPFFDGSIRTGGGGSGGGGSGGGGSGGGG |
| | Fab (SEQ ID NO: | STHTCPPCKCPEAAGGP |
| | 200) | |
| 429 | SEQ ID NO: 425 | KKEDGESTAPTPRPKVLRCKCHHHCPEDSVNNICSTDGYCFTMI |
| | with linker sequence | EEDDSGLPVVTSGCLGLEGSDFQCRDTPIPHQRRSIECCTTNLCN |
| | (SEQ ID NO: 6) and | QYLQPTLPPVVIGPFFDGSIRTGGGGSGGGGGGGGSGGGG |
| | Fab (SEQ ID NO: | SPTIKPCPPCKCPAP |
| | 201) | |
| 430 | SEQ ID NO: 425 | KKEDGESTAPTPRPKVLRCKCHHHCPEDSVNNICSTDGYCFTMI |
| | with linker sequence | EEDDSGLPVVTSGCLGLEGSDFQCRDTPIPHQRRSIECCTTNLCN |
| | (SEQ ID NO: 6) and | QYLQPTLPPVVIGPFFDGSIRTGGGGSGGGGSGGGGSGGGG |
| | Fab (SEQ ID NO: | STHTCPPCPAPEAAGGPSVFLFPPKPKDTLMASRTPEVTCVVVD |
| | 285) | VSHEDPEVKFNWYVDGVEVHNAKTKPREEQYNSTYRVVSVLT |
| | | VLAQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 431 | Truncated ALK6 | KKEDGESTAPTPRPKILRCKCHHHCPEDSVNNICSTDGYCFTMIE |
| | variant ECD with | EDDSGMPVVTSGCLGLEGSDFQCRDTPIPHQRRSIECCTTNLCN |
| | point mutations | QYLOPTLPPVVIGPFFDGSIR |
| | (V16I; L51M) and C | |
| | terminus 29 aa | |
| | deletion and ALK3 C | |
| | terminus sequence) | |
| 432 | SEQ ID NO: 431 with | KKEDGESTAPTPRPKILRCKCHHHCPEDSVNNICSTDGYCFTMIE |
| | linker sequence (SEQ | EDDSGMPVVTSGCLGLEGSDFQCRDTPIPHQRRSIECCTTNLCN |
| | ID NO: 6) and Human | QYLQPTLPPVVIGPFFDGSIRTGGGGSGGGGSGGGGSGGGG |
| | Fc sequence (SEQ ID | STHTCPPCPAPEAAGGPSVFLFPPKPKDTLMISRTPEVTCVVVDV |
| | NO: 106) | SHEDPEVKFNWYVDGVEVHNAKTKPREEQYNSTYRVVSVLTV |
| | | LHQDWLNGKEYKCKVSNKALPAPIEKTISKAKGQPREPQVYTL |
| | | PPSREEMTKNQVSLTCLVKGFYPSDIAVEWESNGQPENNYKTTP |
| | | PVLDSDGSFFLYSKLTVDKSRWQQGNVFSCSVMHEALHNHYT |
| | | QKSLSLSPG |
| 433 | SEQ ID NO: 431 with | KKEDGESTAPTPRPKILRCKCHHHCPEDSVNNICSTDGYCFTMIE |
| | linker sequence (SEQ | EDDSGMPVVTSGCLGLEGSDFQCRDTPIPHQRRSIECCTTNLCN |
| | ID NO: 6) and Fab | QYLQPTLPPVVIGPFFDGSIRTGGGGSGGGGSGGGGSGGGG |
| | (SEQ ID NO: 199) | STHTCPPCPAPEAAGGP |
| 434 | SEQ ID NO: 431 with | KKEDGESTAPTPRPKILRCKCHHHCPEDSVNNICSTDGYCFTMIE |
| | linker sequence (SEQ | EDDSGMPVVTSGCLGLEGSDFQCRDTPIPHQRRSIECCTTNLCN |
| | ID NO: 6) and Fab | QYLQPTLPPVVIGPFFDGSIRTGGGGSGGGGSGGGGSGGGG |
| | (SEQ ID NO: 200) | STHTCPPCKCPEAAGGP |
| 435 | SEQ ID NO: 431 with | KKEDGESTAPTPRPKILRCKCHHHCPEDSVNNICSTDGYCFTMIE |
| | linker sequence (SEQ | EDDSGMPVVTSGCLGLEGSDFQCRDTPIPHQRRSIECCTTNLCN |
| | ID NO: 6) and Fab | QYLQPTLPPVVIGPFFDGSIRTGGGGSGGGGSGGGGSGGGG |
| | (SEQ ID NO: 201) | SPTIKPCPPCKCPAP |
| 436 | SEQ ID NO: 431 with | KKEDGESTAPTPRPKILRCKCHHHCPEDSVNNICSTDGYCFTMIE |
| | linker sequence (SEQ | EDDSGMPVVTSGCLGLEGSDFQCRDTPIPHQRRSIECCTTNLCN |
| | ID NO: 6) and Fab | QYLQPTLPPVVIGPFFDGSIRTGGGGSGGGGSGGGGSGGGG |
| | (SEQ ID NO: 285) | STHTCPPCPAPEAAGGPSVFLFPPKPKDTLMASRTPEVTCVVVD |
| | | VSHEDPEVKFNWYVDGVEVHNAKTKPREEQYNSTYRVVSVLT |
| | | VLAQDWLNGKEYKCKVSNKALPAPIEKTISKAKGQPREPQVYT |
| | | LPPSREEMTKNQVSLTCLVKGFYPSDIAVEWESNGQPENNYKTT |
| | | PPVLDSDGSFFLYSKLTVDKSRWQQGNVFSCSVMHEALHNHYT |
| | | QKSLSLSPG |
| 437 | Wild type ALK 6 ECD | Lys₁-Lys₂-Glu₃-Asp₄-Gly₅-Glu₆-Ser₇-Thr₈-Ala₉-Pro₁₀- |
| | background with | Thr₁₁-Pro₁₂-Arg₁₃-Pro₁₄-Xaa₁₅-Xaa₁₆-Leu₁₇-Arg₁₈- |
| | point mutations with | Cys₁₉-Xaa₂₀-Cys₂₁-Xaa₂₂-Xaa₂₃-His₂₄-Cys₂₅-Pro₂₆-Xaa₂₇- |
| | ALK6 or ALK3 C | Asp₂₈-Xaa₂₉-Xaa₃₀-Asn₃₁-Asn₃₂-Xaa₃₃-Cys₃₄-Xaa₃₅- |
| | terminus sequence | Thr₃₆-Xaa₃₇-Gly₃₈-Xaa₃₉-Cys₄₀-Phe₄₁-Xaa₄₂-Xaa₄₃-Ile₄₄- |
| | | Glu₄₅-Glu₄₆-Asp₄₇-Asp₄₈-Xaa₄₉-Gly₅₀-Xaa₅₁-Xaas₂-Xaa₅₃- |
| | | Xaa₅₄-Xaa₅₅-Ser₅₆-Gly₅₇-Cys₅₈-Xaa₅₉-Xaa₆₀-Xaa₆₁- |
| | | Glu₆₂-Gly₆₃-Ser₆₄-Asp₆₅-Phe₆₆-Gln₆₇-Cys₆₈-Xaa₆₉-Asp₇₀- |
| | | Xaa₇₁-Pro₇₂-Xaa₇₃-Xaa₇₄-Xaa₇₅-Xaa₇₆-Arg₇₇-Arg₇₈- |
| | | Xaa₇₉-Ile₈₀-Glu₈₁-Cys₈₂-Cys₈₃-Xaa₈₄-Xaa₈₅-Xaa₈₆-Xaa₈₇- |
| | | Xaa₈₈-Xaa₈₉-Xaa₉₀-Xaa₉₁-Xaa₉₂-Xaa₉₃-Xaa₉₄-Xaa₉₅-Xaa₉₆- |
| | | Xaa₉₇-Xaa₉₈-Xaa₉₉-Xaa₁₀₀-Xaa₁₀₁-Xaa₁₀₂-Xaa₁₀₃-Xaa₁₀₄- |
| | | Xaa₁₀₅-Xaa₁₀₆-Xaa₁₀₇-Xaa₁₀₈-Xaa₁₀₉-Xaa₁₁₀-Xaa₁₁₁-Xaa₁₁₂- |
| | | Xaa₁₁₃ |
| | | wherein: Xaa₁₅ is K or P; Xaa₁₆ is V or I; Xaa₂₀ is V, F, K or Y; Xaa₂₂ |
| | | is G, H or S; Xaa₂₃ is S, H or G; Xaa₂₇ is E or D; Xaa₂₉ is S or A; |
| | | Xaa₃₀ is V or I; Xaa₃₃ is I or T; Xaa₃₅ is S or I; Xaa₃₇ is D or N; |
| | | Xaa₃₉ is Y or H; Xaa₄₂ is T or A; Xaa₄₃ is M or I; Xaa₄₉ is S or Q; |
| | | Xaa₅₁ is L or E; Xaa₅₂ is P or T; Xaa₅₃ is V or T; Xaa₅₄ is V or L; |
| | | Xaa₅₅ is T or A; Xaa₅₉ is L or M Xaa₆₀ is G or K; Xaa₆₁ is L or Y; |
| | | Xaa₆₉ is R or K; Xaa₇₁ is T or S; Xaa₇₃ is I or K; Xaa₇₄ is P or A; |
| | | Xaa₇₅ is H or Q; Xaa₇₆ is Q or L; Xaa₇₉ is S or T; Xaa₈₄ is T or R; |
| | | Xaa₈₅ is absent, T or E; Xaa₈₆ is R or N; Xaa₈₇ is absent, N or L; |
| | | Xaa₈₈ is absent, E or L; Xaa₈₉ is absent or C; Xaa₉₀ is absent or N; |
| | | Xaa₉₁ is absent, K or Q; Xaa₉₂ is absent, D or L; Xaa₉₃ is absent or |
| | | L; Xaa₉₄ is absent, H or Q; Xaa₉₅ is absent or P; Xaa₉₆ is absent or |
| | | T; Xaa₉₇ is absent or L; Xaa₉₈ is absent or P; Xaa₉₉ is absent or |
| | | P; Xaa₁₀₀ is absent, V or L; Xaa₁₀₁ is absent, V or K; Xaa₁₀₂ is |
| | | absent, I or N; Xaa₁₀₃ is absent, G or R; Xaa₁₀₄ is absent, P, D or A; |
| | | Xaa₁₀₅ is absent or F; Xaa₁₀₆ is absent, F or V; Xaa₁₀₇ is absent or D; |
| | | Xaa₁₀₈ is absent or G; Xaa₁₀₉ is absent, S or P; Xaa₁₁₀ is absent or I; |
| | | Xaa₁₁₁ is absent, R or H; Xaa₁₁₂ is absent or H; and Xaa₁₁₃ is absent |
| | | or R |
| 438 | ALK3 C terminal | TNLCNQYLQPTLPPVVIGPFFDGSIR |
| | sequence | |
| 439 | Truncated ALK6 | KKEDGESTAPTPRPKVLRCKCHHHCPEDSVNNICSTDGYCFTMI |
| | ECD (30 aa deleted) | EEDDSGLPVVTSGCLGLEGSDFQCRDTPIPHQRRSIECC___ |
| | with ALK3 C | __________________ |
| | terminus sequence | |
| 440 | Truncated ALK6 | KKEDGESTAPTPRPKVLRCYCSAGHCPEDSVNNICSTDGYCFTM |
| | ECD (SEQ ID NO: 90 | IEEDDSGMPVVTSGCLGLEGSDFQCRDTPIPHQRRSIECCTNLCN |
| | with 30 aa deleted) | QYLOPTLPPVVIGPFFDGSIR |
| | with ALK3 C | |
| | terminus sequence | |

We claim:

1. A method for treatment of retinopathy in a human subject in need thereof comprising: administering an effective amount of a pharmaceutical composition to the subject, wherein the composition comprises a soluble recombinant bone morphogenetic protein receptor type-1B fusion protein comprising an ALK6 ECD polypeptide and a heterologous sequence, wherein the ALK6 ECD polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 3; SEQ ID NO: 5; SEQ ID NO: 79, SEQ ID NO: 81, SEQ ID NO: 82, SEQ ID NO: 83, SEQ ID NO: 108; SEQ ID NO: 109; SEQ ID NO: 130, SEQ ID NO: 157, SEQ ID NO: 190 and SEQ ID NO: 437.

2. The method of claim 1, wherein the ALK6 ECD polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an ALK6 ECD sequence selected from SEQ ID NO: 1, 4, 11-45, 57, 59, 61, 63, 65, 67, 69, 71, 73, 75, 77, 80, 86, 88, 90, 92, 94, 96, 98, 100, 102, 104, 110, 113, 132-156, 158-189, 191-194, 202, 213, 218, 223, 228, 233, 242, 247, 252, 257, 262, 272, 277, 283, 286, 292, 298, 304, 310, 316, 322, 328, 334 340, 346, 352, 358, 364, 370, 376, 382, 388, 394, 400, 406, 412, 418, 425, or 431.

3. The method of claim 1, wherein the soluble recombinant bone morphogenetic protein receptor type-1B fusion protein comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to an amino acid sequence selected from SEQ ID NO: 91, 291, 297, 303, 309, 315, 321, 327, 333, 339, 345, 351, 357, 363, 369, 375, 381, 387, 393, 399, 405, 411, 417, 423, 424, 430, and 436.

4. The method of claim 3, wherein the fusion protein polypeptide comprises an amino acid sequence having at least 70%, 75%, 80%, 85%, 86%, 87%, 88%, 89%, 90%, 91%, 92%, 93%, 94%, 95%, 96%, 97%, 98%, 99%, or 100% identity to SEQ ID NO: 291.

5. The method of claim 1, wherein the ALK6 ECD sequence comprises at least one point mutation as compared to SEQ ID NO: 1.

6. The method of claim 1, wherein the ALK6 ECD sequence comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 86, SEQ ID NO: 88, SEQ ID NO: 90 or SEQ ID NO: 286.

7. The method of claim 1, wherein the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 69.

8. The method of claim 1, wherein the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 90.

9. The method of claim 1, wherein the ALK6 ECD comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 286.

10. (canceled)

11. The method of claim 1, wherein the ALK6 ECD polypeptide comprises one or more point mutations selected from one or more of V16I; K20Y, K20F, H22S, _22′A, H23G, V30I, I33T, I33S, S35I, S49Q and L51M.

12. The method of claim 1, wherein the polypeptide binds one or more of BMP6, BMP7 and/or BMP10.

13. The method of claim 1, wherein the ALK6 ECD sequence is selected from SEQ ID NO: 190 and the sequence comprises point mutations K20Y and H22S.

14. (canceled)

15. (canceled)

16. (canceled)

17. (canceled)

18. The method of claim 1, wherein the heterologous sequence is an Fc sequence according to SEQ ID NO: 285.

19. The method of claim 1, wherein the retinopathy is selected from diabetic retinopathy, age-related macular degeneration (AMD), choroidal neovascularization (CNV), vision loss, night blindness, photopsia, proliferative retinopathy, non-proliferative retinopathy, retinitis pigmentosa and hypertensive retinopathy.

20-29. (canceled)

30. The method of claim 1, wherein the soluble recombinant bone morphogenetic protein receptor type-1B fusion protein sequence comprises or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 90, wherein the position corresponding to position 60 of SEQ ID NO: 69 is a glycine (G) residue, and wherein the ALK6 fusion protein demonstrates retained BMP10 binding.

31-36. (canceled)

37. A soluble recombinant bone morphogenetic protein receptor type-1B fusion protein comprising an ALK6 ECD polypeptide and a Fc sequence, wherein the ALK6 ECD polypeptide comprises an amino acid sequence selected from a sequence of SEQ ID NO: 130:
Lys₁-Lys₂-Glu₃-Asp₄-Gly₅-Glu₆-Ser₇-Thr₈-Ala₉-Pro₁₀-Thr₁₁-Pro₁₂-
Arg₁₃-Pro₁₄-Lys₁₅-Xaa₁₆-Leu₁₇-Arg₁₈-Cys₁₉-Xaa₂₀-Cys₂₁-Xaa₂₂-Xaa′₂₂-
Xaa”₂₂-Xaa₂₃-Xaa′₂₃-Xaa₂₄-Cys₂₅-Pro₂₆-Xaa₂₇-Asp₂₈-Xaa₂₉-Xaa₃₀-Asn₃₁-
Asn₃₂-Xaa₃₃-Cys₃₄-Xaa₃₅-Thr₃₆-Xaa₃₇-Gly₃₈-Xaa₃₉-Cys₄₀-Phe₄₁-Xaa₄₂-
Xaa₄₃-Ile₄₄-Glu₄₅-Glu₄₆-Asp₄₇-Asp₄₈-Xaa₄₉-Gly₅₀-Xaa₅₁-Xaa₅₂-Xaa₅₃-
Xaa₅₄-Xaa₅₅-Ser₅₆-Gly₅₇-Cys₅₈-Xaa₅₉-Xaa₆₀-Xaa₆₁-Glu₆₂-Gly₆₃-Ser₆₄-
Asp₆₅-Phe₆₆-Gln₆₇-Cys₆₈-Xaa₆₉-Asp₇₀-Xaa₇₁-Pro₇₂-Xaa₇₃-Xaa₇₄-Xaa₇₅-
Xaa₇₆-Arg₇₇-Arg₇₈-Xaa₇₉-Ile₈₀-Glu₈₁-Cys₈₂-Cys₈₃-Xaa₈₄-Xaa₈₅-Xaa₈₆-
Xaa₈₇-Xaa₈₈-Cys₈₉-Asn₉₀-Xaa₉₁-Xaa₉₂-Leu₉₃-Xaa₉₄-Pro₉₅-Thr₉₆-Leu₉₇-
Pro₉₈-Pro₉₉-Leu₁₀₀-Lys₁₀₁-Asn₁₀₂-Arg₁₀₃-Asp₁₀₄-Phe₁₀₅-Val₁₀₆-Xaa₁₀₇-Xaa₁₀₈-
Xaa₁₀₉-Xaa₁₁₀-Xaa₁₁₁-Xaa₁₁₂-Xaa₁₁₃;
wherein
Xaa₁₆ is V or I
Xaa₂₀ is V, F, K, Y, T, or A;
Xaa₂₂ is G, E, H or S;
Xaa′₂₂ is absent, G, S, or A;
Xaa″₂₂ is absent or S;
Xaa₂₃ is S, H, G, or L;
Xaa₂₃′ is absent, L or H;
Xaa₂₄ is H or S;
Xaa₂₇ is E or D;
Xaa₂₉ is S or A;
Xaa₃₀ is V or I;
Xaa₃₃ is I or T;
Xaa₃₅ is S or I;
Xaa₃₇ is D or N;
Xaa₃₉ is Y or H;
Xaa₄₂ is T or A;
Xaa₄₃ is M or I;
Xaa₄₉ is S or Q;
Xaa₅₁ is L or E;
Xaa₅₂ is P or T;
Xaa₅₃ is V or T;
Xaa₅₄ is V or L;
Xaa₅₅ is T or A;
Xaa₅₉ is L or M;
Xaa₆₀ is G or K;
Xaa₆₁ is L or Y;
Xaa₆₉ is R or K;
Xaa₇₁ is T or S;
Xaa₇₃ is I or K;
Xaa₇₄ is P or A;
Xaa₇₅ is H or Q;
Xaa₇₆ is Q or L;
Xaa₇₉ is S or T;
Xaa₈₄ is T or R;
Xaa₈₅ is absent, T or E;
Xaa₈₆ is R or N;
Xaa₈₇ is N or L;
Xaa₈₈ is absent, E or L;
Xaa₉₁ is K or Q;
Xaa₉₂ is D or L;
Xaa₉₄ is H or Q;
Xaa₁₀₇ is absent;
Xaa₁₀₈ is absent;
Xaa₁₀₉ is absent;
Xaa₁₁₀ is absent;
Xaa₁₁₁ is absent;
Xaa₁₁₂ is absent; and,
Xaa₁₁₃ is absent.

38. The fusion protein of claim 37, wherein Xaa60 is a glycine (G) residue and the polypeptide binds BMP10.

39. (canceled)

40. (canceled)

41. The fusion protein of claim 37, further comprising a linker sequence selected from SEQ ID NO: 6, SEQ ID NO: 7 or SEQ ID NO: 8.

42-76. (canceled)

77. A soluble recombinant bone morphogenetic protein receptor type-1B fusion protein comprising:
(i) an ALK6 extra cellular domain (ECD);
(ii) a linker sequence; and,
(iii) an Fc domain sequence;
wherein the ALK6 ECD sequence comprises or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an ALK6 ECD sequence of SEQ ID NO: 90, wherein the position corresponding to position 60 of SEQ ID NO: 69 is a glycine (G) residue, and wherein the ALK6 fusion protein demonstrates retained binding to BMP10.

78. (canceled)

79. (canceled)

80. The fusion protein of claim 77, wherein the ALK6 fusion protein demonstrates an nM IC₅₀ value of about 7.2 for BMP10 binding and/or an nM IC₅₀ value of about 4.8 for BMP7 binding.

81-95. (canceled)


SEQUENCE LISTING (440 sequences)

SEQ ID NO: 1 - protein, 113 aa, Homo sapiens
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 2 - protein, 12 aa, Homo sapiens
VVIGPFFDGSIR

SEQ ID NO: 3 - protein, 113 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = absent, T or E
  VARIANT 86: note = R or N
  VARIANT 87: note = N or L
  VARIANT 88: note = absent, E or L
  VARIANT 91: note = K or Q
  VARIANT 92: note = D or L
  VARIANT 94: note = H or Q
  VARIANT 100: note = absent, V or L
  VARIANT 101: note = absent, V or K
  VARIANT 102: note = absent, I or N
  VARIANT 103: note = absent, G or R
  VARIANT 104: note = absent, P, D or A
  VARIANT 105: note = absent or F
  VARIANT 106: note = absent, F or V
  VARIANT 107: note = absent or D
  VARIANT 108: note = absent or G
  VARIANT 109: note = absent, S or P
  VARIANT 110: note = absent or I
  VARIANT 111: note = absent, R or H
  VARIANT 112: note = absent or H
  VARIANT 113: note = absent or R
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPXXXXXXXXXXXXXX

SEQ ID NO: 4 - protein, 100 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPL

SEQ ID NO: 5 - protein, 100 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = absent, T or E
  VARIANT 86: note = R or N
  VARIANT 87: note = N or L
  VARIANT 88: note = absent, E or L
  VARIANT 91: note = K or Q
  VARIANT 92: note = D or L
  VARIANT 94: note = H or Q
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPL

SEQ ID NO: 6 - protein, 21 aa, synthetic construct
TGGGGSGGGGSGGGGSGGGGS

SEQ ID NO: 7 - protein, 4 aa, synthetic construct
TGGG

SEQ ID NO: 8 - protein, 20 aa, synthetic construct
EAAAKEAAAKEAAAKEAAAK

SEQ ID NO: 9 - protein, 19 aa, synthetic construct
MGWSCIILFLVATATGVHS

SEQ ID NO: 10 - protein, 225 aa, synthetic construct
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 12 - protein, 113 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVDGPIHHR

SEQ ID NO: 13 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFV

SEQ ID NO: 14 - protein, 112 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVGPIHHR

SEQ ID NO: 15 - protein, 111 aa, synthetic construct
KKEDGESTAPTPRPKVLRCKCHGHCPEDSVNNICSTNGYCFTIIEEDDSGLPVVTSGCMGYEGSDFQCRDTPKAHLRRSIECCTERNECNKDLHPTLPPVVIGPFFDGSIR

SEQ ID NO: 16 - protein, 110 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTRNECNKDLHPTLPPVVIGPFFDGSIR

SEQ ID NO: 17 - protein, 111 aa, synthetic construct
KKEDGESTAPTPRPPVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 18 - protein, 111 aa, synthetic construct
KKEDGESTAPTPRPKVLRCYCHHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 19 - protein, 111 aa, synthetic construct
KKEDGESTAPTPRPKVLRCKCSHHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 20 - protein, 111 aa, synthetic construct
KKEDGESTAPTPRPKVLRCKCHGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 21 - protein, 111 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 22 - protein, 111 aa, synthetic construct
KKEDGESTAPTPRPKVLRCKCHHHCPDDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 23 - protein, 111 aa, synthetic construct
KKEDGESTAPTPRPKVLRCKCHHHCPEDSINNICSTDGYCFTMIEEDDSGLPVVTSGCLGLEGSDFQCRDTPIPHQRRSIECCTERNECNKDLHPTLPPVVIGPFFDGSIR

SEQ ID NO: 24 - protein, 111 aa, synthetic construct
KKEDGESTAPTPRPKVLRCKCHHHCPEDSVNNTCSTDGYCFTMIEEDDSGLPVVTSGCLGLEGSDFQCRDTPIPHQRRSIECCTERNECNKDLHPTLPPVVIGPFFDGSIR

SEQ ID NO: 25 - protein, 111 aa, synthetic construct
KKEDGESTAPTPRPKVLRCKCHHHCPEDSVNNICITDGYCFTMIEEDDSGLPVVTSGCLGLEGSDFQCRDTPIPHQRRSIECCTERNECNKDLHPTLPPVVIGPFFDGSIR

SEQ ID NO: 26 - protein, 111 aa, synthetic construct
KKEDGESTAPTPRPKVLRCKCHHHCPEDSVNNICSTNGYCFTMIEEDDSGLPVVTSGCLGLEGSDFQCRDTPIPHQRRSIECCTERNECNKDLHPTLPPVVIGPFFDGSIR

SEQ ID NO: 27 - protein, 111 aa, synthetic construct
KKEDGESTAPTPRPKVLRCKCHHHCPEDSVNNICSTDGHCFTMIEEDDSGLPVVTSGCLGLEGSDFQCRDTPIPHQRRSIECCTERNECNKDLHPTLPPVVIGPFFDGSIR

SEQ ID NO: 28 - protein, 111 aa, synthetic construct
KKEDGESTAPTPRPKVLRCKCHHHCPEDSVNNICSTDGYCFAIIEEDDSGLPVVTSGCLGLEGSDFQCRDTPIPHQRRSIECCTERNECNKDLHPTLPPVVIGPFFDGSIR

SEQ ID NO: 29 - protein, 111 aa, synthetic construct
KKEDGESTAPTPRPKVLRCKCHHHCPEDSVNNICSTDGYCFAMIEEDDSGLPVVTSGCLGLEGSDFQCRDTPIPHQRRSIECCTERNECNKDLHPTLPPVVIGPFFDGSIR

SEQ ID NO: 30 - protein, 111 aa, synthetic construct
KKEDGESTAPTPRPKVLRCKCHHHCPEDSVNNICSTDGYCFTIIEEDDSGLPVVTSGCLGLEGSDFQCRDTPIPHQRRSIECCTERNECNKDLHPTLPPVVIGPFFDGSIR

SEQ ID NO: 31 - protein, 111 aa, synthetic construct
KKEDGESTAPTPRPKVLRCKCHHHCPEDSVNNICSTDGYCFTMIEEDDQGLPVVTSGCLGLEGSDFQCRDTPIPHQRRSIECCTERNECNKDLHPTLPPVVIGPFFDGSIR

SEQ ID NO: 32 - protein, 111 aa, synthetic construct
KKEDGESTAPTPRPKVLRCKCHHHCPEDSVNNICSTDGYCFTMIEEDDQGETTLASGCLGLEGSDFQCRDTPIPHQRRSIECCTERNECNKDLHPTLPPVVIGPFFDGSIR

SEQ ID NO: 33 - protein, 111 aa, synthetic construct
KKEDGESTAPTPRPKVLRCKCHHHCPEDSVNNICSTDGYCFTMIEEDDSGLPVVTSGCMKYEGSDFQCRDTPIPHQRRSIECCTERNECNKDLHPTLPPVVIGPFFDGSIR

SEQ ID NO: 34 - protein, 111 aa, synthetic construct
KKEDGESTAPTPRPKVLRCKCHHHCPEDSVNNICSTDGYCFTMIEEDDSGLPVVTSGCMGLEGSDFQCRDTPIPHQRRSIECCTERNECNKDLHPTLPPVVIGPFFDGSIR

SEQ ID NO: 35 - protein, 111 aa, synthetic construct
KKEDGESTAPTPRPKVLRCKCHHHCPEDSVNNICSTDGYCFTMIEEDDSGLPVVTSGCLKLEGSDFQCRDTPIPHQRRSIECCTERNECNKDLHPTLPPVVIGPFFDGSIR

SEQ ID NO: 36 - protein, 111 aa, synthetic construct
KKEDGESTAPTPRPKVLRCKCHHHCPEDSVNNICSTDGYCFTMIEEDDSGLPVVTSGCLGYEGSDFQCRDTPIPHQRRSIECCTERNECNKDLHPTLPPVVIGPFFDGSIR

SEQ ID NO: 37 - protein, 111 aa, synthetic construct
KKEDGESTAPTPRPKVLRCKCHHHCPEDSVNNICSTDGYCFTMIEEDDSGLPVVTSGCLGLEGSDFQCRDTPKAQLRRSIECCTERNECNKDLHPTLPPVVIGPFFDGSIR

SEQ ID NO: 38 - protein, 111 aa, synthetic construct
KKEDGESTAPTPRPKVLRCKCHHHCPEDSVNNICSTDGYCFTMIEEDDSGLPVVTSGCLGLEGSDFQCRDTPKAHQRRSIECCTERNECNKDLHPTLPPVVIGPFFDGSIR

SEQ ID NO: 39 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPQLRRSIECCTERNECNKDLHPTLPPVVIGPFFDGSIR

SEQ ID NO: 40 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCRERNLCNKDLHPTLPPVVIGPFFDGSIR

SEQ ID NO: 41 - protein, 111 aa, synthetic construct
KKEDGESTAPTPRPKVLRCKCHHHCPEDSVNNICSTDGYCFTMIEEDDSGLPVVTSGCLKLEGSDFQCRDTPKAQLRRSIECCTERNECNKDLHPTLPPVVIGPFFDGSIR

SEQ ID NO: 42 - protein, 110 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCRTNLCNQYLQPTLPPVVIGPFFDGSIR

SEQ ID NO: 43 - protein, 110 aa, synthetic construct; identical to SEQ ID NO: 16

SEQ ID NO: 44 - protein, 111 aa, synthetic construct
KKEDGESTAPTPRPKVLRCKCSGHCPDDAVNNTCITNGYCFAIIEEDDSGLPVVASGCMKYEGSDFQCKDSPKAQLRRTIECCTERNECNKDLHPTLPPVVIGPFFDGSIR

SEQ ID NO: 45 - protein, 111 aa, synthetic construct
KKEDGESTAPTPRPKVLRCKCHGHCPEDSVNNICSTNGYCFTIIEEDDSGLPVVTSGCMKYEGSDFQCRDTPKAHLRRSIECCTERNECNKDLHPTLPPVVIGPFFDGSIR

SEQ ID NO: 46 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 359 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVDGPIHHRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 352 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRAFVTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 378 aa, synthetic construct
MGWSCIILFLVATATGVHSKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 365 aa, synthetic construct
MGWSCIILFLVATATGVHSKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 14 aa, Homo sapiens
LKNRDFVDGPIHHR

SEQ ID NO: 52 - protein, 357 aa, synthetic construct
KKEDGESTAPTPRPKVLRCKCHGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 357 aa, synthetic construct
KKEDGESTAPTPRPKVLRCKCHHHCPEDSVNNICSTNGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 357 aa, synthetic construct
KKEDGESTAPTPRPKVLRCKCHHHCPEDSVNNICSTDGYCFAIIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 357 aa, synthetic construct
KKEDGESTAPTPRPKVLRCKCHHHCPEDSVNNICSTDGYCFTMIEEDDSGLPVVTSGCMKYEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 357 aa, synthetic construct
KKEDGESTAPTPRPKVLRCKCHHHCPEDSVNNICSTDGYCFTMIEEDDSGLPVVTSGCLKLEGSDFQCRDTPKAQLRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 111 aa, synthetic construct; identical to SEQ ID NO: 19

SEQ ID NO: 58 - protein, 357 aa, synthetic construct
KKEDGESTAPTPRPKVLRCKCSHHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 111 aa, synthetic construct
KKEDGESTAPTPRPKVLRCKCSGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 60 - protein, 357 aa, synthetic construct
KKEDGESTAPTPRPKVLRCKCSGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 111 aa, synthetic construct; identical to SEQ ID NO: 21

SEQ ID NO: 62 - protein, 357 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 111 aa, synthetic construct
KKEDGESTAPTPRPKVLRCYCHGHCPEDSVNNICSTDGYCFTMIEEDDSGLPVVTSGCLGLEGSDFQCRDTPIPHQRRSIECCTERNECNKDLHPTLPPVVIGPFFDGSIR

SEQ ID NO: 64 - protein, 357 aa, synthetic construct
KKEDGESTAPTPRPKVLRCYCHGHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 111 aa, synthetic construct; identical to SEQ ID NO: 18

SEQ ID NO: 66 - protein, 357 aa, synthetic construct
KKEDGESTAPTPRPKVLRCYCHHHCPEDSVNNICSTDGYCFTMIEEDDSGLPVVTSGCLGLEGSDFQCRDTPIPHQRRSIECCTERNECNKDLHPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 111 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVAGPIR

SEQ ID NO: 68 - protein, 357 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVAGPIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 70 - protein, 352 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 113 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIHHR

SEQ ID NO: 72 - protein, 359 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIHHRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIR

SEQ ID NO: 74 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVAGPIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGSIR

SEQ ID NO: 76 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFFDGPIR

SEQ ID NO: 78 - protein, 357 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFFDGPIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 111 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = absent, T or E
  VARIANT 86: note = R or N
  VARIANT 87: note = N or L
  VARIANT 88: note = absent, E or L
  VARIANT 91: note = K or Q
  VARIANT 92: note = D or L
  VARIANT 94: note = H or Q
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPVVIGPFFDGSIR

SEQ ID NO: 80 - protein, 111 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIR

SEQ ID NO: 81 - protein, 106 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = absent, T or E
  VARIANT 86: note = R or N
  VARIANT 87: note = N or L
  VARIANT 88: note = absent, E or L
  VARIANT 91: note = K or Q
  VARIANT 92: note = D or L
  VARIANT 94: note = H or Q
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPLKNRDFV

SEQ ID NO: 82 - protein, 111 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = absent, T or E
  VARIANT 86: note = R or N
  VARIANT 87: note = N or L
  VARIANT 88: note = absent, E or L
  VARIANT 91: note = K or Q
  VARIANT 92: note = D or L
  VARIANT 94: note = H or Q
  VARIANT 107: note = D or A
  VARIANT 109: note = P or S
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPLKNRDFVXGXIR

SEQ ID NO: 83 - protein, 113 aa, synthetic construct
  VARIANT 15: note = K or P
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = absent, T or E
  VARIANT 86: note = R or N
  VARIANT 87: note = N or L
  VARIANT 88: note = absent, E or L
  VARIANT 91: note = K or Q
  VARIANT 92: note = D or L
  VARIANT 94: note = H or Q
  VARIANT 104: note = D or A
  VARIANT 107: note = absent or D
  VARIANT 109: note = P or S
KKEDGESTAPTPRPXVLRCXCXXHCPXDXXNNXCXTXGXCFXXIEEDDXGXXXXXSGCXXXEGSDFQCXDXPXXXXRRXIECCXXXXXCNXXLXPTLPPLKNRXFVXGXIHHR

SEQ ID NO: 84 - protein, 356 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIREAAAKEAAAKEAAAKEAAAKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 340 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPVVIGPFFDGSIRTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 106 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 87 - protein, 351 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 111 aa, synthetic construct
KKEDGESTAPTPRPKVLRCYCSGHCPEDSVNNICSTDGYCFTMIEEDDSGLPVVTSGCLKLEGSDFQCRDTPIPHQRRSIECCTERNECNKDLHPTLPPVVIGPFFDGSIR

SEQ ID NO: 89 - protein, 356 aa, synthetic construct
KKEDGESTAPTPRPKVLRCYCSGHCPEDSVNNICSTDGYCFTMIEEDDSGLPVVTSGCLKLEGSDFQCRDTPIPHQRRSIECCTERNECNKDLHPTLPPVVIGPFFDGSIRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 107 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 91 - protein, 352 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 106 aa, synthetic construct
KKEDGESTAPTPRPKVLRCYCSGHCPEDSVNNICSTNGYCFTMIEEDDSGLPVVTSGCLGLEGSDFQCRDTPIPHQRRSIECCTERNECNKDLHPTLPPLKNRDFV

SEQ ID NO: 93 - protein, 351 aa, synthetic construct
KKEDGESTAPTPRPKVLRCYCSGHCPEDSVNNICSTNGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 106 aa, synthetic construct
KKEDGESTAPTPRPKVLRCVCSGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 95 - protein, 351 aa, synthetic construct
KKEDGESTAPTPRPKVLRCVCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 106 aa, synthetic construct
KKEDGESTAPTPRPKVLRCFCSGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 97 - protein, 351 aa, synthetic construct
KKEDGESTAPTPRPKVLRCFCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 106 aa, synthetic construct
KKEDGESTAPTPRPKVLRCYCGSHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 99 - protein, 351 aa, synthetic construct
KKEDGESTAPTPRPKVLRCYCGSHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 106 aa, synthetic construct
KKEDGESTAPTPRPKVLRCFCGSHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 101 - protein, 351 aa, synthetic construct
KKEDGESTAPTPRPKVLRCFCGSHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 107 aa, synthetic construct
KKEDGESTAPTPRPKVLRCYCGASHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 103 - protein, 352 aa, synthetic construct
KKEDGESTAPTPRPKVLRCYCGASHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 107 aa, synthetic construct
KKEDGESTAPTPRPKVLRCYCSAGHCPEDSVNNICSTDGYCFTMIEEDDSGLPVVTSGCLKLEGSDFQCRDTPIPHQRRSIECCTERNECNKDLHPTLPPLKNRDFV

SEQ ID NO: 105 - protein, 352 aa, synthetic construct
KKEDGESTAPTPRPKVLRCYCSAGHCPEDSVNNICSTDGYCFTMIEEDDSGLPVVTSGCLKLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 106 - protein, 224 aa, synthetic construct
THTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 225 aa, synthetic construct
THTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 107 aa, synthetic construct
  VARIANT 15: note = K or P
  VARIANT 16: note = V or I
  VARIANT 20: note = V, F, K or Y
  VARIANT 22: note = G, H or S
  VARIANT 23: note = absent or A
  VARIANT 24: note = S, H or G
  VARIANT 28: note = E or D
  VARIANT 30: note = S or A
  VARIANT 31: note = V or I
  VARIANT 34: note = I or T
  VARIANT 36: note = S or I
  VARIANT 38: note = D or N
  VARIANT 40: note = Y or H
  VARIANT 43: note = T or A
  VARIANT 44: note = M or I
  VARIANT 50: note = S or Q
  VARIANT 52: note = M, L or E
  VARIANT 53: note = P or T
  VARIANT 54: note = V or T
  VARIANT 55: note = V or L
  VARIANT 56: note = T or A
  VARIANT 60: note = L or M
  VARIANT 61: note = G or K
  VARIANT 62: note = L or Y
  VARIANT 70: note = R or K
  VARIANT 72: note = T or S
  VARIANT 74: note = I or K
  VARIANT 75: note = P or A
  VARIANT 76: note = H or Q
  VARIANT 77: note = Q or L
  VARIANT 80: note = S or T
  VARIANT 85: note = T or R
  VARIANT 86: note = absent, T or E
  VARIANT 87: note = R or N
  VARIANT 88: note = N or L
  VARIANT 89: note = absent, E or L
  VARIANT 92: note = K or Q
  VARIANT 93: note = D or L
  VARIANT 95: note = H or Q
KKEDGESTAPTPRPXXLRCXCXXXHCPXDXXNNXCXTXGXCFXXIEEDDXGXXXXXSGCXXXEGSDFQCXDXPXXXXRRXIECCXXXXXCNXXLXPTLPPLKNRDFV

SEQ ID NO: 109 - protein, 112 aa, synthetic construct
  VARIANT 15: note = K or P
  VARIANT 16: note = V or I
  VARIANT 20: note = V, F, K or Y
  VARIANT 22: note = G, H or S
  VARIANT 23: note = absent or A
  VARIANT 24: note = S, H or G
  VARIANT 28: note = E or D
  VARIANT 30: note = S or A
  VARIANT 31: note = V or I
  VARIANT 34: note = I or T
  VARIANT 36: note = S or I
  VARIANT 38: note = D or N
  VARIANT 40: note = Y or H
  VARIANT 43: note = T or A
  VARIANT 44: note = M or I
  VARIANT 50: note = S or Q
  VARIANT 52: note = L or E
  VARIANT 53: note = P or T
  VARIANT 54: note = V or T
  VARIANT 55: note = V or L
  VARIANT 56: note = T or A
  VARIANT 60: note = L or M
  VARIANT 61: note = G or K
  VARIANT 62: note = L or Y
  VARIANT 70: note = R or K
  VARIANT 72: note = T or S
  VARIANT 74: note = I or K
  VARIANT 75: note = P or A
  VARIANT 76: note = H or Q
  VARIANT 77: note = Q or L
  VARIANT 80: note = S or T
  VARIANT 85: note = T or R
  VARIANT 86: note = absent, T or E
  VARIANT 87: note = R or N
  VARIANT 88: note = N or L
  VARIANT 89: note = absent, E or L
  VARIANT 92: note = K or Q
  VARIANT 93: note = D or L
  VARIANT 95: note = H or Q
KKEDGESTAPTPRPXXLRCXCXXXHCPXDXXNNXCXTXGXCFXXIEEDDXGXXXXXSGCXXXEGSDFQCXDXPXXXXRRXIECCXXXXXCNXXLXPTLPPVVIGPFFDGSIR

SEQ ID NO: 110 - protein, 107 aa, synthetic construct
KKEDGESTAPTPRPKVLRCYCSAGHCPEDSVNNICSTDGYCFTMIEEDDSGLPVVTSGCMKYEGSDFQCRDTPIPHQRRSIECCTERNECNKDLHPTLPPLKNRDFV

SEQ ID NO: 111 - protein, 352 aa, synthetic construct
KKEDGESTAPTPRPKVLRCYCSAGHCPEDSVNNICSTDGYCFTMIEEDDSGLPVVTSGCMKYEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 356 aa, synthetic construct
KKEDGESTAPTPRPKVLRCKCHHHCPEDSVNNICSTDGYCFTMIEEDDSGLPVVTSGCMKYEGSDFQCRDTPIPHQRRSIECCTERNECNKDLHPTLPPVVIGPFFDGSIRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 107 aa, synthetic construct
KKEDGESTAPTPRPKVLRCKCHAHHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 114 - protein, 352 aa, synthetic construct
KKEDGESTAPTPRPKVLRCKCHAHHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 351 aa, synthetic construct
KKEDGESTAPTPRPKVLRCYCSGSCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 352 aa, synthetic construct
KKEDGESTAPTPRPKVLRCYCGAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 352 aa, synthetic construct
KKEDGESTAPTPRPKVLRCYCGGGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 352 aa, synthetic construct
KKEDGESTAPTPRPKVLRCVCEGLSCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 353 aa, synthetic construct
KKEDGESTAPTPRPKVLRCVCEGLSHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 351 aa, synthetic construct
KKEDGESTAPTPRPKVLRCTCHHHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 351 aa, synthetic construct
KKEDGESTAPTPRPKVLRCVCHHHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 351 aa, synthetic construct
KKEDGESTAPTPRPKVLRCFCHHHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 351 aa, synthetic construct
KKEDGESTAPTPRPKVLRCACHHHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 351 aa, synthetic construct
KKEDGESTAPTPRPKVLRCVCGSHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 351 aa, synthetic construct; identical to SEQ ID NO: 101

SEQ ID NO: 126 - protein, 351 aa, synthetic construct
KKEDGESTAPTPRPKVLRCACGSHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 352 aa, synthetic construct
KKEDGESTAPTPRPKVLRCYCGSLHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 352 aa, synthetic construct
KKEDGESTAPTPRPKVLRCYCGSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 351 aa, synthetic construct; identical to SEQ ID NO: 87

SEQ ID NO: 130 - protein, 109 aa, synthetic construct
  VARIANT 16: note = V or I
  VARIANT 20: note = V, F, K, Y, T, or A
  VARIANT 22: note = G, E, H or S
  VARIANT 23: note = absent, G, S, or A
  VARIANT 24: note = absent or S
  VARIANT 25: note = S, H, G, or L
  VARIANT 26: note = absent or L
  VARIANT 27: note = H or S
  VARIANT 30: note = E or D
  VARIANT 32: note = S or A
  VARIANT 33: note = V or I
  VARIANT 36: note = I or T
  VARIANT 38: note = S or I
  VARIANT 40: note = D or N
  VARIANT 42: note = Y or H
  VARIANT 45: note = T or A
  VARIANT 46: note = M or I
  VARIANT 52: note = S or Q
  VARIANT 54: note = M, L or E
  VARIANT 55: note = P or T
  VARIANT 56: note = V or T
  VARIANT 57: note = V or L
  VARIANT 58: note = T or A
  VARIANT 62: note = L or M
  VARIANT 63: note = G or K
  VARIANT 64: note = L or Y
  VARIANT 72: note = R or K
  VARIANT 74: note = T or S
  VARIANT 76: note = I or K
  VARIANT 77: note = P or A
  VARIANT 78: note = H or Q
  VARIANT 79: note = Q or L
  VARIANT 82: note = S or T
  VARIANT 87: note = T or R
  VARIANT 88: note = absent, T or E
  VARIANT 89: note = R or N
  VARIANT 90: note = N or L
  VARIANT 91: note = absent, E or L
  VARIANT 94: note = K or Q
  VARIANT 95: note = D or L
  VARIANT 97: note = H or Q
KKEDGESTAPTPRPKXLRCXCXXXXXXCPXDXXNNXCXTXGXCFXXIEEDDXGXXXXXSGCXXXEGSDFQCXDXPXXXXRRXIECCXXXXXCNXXLXPTLPPLKNRDFV

SEQ ID NO: 131 - protein, 375 aa, synthetic construct
QNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 106 aa, synthetic construct; identical to SEQ ID NO: 13

SEQ ID NO: 133 - protein, 106 aa, synthetic construct
KKEDGESTAPTPRPPVLRCKCHHHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 134 - protein, 105 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTRNECNKDLHPTLPPLKNRDFV

SEQ ID NO: 135 - protein, 106 aa, synthetic construct
KKEDGESTAPTPRPKVLRCKCHHHCPEDSINNICSTDGYCFTMIEEDDSGLPVVTSGCLGLEGSDFQCRDTPIPHQRRSIECCTERNECNKDLHPTLPPLKNRDFV

SEQ ID NO: 136 - protein, 106 aa, synthetic construct
KKEDGESTAPTPRPKVLRCKCHHHCPEDSVNNTCSTDGYCFTMIEEDDSGLPVVTSGCLGLEGSDFQCRDTPIPHQRRSIECCTERNECNKDLHPTLPPLKNRDFV

SEQ ID NO: 137 - protein, 106 aa, synthetic construct
KKEDGESTAPTPRPKVLRCKCHHHCPEDSVNNICITDGYCFTMIEEDDSGLPVVTSGCLGLEGSDFQCRDTPIPHQRRSIECCTERNECNKDLHPTLPPLKNRDFV

SEQ ID NO: 138 - protein, 106 aa, synthetic construct
KKEDGESTAPTPRPKVLRCKCHHHCPEDSVNNICSTDGHCFTMIEEDDSGLPVVTSGCLGLEGSDFQCRDTPIPHQRRSIECCTERNECNKDLHPTLPPLKNRDFV

SEQ ID NO: 139 - protein, 106 aa, synthetic construct
KKEDGESTAPTPRPKVLRCKCHHHCPEDSVNNICSTDGYCFTMIEEDDQGLPVVTSGCLGLEGSDFQCRDTPIPHQRRSIECCTERNECNKDLHPTLPPLKNRDFV

SEQ ID NO: 140 - protein, 106 aa, synthetic construct
KKEDGESTAPTPRPKVLRCKCHHHCPEDSVNNICSTDGYCFTMIEEDDSGLPVVTSGCLKLEGSDFQCRDTPIPHQRRSIECCTERNECNKDLHPTLPPLKNRDFV

SEQ ID NO: 141 - protein, 106 aa, synthetic construct
KKEDGESTAPTPRPKVLRCKCHHHCPEDSVNNICSTDGYCFTMIEEDDSGLPVVTSGCMKYEGSDFQCRDTPIPHQRRSIECCTERNECNKDLHPTLPPLKNRDFV

SEQ ID NO: 142 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCRERNECNKDLHPTLPPLKNRDFV

SEQ ID NO: 143 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTENNECNKDLHPTLPPLKNRDFV

SEQ ID NO: 144 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERLECNKDLHPTLPPLKNRDFV

SEQ ID NO: 145 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTTRNECNKDLHPTLPPLKNRDFV

SEQ ID NO: 146 - protein, 106 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNECNKYLHPTLPPLKNRDFV

SEQ ID NO: 147 - protein, 105 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTERNCNKDLHPTLPPLKNRDFV

SEQ ID NO: 148 - protein, 105 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCRTNLCNQYLQPTLPPLKNRDFV

SEQ ID NO: 149 - protein, 106 aa, synthetic construct
KKEDGESTAPTPRPKVLRCKCSGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 150 - protein, 107 aa, synthetic construct
KKEDGESTAPTPRPKVLRCVCEGLSCPEDSVNNICSTDGYCFTMIEEDDSGLPVVTSGCLGLEGSDFQCRDTPIPHQRRSIECCTERNECNKDLHPTLPPLKNRDFV

SEQ ID NO: 151 - protein, 107 aa, synthetic construct
KKEDGESTAPTPRPKVLRCVCEGLHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 152 - protein, 107 aa, synthetic construct
KKEDGESTAPTPRPKVLRCVCEGHSCPEDSVNNICSTDGYCFTMIEEDDSGLPVVTSGCLGLEGSDFQCRDTPIPHQRRSIECCTERNECNKDLHPTLPPLKNRDFV

SEQ ID NO: 153 - protein, 103 aa, synthetic construct
TPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 154 - protein, 100 aa, synthetic construct
PKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 155 - protein, 97 aa, synthetic construct
LRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHR

SEQ ID NO: 156 - protein, 102 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCRTNLCNQYLQPTLPPVVIG

SEQ ID NO: 157 - protein, 116 aa, synthetic construct
  VARIANT 1: note = K or absent
  VARIANT 2: note = K or absent
  VARIANT 3: note = E or absent
  VARIANT 4: note = D or absent
  VARIANT 5: note = G or absent
  VARIANT 6: note = E or absent
  VARIANT 7: note = S or absent
  VARIANT 8: note = T or absent
  VARIANT 9: note = A or absent
  VARIANT 10: note = P or absent
  VARIANT 11: note = T or absent
  VARIANT 12: note = P or absent
  VARIANT 13: note = R or absent
  VARIANT 14: note = P or absent
  VARIANT 15: note = K, P or absent
  VARIANT 16: note = V or absence
  VARIANT 20: note = V, F, K, A, R, N, D, C, E, Q, G, H, I, L, M, P, S, T, W, or Y
  VARIANT 22: note = G, H, V, F, K, A, R, N, D, C, E, Q, I, L, M, P, T, W, Y or S
  VARIANT 23: note = absent, G, S, or A
  VARIANT 24: note = absent or S
  VARIANT 25: note = S, H, V, F, K, A, R, N, D, C, E, Q, I, L, M, P, T, W, Y or G
  VARIANT 26: note = absent, L or H
  VARIANT 27: note = V, F, K, A, R, N, D, C, E, Q, G, H, I, L, M, P, S, T, W, or Y
  VARIANT 30: note = E or D
  VARIANT 32: note = S, G or A
  VARIANT 33: note = V, A, G or I
  VARIANT 36: note = I, S or T
  VARIANT 38: note = S, T, G, A or I
  VARIANT 40: note = D, A, G or N
  VARIANT 42: note = Y, A, G or H
  VARIANT 45: note = T or A
  VARIANT 46: note = M or I
  VARIANT 52: note = S, A, G or Q
  VARIANT 54: note = L, A, G or E
  VARIANT 55: note = P or T
  VARIANT 56: note = V, A, G or T
  VARIANT 57: note = V, A, G or L
  VARIANT 58: note = T, G or A
  VARIANT 62: note = L, M, V, F, K, A, R, N, D, C, E, Q, G, H, I, P, S, T, W, or Y
  VARIANT 63: note = G, K, V, F, A, R, N, D, C, E, Q, H, I, L, M, P, S, T, W, or Y
  VARIANT 64: note = L, Y, V, F, K, A, R, N, D, C, E, Q, G, H, I, M, P, S, T, or W
  VARIANT 72: note = R or K
  VARIANT 74: note = T or S
  VARIANT 76: note = I or K
  VARIANT 77: note = P or A
  VARIANT 78: note = H or Q
  VARIANT 79: note = Q or L
  VARIANT 82: note = S or T
  VARIANT 87: note = T, R or absent
  VARIANT 88: note = absent, T or E
  VARIANT 89: note = R, N or absent
  VARIANT 90: note = N, L or absent
  VARIANT 91: note = absent, E or L
  VARIANT 94: note = K, Q or absent
  VARIANT 95: note = D, L, Y or absent
  VARIANT 97: note = H, Q or absent
  VARIANT 103: note = absent, V or L
  VARIANT 104: note = absent, V or K
  VARIANT 105: note = absent, I or N
  VARIANT 106: note = absent, G or R
  VARIANT 107: note = absent, P, D or A
  VARIANT 108: note = absent or F
  VARIANT 109: note = absent, F, W, Y, N, Q, K, H, D, G, E or V
  VARIANT 110: note = absent, L, M, V, F, K, A, R, N, C, E, Q, G, H, I, P, S, T, W, or Y or D
  VARIANT 111: note = absent, R, T, Q, L, K, W or G
  VARIANT 112: note = absent, S or P
  VARIANT 113: note = absent or I
  VARIANT 114: note = absent, R or H
  VARIANT 115: note = absent or H
  VARIANT 116: note = absent or R
XXXXXXXXXXXXXXXXLRCXCXXXXXXCPXDXXNNXCXTXGXCFXXIEEDDXGXXXXXSGCXXXEGSDFQCXDXPXXXXRRXIECCXXXXXCNXXLXPTLPPXXXXXXXXXXXXXX

SEQ ID NO: 158 - protein, 106 aa, synthetic construct
KKEDGESTAPTPRPKVLRCKCHHHCPEDSINNICITDGYCFTMIEEDDQGLPVVTSGCLGLEGSDFQCRDTPIPHQRRSIECCTERNECNKDLHPTLPPLKNRDFV

SEQ ID NO: 159 - protein, 106 aa, synthetic construct
KKEDGESTAPTPRPKVLRCKCSHHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 160 - protein, 106 aa, synthetic construct
KKEDGESTAPTPRPKVLRCKCHHHCPEDSVNNICTTDGYCFTMIEEDDSGLPVVTSGCLGLEGSDFQCRDTPIPHQRRSIECCTERNECNKDLHPTLPPLKNRDFV

SEQ ID NO: 161 - protein, 106 aa, synthetic construct
KKEDGESTAPTPRPKVLRCKCHHHCPEDSVNNSCSTDGYCFTMIEEDDSGLPVVTSGCLGLEGSDFQCRDTPIPHQRRSIECCTERNECNKDLHPTLPPLKNRDFV

SEQ ID NO: 162 - protein, 106 aa, synthetic construct
KKEDGESTAPTPRPKVLRCKCHHHCPEDGVNNICSTDGYCFTMIEEDDSGLPVVTSGCLGLEGSDFQCRDTPIPHQRRSIECCTERNECNKDLHPTLPPLKNRDFV

SEQ ID NO: 163 - protein, 106 aa, synthetic construct
KKEDGESTAPTPRPKVLRCKCHHHCPEDSGNNICSTDGYCFTMIEEDDSGLPVVTSGCLGLEGSDFQCRDTPIPHQRRSIECCTERNECNKDLHPTLPPLKNRDFV

SEQ ID NO: 164 - protein, 106 aa, synthetic construct
KKEDGESTAPTPRPKVLRCKCHHHCPEDSANNICSTDGYCFTMIEEDDSGLPVVTSGCLGLEGSDFQCRDTPIPHQRRSIECCTERNECNKDLHPTLPPLKNRDFV

SEQ ID NO: 165 - protein, 106 aa, synthetic construct
KKEDGESTAPTPRPKVLRCKCHHHCPEDSVNNICGTDGYCFTMIEEDDSGLPVVTSGCLGLEGSDFQCRDTPIPHQRRSIECCTERNECNKDLHPTLPPLKNRDFV

SEQ ID NO: 166 - protein, 106 aa, synthetic construct
KKEDGESTAPTPRPKVLRCKCHHHCPEDSVNNICATDGYCFTMIEEDDSGLPVVTSGCLGLEGSDFQCRDTPIPHQRRSIECCTERNECNKDLHPTLPPLKNRDFV

SEQ ID NO: 167 - protein, 106 aa, synthetic construct
KKEDGESTAPTPRPKVLRCKCHHHCPEDSVNNICSTAGYCFTMIEEDDSGLPVVTSGCLGLEGSDFQCRDTPIPHQRRSIECCTERNECNKDLHPTLPPLKNRDFV

SEQ ID NO: 168 - protein, 106 aa, synthetic construct
KKEDGESTAPTPRPKVLRCKCHHHCPEDSVNNICSTGGYCFTMIEEDDSGLPVVTSGCLGLEGSDFQCRDTPIPHQRRSIECCTERNECNKDLHPTLPPLKNRDFV

SEQ ID NO: 169 - protein, 106 aa, synthetic construct
KKEDGESTAPTPRPKVLRCKCHHHCPEDSVNNICSTDGACFTMIEEDDSGLPVVTSGCLGLEGSDFQCRDTPIPHQRRSIECCTERNECNKDLHPTLPPLKNRDFV

SEQ ID NO: 170 - protein, 106 aa, synthetic construct
KKEDGESTAPTPRPKVLRCKCHHHCPEDSVNNICSTDGGCFTMIEEDDSGLPVVTSGCLGLEGSDFQCRDTPIPHQRRSIECCTERNECNKDLHPTLPPLKNRDFV

SEQ ID NO: 171 - protein, 106 aa, synthetic construct
KKEDGESTAPTPRPKVLRCKCHHHCPEDSVNNICSTDGYCFTMIEEDDAGLPVVTSGCLGLEGSDFQCRDTPIPHQRRSIECCTERNECNKDLHPTLPPLKNRDFV

SEQ ID NO: 172 - protein, 106 aa, synthetic construct
KKEDGESTAPTPRPKVLRCKCHHHCPEDSVNNICSTDGYCFTMIEEDDGGLPVVTSGCLGLEGSDFQCRDTPIPHQRRSIECCTERNECNKDLHPTLPPLKNRDFV

SEQ ID NO: 173 - protein, 106 aa, synthetic construct
KKEDGESTAPTPRPKVLRCKCHHHCPEDSVNNICSTDGYCFTMIEEDDSGEPVVTSGCLGLEGSDFQCRDTPIPHQRRSIECCTERNECNKDLHPTLPPLKNRDFV

SEQ ID NO: 174 - protein, 106 aa, synthetic construct
KKEDGESTAPTPRPKVLRCKCHHHCPEDSVNNICSTDGYCFTMIEEDDSGAPVVTSGCLGLEGSDFQCRDTPIPHQRRSIECCTERNECNKDLHPTLPPLKNRDFV

SEQ ID NO: 175 - protein, 106 aa, synthetic construct
KKEDGESTAPTPRPKVLRCKCHHHCPEDSVNNICSTDGYCFTMIEEDDSGGPVVTSGCLGLEGSDFQCRDTPIPHQRRSIECCTERNECNKDLHPTLPPLKNRDFV

SEQ ID NO: 176 - protein, 106 aa, synthetic construct
KKEDGESTAPTPRPKVLRCKCHHHCPEDSVNNICSTDGYCFTMIEEDDSGLPTVTSGCLGLEGSDFQCRDTPIPHQRRSIECCTERNECNKDLHPTLPPLKNRDFV

SEQ ID NO: 177 - protein, 106 aa, synthetic construct
KKEDGESTAPTPRPKVLRCKCHHHCPEDSVNNICSTDGYCFTMIEEDDSGLPAVTSGCLGLEGSDFQCRDTPIPHQRRSIECCTERNECNKDLHPTLPPLKNRDFV

SEQ ID NO: 178 - protein, 106 aa, synthetic construct
KKEDGESTAPTPRPKVLRCKCHHHCPEDSVNNICSTDGYCFTMIEEDDSGLPGVTSGCLGLEGSDFQCRDTPIPHQRRSIECCTERNECNKDLHPTLPPLKNRDFV

SEQ ID NO: 179 - protein, 106 aa, synthetic construct
KKEDGESTAPTPRPKVLRCKCHHHCPEDSVNNICSTDGYCFTMIEEDDSGLPVTTSGCLGLEGSDFQCRDTPIPHQRRSIECCTERNECNKDLHPTLPPLKNRDFV

SEQ ID NO: 180 - protein, 106 aa, synthetic construct
KKEDGESTAPTPRPKVLRCKCHHHCPEDSVNNICSTDGYCFTMIEEDDSGLPVATSGCLGLEGSDFQCRDTPIPHQRRSIECCTERNECNKDLHPTLPPLKNRDFV

SEQ ID NO: 181 - protein, 106 aa, synthetic construct
KKEDGESTAPTPRPKVLRCKCHHHCPEDSVNNICSTDGYCFTMIEEDDSGLPVGTSGCLGLEGSDFQCRDTPIPHQRRSIECCTERNECNKDLHPTLPPLKNRDFV

SEQ ID NO: 182 - protein, 106 aa, synthetic construct
KKEDGESTAPTPRPKVLRCKCHHHCPEDSVNNICSTDGYCFTMIEEDDSGLPVVASGCLGLEGSDFQCRDTPIPHQRRSIECCTERNECNKDLHPTLPPLKNRDFV

SEQ ID NO: 183 - protein, 106 aa, synthetic construct
KKEDGESTAPTPRPKVLRCKCHHHCPEDSVNNICSTDGYCFTMIEEDDSGLPVVGSGCLGLEGSDFQCRDTPIPHQRRSIECCTERNECNKDLHPTLPPLKNRDFV

SEQ ID NO: 184 - protein, 106 aa, synthetic construct
KKEDGESTAPTPRPKVLRCKCHHHCPEDSVNNICSTDGYCFTMIEEDDSGLPVVTSGCMGLEGSDFQCRDTPIPHQRRSIECCTERNECNKDLHPTLPPLKNRDFV

SEQ ID NO: 185 - protein, 106 aa, synthetic construct
KKEDGESTAPTPRPKVLRCKCHHHCPEDSVNNICSTDGYCFTMIEEDDSGLPVVTSGCAGLEGSDFQCRDTPIPHQRRSIECCTERNECNKDLHPTLPPLKNRDFV

SEQ ID NO: 186 - protein, 106 aa, synthetic construct
KKEDGESTAPTPRPKVLRCKCHHHCPEDSVNNICSTDGYCFTMIEEDDSGLPVVTSGCGGLEGSDFQCRDTPIPHQRRSIECCTERNECNKDLHPTLPPLKNRDFV

SEQ ID NO: 187 - protein, 106 aa, synthetic construct
KKEDGESTAPTPRPKVLRCYCSHHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 188 - protein, 106 aa, synthetic construct
KKEDGESTAPTPRPKVLRCYCSHHCPEDSVNNTCSTDGYCFTMIEEDDSGLPVVTSGCLGLEGSDFQCRDTPIPHQRRSIECCTERNECNKDLHPTLPPLKNRDFV

SEQ ID NO: 189 - protein, 106 aa, synthetic construct
KKEDGESTAPTPRPKVLRCKCSHHCPEDSVNNSCSTDGYCFTMIEEDDSGLPVVTSGCLGLEGSDFQCRDTPIPHQRRSIECCTERNECNKDLHPTLPPLKNRDFV

SEQ ID NO: 190 - protein, 109 aa, synthetic construct
  VARIANT 15: note = K or P
  VARIANT 16: note = V or I
  VARIANT 20: note = V, F, K, A, R, N, D, C, E, Q, G, H, I, L, M, P, S, T, W, or Y
  VARIANT 22: note = G, H, V, F, K, A, R, N, D, C, E, Q, I, L, M, P, T, W, Y or S
  VARIANT 23: note = absent, G, S, or A
  VARIANT 24: note = absent or S
  VARIANT 25: note = S, H, V, F, K, A, R, N, D, C, E, Q, I, L, M, P, T, W, Y or G
  VARIANT 26: note = absent, L or H
  VARIANT 27: note = V, F, K, A, R, N, D, C, E, Q, G, H, I, L, M, P, S, T, W, or Y
  VARIANT 30: note = E or D
  VARIANT 32: note = S, G or A
  VARIANT 33: note = V, A, G or I
  VARIANT 36: note = I, S or T
  VARIANT 38: note = S, T, G, A or I
  VARIANT 40: note = D, A, G or N
  VARIANT 42: note = Y, A, G or H
  VARIANT 45: note = T or A
  VARIANT 46: note = M or I
  VARIANT 52: note = S, A, G or Q
  VARIANT 54: note = L, A, G or E
  VARIANT 55: note = P or T
  VARIANT 56: note = V, A, G or T
  VARIANT 57: note = V, A, G or L
  VARIANT 58: note = T, G or A
  VARIANT 62: note = L, M, V, F, K, A, R, N, D, C, E, Q, G, H, I, P, S, T, W, or Y
  VARIANT 63: note = G, K, V, F, A, R, N, D, C, E, Q, H, I, L, M, P, S, T, W, or Y
  VARIANT 64: note = L, Y, V, F, K, A, R, N, D, C, E, Q, G, H, I, M, P, S, T, or W
  VARIANT 72: note = R or K
  VARIANT 74: note = T or S
  VARIANT 76: note = I or K
  VARIANT 77: note = P or A
  VARIANT 78: note = H or Q
  VARIANT 79: note = Q or L
  VARIANT 82: note = S or T
  VARIANT 87: note = T, R or absent
  VARIANT 88: note = absent, T or E
  VARIANT 89: note = absent, R or N
  VARIANT 90: note = N or L
  VARIANT 91: note = absent, E or L
  VARIANT 94: note = absent, K or Q
  VARIANT 95: note = absent, D, Y or L
  VARIANT 97: note = absent, H or Q
  VARIANT 107: note = D or A
KKEDGESTAPTPRPXXLRCXCXXXXXXCPXDXXNNXCXTXGXCFXXIEEDDXGXXXXXSGCXXXEGSDFQCXDXPXXXXRRXIECCXXXXXCNXXLXPTLPPLKNRXFV

SEQ ID NO: 191 - protein, 106 aa, synthetic construct
KKEDGESTAPTPRPKVLRCKCHHHCPEDSINNICITDGYCFTMIEEDDQGLPVVTSGCLKLEGSDFQCRDTPIPHQRRSIECCTERNECNKDLHPTLPPLKNRDFV

SEQ ID NO: 192 - protein, 106 aa, synthetic construct
KKEDGESTAPTPRPKVLRCKCSHHCPEDSVNNICSTDGYCFTMIEEDDSGLPVVTSGCLKLEGSDFQCRDTPIPHQRRSIECCTERNECNKDLHPTLPPLKNRDFV

SEQ ID NO: 193 - protein, 106 aa, synthetic construct
KKEDGESTAPTPRPKVLRCYCSHHCPEDSVNNTCSTDGYCFTMIEEDDSGLPVVTSGCLKLEGSDFQCRDTPIPHQRRSIECCTERNECNKDLHPTLPPLKNRDFV

SEQ ID NO: 194 - protein, 106 aa, synthetic construct
KKEDGESTAPTPRPKVLRCKCSHHCPEDSVNNSCSTDGYCFTMIEEDDSGLPVVTSGCLKLEGSDFQCRDTPIPHQRRSIECCTERNECNKDLHPTLPPLKNRDFV

SEQ ID NO: 195 - protein, 19 aa, Homo sapiens
RTNLCNQYLQPTLPPVVIG

SEQ ID NO: 196 - protein, 23 aa, synthetic construct
EPKSCDKTHTCPPCPAPELLGGP

SEQ ID NO: 197 - protein, 19 aa, synthetic construct
ERKCCVECPPCPAPPVAGP

SEQ ID NO: 198 - protein, 20 aa, synthetic construct
ESKYGPPCPSCPAPEFLGGP

SEQ ID NO: 199 - protein, 16 aa, synthetic construct
THTCPPCPAPEAAGGP

SEQ ID NO: 200 - protein, 16 aa, synthetic construct
THTCPPCKCPEAAGGP

SEQ ID NO: 201 - protein, 14 aa, synthetic construct
PTIKPCPPCKCPAP

SEQ ID NO: 202 - protein, 106 aa, synthetic construct
KKEDGESTAPTPRPKVLRCKCSHHCPEDSINNTCITDGYCFTMIEEDDSGLPVVTSGCLGLEGSDFQCRDTPIPHQRRSIECCTERNECNKDLHPTLPPLKNRDFV

SEQ ID NO: 203 - protein, 351 aa, synthetic construct
KKEDGESTAPTPRPKVLRCKCSHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 204 - protein, 143 aa, synthetic construct
KKEDGESTAPTPRPKVLRCKCSHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 205 - protein, 143 aa, synthetic construct
KKEDGESTAPTPRPKVLRCKCSHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 206 - protein, 141 aa, synthetic construct
KKEDGESTAPTPRPKVLRCKCSHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 207 - protein, 143 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 208 - protein, 143 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 209 - protein, 141 aa, synthetic construct
KKEDGESTAPTPRPKVLRCYCSGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 210 - protein, 144 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 211 - protein, 144 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 212 - protein, 142 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 213 - protein, 106 aa, synthetic construct
KKEDGESTAPTPRPKVLRCKCHHHCPEDSINNTCITDGYCFTMIEEDDSGLPVVTSGCLGLEGSDFQCRDTPIPHQRRSIECCTERNECNKDLHPTLPPLKNRDFV

SEQ ID NO: 214 - protein, 351 aa, synthetic construct
KKEDGESTAPTPRPKVLRCKCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 215 - protein, 143 aa, synthetic construct
KKEDGESTAPTPRPKVLRCKCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 216 - protein, 143 aa, synthetic construct
KKEDGESTAPTPRPKVLRCKCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 217 - protein, 141 aa, synthetic construct
KKEDGESTAPTPRPKVLRCKCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 218 - protein, 106 aa, synthetic construct
KKEDGESTAPTPRPKVLRCKCSHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 219 - protein, 351 aa, synthetic construct
KKEDGESTAPTPRPKVLRCKCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 220 - protein, 143 aa, synthetic construct
KKEDGESTAPTPRPKVLRCKCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 221 - protein, 143 aa, synthetic construct
KKEDGESTAPTPRPKVLRCKCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 222 - protein, 141 aa, synthetic construct
KKEDGESTAPTPRPKVLRCKCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 223 - protein, 106 aa, synthetic construct
KKEDGESTAPTPRPKVLRCKCSHHCPEDSINNTCSTDGYCFTMIEEDDSGLPVVTSGCLGLEGSDFQCRDTPIPHQRRSIECCTERNECNKDLHPTLPPLKNRDFV

SEQ ID NO: 224 - protein, 351 aa, synthetic construct
KKEDGESTAPTPRPKVLRCKCSHHCPEDSINNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 225 - protein, 143 aa, synthetic construct
KKEDGESTAPTPRPKVLRCKCSHHCPEDSINNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 226 - protein, 143 aa, synthetic construct
KKEDGESTAPTPRPKVLRCKCSHHCPEDSINNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 227 - protein, 141 aa, synthetic construct
KKEDGESTAPTPRPKVLRCKCSHHCPEDSINNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 228 - protein, 106 aa, synthetic construct
KKEDGESTAPTPRPKVLRCKCSHHCPEDSVNNTCSTDGYCFTMIEEDDSGLPVVTSGCLGLEGSDFQCRDTPIPHQRRSIECCTERNECNKDLHPTLPPLKNRDFV

SEQ ID NO: 229 - protein, 351 aa, synthetic construct
KKEDGESTAPTPRPKVLRCKCSHHCPEDSVNNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 230 - protein, 143 aa, synthetic construct
KKEDGESTAPTPRPKVLRCKCSHHCPEDSVNNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 231 - protein, 143 aa, synthetic construct
KKEDGESTAPTPRPKVLRCKCSHHCPEDSVNNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 232 - protein, 141 aa, synthetic construct
KKEDGESTAPTPRPKVLRCKCSHHCPEDSVNNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 233 - protein, 106 aa, synthetic construct
KKEDGESTAPTPRPKVLRCKCHHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 234 - protein, 351 aa, synthetic construct
KKEDGESTAPTPRPKVLRCKCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 235 - protein, 143 aa, synthetic construct
KKEDGESTAPTPRPKVLRCKCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 236 - protein, 143 aa, synthetic construct
KKEDGESTAPTPRPKVLRCKCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 237 - protein, 141 aa, synthetic construct
KKEDGESTAPTPRPKVLRCKCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 238 - protein, 351 aa, synthetic construct
KKEDGESTAPTPRPKVLRCKCHHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 239 - protein, 143 aa, synthetic construct
KKEDGESTAPTPRPKVLRCKCHHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 240 - protein, 143 aa, synthetic construct
KKEDGESTAPTPRPKVLRCKCHHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 241 - protein, 141 aa, synthetic construct
KKEDGESTAPTPRPKVLRCKCHHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 242 - protein, 106 aa, synthetic construct
KKEDGESTAPTPRPKVLRCFCHHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 243 - protein, 351 aa, synthetic construct
KKEDGESTAPTPRPKVLRCFCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 244 - protein, 143 aa, synthetic construct
KKEDGESTAPTPRPKVLRCFCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 245 - protein, 143 aa, synthetic construct
KKEDGESTAPTPRPKVLRCFCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 246 - protein, 141 aa, synthetic construct
KKEDGESTAPTPRPKVLRCFCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 247 - protein, 106 aa, synthetic construct
KKEDGESTAPTPRPKVLRCKCSHHCPEDSVNNICITDGYCFTMIEEDDSGLPVVTSGCLGLEGSDFQCRDTPIPHQRRSIECCTERNECNKDLHPTLPPLKNRDFV

SEQ ID NO: 248 - protein, 351 aa, synthetic construct
KKEDGESTAPTPRPKVLRCKCSHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 249 - protein, 143 aa, synthetic construct
KKEDGESTAPTPRPKVLRCKCSHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 250 - protein, 143 aa, synthetic construct
KKEDGESTAPTPRPKVLRCKCSHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 251 - protein, 141 aa, synthetic construct
KKEDGESTAPTPRPKVLRCKCSHHCPEDSVNNICITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 252 - protein, 106 aa, synthetic construct
KKEDGESTAPTPRPKVLRCFCSHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 253 - protein, 351 aa, synthetic construct
KKEDGESTAPTPRPKVLRCFCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 254 - protein, 143 aa, synthetic construct
KKEDGESTAPTPRPKVLRCFCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 255 - protein, 143 aa, synthetic construct
KKEDGESTAPTPRPKVLRCFCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 256 - protein, 141 aa, synthetic construct
KKEDGESTAPTPRPKVLRCFCSHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 257 - protein, 106 aa, synthetic construct
KKEDGESTAPTPRPKVLRCICHHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 258 - protein, 351 aa, synthetic construct
KKEDGESTAPTPRPKVLRCICHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 259 - protein, 143 aa, synthetic construct
KKEDGESTAPTPRPKVLRCICHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 260 - protein, 143 aa, synthetic construct
KKEDGESTAPTPRPKVLRCICHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 261 - protein, 141 aa, synthetic construct
KKEDGESTAPTPRPKVLRCICHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 262 - protein, 106 aa, synthetic construct
KKEDGESTAPTPRPKVLRCACHHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 263 - protein, 351 aa, synthetic construct
KKEDGESTAPTPRPKVLRCACHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 264 - protein, 143 aa, synthetic construct
KKEDGESTAPTPRPKVLRCACHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 265 - protein, 143 aa, synthetic construct
KKEDGESTAPTPRPKVLRCACHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 266 - protein, 141 aa, synthetic construct
KKEDGESTAPTPRPKVLRCACHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 267 - protein, 106 aa, synthetic construct
KKEDGESTAPTPRPKVLRCTCHHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 268 - protein, 351 aa, synthetic construct
KKEDGESTAPTPRPKVLRCTCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 269 - protein, 143 aa, synthetic construct
KKEDGESTAPTPRPKVLRCTCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 270 - protein, 143 aa, synthetic construct
KKEDGESTAPTPRPKVLRCTCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 271 - protein, 141 aa, synthetic construct
KKEDGESTAPTPRPKVLRCTCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 272 - protein, 106 aa, synthetic construct
KKEDGESTAPTPRPKVLRCVCHHHCPEDSVNNTCITDGYCFTMIEEDDSGLPVVTSGCLGLEGSDFQCRDTPIPHQRRSIECCTERNECNKDLHPTLPPLKNRDFV

SEQ ID NO: 273 - protein, 351 aa, synthetic construct
KKEDGESTAPTPRPKVLRCVCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 274 - protein, 143 aa, synthetic construct
KKEDGESTAPTPRPKVLRCVCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 275 - protein, 143 aa, synthetic construct
KKEDGESTAPTPRPKVLRCVCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 276 - protein, 141 aa, synthetic construct
KKEDGESTAPTPRPKVLRCVCHHHCPEDSVNNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 277 - protein, 106 aa, synthetic construct
KKEDGESTAPTPRPKVLRCYCHHHCPEDSINNTCITDGYCFTMIEEDDSGLPVVTSGCLGLEGSDFQCRDTPIPHQRRSIECCTERNECNKDLHPTLPPLKNRDFV

SEQ ID NO: 278 - protein, 351 aa, synthetic construct
KKEDGESTAPTPRPKVLRCYCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 279 - protein, 143 aa, synthetic construct
KKEDGESTAPTPRPKVLRCYCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 280 - protein, 143 aa, synthetic construct
KKEDGESTAPTPRPKVLRCYCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 281 - protein, 141 aa, synthetic construct
KKEDGESTAPTPRPKVLRCYCHHHCPEDSINNTCITDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 282 - protein, 351 aa, synthetic construct
KKEDGESTAPTPRPKVLRCKCHHHCPEDSVNNTCSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 283 - protein, 113 aa, synthetic construct
KKEDGESTAPTPRPKVLRCKCHHHCPEDSVNNTCITDGYCFTMIEEDDSGLPVVTSGCLGLEGSDFQCRDTPIPHQRRSIECCTERNECNKDLHPTLPPLKNRDFFDGPIHHR

SEQ ID NO: 284 - protein, 358 aa, synthetic construct
KKEDGESTAPTPRPKVLRCKCHHHCPEDSVNNTCITDGYCFTMIEEDDSGLPVVTSGCLGLEGSDFQCRDTPIPHQRRSIECCTERNECNKDLHPTLPPLKNRDFFDGPIHHRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 285 - protein, 224 aa, synthetic construct
THTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 286 - protein, 107 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTERNECNKDLHPTLPPLKNRDFV

SEQ ID NO: 287 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 288 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 289 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 290 - protein, 142 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 291 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 292 - protein, 107 aa, synthetic construct
KKEDGESTAPTPRPKILRCYCSAGHCPDDSVNNICSTDGYCFTMIEEDDSGMPVVTSGCLGLEGSDFQCRDTPIPHQRRSIECCTERNECNKDLHPTLPPLKNRDFV

SEQ ID NO: 293 - protein, 352 aa, synthetic construct
KKEDGESTAPTPRPKILRCYCSAGHCPDDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 294 - protein, 144 aa, synthetic construct
KKEDGESTAPTPRPKILRCYCSAGHCPDDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 295 - protein, 144 aa, synthetic construct
KKEDGESTAPTPRPKILRCYCSAGHCPDDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 296 - protein, 142 aa, synthetic construct
KKEDGESTAPTPRPKILRCYCSAGHCPDDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 297 - protein, 352 aa, synthetic construct
KKEDGESTAPTPRPKILRCYCSAGHCPDDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 298 - protein, 107 aa, synthetic construct
KKEDGESTAPTPRPKILRCYCSAGHCPEDSINNICSTDGYCFTMIEEDDSGMPVVTSGCLGLEGSDFQCRDTPIPHQRRSIECCTERNECNKDLHPTLPPLKNRDFV

SEQ ID NO: 299 - protein, 352 aa, synthetic construct
KKEDGESTAPTPRPKILRCYCSAGHCPEDSINNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 300 - protein, 144 aa, synthetic construct
KKEDGESTAPTPRPKILRCYCSAGHCPEDSINNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 301 - protein, 144 aa, synthetic construct
KKEDGESTAPTPRPKILRCYCSAGHCPEDSINNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 302 - protein, 142 aa, synthetic construct
KKEDGESTAPTPRPKILRCYCSAGHCPEDSINNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 303 - protein, 352 aa, synthetic construct
KKEDGESTAPTPRPKILRCYCSAGHCPEDSINNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 304 - protein, 107 aa, synthetic construct
KKEDGESTAPTPRPKILRCYCSAGHCPEDSVNNTCSTDGYCFTMIEEDDSGMPVVTSGCLGLEGSDFQCRDTPIPHQRRSIECCTERNECNKDLHPTLPPLKNRDFV

SEQ ID NO: 305 - protein, 352 aa, synthetic construct
KKEDGESTAPTPRPKILRCYCSAGHCPEDSVNNTCSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 306 - protein, 144 aa, synthetic construct
KKEDGESTAPTPRPKILRCYCSAGHCPEDSVNNTCSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 307 - protein, 144 aa, synthetic construct
KKEDGESTAPTPRPKILRCYCSAGHCPEDSVNNTCSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 308 - protein, 142 aa, synthetic construct
KKEDGESTAPTPRPKILRCYCSAGHCPEDSVNNTCSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 309 - protein, 352 aa, synthetic construct
KKEDGESTAPTPRPKILRCYCSAGHCPEDSVNNTCSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 310 - protein, 107 aa, synthetic construct
KKEDGESTAPTPRPKILRCYCSAGHCPEDSVNNICITDGYCFTMIEEDDSGMPVVTSGCLGLEGSDFQCRDTPIPHQRRSIECCTERNECNKDLHPTLPPLKNRDFV

SEQ ID NO: 311 - protein, 352 aa, synthetic construct
KKEDGESTAPTPRPKILRCYCSAGHCPEDSVNNICITDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 312 - protein, 144 aa, synthetic construct
KKEDGESTAPTPRPKILRCYCSAGHCPEDSVNNICITDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 313 - protein, 144 aa, synthetic construct
KKEDGESTAPTPRPKILRCYCSAGHCPEDSVNNICITDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 314 - protein, 142 aa, synthetic construct
KKEDGESTAPTPRPKILRCYCSAGHCPEDSVNNICITDGYCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 315 - protein, 352 aa, synthetic construct
KKEDGESTAPTPRPKILRCYCSAGHCPEDSVNNICITDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 316 - protein, 107 aa, synthetic construct
KKEDGESTAPTPRPKILRCYCSAGHCPEDSVNNICSTNGYCFTMIEEDDSGMPVVTSGCLGLEGSDFQCRDTPIPHQRRSIECCTERNECNKDLHPTLPPLKNRDFV

SEQ ID NO: 317 - protein, 352 aa, synthetic construct
KKEDGESTAPTPRPKILRCYCSAGHCPEDSVNNICSTNGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 318 - protein, 144 aa, synthetic construct
KKEDGESTAPTPRPKILRCYCSAGHCPEDSVNNICSTNGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 319 - protein, 144 aa, synthetic construct
KKEDGESTAPTPRPKILRCYCSAGHCPEDSVNNICSTNGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 320 - protein, 142 aa, synthetic construct
KKEDGESTAPTPRPKILRCYCSAGHCPEDSVNNICSTNGYCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 321 - protein, 352 aa, synthetic construct
KKEDGESTAPTPRPKILRCYCSAGHCPEDSVNNICSTNGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 322 - protein, 107 aa, synthetic construct
KKEDGESTAPTPRPKILRCYCSAGHCPEDSVNNICSTDGHCFTMIEEDDSGMPVVTSGCLGLEGSDFQCRDTPIPHQRRSIECCTERNECNKDLHPTLPPLKNRDFV

SEQ ID NO: 323 - protein, 352 aa, synthetic construct
KKEDGESTAPTPRPKILRCYCSAGHCPEDSVNNICSTDGHCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 324 - protein, 144 aa, synthetic construct
KKEDGESTAPTPRPKILRCYCSAGHCPEDSVNNICSTDGHCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 325 - protein, 144 aa, synthetic construct
KKEDGESTAPTPRPKILRCYCSAGHCPEDSVNNICSTDGHCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 326 - protein, 142 aa, synthetic construct
KKEDGESTAPTPRPKILRCYCSAGHCPEDSVNNICSTDGHCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 327 - protein, 352 aa, synthetic construct
KKEDGESTAPTPRPKILRCYCSAGHCPEDSVNNICSTDGHCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 328 - protein, 107 aa, synthetic construct
KKEDGESTAPTPRPKILRCYCSAGHCPEDSVNNICSTDGYCFAMIEEDDSGMPVVTSGCLGLEGSDFQCRDTPIPHQRRSIECCTERNECNKDLHPTLPPLKNRDFV

SEQ ID NO: 329 - protein, 352 aa, synthetic construct
KKEDGESTAPTPRPKILRCYCSAGHCPEDSVNNICSTDGYCFAMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 330 - protein, 144 aa, synthetic construct
KKEDGESTAPTPRPKILRCYCSAGHCPEDSVNNICSTDGYCFAMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 331 - protein, 144 aa, synthetic construct
KKEDGESTAPTPRPKILRCYCSAGHCPEDSVNNICSTDGYCFAMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 332 - protein, 142 aa, synthetic construct
KKEDGESTAPTPRPKILRCYCSAGHCPEDSVNNICSTDGYCFAMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 333 - protein, 352 aa, synthetic construct
KKEDGESTAPTPRPKILRCYCSAGHCPEDSVNNICSTDGYCFAMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 334 - protein, 107 aa, synthetic construct
KKEDGESTAPTPRPKILRCYCSAGHCPEDSVNNICSTDGYCFTIIEEDDSGMPVVTSGCLGLEGSDFQCRDTPIPHQRRSIECCTERNECNKDLHPTLPPLKNRDFV

SEQ ID NO: 335 - protein, 352 aa, synthetic construct
KKEDGESTAPTPRPKILRCYCSAGHCPEDSVNNICSTDGYCFTIIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 336 - protein, 144 aa, synthetic construct
KKEDGESTAPTPRPKILRCYCSAGHCPEDSVNNICSTDGYCFTIIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 337 - protein, 144 aa, synthetic construct
KKEDGESTAPTPRPKILRCYCSAGHCPEDSVNNICSTDGYCFTIIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 338 - protein, 142 aa, synthetic construct
KKEDGESTAPTPRPKILRCYCSAGHCPEDSVNNICSTDGYCFTIIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 339 - protein, 352 aa, synthetic construct
KKEDGESTAPTPRPKILRCYCSAGHCPEDSVNNICSTDGYCFTIIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 340 - protein, 107 aa, synthetic construct
KKEDGESTAPTPRPKILRCYCSAGHCPEDSVNNICSTDGYCFTMIEEDDSGMPVVASGCLGLEGSDFQCRDTPIPHQRRSIECCTERNECNKDLHPTLPPLKNRDFV

SEQ ID NO: 341 - protein, 352 aa, synthetic construct
KKEDGESTAPTPRPKILRCYCSAGHCPEDSVNNICSTDGYCFTMIEEDDSGMPVVASGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 144 aa, synthetic construct
KKEDGESTAPTPRPKILRCYCSAGHCPEDSVNNICSTDGYCFTMIEEDDSGMPVVASGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 343 - protein, 144 aa, synthetic construct
KKEDGESTAPTPRPKILRCYCSAGHCPEDSVNNICSTDGYCFTMIEEDDSGMPVVASGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 344 - protein, 142 aa, synthetic construct
KKEDGESTAPTPRPKILRCYCSAGHCPEDSVNNICSTDGYCFTMIEEDDSGMPVVASGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 345 - protein, 352 aa, synthetic construct
KKEDGESTAPTPRPKILRCYCSAGHCPEDSVNNICSTDGYCFTMIEEDDSGMPVVASGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 346 - protein, 107 aa, synthetic construct
KKEDGESTAPTPRPKILRCYCSAGHCPEDSVNNICSTDGYCFTMIEEDDSGMPVVTSGCMGLEGSDFQCRDTPIPHQRRSIECCTERNECNKDLHPTLPPLKNRDFV

SEQ ID NO: 347 - protein, 352 aa, synthetic construct
KKEDGESTAPTPRPKILRCYCSAGHCPEDSVNNICSTDGYCFTMIEEDDSGMPVVTSGCMGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 348 - protein, 144 aa, synthetic construct
KKEDGESTAPTPRPKILRCYCSAGHCPEDSVNNICSTDGYCFTMIEEDDSGMPVVTSGCMGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 349 - protein, 144 aa, synthetic construct
KKEDGESTAPTPRPKILRCYCSAGHCPEDSVNNICSTDGYCFTMIEEDDSGMPVVTSGCMGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 350 - protein, 142 aa, synthetic construct
KKEDGESTAPTPRPKILRCYCSAGHCPEDSVNNICSTDGYCFTMIEEDDSGMPVVTSGCMGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 351 - protein, 352 aa, synthetic construct
KKEDGESTAPTPRPKILRCYCSAGHCPEDSVNNICSTDGYCFTMIEEDDSGMPVVTSGCMGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 352 - protein, 107 aa, synthetic construct
KKEDGESTAPTPRPKILRCYCSAGHCPEDSVNNICSTDGYCFTMIEEDDSGMPVVTSGCLKLEGSDFQCRDTPIPHQRRSIECCTERNECNKDLHPTLPPLKNRDFV

SEQ ID NO: 353 - protein, 352 aa, synthetic construct
KKEDGESTAPTPRPKILRCYCSAGHCPEDSVNNICSTDGYCFTMIEEDDSGMPVVTSGCLKLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 354 - protein, 144 aa, synthetic construct
KKEDGESTAPTPRPKILRCYCSAGHCPEDSVNNICSTDGYCFTMIEEDDSGMPVVTSGCLKLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 355 - protein, 144 aa, synthetic construct
KKEDGESTAPTPRPKILRCYCSAGHCPEDSVNNICSTDGYCFTMIEEDDSGMPVVTSGCLKLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 356 - protein, 142 aa, synthetic construct
KKEDGESTAPTPRPKILRCYCSAGHCPEDSVNNICSTDGYCFTMIEEDDSGMPVVTSGCLKLEGSDFQCRDTPIPHQRRSIECCTERNECNKDLHPTLPPLKNRDFVTGGGGSGGGGSGGGGSGGGGSPTIKPCPPCKCPAP

SEQ ID NO: 357 - protein, 352 aa, synthetic construct
KKEDGESTAPTPRPKILRCYCSAGHCPEDSVNNICSTDGYCFTMIEEDDSGMPVVTSGCLKLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 358 - protein, 107 aa, synthetic construct
KKEDGESTAPTPRPKILRCYCSAGHCPEDSVNNICSTDGYCFTMIEEDDSGMPVVTSGCLGYEGSDFQCRDTPIPHQRRSIECCTERNECNKDLHPTLPPLKNRDFV

SEQ ID NO: 359 - protein, 352 aa, synthetic construct
KKEDGESTAPTPRPKILRCYCSAGHCPEDSVNNICSTDGYCFTMIEEDDSGMPVVTSGCLGYEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 360 - protein, 144 aa, synthetic construct
KKEDGESTAPTPRPKILRCYCSAGHCPEDSVNNICSTDGYCFTMIEEDDSGMPVVTSGCLGYEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 361 - protein, 144 aa, synthetic construct
KKEDGESTAPTPRPKILRCYCSAGHCPEDSVNNICSTDGYCFTMIEEDDSGMPVVTSGCLGYEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 362 - protein, 142 aa, synthetic construct
KKEDGESTAPTPRPKILRCYCSAGHCPEDSVNNICSTDGYCFTMIEEDDSGMPVVTSGCLGYEGSDFQCRDTPIPHQRRSIECCTERNECNKDLHPTLPPLKNRDFVTGGGGSGGGGSGGGGSGGGGSPTIKPCPPCKCPAP

SEQ ID NO: 363 - protein, 352 aa, synthetic construct
KKEDGESTAPTPRPKILRCYCSAGHCPEDSVNNICSTDGYCFTMIEEDDSGMPVVTSGCLGYEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 364 - protein, 107 aa, synthetic construct
KKEDGESTAPTPRPKILRCYCSAGHCPEDSVNNICSTDGYCFTMIEEDDSGMPVVTSGCLGLEGSDFQCKDTPIPHQRRSIECCTERNECNKDLHPTLPPLKNRDFV

SEQ ID NO: 365 - protein, 352 aa, synthetic construct
KKEDGESTAPTPRPKILRCYCSAGHCPEDSVNNICSTDGYCFTMIEEDDSGMPVVTSGCLGLEGSDFQCKDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 366 - protein, 144 aa, synthetic construct
KKEDGESTAPTPRPKILRCYCSAGHCPEDSVNNICSTDGYCFTMIEEDDSGMPVVTSGCLGLEGSDFQCKDTPIPHQRRSIECCTERNECNKDLHPTLPPLKNRDFVTGGGGSGGGGSGGGGSGGGGSTHTCPPCPAPEAAGGP

SEQ ID NO: 367 - protein, 144 aa, synthetic construct
KKEDGESTAPTPRPKILRCYCSAGHCPEDSVNNICSTDGYCFTMIEEDDSGMPVVTSGCLGLEGSDFQCKDTPIPHQRRSIECCTERNECNKDLHPTLPPLKNRDFVTGGGGSGGGGSGGGGSGGGGSTHTCPPCKCPEAAGGP

SEQ ID NO: 368 - protein, 142 aa, synthetic construct
KKEDGESTAPTPRPKILRCYCSAGHCPEDSVNNICSTDGYCFTMIEEDDSGMPVVTSGCLGLEGSDFQCKDTPIPHQRRSIECCTERNECNKDLHPTLPPLKNRDFVTGGGGSGGGGSGGGGSGGGGSPTIKPCPPCKCPAP

SEQ ID NO: 369 - protein, 352 aa, synthetic construct
KKEDGESTAPTPRPKILRCYCSAGHCPEDSVNNICSTDGYCFTMIEEDDSGMPVVTSGCLGLEGSDFQCKDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 370 - protein, 107 aa, synthetic construct
KKEDGESTAPTPRPKILRCYCSAGHCPEDSVNNICSTDGYCFTMIEEDDSGMPVVTSGCLGLEGSDFQCRDSPIPHQRRSIECCTERNECNKDLHPTLPPLKNRDFV

SEQ ID NO: 371 - protein, 352 aa, synthetic construct
KKEDGESTAPTPRPKILRCYCSAGHCPEDSVNNICSTDGYCFTMIEEDDSGMPVVTSGCLGLEGSDFQCRDSPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 372 - protein, 144 aa, synthetic construct
KKEDGESTAPTPRPKILRCYCSAGHCPEDSVNNICSTDGYCFTMIEEDDSGMPVVTSGCLGLEGSDFQCRDSPIPHQRRSIECCTERNECNKDLHPTLPPLKNRDFVTGGGGSGGGGSGGGGSGGGGSTHTCPPCPAPEAAGGP

SEQ ID NO: 373 - protein, 144 aa, synthetic construct
KKEDGESTAPTPRPKILRCYCSAGHCPEDSVNNICSTDGYCFTMIEEDDSGMPVVTSGCLGLEGSDFQCRDSPIPHQRRSIECCTERNECNKDLHPTLPPLKNRDFVTGGGGSGGGGSGGGGSGGGGSTHTCPPCKCPEAAGGP

SEQ ID NO: 374 - protein, 142 aa, synthetic construct
KKEDGESTAPTPRPKILRCYCSAGHCPEDSVNNICSTDGYCFTMIEEDDSGMPVVTSGCLGLEGSDFQCRDSPIPHQRRSIECCTERNECNKDLHPTLPPLKNRDFVTGGGGSGGGGSGGGGSGGGGSPTIKPCPPCKCPAP

SEQ ID NO: 375 - protein, 352 aa, synthetic construct
KKEDGESTAPTPRPKILRCYCSAGHCPEDSVNNICSTDGYCFTMIEEDDSGMPVVTSGCLGLEGSDFQCRDSPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 376 - protein, 107 aa, synthetic construct
KKEDGESTAPTPRPKILRCYCSAGHCPEDSVNNICSTDGYCFTMIEEDDSGMPVVTSGCLGLEGSDFQCRDTPKPHQRRSIECCTERNECNKDLHPTLPPLKNRDFV

SEQ ID NO: 377 - protein, 352 aa, synthetic construct
KKEDGESTAPTPRPKILRCYCSAGHCPEDSVNNICSTDGYCFTMIEEDDSGMPVVTSGCLGLEGSDFQCRDTPKPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 378 - protein, 144 aa, synthetic construct
KKEDGESTAPTPRPKILRCYCSAGHCPEDSVNNICSTDGYCFTMIEEDDSGMPVVTSGCLGLEGSDFQCRDTPKPHQRRSIECCTERNECNKDLHPTLPPLKNRDFVTGGGGSGGGGSGGGGSGGGGSTHTCPPCPAPEAAGGP

SEQ ID NO: 379 - protein, 144 aa, synthetic construct
KKEDGESTAPTPRPKILRCYCSAGHCPEDSVNNICSTDGYCFTMIEEDDSGMPVVTSGCLGLEGSDFQCRDTPKPHQRRSIECCTERNECNKDLHPTLPPLKNRDFVTGGGGSGGGGSGGGGSGGGGSTHTCPPCKCPEAAGGP

SEQ ID NO: 380 - protein, 142 aa, synthetic construct
KKEDGESTAPTPRPKILRCYCSAGHCPEDSVNNICSTDGYCFTMIEEDDSGMPVVTSGCLGLEGSDFQCRDTPKPHQRRSIECCTERNECNKDLHPTLPPLKNRDFVTGGGGSGGGGSGGGGSGGGGSPTIKPCPPCKCPAP

SEQ ID NO: 381 - protein, 352 aa, synthetic construct
KKEDGESTAPTPRPKILRCYCSAGHCPEDSVNNICSTDGYCFTMIEEDDSGMPVVTSGCLGLEGSDFQCRDTPKPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 382 - protein, 107 aa, synthetic construct
KKEDGESTAPTPRPKILRCYCSAGHCPEDSVNNICSTDGYCFTMIEEDDSGMPVVTSGCLGLEGSDFQCRDTPIAHQRRSIECCTERNECNKDLHPTLPPLKNRDFV

SEQ ID NO: 383 - protein, 352 aa, synthetic construct
KKEDGESTAPTPRPKILRCYCSAGHCPEDSVNNICSTDGYCFTMIEEDDSGMPVVTSGCLGLEGSDFQCRDTPIAHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 384 - protein, 144 aa, synthetic construct
KKEDGESTAPTPRPKILRCYCSAGHCPEDSVNNICSTDGYCFTMIEEDDSGMPVVTSGCLGLEGSDFQCRDTPIAHQRRSIECCTERNECNKDLHPTLPPLKNRDFVTGGGGSGGGGSGGGGSGGGGSTHTCPPCPAPEAAGGP

SEQ ID NO: 385 - protein, 144 aa, synthetic construct
KKEDGESTAPTPRPKILRCYCSAGHCPEDSVNNICSTDGYCFTMIEEDDSGMPVVTSGCLGLEGSDFQCRDTPIAHQRRSIECCTERNECNKDLHPTLPPLKNRDFVTGGGGSGGGGSGGGGSGGGGSTHTCPPCKCPEAAGGP

SEQ ID NO: 386 - protein, 142 aa, synthetic construct
KKEDGESTAPTPRPKILRCYCSAGHCPEDSVNNICSTDGYCFTMIEEDDSGMPVVTSGCLGLEGSDFQCRDTPIAHQRRSIECCTERNECNKDLHPTLPPLKNRDFVTGGGGSGGGGSGGGGSGGGGSPTIKPCPPCKCPAP

SEQ ID NO: 387 - protein, 352 aa, synthetic construct
KKEDGESTAPTPRPKILRCYCSAGHCPEDSVNNICSTDGYCFTMIEEDDSGMPVVTSGCLGLEGSDFQCRDTPIAHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 388 - protein, 107 aa, synthetic construct
KKEDGESTAPTPRPKILRCYCSAGHCPEDSVNNICSTDGYCFTMIEEDDSGMPVVTSGCLGLEGSDFQCRDTPIPQQRRSIECCTERNECNKDLHPTLPPLKNRDFV

SEQ ID NO: 389 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPQQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 390 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPQQRRSIECCTERNECNKDLHPTLPPLKNRDFVTGGGGSGGGGSGGGGSGGGGSTHTCPPCPAPEAAGGP

SEQ ID NO: 391 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPQQRRSIECCTERNECNKDLHPTLPPLKNRDFVTGGGGSGGGGSGGGGSGGGGSTHTCPPCKCPEAAGGP

SEQ ID NO: 392 - protein, 142 aa, synthetic construct
KKEDGESTAPTPRPKILRCYCSAGHCPEDSVNNICSTDGYCFTMIEEDDSGMPVVTSGCLGLEGSDFQCRDTPIPQQRRSIECCTERNECNKDLHPTLPPLKNRDFVTGGGGSGGGGSGGGGSGGGGSPTIKPCPPCKCPAP

SEQ ID NO: 393 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPQQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 394 - protein, 107 aa, synthetic construct
KKEDGESTAPTPRPKILRCYCSAGHCPEDSVNNICSTDGYCFTMIEEDDSGMPVVTSGCLGLEGSDFQCRDTPIPHLRRSIECCTERNECNKDLHPTLPPLKNRDFV

SEQ ID NO: 395 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPHLRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 396 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPHLRRSIECCTERNECNKDLHPTLPPLKNRDFVTGGGGSGGGGSGGGGSGGGGSTHTCPPCPAPEAAGGP

SEQ ID NO: 397 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPHLRRSIECCTERNECNKDLHPTLPPLKNRDFVTGGGGSGGGGSGGGGSGGGGSTHTCPPCKCPEAAGGP

SEQ ID NO: 398 - protein, 142 aa, synthetic construct
KKEDGESTAPTPRPKILRCYCSAGHCPEDSVNNICSTDGYCFTMIEEDDSGMPVVTSGCLGLEGSDFQCRDTPIPHLRRSIECCTERNECNKDLHPTLPPLKNRDFVTGGGGSGGGGSGGGGSGGGGSPTIKPCPPCKCPAP

SEQ ID NO: 399 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPHLRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 400 - protein, 107 aa, synthetic construct
KKEDGESTAPTPRPKILRCYCSAGHCPEDSVNNICSTDGYCFTMIEEDDSGMPVVTSGCLGLEGSDFQCRDTPIPHQRRTIECCTERNECNKDLHPTLPPLKNRDFV

SEQ ID NO: 401 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPHQRRTIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 402 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPHQRRTIECCTERNECNKDLHPTLPPLKNRDFVTGGGGSGGGGSGGGGSGGGGSTHTCPPCPAPEAAGGP

SEQ ID NO: 403 - protein, 144 aa, synthetic construct
KKEDGESTAPTPRPKILRCYCSAGHCPEDSVNNICSTDGYCFTMIEEDDSGMPVVTSGCLGLEGSDFQCRDTPIPHQRRTIECCTERNECNKDLHPTLPPLKNRDFVTGGGGSGGGGSGGGGSGGGGSTHTCPPCKCPEAAGGP

SEQ ID NO: 404 - protein, 142 aa, synthetic construct
KKEDGESTAPTPRPKILRCYCSAGHCPEDSVNNICSTDGYCFTMIEEDDSGMPVVTSGCLGLEGSDFQCRDTPIPHQRRTIECCTERNECNKDLHPTLPPLKNRDFVTGGGGSGGGGSGGGGSGGGGSPTIKPCPPCKCPAP

SEQ ID NO: 405 - protein, 352 aa, synthetic construct
KKEDGESTAPTPRPKILRCYCSAGHCPEDSVNNICSTDGYCFTMIEEDDSGMPVVTSGCLGLEGSDFQCRDTPIPHQRRTIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 406 - protein, 111 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTTNLCNQYLQPTLPPVVIGPFFDGSIR

SEQ ID NO: 407 - protein, 356 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 408 - protein, 148 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGP

SEQ ID NO: 409 - protein, 148 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCKCPEAAGGP

SEQ ID NO: 410 - protein, 146 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSPTIKPCPPCKCPAP

SEQ ID NO: 411 - protein, 356 aa, synthetic construct
KKEDGESTAPTPRPKILRCYCSAGHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 412 - protein, 107 aa, synthetic construct
KKEDGESTAPTPRPKILRCYCSAGHCPEDSVNNICSTDGYCFTMIEEDDSGLPVVTSGCLGLEGSDFQCRDTPIPHQRRSIECCTERNECNKDLHPTLPPLKNRDFV

SEQ ID NO: 413 - protein, 352 aa, synthetic construct
KKEDGESTAPTPRPKILRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 414 - protein, 144 aa, synthetic construct
KKEDGESTAPTPRPKILRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 415 - protein, 144 aa, synthetic construct
KKEDGESTAPTPRPKILRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 416 - protein, 142 aa, synthetic construct
KKEDGESTAPTPRPKILRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 417 - protein, 352 aa, synthetic construct
KKEDGESTAPTPRPKILRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 418 - protein, 107 aa, synthetic construct
KKEDGESTAPTPRPKVLRCYCSAGHCPEDSVNNICSTDGYCFTMIEEDDSGMPVVTSGCLGLEGSDFQCRDTPIPHQRRSIECCTERNECNKDLHPTLPPLKNRDFV

SEQ ID NO: 419 - protein, 352 aa, synthetic construct
KKEDGESTAPTPRPKVLRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 420 - protein, 144 aa, synthetic construct
KKEDGESTAPTPRPKVLRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGP

SEQ ID NO: 421 - protein, 144 aa, synthetic construct
KKEDGESTAPTPRPKVLRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCKCPEAAGGP

SEQ ID NO: 422 - protein, 142 aa, synthetic construct
KKEDGESTAPTPRPKVLRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSPTIKPCPPCKCPAP

SEQ ID NO: 423 - protein, 352 aa, synthetic construct
KKEDGESTAPTPRPKVLRCYCSAGHCPEDSVNNICSTDGYCFTMIEEDDSGMPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 424 - protein, 352 aa, synthetic construct
KKEDGESTAPTPRPKVLRCYCSAGHCPEDSVNNICSTDGYCFTMIEEDDSGLPVVTSGCLGLEGSDFQCRDTPIPHQRRSIECCTERNECNKDLHPTLPPLKNRDFVTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 425 - protein, 110 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTTNLCNQYLQPTLPPVVIGPFFDGSIR

SEQ ID NO: 426 - protein, 355 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 427 - protein, 147 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGP

SEQ ID NO: 428 - protein, 147 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTTNLCNQYLQPTLPPVVIGPFFDGSIRTGGGGSGGGGSGGGGSGGGGSTHTCPPCKCPEAAGGP

SEQ ID NO: 429 - protein, 145 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTTNLCNQYLQPTLPPVVIGPFFDGSIRTGGGGSGGGGSGGGGSGGGGSPTIKPCPPCKCPAP

SEQ ID NO: 430 - protein, 355 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 431 - protein, 110 aa, synthetic construct
KKEDGESTAPTPRPKILRCKCHHHCPEDSVNNICSTDGYCFTMIEEDDSGMPVVTSGCLGLEGSDFQCRDTPIPHQRRSIECCTTNLCNQYLQPTLPPVVIGPFFDGSIR

SEQ ID NO: 432 - protein, 355 aa, synthetic construct
KKEDGESTAPTPRPKILRCKCHHHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 433 - protein, 147 aa, synthetic construct
KKEDGESTAPTPRPKILRCKCHHHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGP

SEQ ID NO: 434 - protein, 147 aa, synthetic construct
KKEDGESTAPTPRPKILRCKCHHHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCKCPEAAGGP

SEQ ID NO: 435 - protein, 145 aa, synthetic construct
KKEDGESTAPTPRPKILRCKCHHHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSPTIKPCPPCKCPAP

SEQ ID NO: 436 - protein, 355 aa, synthetic construct
KKEDGESTAPTPRPKILRCKCHHHCPEDSVNNICSTDGYCFTMIEEDDSGMPVVTSGCLGLEGSDFQCRDTPIPHQRRSIECCTTNLCNQYLQPTLPPVVIGPFFDGSIRTGGGGSGGGGSGGGGSGGGGSTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 437 - protein, 113 aa, synthetic construct
  VARIANT 15: note = K or P
  VARIANT 16: note = V or I
  VARIANT 20: note = V, F, K or Y
  VARIANT 22: note = G, H or S
  VARIANT 23: note = S, H or G
  VARIANT 27: note = E or D
  VARIANT 29: note = S or A
  VARIANT 30: note = V or I
  VARIANT 33: note = I or T
  VARIANT 35: note = S or I
  VARIANT 37: note = D or N
  VARIANT 39: note = Y or H
  VARIANT 42: note = T or A
  VARIANT 43: note = M or I
  VARIANT 49: note = S or Q
  VARIANT 51: note = L or E
  VARIANT 52: note = P or T
  VARIANT 53: note = V or T
  VARIANT 54: note = V or L
  VARIANT 55: note = T or A
  VARIANT 59: note = L or M
  VARIANT 60: note = G or K
  VARIANT 61: note = L or Y
  VARIANT 69: note = R or K
  VARIANT 71: note = T or S
  VARIANT 73: note = I or K
  VARIANT 74: note = P or A
  VARIANT 75: note = H or Q
  VARIANT 76: note = Q or L
  VARIANT 79: note = S or T
  VARIANT 84: note = T or R
  VARIANT 85: note = absent, T or E
  VARIANT 86: note = R or N
  VARIANT 87: note = absent, N or L
  VARIANT 88: note = absent, E or L
  VARIANT 89: note = absent or C
  VARIANT 90: note = absent or N
  VARIANT 91: note = absent, K or Q
  VARIANT 92: note = absent, D or L
  VARIANT 93: note = absent or L
  VARIANT 94: note = absent, H or Q
  VARIANT 95: note = absent or P
  VARIANT 96: note = absent or T
  VARIANT 97: note = absent or L
  VARIANT 98: note = absent or P
  VARIANT 99: note = absent or P
  VARIANT 100: note = absent, V or L
  VARIANT 101: note = absent, V or K
  VARIANT 102: note = absent, I or N
  VARIANT 103: note = absent, G or R
  VARIANT 104: note = absent, P, D or A
  VARIANT 105: note = absent or F
  VARIANT 106: note = absent, F or V
  VARIANT 107: note = absent or D
  VARIANT 108: note = absent or G
  VARIANT 109: note = absent, S or P
  VARIANT 110: note = absent or I
  VARIANT 111: note = absent, R or H
  VARIANT 112: note = absent or H
  VARIANT 113: note = absent or R
KKEDGESTAPTPRPXXLRCXCXXHCPXDXXNNXCXTXGXCFXXIEEDDXGXXXXXSGCXXXEGSDFQCXDXPXXXXRRXIECCXXXXXXXXXXXXXXXXXXXXXXXXXXXXXX

SEQ ID NO: 438 - protein, 26 aa, Homo sapiens
TNLCNQYLQPTLPPVVIGPFFDGSIR

SEQ ID NO: 439 - protein, 83 aa, synthetic construct
KKEDGESTAPTPRPKVLRCKCHHHCPEDSVNNICSTDGYCFTMIEEDDSGLPVVTSGCLGLEGSDFQCRDTPIPHQRRSIECC

SEQ ID NO: 440 - protein, 110 aa, synthetic construct
KKEDGESTAPTPRPKVLRCYCSAGHCPEDSVNNICSTDGYCFTMIEEDDSGMPVVTSGCLGLEGSDFQCRDTPIPHQRRSIECCTNLCNQYLQPTLPPVVIGPFFDGSIR